(19) United States
(12) Patent Application Publication
(10) Pub. No.: US 2023/0183716 A1
(43) Pub. Date: Jun. 15, 2023

(54) MOLECULES AND METHODS FOR INCREASED TRANSLATION

(71) Applicant: RAMOT AT TEL-AVIV UNIVERSITY LTD., Tel Aviv (IL)

(72) Inventors: Tamir TULLER, Tel Aviv (IL); Michael PEERI, Tel Aviv (IL)

(21) Appl. No.: 17/870,029

(22) Filed: Jul. 21, 2022

Related U.S. Application Data
(63) Continuation of application No. PCT/IL2021/050074, filed on Jan. 24, 2021.
(60) Provisional application No. 62/964,859, filed on Jan. 23, 2020.

(51) Int. Cl. C12N 15/68; C12N 15/69; C12N 15/10

(52) U.S. Cl. CPC C12N 15/68; C12N 15/69; C12N 15/102

(57) ABSTRACT

Nucleic acid molecule comprising a coding sequence and a region of increased folding energy upstream of a stop codon are provided. Expression vectors and cells comprising the nucleic acid molecule are also provided. Methods for optimizing a coding sequence comprising increasing folding energy in a region upstream of the stop codon are also provided.

### CROSS-REFERENCE TO RELATED APPLICATIONS

This application is a Bypass continuation of PCT Patent Application No. PCT/IL2021/050074, having International filing date of Jan. 24, 2021, which claims the benefit of priority of U.S. Provisional Patent Application No. 62/964,859 filed Jan. 23, 2020, both entitled “MOLECULES AND METHODS FOR INCREASED TRANSLATION”, the contents of which are all incorporated herein by reference in their entirety.

### FIELD OF INVENTION

The present invention is in the field of nucleic acid editing and translation optimization.

### BACKGROUND OF THE INVENTION

There is growing evidence that local mRNA folding (i.e., short-range secondary-structure) inside the coding region is often stronger or weaker than expected, but the explanation for this phenomenon is yet to be fully understood. mRNA folding strength affects many central cellular processes, including the transcription rate and termination, translation initiation, translation elongation and ribosomal traffic jams, co-translational folding, mRNA aggregation, mRNA stability and mRNA splicing. Many of these effects are mediated by interactions of mRNA within the CDS (protein-coding sequence) with proteins and other RNAs and may include structure-specific or non-structure-specific interactions.

In recent years several studies showed evidence for selection acting directly to affect mRNA folding strength within the CDS (FIG. 1A). Studies looking at the CDS as a whole found selection for strong mRNA folding in most species. Studies focusing on the beginning of the coding region (i.e. the first 40-50 nucleotides) found evidence for the inverse, with selection acting to weaken mRNA folding in that region. In addition, there is some evidence for specifically strong folding in nucleotides 30-70, which may slow down translation elongation near the 5′ end of the mRNA, possibly to prevent ribosomal traffic jams. These results are generally in agreement with available small-scale and large-scale experimental validation performed in model organisms. Some of these characteristic regions were found to be correlated with genomic GC-content and to be stronger in highly expressed genes. However, the previous studies cited did not systematically examine how the selection on folding strength changes along the coding sequence and how this phenomenon varies across the tree of life. Methods of optimizing translation by modifying folding strength and folding free energy are greatly needed.

### SUMMARY OF THE INVENTION

The present invention provides nucleic acid molecules comprising a coding sequence and a region of increased folding energy upstream of a stop codon. Expression vectors and cells comprising the nucleic acid molecule are also provided. Methods for optimizing a coding sequence comprising increasing folding energy in a region upstream of that stop codon are also provided.

According to a first aspect, there is provided a method for optimizing a coding sequence, the method comprising introducing a mutation into a first region from 90 nucleotides upstream of a stop codon of the coding sequence to the stop codon; wherein the mutation increases folding energy of the first region or of RNA encoded by the first region, thereby optimizing a coding sequence.

According to another aspect, there is provided a nucleic acid molecule comprising a coding sequence, the coding sequence comprises at least one codon substituted to a synonymous codon within a first region from 90 nucleotides upstream of a stop codon of the coding sequence to the stop codon, wherein the substitution increases folding energy of the first region or of RNA encoded by the first region.

According to another aspect, there is provided an expression vector comprising a nucleic acid molecule of the invention.

According to another aspect, there is provided a cell comprising a nucleic acid molecule of the invention or an expression vector of the invention.

According to another aspect, there is provided a computer program product comprising a non-transitory computer-readable storage medium having program instructions embodied therewith, the program instructions executable by at least one hardware processor to execute a genetic-type machine learning algorithm configured to:
a. receive a coding sequence;
b. determine within a first region from 90 nucleotides upstream of a stop codon of the coding sequence to the stop codon at least one mutation that increases folding energy of the first region or RNA encoded by the first region; and
c. output
   i. a mutated coding sequence comprising the at least one mutation; or
   ii. a list of possible mutations comprising the at least one mutation.

According to some embodiments, the optimizing comprises optimizing expression of protein encoded by the coding sequence.

According to some embodiments, the optimizing is optimizing in a target cell.

According to some embodiments, the target cells is selected from:
a. an archaea cell and the first region is from 90 nucleotides upstream of a stop codon of the coding sequence to the stop codon;
b. a bacteria cell and the first region is from 50 nucleotides upstream of a stop codon of the coding sequence to the stop codon; and
c. a eukaryote cell and the first region is from 40 nucleotides upstream of a stop codon of the coding sequence to the stop codon.

According to some embodiments, the mutation is a synonymous mutation.

According to some embodiments, the introducing comprises providing a mutated sequence or providing a mutation to be made in the coding sequence.

According to some embodiments, the mutation increases folding energy of the first region to above a predetermined threshold.

According to some embodiments, the predetermined threshold is a value above which the difference as compared to folding energy of the region without the substitution would be significant.

According to some embodiments, the threshold is species-specific and is selected from a threshold provided in Tables 5 or the threshold is domain-specific and is selected from a threshold provided in Table 1.

According to some embodiments, the method comprises introducing a plurality of mutations wherein each mutation increases folding energy of the first region or of RNA encoded by the first region or wherein the plurality of mutations in combination increases folding energy of the first region or of RNA encoded by the first region.

According to some embodiments, the method comprises mutating all possible codons within the region to a synonymous codon that increases folding energy of the first region or of RNA encoded by the first region.

According to some embodiments, the method comprises introducing synonymous mutations to produce a first region or RNA encoded by the first region with the maximum possible folding energy.

According to some embodiments, the method further comprises introducing a mutation into a second region from a translational start site (TSS) to 20 nucleotides downstream of the TSS, wherein the mutation increases folding energy of the second region or of RNA encoded by the second region.

According to some embodiments, the method is a method for optimizing expression in a target cell, and wherein the target cells is selected from:
a. an archaea cell and the second region is from the TSS to 10 nucleotides downstream of the TSS; and
b. a bacteria cell or a eukaryote cell and the second region is from the TSS to 20 nucleotides downstream of the TSS.

According to some embodiments, the method is a method for optimizing expression in a target cell, and wherein the target cell is a bacterial or archeal cell and the method further comprises introducing a mutation into a third region between the first and the second regions, wherein the mutation decreases folding energy of the third region or of RNA encoded by the third region.

According to some embodiments, the method is a method for optimizing expression in a target cell, and wherein the target cell is a eukaryotic cell and the method further comprises introducing a mutation into a third region between the first and the second regions, wherein the mutation increases folding energy of the third region or of RNA encoded by the third region.

According to some embodiments, the third region is from 20 to 50 nucleotides downstream of the TSS.

According to some embodiments, the third region is from 20 to 300 nucleotides downstream of the TSS or from 300 to 90 upstream of the stop codon.

According to some embodiments, the nucleic acid molecule is an RNA molecule, or a DNA molecule.

According to some embodiments, the first region is from 50 nucleotides upstream of the stop codon to the stop codon.

According to some embodiments, the first region is from 40 nucleotides upstream of the stop codon to the stop codon.

According to some embodiments, the substitution increases folding energy of the first region to above a predetermined threshold.

According to some embodiments, the predetermined threshold is a value above which the difference as compared to folding energy of the region without the substitution would be significant.

According to some embodiments, the threshold is species-specific and is selected from a threshold provided in Tables 5 or the threshold is domain-specific and is selected from a threshold provided in Table 1.

According to some embodiments, the nucleic acid molecule comprises a plurality of synonymous substitutions, wherein each substitution increases folding energy of the first region or of RNA encoded by the first region or wherein the plurality of synonymous substitutions in combination increases folding energy of the first region or of RNA encoded by the first region.

According to some embodiments, all possible codons within the first region are substituted to a synonymous codon that increases folding energy of the first region or of RNA encoded by the first region.

According to some embodiments, the region comprises synonymous codons substituted to increase folding energy to a maximum possible.

According to some embodiments, a second region of the coding sequence from a translational start site (TSS) to 20 nucleotides downstream of the TSS comprises at least one codon substituted to a synonymous codon, and wherein the substitution increases folding energy of the second region or of RNA encoded by the second region.

According to some embodiments, the coding sequence encodes a bacterial or archeal gene and further comprises a third region of the coding sequence between the first region and the second region comprises at least one codon substituted to a synonymous codon, and wherein the substitution decreases folding energy of the third region or of RNA encoded by the third region.

According to some embodiments, the coding sequence encodes a eukaryotic gene and further comprises a third region of the coding sequence between the first region and the second region comprises at least one codon substituted to a synonymous codon, and wherein the substitution increases folding energy of the third region or of RNA encoded by the third region.

According to some embodiments, the third region is from 20 to 50 nucleotides downstream of the TSS.

According to some embodiments, the third region is from 20 to 300 nucleotides downstream of the TSS or from 300 to 90 upstream of the stop codon.

According to some embodiments, the folding energy is the RNA secondary structure folding Gibbs free energy.

According to some embodiments, the cell is a target cell.

According to some embodiments, the nucleic acid molecule, expression vector or both are optimized for expression in the cell.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-E: Common regions of ΔLFE bias are represented across the tree of life but are not universal. There is correlation between the strengths of these regions in different species, indicating there are factors influencing the bias throughout the coding sequence. (1A) Summary of profile features with the fraction of species in which each feature appears in each domain (based on Model 1 rules, see Materials and Methods for details). The results based on the less restrictive Model 2 rules (with weaker ΔLFE near the CDS edges not required to be positive, see Materials and Methods) are shown in bright blue below each bar. References shown here are based on comparison to randomized sequences (i.e., equivalent to ΔLFE). (1B) Scheme illustrating profile features reported separately in previous studies within the CDS, showing features [A]-[D] from 1A. (1C) Observed distribution of ΔLFE profile values at different positions relative to CDS start (left) and end (right). (1D) The distances (in nt) from the start codon where ΔLFE transitions from positive to negative, for species belonging to different domains. The lengths of the initial weak folding region range up to 150 nt in some bacteria. (1E) Spearman correlations between mean ΔLFE profile values in regions [A], [C], [D]. White dots indicate significant correlation (p-value<0.01).

FIGS. 2A-C: Overview of the computational analysis to measure ΔLFE while controlling for other factors known to be under selection at different regions of the coding sequence and find factors correlated with it. (2A) An illustration of the variables and concepts involved in changing local folding strength and calculating ΔLFE. The effects of the compositional factors on the left side are removed in order to specifically measure the contribution of codon arrangements to the native folding energy. Blue arrows indicate possible selection forces. (2B) Illustration of the different steps in the computational pipeline used to estimate ΔLFE and the factors affecting it (see Materials and Methods). For each genome, the CDSs are randomized based on each null-model (CDS-wide and position specific), to calculate a mean ΔLFE profile based on that null-model. At the next step, based on GLS, correlations between features of the ΔLFE profile and genomic/environmental features are computed. Input data sources (native CDS sequences, species trait values, species tree) are shown in green. (2C) The distributions of some genomic properties within the dataset—CDS count, genomic GC-content, genomic ENc′ (measure of CUB). The dataset was designed to represent a wide range of values (among other considerations, see Materials and Methods, “Species selection and sequence filtering”).

FIGS. 3A-B: Two summaries of the ΔLFE profiles demonstrate the consistency and diversity found. (3A) Characteristic ΔLFE profiles for species belonging to different taxa. The format of the plots appears in the upper left corner: ΔLFE bias is shown (by color) for windows starting in the range 0-150 nt relative to the CDS start, on the left, and CDS end, on the right; red denotes negative ΔLFE (stronger-than-expected folding) while blue denotes positive ΔLFE (weaker-than-expected folding; see the scale at the lower right corner of the figure). The characteristic profiles for each taxon were calculated using clustering analysis, which groups similar species according to the correlation between their profiles (see section 0 and Methods for details). The bars (in turquoise) appearing to the right of each characteristic profile indicate the relative number of species it represents. The full ΔLFE profiles for all species appear in FIG. 17. (3B) Summary of ΔLFE profile diversity for all species using dimensionality reduction to 2 dimensions with PCA (see explanations about PCA in the main text), with similar values (profiles) mapped to nearby positions. Background shading (blue) indicates density (see Materials and Methods for details). This shows most species have similar profiles (located near the center), but different kinds of less typical profiles are also represented. Top: CDS start, Bottom, CDS end. Short species names are listed in Table 4.

FIGS. 4A-C: The conserved ΔLFE profile elements are positively correlated with genomic CUB (measured as ENc′) throughout the CDS. (4A) Correlation strength (R², measured using GLS regression) between genomic ENc′ and ΔLFE at different positions relative to the CDS start (Left) and end (Right). R² values below the X-axis indicate negative regression slope (i.e. negative correlation with ΔLFE). The regression slope generally mirrors the sign of ΔLFE, indicating strong ΔLFE is correlated with strong codon bias throughout the CDS. Major taxonomic groups are plotted as different colored lines. White dots indicate regression p-value<0.01. (4B) Comparison of ΔLFE profile values in species with strong vs. weak CUB. Species with strong CUB (yellow, ENc′≤56.5) tend to have more extreme ΔLFE and show the conserved ΔLFE regions more clearly, while species with weak CUB (blue, ENc′>56.6) tend to also have weak ΔLFE. (4C) Genomic ENc′ plotted using PCA coordinates for profile positions 0-300 nt relative to CDS start (Left) and end (Right). The ΔLFE profiles (shown in insets, N=513) are plotted using the same PCA coordinates of FIG. 3B. Species with strong CUB (low ENc′, left plot, lower left quadrant and right plot, right side) have stronger ΔLFE profiles that more strongly adhere to the conserved ΔLFE regions.

FIGS. 5A-D: The conserved ΔLFE profile elements are correlated with genomic GC-content throughout the CDS. (5A) The effect of genomic-GC on ΔLFE at each position along the CDS start (Left) and end (Right), measured using GLS regression R² values. R² values above the X-axis indicate positive regression slope (indicating moderating effect of GC-content); R² values below the X-axis indicate negative regression slope. (i.e. reinforcing effect of GC-content). Near the CDS edges (where ΔLFE is usually positive), genomic-GC generally has a moderating effect on ΔLFE. In the mid-CDS region (where ΔLFE is usually negative), genomic-GC generally has a reinforcing effect on ΔLFE. Major taxonomic groups are plotted as different colored lines. White dots indicate regression p-value<0.01. (5B) Comparison of ΔLFE profile values in species with high vs. low genomic GC-content. Species with high GC-content (blue, genomic-GC>45%) tend to have more extreme ΔLFE and show the conserved ΔLFE regions more clearly, while species with low GC-content (yellow, genomic-GC≤45%) tend to also have weak ΔLFE. (5C) Genomic GC-content for all species plotted on the PCA coordinates of their ΔLFE profiles (same coordinates as in FIG. 3B and also shown in insets. N=513) for CDS start (Left) and end (Right). Low-GC species are generally clustered in a small region, indicating they have similar ΔLFE profiles, and that region is characterized by weak ΔLFE. (5D) Qualitative summary of ΔLFE in relation to GC-content in the mid-CDS.

FIGS. 6A-B: Genomic-GC effect on ΔLFE in eukaryotes shows divergence in high GC-content species that is not observed in other domains, while low GC-content species have weak ΔLFE. (6A) mean ΔLFE values for eukaryotes in the range 100-300 nt from CDS start, plotted against genomic-GC. Fungi are highlighted in blue. There is no linear relation between the variables (R²=0.01), but there is strong statistical dependence nevertheless (MIC=0.582, p-value<2e-5, N=78); see some explanation on MIC in the main text. (6B) PCA plot for the same species (see Material and Methods for details). On the left, ΔLFE profiles are plotted in the positions given by their first 2 PCA components. On the right, genomic-GC values for the profiles plotted at the same coordinates. Low-GC species are clustered in the middle region, while high-GC species are split between two distinct ΔLFE profile types. Short species names are listed in Table 4.

FIGS. 7A-D: Endosymbionts and intracellular parasites have generally weak ΔLFE. (7A) Comparison of ΔLFE values at different CDS positions between endosymbionts (Green) vs. other species (Pink). As can be seen, the ΔLFE values are less extreme in endosymbionts suggesting lower selection levels on local folding strength. (7B) Comparison of ΔLFE distributions at different CDS positions between endosymbionts (Green) vs. other species (Pink) within gammaproteobacterial (N=44). (7C) ΔLFE for species included in the tree within gammaproteobacteria; the endosymbionts and intracellular parasites (marked) have weaker ΔLFE bias compared to their relatives. (7D) PCA plot for ΔLFE profiles (Left, see 0) and the intracellular classification (Right) for the species in gammaproteobacteria (N=44). For clarity, overlapping profiles are hidden on the left (as in all PCA plots for ΔLFE profiles); all species are plotted on the right. Short species names in the PCA plot on the left panel are listed in Table 4.

FIGS. 8A-E: Hyperthermophiles have weak ΔLFE. (8A) ΔLFE profiles (for CDS beginning and end) for members of euryarchaeota covered by the phylogenetic tree (N=28), with the ultrametric species tree and their annotated genomic GC-contents and optimum growth temperatures classification (mesophile—Green, moderate thermophile—Orange, hyperthermophile—Red). Hyperthermophiles have weak ΔLFE that cannot be explained by the tree or their genomic GC-contents. (8B) ΔLFE profiles (left) and optimum growth temperatures (right) for all members of euryarchaeota having annotated optimum growth temperatures (N=25), plotted using their PCA coordinates (see Materials and Methods). Hyperthermophiles seems to be clustered in a small region characterized by weak ΔLFE. (8C) ΔLFE profiles (left) and optimum growth temperature (right) for all species having annotated optimum growth temperature (N=173), plotted using their PCA coordinates (see Materials and Methods). Short species names from PCA plots are listed in Table 4. (8D) Comparison of ΔLFE values for species having optimum temperature above (Blue) or below 75° C. (Yellow), for positions relative to CDS start (Left) or end (Right). (8E) Regression for optimum growth temperature vs. mean ΔLFE (average for positions 100-300 nt after CDS start) using GLS (Green regression line, N=96, R²=0.004, p-value=0.6) and OLS (Red regression line, N=173, R²=0.45). The apparent linear relation is no longer significant when controlling for the phylogenetic relationships. Points plotted in red are included only in OLS.

FIG. 9: Summary of trait correlations with ΔLFE in the mid-CDS region for different taxonomic groups. Many of these correlations are discussed in sections 3.3-3.6. For each group and trait combination, correlations are measured using R² with GLS (phylogenetically-corrected, green bars) and OLS (uncorrected linear relationship, red bars). Significant correlations are marked with * (p-value<0.05) or ** (p-value<0.001). Correlations with genomic-GC % and genomic-ENc′ are robust in prokaryotes, whereas other traits don't have consistent linear relationships. All correlations are for the region 100-300 nt after CDS start. Notes: (a) No linear dependence, but a significant relationship does exist (see FIG. 6). (b) Linear dependence appears in GLS but not in OLS. Small sample size exists in some taxa. (c) No significant linear relationship found over the entire range of values, but hyperthermophiles have significantly lower ΔLFE (see Example 7).

FIGS. 10A-C: Classification model for weak ΔLFE based on four species traits. (10A) PCA plot of ΔLFE profiles relative to CDS start (see Materials and Methods). Short species names are listed in Table 4. (10B) ΔLFE profile strength, measured using standard deviation, for profile positions 0-300 nt relative to CDS start. (10C) Predicted ΔLFE strength for each species using binary model for weak ΔLFE (precision=0.66, recall=0.82, N=513, see Materials and Methods under “Binary model for ΔLFE strength”).

FIG. 11: Coefficient of determination (R²) for GLS regression of the specified trait with ΔLFE and its components (ΔLFE—red; native LFE—green; randomized LFE—blue), at different positions relative to CDS start. Negative R² values indicate negative regression slope. The observed correlation between each trait and ΔLFE is not observed with the individual components (native or randomized LFE).

FIG. 12: Correlation (expressed using Moran's I coefficient) between the values of different traits, for pairs of species of different phylogenetic distances. Genomic-GC % is positively correlated at short distances. ΔLFE values (at different positions relative to CDS start) are more strongly correlated than genomic-GC % at most phylogenetic distances, but less correlated than genome sizes. Confidence intervals represent 95% confidence calculated using 500 bootstrap samples. The ‘Random’ trait is a normally distributed uncorrelated variable.

FIG. 13: Spearman correlations between the ΔLFE profile (i.e., mean value for a given species at each position relative to CDS start) and the corresponding CUB profiles (i.e., CUB for all CDSs for a given species at this position relative to CDS start) show no direct correspondence, indicating the ΔLFE profiles are not simply a side-effect of direct selection operating on CUB in different CDS regions. CUB measures were calculated for the sequences contained in the same 40 nt windows, starting at positions 0-300 nt relative to CDS start, with all the sequences for each species concatenated, for a random sample of N=256 species. From top to bottom, Nc (Effective Number of Codons), CAI (Codon Adaptation Index), Fop (Frequency of Optimal Codons), GC % (GC-content).

FIGS. 14A-B: Position-specific randomization (maintaining the encoded AA sequences as well as the codon frequency in each position (across all CDSs belonging to the same species) yields qualitatively similar results to the CDS-wide randomization used throughout the rest of this paper. This supports the conclusion that the observed ΔLFE profiles are not merely a result of position-dependent biases in codon composition. (14A) Correlation between ΔLFE calculated using “CDS-wide” and “position-specific” randomizations (see methods), at each position relative to CDS start. Correlations were calculated for a random sample (N=23) of species. (14B) Comparison of individual mean ΔLFE profiles calculated using “CDS-wide” (LFE-0) and “position-specific” (LFE-1) randomizations.

FIGS. 15A-B: The observed average ΔLFE features are generally more prominent in highly expressed genes and in genes encoding for highly abundant proteins. (15A) This figure shows results for 32 species, plotted according to their position on a taxonomic tree (Left). Results are summarized for highly expressed genes based on transcriptomic RNA-sequencing for 29 species (green region) and for experimentally measured protein-abundance (PA) for 12 species (blue region). Also shown are results for purely computational translation elongation optimization scores, I_TE(34) (cyan region). For each evidence type, results are shown for regions [A]-[C] (as defined in FIG. 1A). (15B) sources for RNA-seq data.

For each region, the following symbols identify the relation between the “high” and “low” groups: (+) The trend observed in this region (i.e., increased or decreased folding strength) is more extreme in highly expressed or highly abundant genes. (−) The trend observed in this region (i.e., increased or decreased folding strength) is less extreme in highly expressed or highly abundant genes (or the opposite trend is observed). (no symbol) There is no consistent and statistically significant difference between the groups (or there is no ΔLFE trend in this region). (+/−) Inconsistent or contradictory results in different positions. (NA) Data was not available for this species.

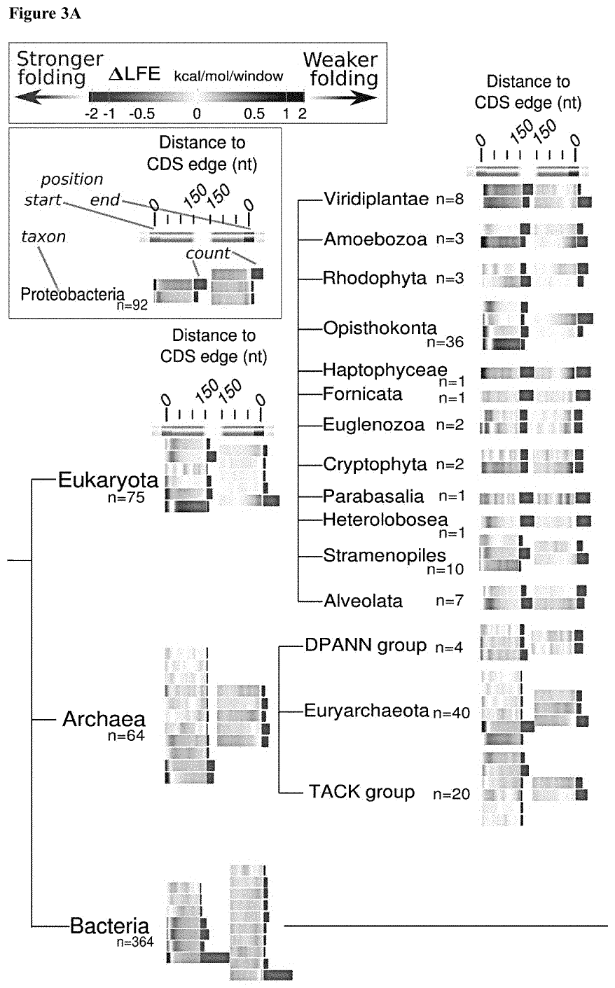

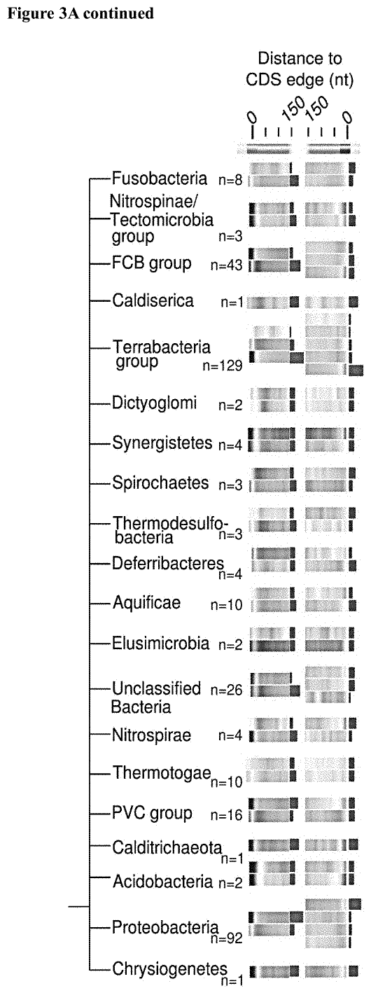

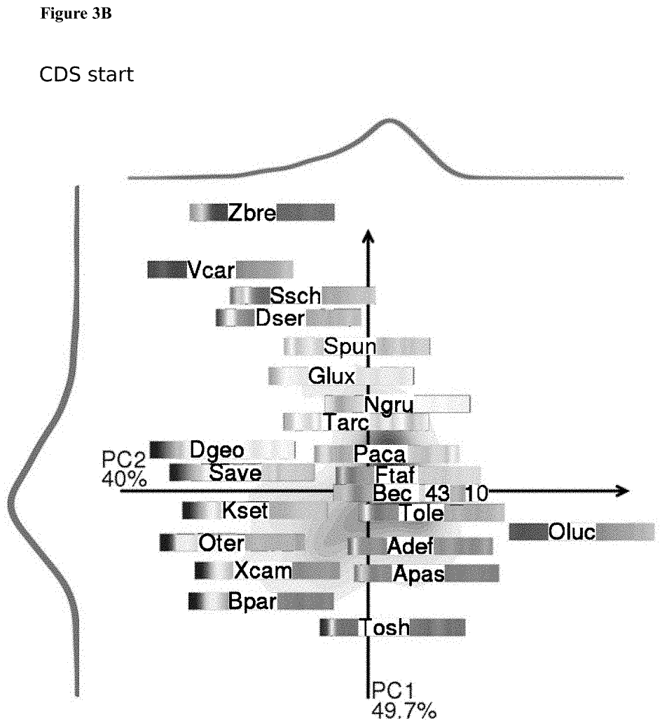

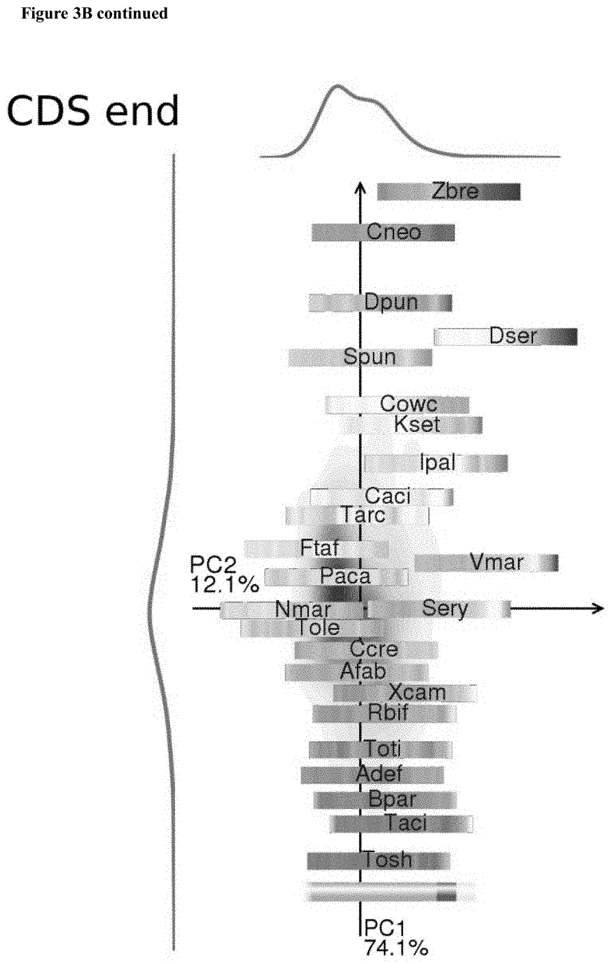

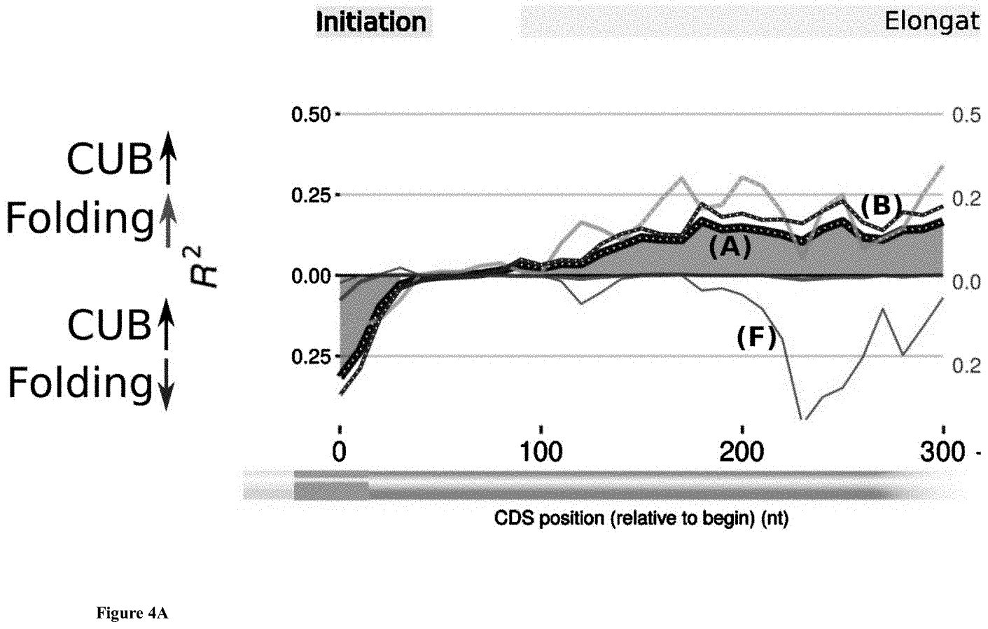

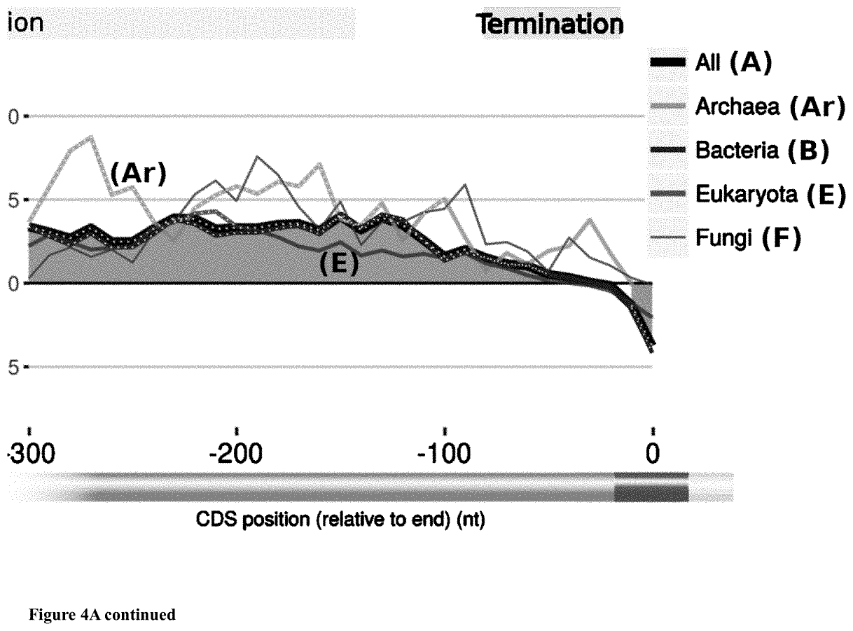

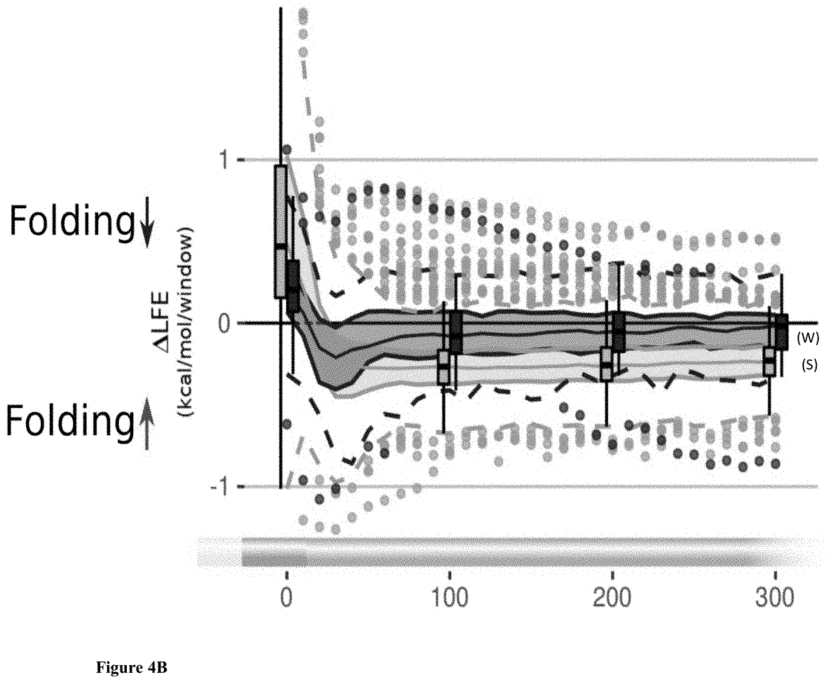

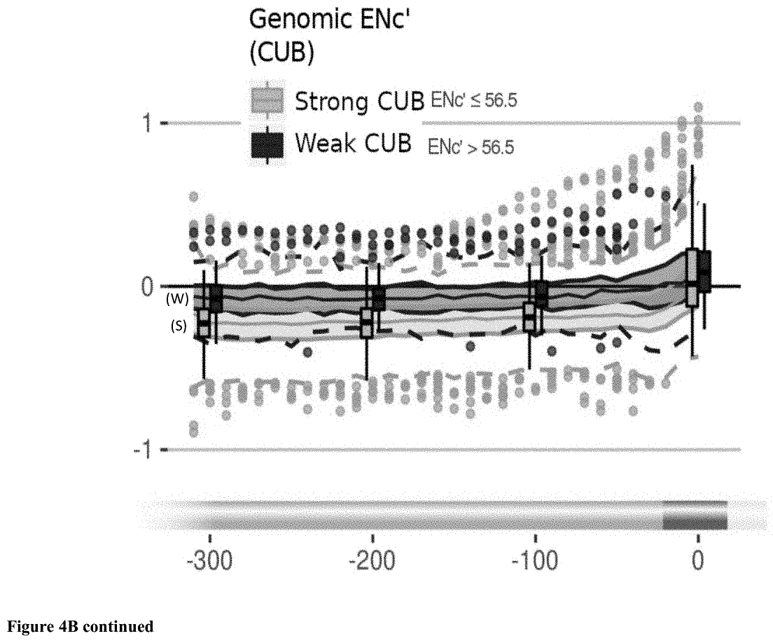

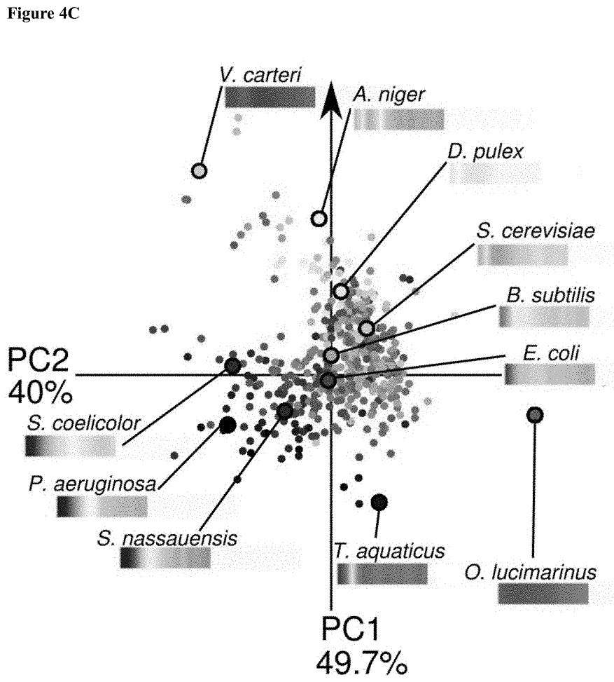

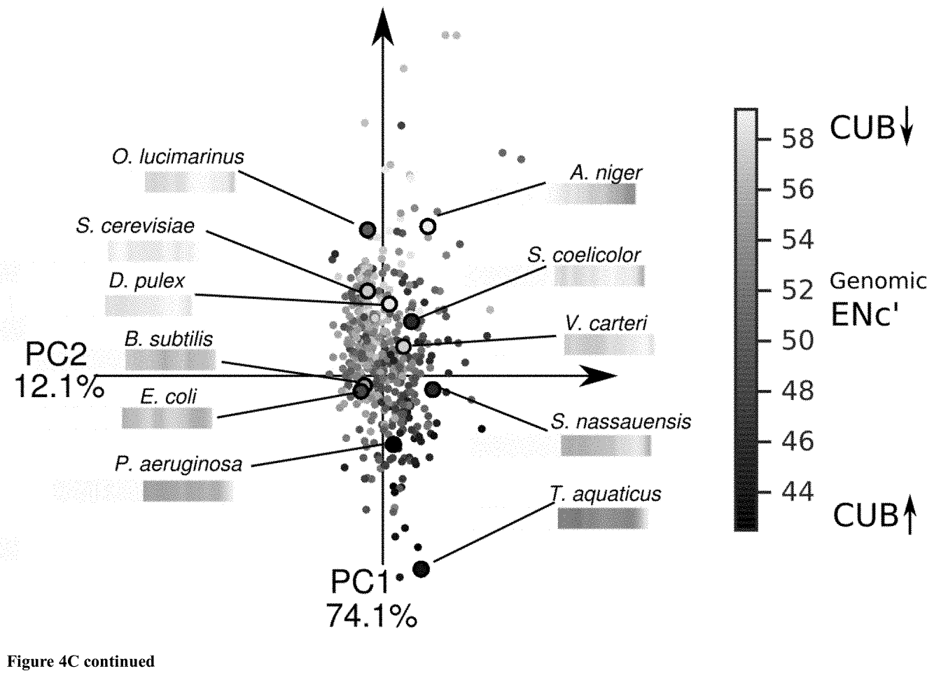

FIGS. 16A-C: Principal Component Analysis (PCA) of the ΔLFE profiles uncovers two components, with different relative weights for the CDS-edge and mid-CDS regions. (16A) PCA plot for ΔLFE profiles at positions 0-300 nt relative to CDS start (represented as vectors of length 31), shown by plotting each ΔLFE profile in its position in PCA space (with 2 dimensions), with overlapping profiles hidden to avoid clutter. The density of profiles in each region is illustrated using shading and the marginal distributions are shown on the axes. Loading vectors for positions 0 nt and 250 nt (relative to CDS start) are shown. To verify this analysis is robust, bootstrapping using 1000 repeats was used to measure the following values: RSD1—Relative standard-deviation (SD/mean) for the angle between the loading vectors shown (i.e., those for ΔLFE profile positions 0 nt and 250 nt). Distribution of angles shown in 16C. RSD2—Relative standard-deviation (SD/mean) for the explained variance of PC1. (16B) PCA plot for ΔLFE profiles at positions 0-300 nt relative to CDS end (created using the same method as 16A). (16C) Distribution of angles between shown loading vectors (i.e., those for ΔLFE profile positions 0 nt and 250 nt) using 1000 bootstrap samples. The distribution mean is 2.08 radians (119°) and the relative standard deviation (also shown as RSD1 on 16A) is 1.4%. This procedure was repeated for all species and for each domain individually (see also FIG. 4D). In each case, the first two PCs explain >80% of the variation. The loading vectors for positions 0 nt and 250 nt are not parallel nor orthogonal (and this is robust to sampling and persists in smaller groups, see FIG. 4D), indicating some level of dependence between the two positions (also indicated in FIG. 3E).

FIG. 17: ΔLFE profiles calculated using the CDS-wide randomization for individual species arranged by NCBI taxonomy. The ΔLFE profiles shown are for positions 0-300 nt relative to CDS start (left) and CDS end (right). The numbers of species included in each group is shown to the left of the group name.

FIG. 18: Distribution of ΔLFE profiles relative to CDS start (left) and end (right), for species belonging to each domain. In bacteria and archaea, only one species has positive ΔLFE in the mid-CDS region, despite this being common in eukaryotes.

FIGS. 19A-B: (19A) Autocorrelation for ΔLFE between positions relative to CDS start. Above main diagonal—Pearson's correlation. Below main diagonal—coefficient of determination (R²) for GLS regression. Values for positions a-h indicated in FIG. 19B. Significant positions (p-value<0.01) indicated by white dots. (19B) Numerical values (a-d—R², e-h—Pearson's-r) and p-values for positions marked in 19A. This supports the robustness of the values in FIG. 3E.

FIGS. 20A-C: Coefficient of determination (R²) and regression direction for GLS regression between genomic-GC % and mean ΔLFE in different taxonomic subgroups, for two regions relative to CDS-start. Top bar. 0-20 nt; Bottom bar, 70-300 nt. Sign of regression slope is indicated by color—Red—positive (reinforcing) effect; Blue—negative (compensating) effect. Significant results (FDR, p-value<0.01) are indicated by color intensity and marked with a ‘*’. Included taxonomic groups have 9 or more species in the dataset. (20A) Genomic GC. (20B) Genomic ENc′. (20C) Optimum Temperature.

FIG. 21: Using different measures of CUB generally leads to the same conclusion about the interaction between CUB and ΔLFE. Note that for CAI and DCBS, increasing values indicate stronger bias, whereas for ENc′, decreasing values indicate stronger bias. The following measures were used to estimate genomic CUB. CAI was computed using codonw version 1.4.4, using the entire genome as the reference set. ENc′ was calculated using ENCprime (github user jnovembre, commit 0ead568, Oct. 2016). DCBS was calculated as described in the paper. All CUB measures were averaged for each genome and the resulting values were used in GLS regression against the ΔLFE at each position.

FIGS. 22A-D: To test if correlation between genomic-ENc′ and ΔLFE is related to the general magnitude of ΔLFE or to position-specific aspects of the ΔLFE profile, we performed the following test: we decomposed the values by normalizing each genomic profile by its standard-deviation (as a measure of its scale), thus getting profiles of equal scale. We then checked for correlation between the normalized ΔLFE profiles with genomic-ENc′. There was no correlation after this normalization (FIG. 19), but the correlation between genomic-ENc′ and the scaling factor was strong. This suggests that the correlation of ENc′ (in contrast to GC-content) is indeed caused by the magnitude of ΔLFE. The observed correlation of ΔLFE with Genomic-ENc′ (FIG. 6) is due to correlation with the magnitude of the ΔLFE profile. When all profiles are normalized to have the same scale (by dividing the values of each profile by their standard deviation so the resulting profiles all have standard deviation 1), most of the correlation is removed (20A-B). For comparison, the same procedure is followed for genomic-GC (20C-D). Values represent coefficient of determination (R²) for GLS regression of each trait (genomic-ENc′ or genomic-GC %) vs. the normalized ΔLFE profile at different position relative to CDS edges, with the sign representing the regression coefficient. Regressions for different taxa are shown using different line colors and widths (black is for all species), and white dots show areas in which the regression is significant (p-value<0.01). The dashed red line represents R² for regression against the standard deviation for each ΔLFE profile (i.e., the scaling factor). (20A) Genomic-ENc′ vs. ΔLFE, CDS start. (20B) Genomic-ENc′ vs. ΔLFE, CDS end. (20C) Genomic-GC vs. ΔLFE, CDS start. (20D) Genomic-GC vs. ΔLFE, CDS end.

FIGS. 23A-B: (23A) Comparison of R² values for GLS regression using genomic-GC (blue), genomic-ENc′ (green), and both factors (red). Significance of the regression slope (determined using t-test) is indicated by white dots. Genomic-GC and genomic-ENc′ have similar explanatory power in the mid-CDS region, but they explain somewhat different parts of the variation, so adding the second factor improved the regression fit and the slope of the second factor (in this case, ENc′) is significant in most position within the CDS. (23B) Numeric regression results for multiple regression using genomic-GC and genomic-ENc′ in 4 regions of the CDS shows slopes for both factors are significant in most regions. This indicates each factor improves upon the prediction of the other factor. Significance is determined using t-test. CDS Reference—point in CDS (start/end) for defining relative positions within all CDSs. Positions: range of positions within CDS (relative to the reference) for which ΔLFE values are averaged. p-value (GC): p-value (using t-test) for Genomic-GC factor, in multiple regression (including factors GenmoicGC, GenomicENc′) using GLS. p-value (ENc′): p-value (using t-test) for Genomic-ENc′ factor, in multiple regression (including factors GenmoicGC, GenomicENc′) using GLS. R² (GLS): coefficient of determination (R²) for regression using the factors GenmoicGC+GenomicENc′. N: number of species included in GLS regression. Group: taxonomic group for this analysis.

FIG. 24: Numeric regression results for GLS multiple regression using genomic-GC, genomic-ENc′ and intracellular classification in 4 regions of the CDS, for several taxonomic groups (which contain a sufficient number of intracellular species). p-values shown for GLS are for the categorical Is-intracellular classification factor (determined using t-test), indicating this factor improves upon the predictions made using the two numerical factors in some cases (even after controlling for evolutionary relatedness using GLS), but not in others. R² values are shown for the regression without and with intracellular classification. CDS Reference—point in CDS (start/end) for defining relative positions within all CDSs. Positions: range of positions within CDS (relative to the reference) for which ΔLFE values are averaged. OLS p-value: p-value (using t-test) for Is-intracellular factor, in single regression using OLS (uncorrected for phylogenetic distances). This regression includes all available species (including those which are not contained in the phylogenetic tree so are not used in GLS regression). GLS p-value: p-value (using t-test) for Is-intracellular factor, in multiple regression (including factors GenmoicGC, GenomicENc′) using GLS. R² without Is-intracellular: coefficient of determination (R²) for regression using the factors GenmoicGC+GenomicENc′, as baseline for comparing improvement from the additional factor Is-intracellular. R² with Is-intracellular: coefficient of determination (R²) for regression using the factors GenmoicGC+GenomicENc′+Is-intracellular. Slope: direction of slope for factor Is-intracellular (positive or negative). This indicates intracellular species have weaker ΔLFE in the ranges shown. N: number of species included in GLS regression. Group: taxonomic group for this analysis.

FIG. 25: Coefficient of determination (R²) and regression direction (red—positive slope, blue, negative slope) for GLS regression between Genomic-GC % and mean ΔLFE in regions relative to CDS start and end, for different taxonomic subgroups. Significant values (p-value <0.01) are marked with white dots.

FIGS. 26A-C: Additional controls for two potentially confounding effects relating to translation initiation. Genes having weak SD sequence may require stronger contribution of other initiation-promoting mechanisms to ensure efficient translation initiation, and therefore might have stronger ΔLFE at the CDS start (feature [26A]). This effect, previously reported in the 5′UTRs of S. sp. PCC6803, is also observed here. CDS that overlap with a previous CDS may have biased ΔLFE results close to the overlapping region (this phenomenon is known, for example, in E. coli). As a simple control for this, we show the difference between genes with 5′ intergenic distances shorter than 50 nt (including overlapping genes) and other genes. Results show significant but small differences near the CDS start in some but not all species (see e.g., S. sp. and E. coli, panels 26B, 26C). Additional differences observed at other points in the CDS may be related to operonic structure. In E. coli, for example, a large decrease in mean ΔLFE is observed in genes with long intergenic distances, but the distributions of the two groups remain similar (inset on the right shows the distributions at the position 40 nt from CDS start, where the effect is strongest). SD strength was calculated using the minimum anti-SD hybridization energy in the 20 nt upstream of the start codon. The “weak SD” group includes genes with minimum energy greater than −1 kcal/mol.

FIGS. 27A-B: (27A) Correlation between ΔLFE calculated using standard temperature (37° C.) and native temperature (see methods), at each position relative to CDS start, for species grouped by native temperature range. Correlations were calculated for a random sample (N=71) of species (bacteria and archaea) for which native temperature data is available. (27B) Comparison of individual mean ΔLFE profiles using calculated using standard temperature (37° C.) and native temperature.

### DETAILED DESCRIPTION OF THE INVENTION

The present invention, in some embodiments, provides nucleic acid molecules comprising a coding sequence, wherein the coding sequence comprises at least one codon substituted to a synonymous codon within a region upstream of the stop codon and wherein the substitution increases folding energy of the region. The present invention further concerns a method of optimizing a coding sequence by introducing a mutation that increases folding energy into a region upstream of the stop codon.

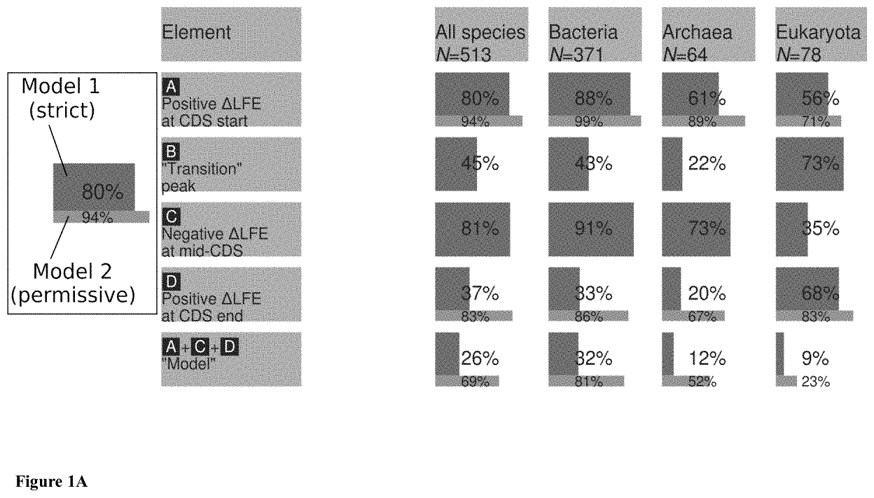

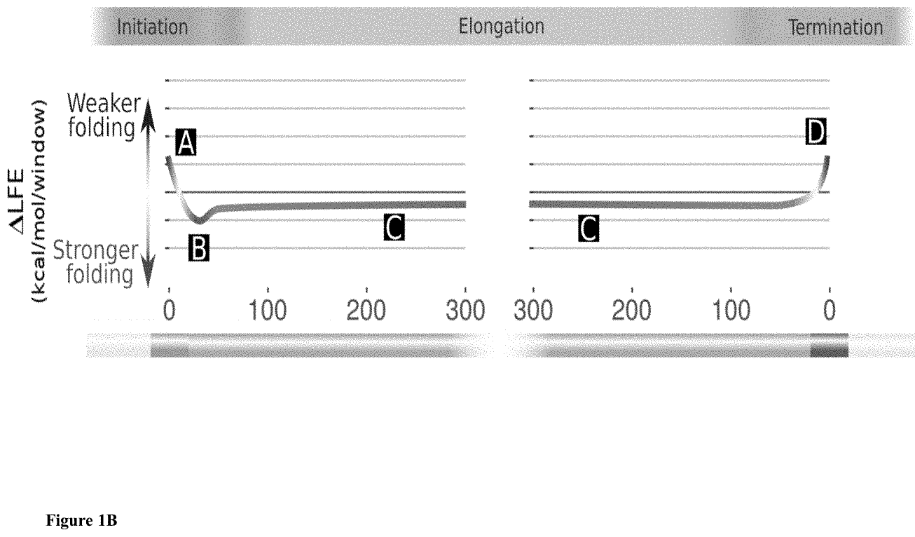

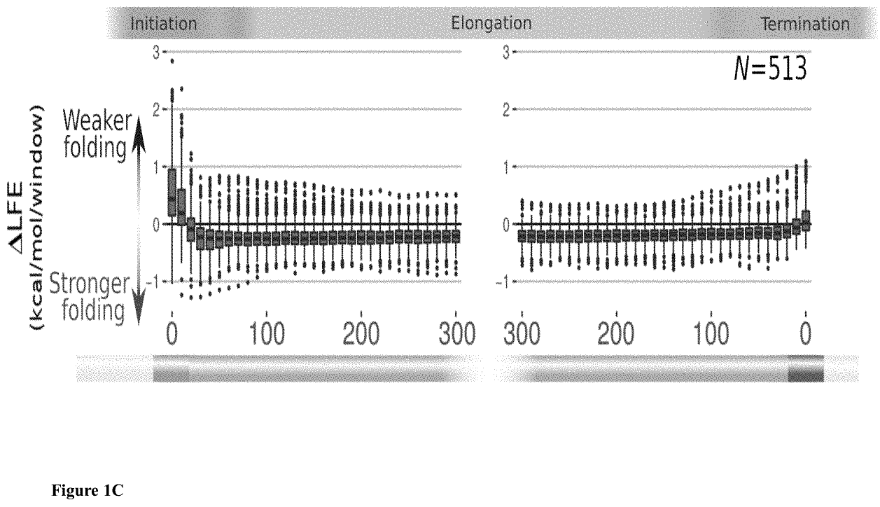

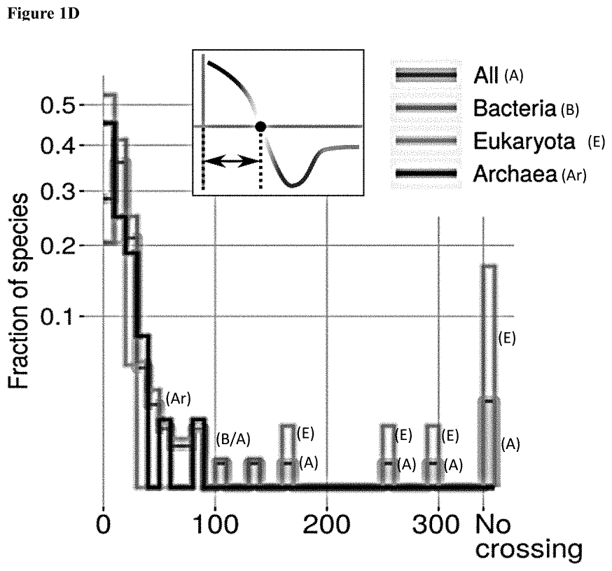

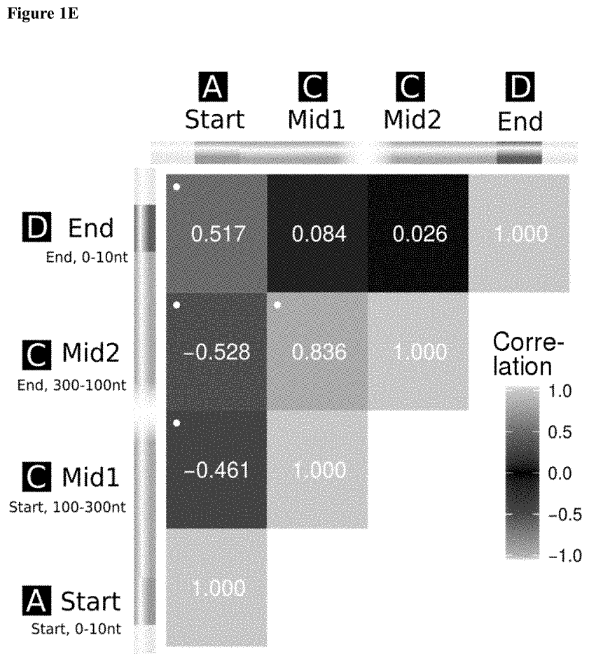

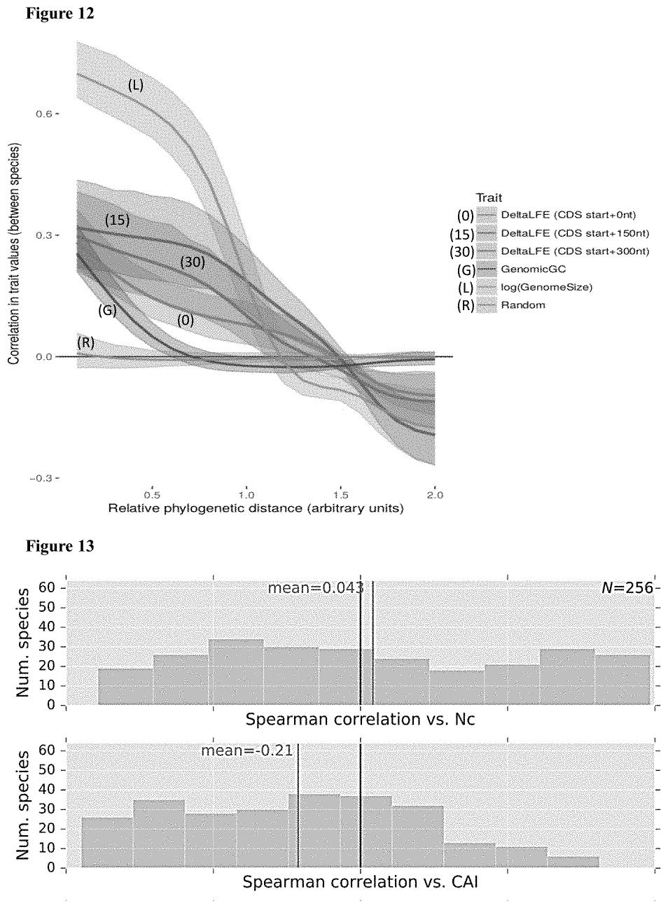

The invention is based on the following suppressing findings. First, it was found that selection on mRNA folding strength in most (but not all) species follows a conserved structure with three distinct regions (FIG. 1)—decreased local folding strength at the beginning and end of the coding region and increased folding strength in mid-CDS. The fact that this structure is more conserved than other genomic traits like GC-content (FIG. 12), as well as its alignment to the coding regions, suggest these features are related, at least in part, to translation regulation. Statistical tests demonstrate that these features cannot be merely side effects of factors known to be under selection like codon usage bias and amino-acid composition.

Conformance to different model elements varies significantly between the three domains: weak folding at the beginning of the coding regions appears in the great majority of bacterial species (88%) but only in 56%/60% of eukaryotes/archaea respectively (FIG. 1A, 3A). These differences may be related to polycistronic gene expression (see FIG. 26) or to generally higher effective population sizes and selection for high growth rate in bacteria; they may also indicate complementary constraints imposed by eukaryotic gene expression mechanisms (e.g., Cap-dependent translation initiation) and unique environmental constrains in archaea. On the other hand, selection for weak mRNA folding at the end of coding region (first conclusively shown here) is much more frequent in eukaryotes (appearing in 68% of the analyzed organism) than in prokaryotes (20% in archaea and 33% in bacteria).

Second, it was found that in some eukaryotes (in 13% of the analyzed eukaryotes and in one bacterium: D. puniceus) there is significant positive ΔLFE throughout the mid-CDS region (i.e., opposite to the general trend in prokaryotes, FIGS. 1A, 6A-B, and 18).

Third, it was shown that the “transition peak”, a region of selection for strong mRNA folding beginning around 30-70 nt downstream of the start codon that was reported elsewhere to be associated with translation efficiency, appears frequently (45%) in the analyzed organisms, indicating this mechanism is common (FIG. 1A, 1C). This feature appears much more frequently in eukaryotes (73%) than in prokaryotes (22% in archaea and 43% in bacteria).

Fourth, despite these differences, there was found a strong correlation between the strengths of three profile elements (found at the beginning, middle and end of the coding regions, FIG. 1E) across the analyzed organisms. This supports that much of the variation in their strength among organisms is caused by common factors acting jointly on the level of ΔLFE at all regions of the CDS.

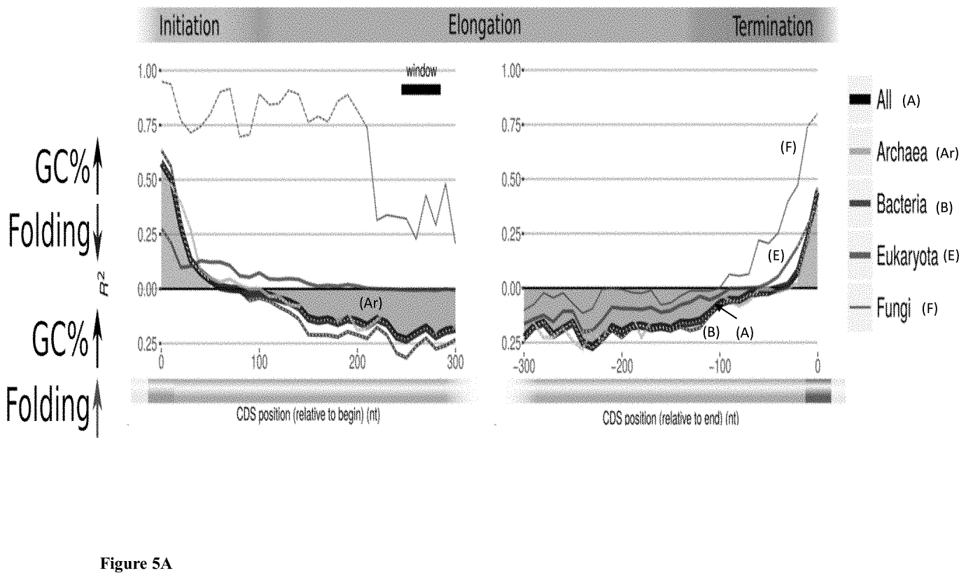

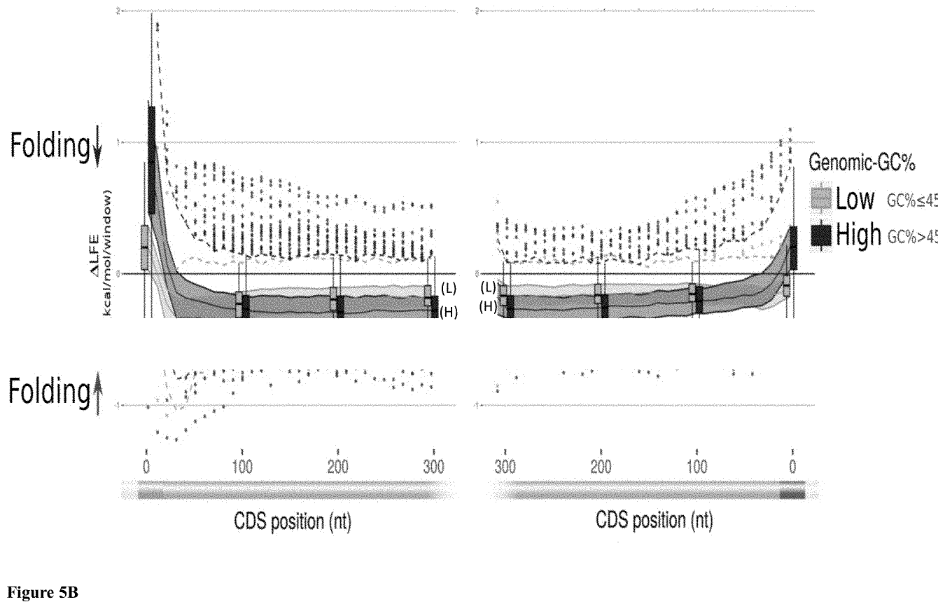

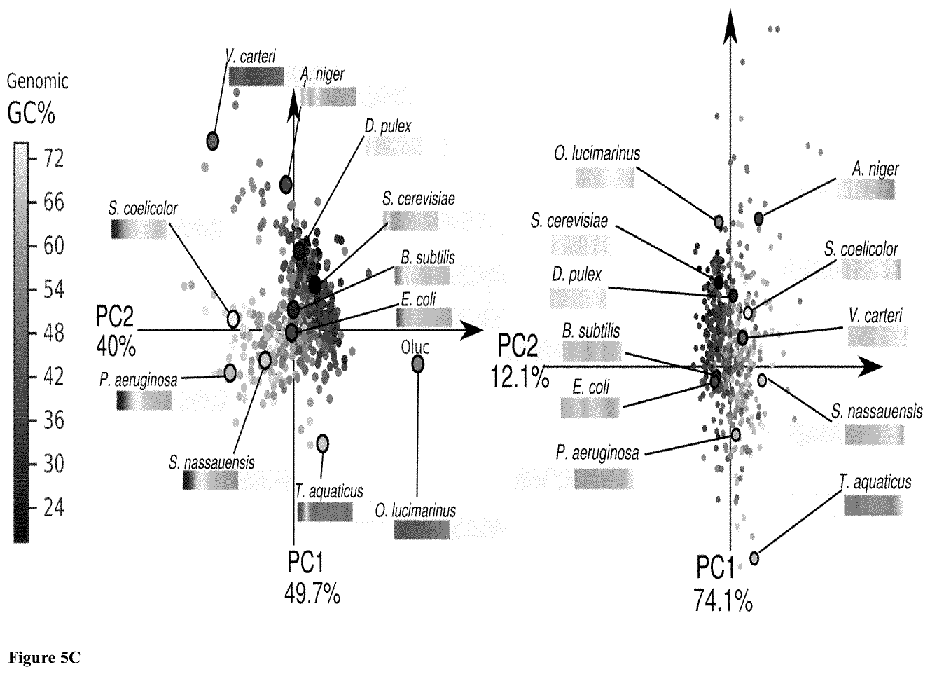

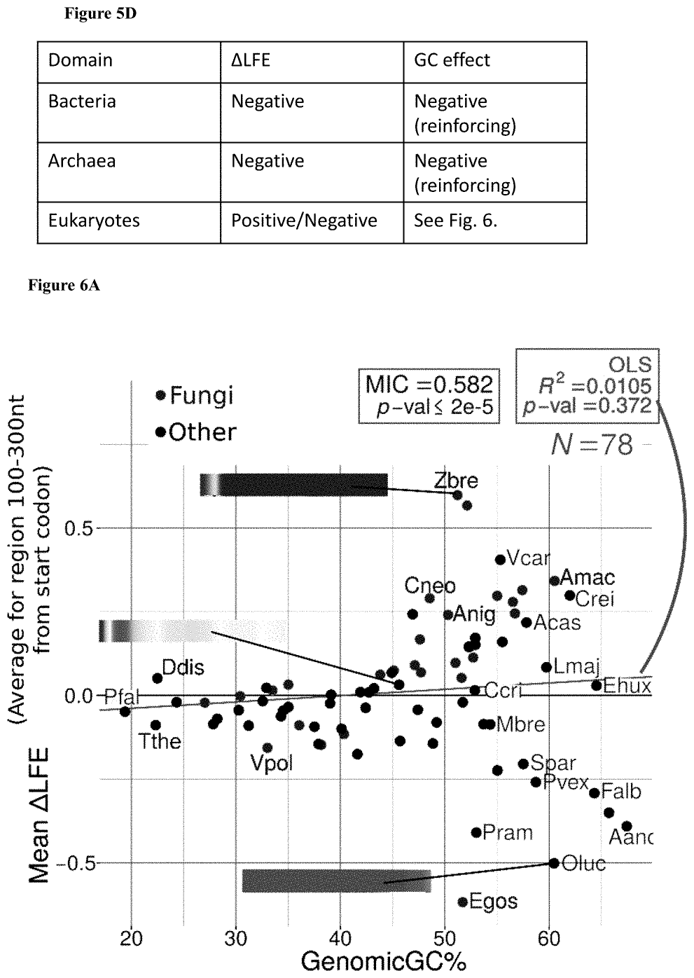

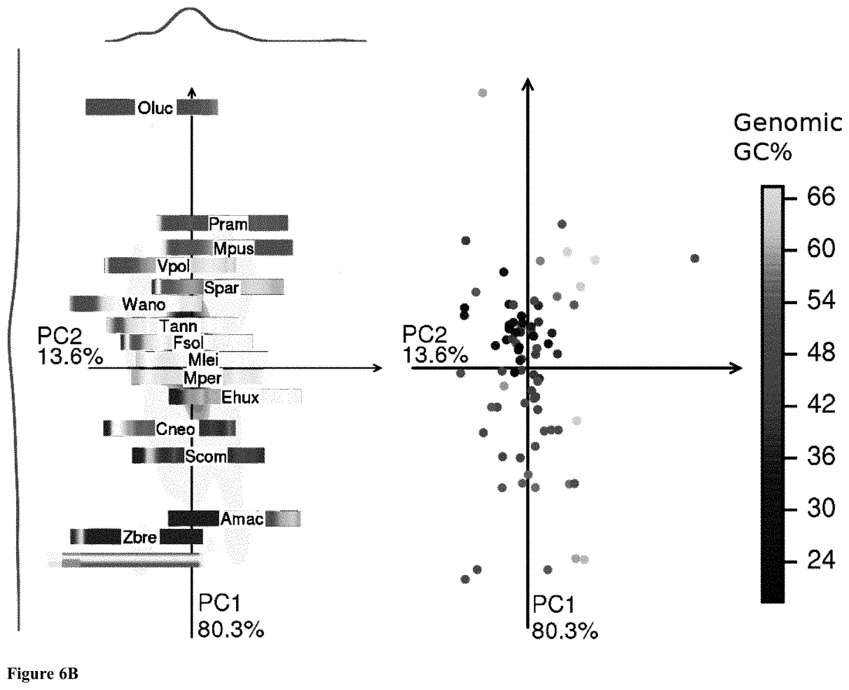

Fifth, there were found several variables that correlate with ΔLFE (and account for much of the variation mentioned above). The variables showing the strongest correlation are genomic GC-content (despite being explicitly controlled for by the randomizations as explained above, FIG. 5A-C) and CUB (measured using ENc′, FIG. 4A-C). Strong CUB and higher GC-content tend to be associated with more efficient selection on translation efficiency, and the fact that ΔLFE is correlated with them suggests the same underlying mechanism (or mechanisms) contribute to their selection.

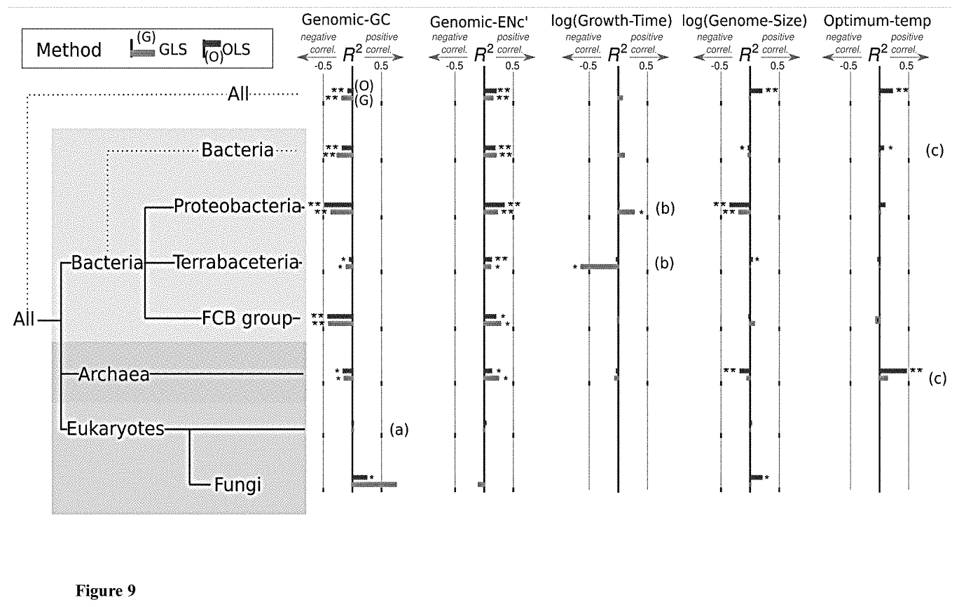

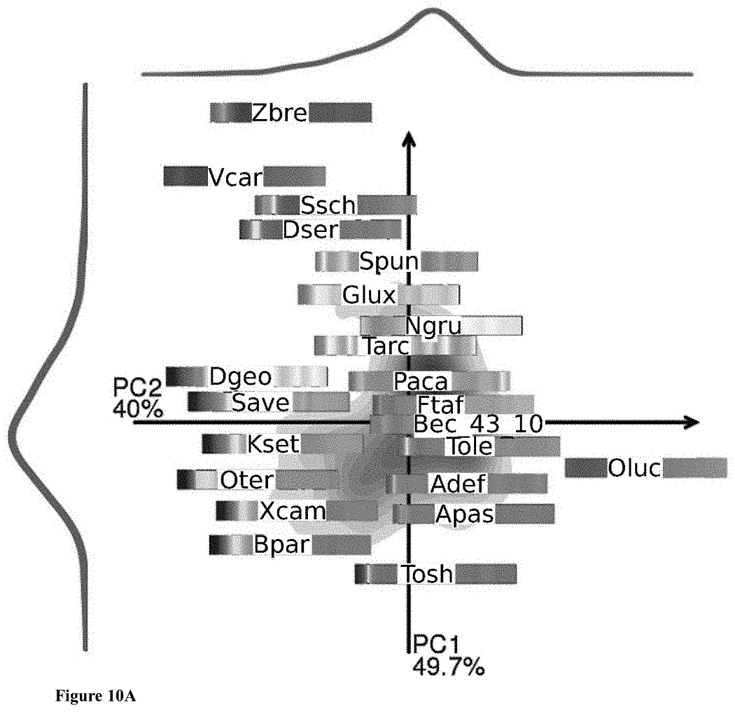

The influence on ΔLFE of all traits analyzed in the mid-CDS region can be compared in FIG. 9. Other genomic and environmental traits analyzed (including genome size and growth time) were not found to have significant linear interaction with ΔLFE at the domain level. In many cases there appear to be potential interactions with ΔLFE in smaller taxa (which may or may not be due to real interactions specific to those taxa, FIG. 20).

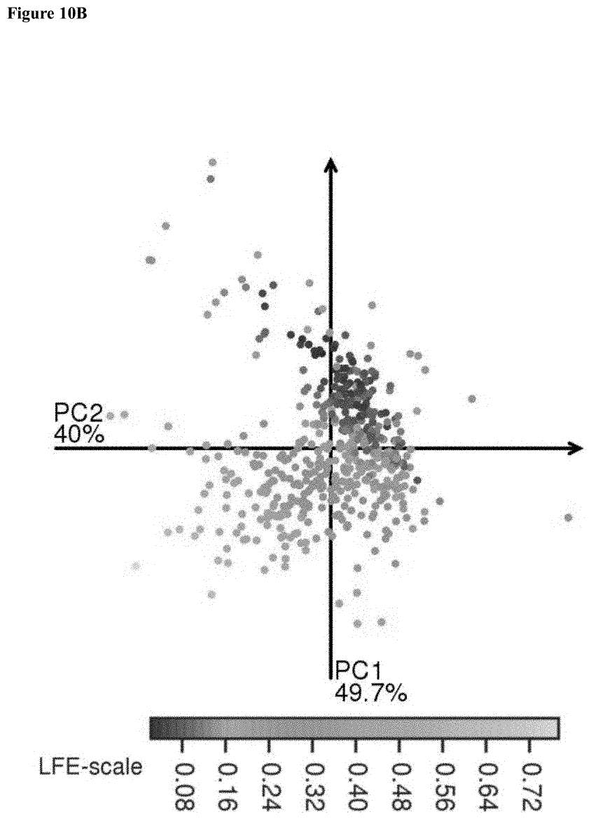

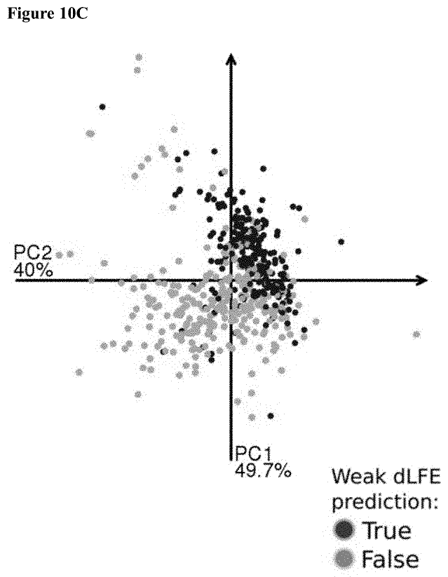

Sixth, there were identified four specific conditions that tend to prevent strong ΔLFE from occurring (separately and together). The first two conditions are based on the correlated traits described above: low GC-content and low CUB. Another characteristic is optimum growth temperature, since in higher temperatures base-pairing is weakened and consequently the influence of codons arrangement and composition must also be reduced, and so is any possible effect of ΔLFE. The last disrupting factor, an intracellular life phase, stems from the fact that such organisms generally have lower effective population size (due to recurring population bottlenecks) and lower selection pressure on gene expression (because they partly rely on the host). A binary classification model based on these four features has precision 0.66 and recall 0.82 in classification of ΔLFE strength (see Example 2 and FIG. 10). It should be noted that this binary classification discriminates species with very weak ΔLFE and has weak predictive value for ΔLFE strength in species where none of the factors hold, giving R²=0.2 (p-value=5e-25, OLS) against mean |ΔLFE| in the 150-300 nt region relative to CDS start. These conditions support the proposed mechanism of ΔLFE being the result of selection on secondary structure strength related to gene expression regulation and efficiency.

These results point to cases where evolutionary close organisms exhibit very different ΔLFE patterns and selection levels. For example, in fungi, members of Pezizomycotina (such as Aspergillus niger or Zymoseptoria brevis) have much more positive ΔLFE compared to members of Saccharomycotina (including Eremothecium gossyppi and Candida albicans). Notably, a few eukaryotic species (e.g., the unrelated species Fonticula alba and Saprolegnia parasitica) have a ΔLFE profile that looks typical for bacteria (FIG. 17). This highlights the variety of gene expression mechanisms in eukaryotes, as well as the risk in generalizing about disparate groups based on observations on model organisms.

Finally, it should be noted that this analysis is based on average values over entire genomes. This provides important statistical power and reduces the random effects of other factors on specific genes. It is important to remember, however, that some of the gene-level factors filtered this way are nevertheless important and there is considerable variation between genes.

By a first aspect, there is provided a nucleic acid molecule comprising a coding sequence comprising at least one codon substituted to a different codon within a first region of said coding sequence, wherein said substitution increases or decreases folding energy of the first region or of RNA encoded by the first region.

In some embodiments, the nucleic acid molecule is an RNA molecule or a DNA molecule. In some embodiments, the nucleic acid molecule is an RNA molecule. In some embodiments, the nucleic acid molecule is a DNA molecule. In some embodiments, the DNA is genomic DNA. In some embodiments, the DNA is cDNA. In some embodiments, the nucleic acid molecule is a vector. In some embodiments, the vector is an expression vector. In some embodiments, the expression vector is a prokaryotic expression vector. In some embodiments, the expression vector is a eukaryotic expression vector. In some embodiments, the prokaryote is a bacterium. In some embodiments, the prokaryote is an archaeon. In some embodiments, the eukaryote is a mammal. In some embodiments, the mammal is a human. In some embodiments, the eukaryote is not a fungus.

In some embodiments, the nucleic acid molecule comprises a coding region. In some embodiments, the nucleic acid molecule comprises a coding sequence. In some embodiments, the coding region comprises a start codon. In some embodiments, the nucleic acid molecule comprises a stop codon. It will be understood by a skilled artisan that both DNA and RNA can be considered to have codons. Within a DNA molecule a codon refers to the 3 bases that will be transcribed into RNA bases that will act as a codon for recognition by a ribosome and will thus translate an amino acid. In some embodiments, the nucleic acid molecule further comprises an untranslated region (UTR). In some embodiments, the UTR is a 5′ UTR. In some embodiments, the UTR is a 3′ UTR.

As used herein, the term “coding sequence” refers to a nucleic acid sequence that when translated results in an expressed protein. In some embodiments, the coding sequence is to be used as a basis for making codon alterations. In some embodiments, the coding sequence is a gene. In some embodiments, the coding sequence is a viral gene. In some embodiments, the coding sequence is a prokaryotic gene. In some embodiments, the coding sequence is a bacterial gene. In some embodiments, the coding sequence is a eukaryotic gene. In some embodiments, the coding sequence is a mammalian gene. In some embodiments, the coding sequence is a human gene. In some embodiments, the coding sequence is a portion of one of the above listed genes. In some embodiments, the coding sequence is a heterologous transgene. In some embodiments, the above listed genes are wild type, endogenously expressed genes. In some embodiments, the above listed genes have been genetically modified or in some way altered from their endogenous formulation. These alterations may be changes to the coding region such that the protein the gene codes for is altered.

The term “heterologous transgene” as used herein refers to a gene that originated in one species and is being expressed in another. In some embodiments, the transgene is a part of a gene originating in another organism. In some embodiments, the heterologous transgene is a gene to be overexpressed. In some embodiments, expression of the heterologous transgene in a wild-type cell reduces global translation in the wild-type cell.

In some embodiments, the nucleic acid molecule further comprises a regulatory element. In some embodiments, regulatory element is configured to induce transcription of the coding sequence. In some embodiments, the regulatory element is a promoter. In some embodiments, the regulatory element is selected from an activator, a repressor, an enhancer, and an insulator. In some embodiments, the coding region is operably linked to the regulatory element. The term “operably linked” is intended to mean that the coding sequence is linked to the regulatory element or elements in a manner that allows for expression of the coding sequence (e.g., in an in vitro transcription/translation system or in a host cell when the vector is introduced into the host cell). In some embodiments, the promoter is a promoter specific to the expression vector. In some embodiments, the promoter is a viral promoter. In some embodiments, the promoter is a bacterial promoter. In some embodiments, the promoter is a eukaryotic promoter.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

In some embodiments, another codon is a synonymous codon. In some embodiments, a codon is substituted to a synonymous codon. In some embodiments, the substitution is a silent substitution. In some embodiments, the substitution is a mutation. In some embodiments, a codon is mutated to another codon. In some embodiments, the other codon is a synonymous codon. In some embodiments, the mutation is a silent mutation.

The term “codon” refers to a sequence of three DNA or RNA nucleotides that correspond to a specific amino acid or stop signal during protein synthesis. The codon code is degenerate, in that more than one codon can code for the same amino acid. Such codons that code for the same amino acid are known as “synonymous” codons. Thus, for example, CUU, CUC, CUA, CUG, UUA, and UUG are synonymous codons that code for Leucine. Synonymous codons are not used with equal frequency. In general, the most frequently used codons in a particular cell are those for which the cognate tRNA is abundant, and the use of these codons enhances the rate of protein translation. Conversely, tRNAs for rarely used codons are found at relatively low levels, and the use of rare codons is thought to reduce translation rate. “Codon bias” as used herein refers generally to the non-equal usage of the various synonymous codons, and specifically to the relative frequency at which a given synonymous codon is used in a defined sequence or set of sequences.

Synonymous codons are provided in Table 6. The first nucleotide in each codon encoding a particular amino acid is shown in the left-most column; the second nucleotide is shown in the top row; and the third nucleotide is shown in the right-most column.

Table 6: Codon table showing synonymous codons

TABLE 6
Codon table showing synonymous codons

| | U | C | A | G | |
|---|---|---|---|---|---|
| U | Phe | Ser | Tyr | Cys | U |
| | Phe | Ser | Tyr | Cys | C |
| | Leu | Ser | STOP | STOP | A |
| | Leu | Ser | STOP | Trp | G |
| C | Leu | Pro | His | Arg | U |
| | Leu | Pro | His | Arg | C |
| | Leu | Pro | Gln | Arg | A |
| | Leu | Pro | Gln | Arg | G |
| A | Ile | Thr | Asn | Ser | U |
| | Ile | Thr | Asn | Ser | C |
| | Ile | Thr | Lys | Arg | A |
| | Met | Thr | Lys | Arg | G |
| G | Val | Ala | Asp | Gly | U |
| | Val | Ala | Asp | Gly | C |
| | Val | Ala | Glu | Gly | A |
| | Val | Ala | Glu | Gly | G |

As used herein, the term “silent mutation” refers to a mutation that does not affect or has little effect on protein functionality. A silent mutation can be a synonymous mutation and therefore not change the amino acids at all, or a silent mutation can change an amino acid to another amino acid with the same functionality or structure, thereby having no or a limited effect on protein functionality.

In some embodiments, the first region is from 90 nucleotides upstream of a stop codon of the coding sequence to the stop codon. In some embodiments, the first region is from 50 nucleotides upstream of the stop codon to the stop codon. In some embodiments, the first region is from 40 nucleotides upstream of the stop codon to the stop codon. It will be understood by a skilled artisan that “upstream from the stop codon” refers to from the first base of the stop codon. Thus, the first base of the stop codon is considered to be nucleotide zero, and the base directly 5′ to that first base of the stop codon is therefore 1 nucleotide upstream of the stop codon. Thus, the first region may be from 90, 50 or 40 nucleotides upstream of the stop codon. In some embodiments, the first region does not include the stop codon. In some embodiments, the first region does include the stop codon. In some embodiments, the first region is from 90 nucleotides upstream of the stop codon to 1 nucleotide upstream of the stop codon. In some embodiments, the first region is from 50 nucleotides upstream of the stop codon to 1 nucleotide upstream of the stop codon. In some embodiments, the first region is from 40 nucleotides upstream of the stop codon to 1 nucleotide upstream of the stop codon. In some embodiments, the first region does not comprise the two codons closest to the stop codon. In some embodiments, the first region is from 90 nucleotides upstream of the stop codon to 7 nucleotides upstream of the stop codon. In some embodiments, the first region is from 50 nucleotides upstream of the stop codon to 7 nucleotides upstream of the stop codon. In some embodiments, the first region is from 40 nucleotides upstream of the stop codon to 7 nucleotides upstream of the stop codon.

In some embodiments, the first region is upstream and proximal to the stop codon and folding energy of the first region or of RNA encoded by the first region is increased. In some embodiments, the folding energy is RNA secondary structure folding Gibbs free energy. In some embodiments, the region is DNA and the folding energy of the RNA encoded by the region is increased. It will be understood by a skilled artisan that the measure of folding energy is generally negative, and that an area with complex secondary structure, i.e., abundant folding, will have a very low, negative folding energy. Thus, increasing folding energy is decreasing secondary structure complexity and decreasing folding. In some embodiments, the substitution increases folding energy of the first region or RNA encoded by the first region to above a predetermined threshold. In some embodiments, the predetermined threshold is −5 kcal/mol/40 bp. In some embodiments, the predetermined threshold is −6 kcal/mol/40 bp. In some embodiments, the predetermined threshold is −6.09 kcal/mol/40 bp. In some embodiments, the predetermined threshold is −6.8 kcal/mol/40 bp. In some embodiments, the threshold is a statistically significant increase. In some embodiments, the threshold is derived from a randomized sequence. In some embodiments, threshold is derived from a null hypothesis. In some embodiments, the threshold is the folding energy of a random sequence. In some embodiments, the threshold is 0 kcal/mol/40 bp. In some embodiments, the threshold is a value above which the difference as compared to the already existing folding energy would be significant. In some embodiments, the threshold is a level that is statistically significant as compared to a null model for folding energy of the region. In some embodiments, the threshold is organism specific. In some embodiments, the threshold is selected from a threshold provided in Table 1. In some embodiments, the threshold is domain-specific and selected from a threshold provided in Table 1. In some embodiments, the threshold is species-specific and is selected from a threshold provided in Table 5. In embodiments, wherein the species is not provided in Table 5, the more general thresholds from Table 1 are used. In some embodiments, the threshold is selected from a threshold provided in Table 5. In some embodiments, the domain is Archaea, and the threshold is −5.76 kcal/mol/40 bp. In some embodiments, the threshold is an archaeal threshold, and the threshold is −5.76 kcal/mol/40 bp. In some embodiments, the domain is Bacteria, and the threshold is −6.17 kcal/mol/40 bp. In some embodiments, the threshold is a bacterial threshold, and the threshold is −6.17 kcal/mol/40 bp. In some embodiments, the domain is Eukaryotes, and the threshold is −5.95 kcal/mol/40 bp. In some embodiments, the threshold is a eukaryotic threshold, and the threshold is −5.95 kcal/mol/40 bp. In some embodiments, the threshold is the native LFE mean aat 0 nt. In some embodiments, the mean at 0 nt in the table is the threshold for a given domain or species.

TABLE 1
Native LFE (40 nt window), at the stop codon, for domains

| | | | Species |
|---|---|---|---|
| Domain | Mean at 0 nt | Std at 0 nt | Examined |
| All | −6.09 | 3.26 | 513 |
| Archaea | −5.76 | 3.21 | 64 |
| Bacteria | −6.17 | 3.27 | 371 |
| Eukaryotes | −5.95 | 3.26 | 78 |

TABLE 5
Native LFE (40 nt window), at the stop codon, for species

| | | | Mean | Std |
|---|---|---|---|---|
| TaxId | Species | Domain | at 0 | at 0 |
| 507754 | Acidiplasma aeolicum str. VT | Archaea | −3.03 | 2.48 |
| 1198449 | Aeropyrum camini SY1 = JCM 12091 | Archaea | −7.99 | 3.95 |
| 272557 | Aeropyrum pernix K1 | Archaea | −7.87 | 4.11 |
| 224325 | Archaeoglobus fulgidus DSM 4304 | Archaea | −5.74 | 3.20 |
| 1056495 | Caldisphaera lagunensis DSM 15908 | Archaea | −2.79 | 2.36 |
| 1072681 | Candidatus Haloredivivus sp. G17 | Archaea | −4.48 | 2.88 |
| 374847 | Candidatus Korarchaeum cryptofilum OPF8 | Archaea | −6.51 | 3.50 |
| 1295009 | Candidatus Methanomassiliicoccus intestinalis | Archaea | −4.75 | 2.90 |
| | Issoire-Mx1 str. Mx1-Issoire | | | |
| 1236689 | Candidatus Methanomethylophilus alvus Mx1201 | Archaea | −7.61 | 3.66 |
| 1577684 | Candidatus Nanopusillus acidilobi | Archaea | −1.99 | 1.87 |
| 859192 | Candidatus Nitrosoarchaeum limnia BG20 | Archaea | −3.00 | 2.40 |
| 1229908 | Candidatus Nitrosopumilus koreensis AR1 | Archaea | −3.30 | 2.49 |
| 1237085 | Candidatus Nitrososphaera gargensis Ga9.2 | Archaea | −5.83 | 3.34 |
| 414004 | Cenarchaeum symbiosum A | Archaea | −8.26 | 4.25 |
| 589924 | Ferroglobus placidus DSM 10642 | Archaea | −4.91 | 2.90 |
| 333146 | Ferroplasma acidarmanus fer1 | Archaea | −3.48 | 2.65 |
| 64091 | Halobacterium salinarum NRC-1 | Archaea | −10.42 | 4.28 |
| 478009 | Halobacterium salinarum R1 | Archaea | −10.34 | 4.32 |
| 523841 | Haloferax mediterranei ATCC 33500 | Archaea | −8.76 | 3.67 |
| 469382 | Halogeometricum borinquense DSM 11551 | Archaea | −8.76 | 3.66 |
| 797210 | Halopiger xanaduensis SH-6 | Archaea | −10.34 | 3.92 |
| 362976 | Haloquadratum walsbyi DSM 16790 | Archaea | −5.86 | 3.13 |
| 797114 | Halosimplex carlsbadense 2-9-1 | Archaea | −11.19 | 4.12 |
| 583356 | Ignisphaera aggregans DSM 17230 | Archaea | −3.88 | 2.68 |
| 1502293 | Marine Group I thaumarchaeote SCGC AAA799- | Archaea | −3.45 | 2.61 |
| | N04 | | | |
| 420247 | Methanobrevibacter smithii ATCC 35061 | Archaea | −2.95 | 2.41 |
| 243232 | Methanocaldococcus jannaschii DSM 2661 | Archaea | −2.67 | 2.31 |
| 267377 | Methanococcus maripaludis S2 | Archaea | −2.89 | 2.44 |
| 410358 | Methanocorpusculum labreanum Z | Archaea | −6.38 | 3.54 |
| 1201294 | Methanoculleus bourgensis MS2 | Archaea | −9.18 | 4.16 |
| 28892 | Methanofollis liminatans DSM 4140 | Archaea | −8.96 | 4.26 |
| 644295 | Methanohalobium evestigatum Z-7303 | Archaea | −3.62 | 2.66 |
| 867904 | Methanomethylovorans hollandica DSM 15978 | Archaea | −4.73 | 3.01 |
| 190192 | Methanopyrus kandleri AV19 | Archaea | −9.27 | 3.97 |
| 188937 | Methanosarcina acetivorans C2A | Archaea | −4.83 | 3.05 |
| 213585 | Methanosarcina mazei S-6 | Archaea | −4.89 | 3.28 |
| 339860 | Methanosphaera stadtmanae DSM 3091 | Archaea | −2.47 | 2.20 |
| 521011 | Methanosphaerula palustris E1-9c | Archaea | −7.57 | 3.75 |
| 187420 | Methanothermobacter thermautotrophicus | Archaea | −6.11 | 3.37 |
| | str. Delta H | | | |
| 228908 | Nanoarchaeum equitans | Archaea | −2.93 | 2.39 |
| 1737403 | Nanohaloarchaea archaeon SG9 | Archaea | −5.12 | 3.07 |
| 797304 | Natronobacterium gregoryi SP2 | Archaea | −9.34 | 3.78 |
| 436308 | Nitrosopumilus maritimus SCM1 | Archaea | −3.37 | 2.47 |
| 926571 | Nitrososphaera viennensis EN76 | Archaea | −6.71 | 3.68 |
| 1343739 | Palaeococcus pacificus DY20341 | Archaea | −4.82 | 3.02 |
| 263820 | Picrophilus torridus DSM 9790 | Archaea | −3.41 | 2.70 |
| 178306 | Pyrobaculum aerophilum str. IM2 | Archaea | −6.28 | 3.67 |
| 272844 | Pyrococcus abyssi GE5 | Archaea | −5.18 | 3.08 |
| 186497 | Pyrococcus furiosus DSM 3638 | Archaea | −4.45 | 3.00 |
| 70601 | Pyrococcus horikoshii OT3 | Archaea | −4.60 | 3.13 |
| 1273541 | Pyrodictium delaneyi | Archaea | −7.04 | 3.86 |
| 694429 | Pyrolobus fumarii 1A | Archaea | −7.48 | 3.67 |
| 429572 | Sulfolobus islandicus L.S.2.15 | Archaea | −3.32 | 2.51 |
| 273063 | Sulfolobus tokodaii str. 7 | Archaea | −3.14 | 2.55 |
| 1198115 | Thaumarchaeota archaeon SCGC AB-539-E09 | Archaea | −4.62 | 3.23 |
| 391623 | Thermococcus barophilus MP | Archaea | −4.68 | 2.93 |
| 163003 | Thermococcus cleftensis | Archaea | −7.89 | 3.71 |
| 593117 | Thermococcus gammatolerans EJ3 | Archaea | −7.16 | 3.46 |
| 1432656 | Thermococcus guaymasensis DSM 11113 | Archaea | −7.08 | 3.58 |
| 195522 | Thermococcus nautili | Archaea | −7.64 | 3.61 |
| 273075 | Thermoplasma acidophilum DSM 1728 | Archaea | −5.26 | 3.21 |
| 273116 | Thermoplasma volcanium GSS1 | Archaea | −4.08 | 2.82 |
| 768679 | Thermoproteus tenax Kra 1 | Archaea | −7.02 | 3.80 |
| 572478 | Vulcanisaeta distributa DSM 14429 | Archaea | −5.08 | 3.08 |
| 592010 | Abiotrophia defectiva ATCC 49176 | Bacteria | −5.38 | 3.44 |
| 1266844 | Acetobacter pasteurianus 386B | Bacteria | −7.51 | 3.75 |
| 574087 | Acetohalobium arabaticum DSM 5501 | Bacteria | −3.43 | 2.52 |
| 1009370 | Acetonema longum DSM 6540 | Bacteria | −6.09 | 3.55 |
| 441768 | Acholeplasma laidlawii PG-8A | Bacteria | −2.83 | 2.42 |
| 525909 | Acidimicrobium ferrooxidans DSM 10331 | Bacteria | −11.67 | 3.95 |
| 743299 | Acidithiobacillus ferrivorans SS3 | Bacteria | −7.82 | 3.71 |
| 243159 | Acidithiobacillus ferrooxidans ATCC 23270 | Bacteria | −8.30 | 3.84 |
| 240015 | Acidobacterium capsulatum ATCC 51196 | Bacteria | −8.77 | 3.89 |
| 351607 | Acidothermus cellulolyticus 11B | Bacteria | −11.51 | 4.18 |
| 400667 | Acinetobacter baumannii ATCC 17978 | Bacteria | −4.28 | 2.77 |
| 746697 | Aequorivita sublithincola DSM 14238 | Bacteria | −3.28 | 2.47 |
| 176299 | Agrobacterium fabrum str. C58 | Bacteria | −8.91 | 3.82 |
| 1435057 | Agrobacterium tumefaciens LBA4213 (Ach5) | Bacteria | −8.69 | 3.76 |
| 1514904 | Ahrensia marina str. LZD062 | Bacteria | −6.57 | 3.27 |
| 349741 | Akkermansia muciniphila ATCC BAA-835 | Bacteria | −7.40 | 3.94 |
| 393595 | Alcanivorax borkumensis SK2 | Bacteria | −7.41 | 3.55 |
| 543302 | Alicyclobacillus acidocaldarius LAA1 | Bacteria | −9.42 | 4.02 |
| 187272 | Alkalilimnicola ehrlichii MLHE-1 | Bacteria | −11.32 | 4.38 |
| 46234 | Anabaena sp. 90 | Bacteria | −3.98 | 2.94 |
| 891968 | Anaerobaculum mobile DSM 13181 | Bacteria | −5.67 | 3.18 |
| 525919 | Anaerococcus prevotii DSM 20548 | Bacteria | −2.99 | 2.40 |
| 926569 | Anaerolinea thermophila UNI-1 | Bacteria | −6.75 | 3.68 |
| 491915 | Anoxybacillus flavithermus WK1 | Bacteria | −4.03 | 2.77 |
| 224324 | Aquifex aeolicus VF5 | Bacteria | −4.45 | 2.90 |
| 696747 | Arthrospira platensis NIES-39 | Bacteria | −5.02 | 3.17 |
| 322098 | Aster yellows witches'-broom phytoplasma AYWB | Bacteria | −1.93 | 1.85 |
| 573065 | Asticcacaulis excentricus CB 48 | Bacteria | −8.69 | 3.79 |
| 1121088 | Bacillus coagulans DSM 1 = ATCC 7050 | Bacteria | −5.12 | 3.31 |
| 272558 | Bacillus halodurans C-125 | Bacteria | −4.43 | 2.84 |
| 439292 | Bacillus selenitireducens MLS10 | Bacteria | −5.55 | 3.20 |
| 224308 | Bacillus subtilis subsp. subtilis str. 168 | Bacteria | −4.89 | 3.06 |
| 295405 | Bacteroides fragilis YCH46 | Bacteria | −4.05 | 2.96 |
| 997884 | Bacteroides nordii | Bacteria | −3.83 | 2.79 |
| 226186 | Bacteroides thetaiotaomicron VPI-5482 | Bacteria | −4.10 | 2.89 |
| 283166 | Bartonella henselae str. Houston-1 | Bacteria | −4.00 | 2.74 |
| 264462 | Bdellovibrio bacteriovorus HD100 | Bacteria | −6.08 | 3.42 |
| 1618331 | Berkelbacteria bacterium GW2011_GWA1_36_9 | Bacteria | −3.23 | 2.71 |
| 703613 | Bifidobacterium animalis subsp. animalis ATCC | Bacteria | −8.94 | 3.83 |
| | 25527 | | | |
| 1046627 | Bizionia argentinensis JUB59 | Bacteria | −3.04 | 2.47 |
| 331104 | Blattabacterium sp. (Blattella germanica) str. Bge | Bacteria | −2.52 | 2.21 |
| 1208660 | Bordetella parapertussis Bpp5 | Bacteria | −11.64 | 4.89 |
| 526224 | Brachyspira murdochii DSM 12563 | Bacteria | −2.55 | 2.18 |
| 476282 | Bradyrhizobium japonicum SEMIA 5079 | Bacteria | −10.30 | 3.97 |
| 358681 | Brevibacillus brevis NBRC 100599 | Bacteria | −5.21 | 3.03 |
| 633149 | Brevundimonas subvibrioides ATCC 15264 | Bacteria | −11.64 | 4.23 |
| 224914 | Brucella melitensis bv. 1 str. 16M | Bacteria | −8.45 | 3.75 |
| 107806 | Buchnera aphidicola str. APS (Acyrthosiphon | Bacteria | −2.37 | 2.07 |
| | pisum) | | | |
| 926550 | Caldilinea aerophila DSM 14535 = NBRC 104270 | Bacteria | −7.95 | 3.64 |
| 511051 | Caldisericum exile AZM16c01 | Bacteria | −3.24 | 2.61 |
| 768670 | Calditerrivibrio nitroreducens DSM 19672 | Bacteria | −3.17 | 2.42 |
| 880073 | Caldithrix abyssi DSM 13497 | Bacteria | −4.28 | 2.97 |
| 192222 | Campylobacter jejuni subsp. jejuni NCTC 11168 = | Bacteria | −2.86 | 2.36 |
| | ATCC 700819 | | | |
| 1619079 | candidate division TM6 bacterium | Bacteria | −3.19 | 2.55 |
| | GW2011_GWF2_32_72 | | | |
| 1618609 | Candidatus Azambacteria bacterium | Bacteria | −3.95 | 3.38 |
| | GW2011_GWA1_42_19 | | | |
| 1618623 | Candidatus Azambacteria bacterium | Bacteria | −4.55 | 3.56 |
| | GW2011_GWD2_46_48 | | | |
| 1618369 | Candidatus Beckwithbacteria bacterium | Bacteria | −4.21 | 3.32 |
| | GW2011_GWA2_43_10 | | | |
| 203907 | Candidatus Blochmannia floridanus | Bacteria | −2.58 | 2.33 |
| 1618380 | Candidatus Collierbacteria bacterium | Bacteria | −4.41 | 3.20 |
| | GW2011_GWA2_44_99 | | | |
| 1618405 | Candidatus Curtissbacteria bacterium | Bacteria | −4.02 | 3.01 |
| | GW2011_GWA1_40_16 | | | |
| 477974 | Candidatus Desulforudis audaxviator MP104C | Bacteria | −8.62 | 4.07 |
| 1408204 | Candidatus Endomicrobium trichonymphae | Bacteria | −3.51 | 2.65 |
| 1429438 | Candidatus Entotheonella sp. TSY1 | Bacteria | −7.73 | 3.71 |
| 1429439 | Candidatus Entotheonella sp. TSY2 | Bacteria | −7.77 | 3.75 |
| 1618643 | Candidatus Falkowbacteria bacterium | Bacteria | −4.34 | 3.15 |
| | GW2011_GWF2_43_32 | | | |
| 1618443 | Candidatus Gottesmanbacteria bacterium | Bacteria | −4.33 | 3.01 |
| | GW2011_GWA2_43_14 | | | |
| 1427984 | Candidatus Hepatoplasma crinochetorum Av | Bacteria | −1.88 | 2.01 |
| 1618662 | Candidatus Jorgensenbacteria bacterium | Bacteria | −4.77 | 3.39 |
| | GW2011_GWA2_45_13 | | | |
| 1618671 | Candidatus Kaiserbacteria bacterium | Bacteria | −6.07 | 3.43 |
| | GW2011_GWA2_52_12 | | | |
| 1618673 | Candidatus Kaiserbacteria bacterium | Bacteria | −5.94 | 3.33 |
| | GW2011_GWB1_50_17 | | | |
| 1208920 | Candidatus Kinetoplastibacterium oncopeltii | Bacteria | −3.27 | 2.59 |
| | TCC290E | | | |
| 1619051 | Candidatus Magasanikbacteria bacterium | Bacteria | −4.41 | 3.26 |
| | GW2011_GWD2_43_18 | | | |
| 29290 | Candidatus Magnetobacterium bavaricum | Bacteria | −5.15 | 3.29 |
| 903503 | Candidatus Moranella endobia PCIT | Bacteria | −5.00 | 3.11 |
| 1618729 | Candidatus Nomurabacteria bacterium | Bacteria | −3.51 | 3.10 |
| | GW2011_GWA1_37_20 | | | |
| 1618742 | Candidatus Nomurabacteria bacterium | Bacteria | −3.56 | 3.16 |
| | GW2011_GWB1_37_5 | | | |
| 1618775 | Candidatus Nomurabacteria bacterium | Bacteria | −3.10 | 2.66 |
| | GW2011_GWF2_36_19 | | | |
| 1618777 | Candidatus Nomurabacteria bacterium | Bacteria | −3.64 | 3.04 |
| | GW2011_GWF2_40_31 | | | |
| 1002672 | Candidatus Pelagibacter sp. IMCC9063 | Bacteria | −2.65 | 2.38 |
| 1619068 | Candidatus Peregrinibacteria bacterium | Bacteria | −4.06 | 3.01 |
| | GW2011_GWF2_43_17 | | | |
| 1236703 | Candidatus Photodesmus katoptron Akat1 | Bacteria | −2.89 | 2.40 |
| 234267 | Candidatus Solibacter usitatus Ellin6076 | Bacteria | −8.60 | 3.91 |
| 1618595 | Candidatus Woesebacteria bacterium | Bacteria | −3.86 | 2.65 |
| | GW2011_GWD2_40_19 | | | |
| 1619005 | Candidatus Wolfebacteria bacterium | Bacteria | −5.21 | 3.37 |
| | GW2011_GWA2_47_9b | | | |
| 1619029 | Candidatus Yanofskybacteria bacterium | Bacteria | −4.16 | 3.37 |
| | GW2011_GWC2_41_9 | | | |
| 521097 | Capnocytophaga ochracea DSM 7271 | Bacteria | −3.17 | 2.59 |
| 479433 | Catenulispora acidiphila DSM 44928 | Bacteria | −11.57 | 4.19 |
| 190650 | Caulobacter crescentus CB15 | Bacteria | −11.35 | 4.15 |
| 979 | Cellulophaga lytica | Bacteria | −2.79 | 2.31 |
| 1319815 | Cetobacterium somerae ATCC BAA-474 | Bacteria | −2.44 | 2.20 |
| 218497 | Chlamydia abortus S26-3 | Bacteria | −4.05 | 2.77 |
| 115713 | Chlamydophila pneumoniae CWL029 | Bacteria | −4.14 | 2.78 |
| 138677 | Chlamydophila pneumoniae J138 | Bacteria | −4.13 | 2.79 |
| 517417 | Chlorobaculum parvum NCIB 8327 | Bacteria | −7.04 | 3.52 |
| 194439 | Chlorobium tepidum TLS | Bacteria | −6.87 | 3.67 |
| 326427 | Chloroflexus aggregans DSM 9485 | Bacteria | −7.93 | 3.49 |
| 324602 | Chloroflexus aurantiacus J-10-fl | Bacteria | −7.99 | 3.50 |
| 517418 | Chloroherpeton thalassium ATCC 35110 | Bacteria | −4.86 | 3.02 |
| 243365 | Chromobacterium violaceum ATCC 12472 | Bacteria | −10.55 | 4.73 |
| 345663 | Chryseobacterium greenlandense | Bacteria | −3.04 | 2.39 |
| 1303518 | Chthonomonas calidirosea T49 | Bacteria | −6.82 | 3.56 |
| 443906 | Clavibacter michiganensis subsp. michiganensis | Bacteria | −13.11 | 4.54 |
| | NCPPB 382 | | | |
| 866499 | Cloacibacillus evryensis DSM 19522 | Bacteria | −7.15 | 3.84 |
| 642492 | Clostridium lentocellum DSM 5427 | Bacteria | −3.17 | 2.45 |
| 212717 | Clostridium tetani E88 | Bacteria | −2.38 | 2.16 |
| 1055104 | Cobetia amphilecti str. KMM 296 | Bacteria | −9.78 | 3.83 |
| 469383 | Conexibacter woesei DSM 14684 | Bacteria | −13.54 | 4.68 |
| 583355 | Coraliomargarita akajimensis DSM 45221 | Bacteria | −6.88 | 3.36 |
| 196164 | Corynebacterium efficiens YS-314 | Bacteria | −9.51 | 4.07 |
| 196627 | Corynebacterium glutamicum ATCC 13032 | Bacteria | −7.04 | 3.39 |
| 227377 | Coxiella burnetii RSA 493 | Bacteria | −4.71 | 3.31 |
| 216432 | Croceibacter atlanticus HTCC2559 | Bacteria | −3.14 | 2.45 |
| 1529318 | Cryobacterium sp. MLB-32 | Bacteria | −9.80 | 4.10 |
| 1292022 | Curtobacterium flaccumfaciens UCD-AKU | Bacteria | −12.26 | 4.22 |
| 639282 | Deferribacter desulfuricans SSM1 | Bacteria | −2.61 | 2.26 |
| 255470 | Dehalococcoides mccartyi CBDB1 | Bacteria | −5.59 | 3.32 |
| 1432061 | Dehalococcoides mccartyi CG5 | Bacteria | −5.60 | 3.36 |
| 552811 | Dehalogenimonas lykanthroporepellens BL-DC-9 | Bacteria | −7.67 | 3.97 |
| 319795 | Deinococcus geothermalis DSM 11300 str. | Bacteria | −10.52 | 4.13 |
| | DSM11300 | | | |
| 937777 | Deinococcus peraridilitoris DSM 19664 | Bacteria | −9.68 | 4.09 |
| 1182568 | Deinococcus puniceus | Bacteria | −8.80 | 3.60 |
| 243230 | Deinococcus radiodurans R1 | Bacteria | −10.48 | 4.14 |
| 522772 | Denitrovibrio acetiphilus DSM 12809 | Bacteria | −4.53 | 2.96 |
| 651182 | Desulfobacula toluolica Tol2 | Bacteria | −4.21 | 2.96 |
| 555779 | Desulfonatronospira thiodismutans ASO3-1 | Bacteria | −6.33 | 3.57 |
| 768706 | Desulfosporosinus orientis DSM 765 | Bacteria | −4.57 | 2.94 |
| 882 | Desulfovibrio vulgaris str. Hildenborough | Bacteria | −9.16 | 4.02 |
| 653733 | Desulfurispirillum indicum S5 | Bacteria | −7.34 | 3.84 |
| 868864 | Desulfurobacterium thermolithotrophum DSM | Bacteria | −3.58 | 2.58 |
| | 11699 | | | |
| 910314 | Dialister microaerophilus UPII 345-E | Bacteria | −3.14 | 2.53 |
| 309799 | Dictyoglomus thermophilum H-6-12 | Bacteria | −3.18 | 2.60 |
| 515635 | Dictyoglomus turgidum DSM 6724 | Bacteria | −3.31 | 2.67 |
| 999415 | Eggerthia catenaformis OT 569 = DSM 20559 | Bacteria | −3.07 | 2.41 |
| 445932 | Elusimicrobium minutum Pei191 | Bacteria | −3.75 | 2.91 |
| 226185 | Enterococcus faecalis V583 | Bacteria | −3.39 | 2.61 |
| 1185651 | Enterovibrio norvegicus FF-454 | Bacteria | −5.80 | 3.10 |
| 314225 | Erythrobacter litoralis HTCC2594 | Bacteria | −9.85 | 3.90 |
| 511145 | Escherichia coli str. K-12 substr. MG1655 | Bacteria | −6.58 | 3.40 |
| 316407 | Escherichia coli str. K-12 substr. W3110 | Bacteria | −6.57 | 3.40 |
| 360911 | Exiguobacterium sp. AT1b | Bacteria | −5.33 | 3.11 |
| 381764 | Fervidobacterium nodosum Rt17-B1 | Bacteria | −3.15 | 2.44 |
| 59374 | Fibrobacter succinogenes subsp. succinogenes S85 | Bacteria | −5.45 | 3.10 |
| 661478 | Fimbriimonas ginsengisoli Gsoil 348 | Bacteria | −8.61 | 3.73 |
| 391603 | Flavobacteriales bacterium ALC-1 | Bacteria | −3.00 | 2.35 |
| 1341181 | Flavobacterium limnosediminis JC2902 | Bacteria | −3.44 | 2.58 |
| 402612 | Flavobacterium psychrophilum JIP02/86 | Bacteria | −2.55 | 2.31 |
| 755732 | Fluviicola taffensis DSM 16823 | Bacteria | −3.44 | 2.49 |
| 1347342 | Formosa agariphila KMM 3901 | Bacteria | −2.87 | 2.32 |
| 767434 | Frateuria aurantia DSM 6220 | Bacteria | −10.57 | 4.12 |
| 930946 | Fructobacillus fructosus KCTC 3544 | Bacteria | −4.85 | 3.07 |
| 469615 | Fusobacterium gonidiaformans ATCC 25563 | Bacteria | −2.73 | 2.32 |
| 190304 | Fusobacterium nucleatum subsp. nucleatum ATCC | Bacteria | −2.19 | 2.12 |
| | 25586 | | | |
| 469599 | Fusobacterium periodonticum 2_1_31 | Bacteria | −2.25 | 2.17 |
| 555500 | Galbibacter marinus | Bacteria | −3.42 | 2.68 |
| 553190 | Gardnerella vaginalis 409-05 | Bacteria | −5.25 | 3.13 |
| 49280 | Gelidibacter algens | Bacteria | −3.33 | 2.53 |
| 1630693 | Gemmata sp. SH-PL17 | Bacteria | −9.47 | 4.10 |
| 379066 | Gemmatimonas aurantiaca T-27 | Bacteria | −10.48 | 4.09 |
| 1379270 | Gemmatimonas phototrophica | Bacteria | −10.45 | 4.03 |
| 861299 | Gemmatirosa kalamazoonesis | Bacteria | −13.25 | 4.45 |
| 1121915 | Geoalkalibacter ferrihydriticus DSM 17813 | Bacteria | −7.90 | 3.85 |
| 235909 | Geobacillus kaustophilus HTA426 | Bacteria | −6.55 | 3.75 |
| 272567 | Geobacillus stearothermophilus 10 | Bacteria | −6.83 | 3.67 |
| 398767 | Geobacter lovleyi SZ | Bacteria | −7.17 | 3.66 |
| 1183438 | Gloeobacter kilaueensis JS1 | Bacteria | −8.61 | 3.97 |
| 251221 | Gloeobacter violaceus PCC 7421 | Bacteria | −9.22 | 4.15 |
| 290633 | Gluconobacter oxydans 621H | Bacteria | −9.07 | 3.92 |
| 411154 | Gramella forsetii KT0803 | Bacteria | −3.31 | 2.56 |
| 391165 | Granulibacter bethesdensis CGDNIH1 | Bacteria | −9.08 | 3.99 |
| 233412 | Haemophilus ducreyi 35000HP | Bacteria | −3.95 | 2.69 |
| 866895 | Halobacillus halophilus DSM 2266 | Bacteria | −4.07 | 2.87 |
| 862908 | Halobacteriovorax marinus SJ | Bacteria | −3.91 | 2.76 |
| 1033810 | Haloplasma contractile SSD-17B | Bacteria | −2.88 | 2.38 |
| 373903 | Halothermothrix orenii H 168 | Bacteria | −3.77 | 2.86 |
| 555778 | Halothiobacillus neapolitanus c2 | Bacteria | −7.18 | 3.67 |
| 85962 | Helicobacter pylori 26695 | Bacteria | −3.79 | 2.74 |
| 316274 | Herpetosiphon aurantiacus DSM 785 | Bacteria | −6.46 | 3.46 |
| 760142 | Hippea maritima DSM 10411 | Bacteria | −3.59 | 2.60 |
| 1321371 | Holospora undulata HU1 | Bacteria | −3.79 | 2.71 |
| 1172194 | Hydrocarboniphaga effusa AP103 | Bacteria | −10.69 | 4.24 |
| 608538 | Hydrogenobacter thermophilus TK-6 | Bacteria | −4.88 | 3.00 |
| 547144 | Hydrogenobaculum sp. HO | Bacteria | −3.55 | 2.53 |
| 945713 | Ignavibacterium album JCM 16511 | Bacteria | −2.97 | 2.36 |
| 1313172 | Ilumatobacter coccineus YM16-304 | Bacteria | −10.28 | 4.10 |
| 572544 | Ilyobacter polytropus DSM 2926 | Bacteria | −2.99 | 2.41 |
| 946077 | Imtechella halotolerans K1 | Bacteria | −3.13 | 2.45 |
| 743718 | Isoptericola variabilis 225 | Bacteria | −13.67 | 4.28 |
| 575540 | Isosphaera pallida ATCC 43644 | Bacteria | −8.48 | 3.98 |
| 926559 | Joostella marina DSM 19592 | Bacteria | −2.90 | 2.43 |
| 266940 | Kineococcus radiotolerans SRS30216 = ATCC | Bacteria | −13.51 | 4.74 |
| | BAA-149 | | | |
| 452652 | Kitasatospora setae KM-6054 | Bacteria | −12.91 | 4.84 |
| 1125630 | Klebsiella pneumoniae subsp. pneumoniae HS11286 | Bacteria | −7.91 | 4.12 |
| 1006000 | Kluyvera ascorbata ATCC 33433 | Bacteria | −7.34 | 3.68 |
| 521045 | Kosmotoga olearia TBF 19.5.1 | Bacteria | −4.41 | 2.80 |
| 1330330 | Kosmotoga pacifica | Bacteria | −4.61 | 2.91 |
| 485913 | Ktedonobacter racemifer DSM 44963 | Bacteria | −6.80 | 3.64 |
| 983544 | Lacinutrix sp. 5H-3-7-4 | Bacteria | −2.67 | 2.23 |
| 257314 | Lactobacillus johnsonii NCC 533 | Bacteria | −3.13 | 2.46 |
| 220668 | Lactobacillus plantarum WCFS1 | Bacteria | −4.87 | 3.00 |
| 420890 | Lactococcus garvieae Lg2 | Bacteria | −3.62 | 2.71 |
| 272623 | Lactococcus lactis subsp. lactis Il1403 | Bacteria | −3.40 | 2.52 |
| 911008 | Leclercia adecarboxylata ATCC 23216 = NBRC | Bacteria | −7.40 | 3.62 |
| | 102595 | | | |
| 398720 | Leeuwenhoekiella blandensis MED217 | Bacteria | −3.84 | 2.84 |
| 281090 | Leifsonia xyli subsp. xyli str. CTCB07 | Bacteria | −10.99 | 4.53 |
| 1439331 | Lelliottia amnigena CHS 78 | Bacteria | −7.27 | 3.61 |
| 313628 | Lentisphaera araneosa HTCC2155 | Bacteria | −3.98 | 2.93 |
| 456481 | Leptospira biflexa serovar Patoc strain ‘Patoc 1 | Bacteria | −3.92 | 2.72 |
| | (Paris)’ | | | |
| 267671 | Leptospira interrogans serovar Copenhageni str. | Bacteria | −3.73 | 2.64 |
| | Fiocruz L1-130 | | | |
| 1441628 | Leptospirillum ferriphilum YSK | Bacteria | −7.37 | 3.77 |
| 596323 | Leptotrichia goodfellowii F0264 | Bacteria | −2.52 | 2.33 |
| 272626 | Listeria innocua Clip11262 | Bacteria | −3.25 | 2.52 |
| 169963 | Listeria monocytogenes EGD-e | Bacteria | −3.24 | 2.53 |
| 1574623 | Lyngbya confervoides BDU141951 | Bacteria | −7.67 | 3.85 |
| 156889 | Magnetococcus marinus MC-1 | Bacteria | −7.23 | 3.59 |
| 869210 | Marinithermus hydrothermalis DSM 14884 | Bacteria | −11.07 | 4.16 |
| 443254 | Marinitoga piezophila KA3 | Bacteria | −2.65 | 2.32 |
| 504728 | Meiothermus ruber DSM 1279 | Bacteria | −9.75 | 4.13 |
| 754035 | Mesorhizobium australicum WSM2073 | Bacteria | −10.03 | 3.92 |
| 660470 | Mesotoga prima MesG1.Ag.4.2 | Bacteria | −5.20 | 2.92 |
| 481448 | Methylacidiphilum infernorum V4 | Bacteria | −4.76 | 3.16 |
| 419610 | Methylobacterium extorquens PA1 | Bacteria | −11.86 | 4.32 |
| 243233 | Methylococcus capsulatus str. Bath | Bacteria | −9.69 | 4.19 |
| 449447 | Microcystis aeruginosa NIES-843 | Bacteria | −4.61 | 3.33 |
| 500635 | Mitsuokella multacida DSM 20544 | Bacteria | −7.35 | 3.92 |
| 548479 | Mobiluncus curtisii ATCC 43063 | Bacteria | −7.38 | 3.65 |
| 1379858 | Mucispirillum schaedleri ASF457 | Bacteria | −2.97 | 2.46 |
| 886377 | Muricauda ruestringensis DSM 13258 | Bacteria | −3.99 | 2.82 |
| 272631 | Mycobacterium leprae TN | Bacteria | −8.92 | 3.78 |
| 83332 | Mycobacterium tuberculosis H37Rv | Bacteria | −10.58 | 4.11 |
| 347257 | Mycoplasma agalactiae PG2 | Bacteria | −2.66 | 2.24 |
| 243273 | Mycoplasma genitalium G37 | Bacteria | −2.67 | 2.31 |
| 272632 | Mycoplasma mycoides subsp. mycoides SC str. PG1 | Bacteria | −2.03 | 2.06 |
| 272633 | Mycoplasma penetrans HF-2 | Bacteria | −2.45 | 2.12 |
| 272634 | Mycoplasma pneumoniae M129 | Bacteria | −3.83 | 2.95 |
| 272635 | Mycoplasma pulmonis UAB CTIP | Bacteria | −2.36 | 2.15 |
| 457570 | Natranaerobius thermophilus JW/NM-WN-LF | Bacteria | −3.47 | 2.58 |
| 122586 | Neisseria meningitidis MC58 | Bacteria | −6.21 | 3.68 |
| 1028800 | Neorhizobium galegae bv. orientalis str. HAMBI | Bacteria | −9.33 | 3.79 |
| | 540 | | | |
| 1189621 | Nitritalea halalkaliphila LW7 | Bacteria | −5.65 | 3.53 |
| 314278 | Nitrococcus mobilis Nb-231 | Bacteria | −8.91 | 3.85 |
| 1129897 | Nitrolancea hollandica Lb | Bacteria | −9.68 | 3.95 |
| 228410 | Nitrosomonas europaea ATCC 19718 | Bacteria | −6.22 | 3.35 |
| 1266370 | Nitrospina gracilis 3-211 | Bacteria | −7.03 | 3.78 |
| 330214 | Nitrospira defluvii | Bacteria | −8.11 | 3.68 |
| 196162 | Nocardioides sp. JS614 | Bacteria | −12.35 | 4.28 |
| 592029 | Nonlabens dokdonensis DSW-6 | Bacteria | −3.39 | 2.53 |
| 63737 | Nostoc punctiforme PCC 73102 | Bacteria | −4.57 | 2.91 |
| 670487 | Oceanithermus profundus DSM 14977 | Bacteria | −11.60 | 4.51 |
| 221109 | Oceanobacillus iheyensis HTE831 | Bacteria | −3.26 | 2.54 |
| 203123 | Oenococcus oeni PSU-1 | Bacteria | −3.60 | 2.60 |
| 633147 | Olsenella uli DSM 7084 | Bacteria | −10.15 | 3.90 |
| 262768 | Onion yellows phytoplasma OY-M | Bacteria | −2.02 | 2.07 |
| 452637 | Opitutus terrae PB90-1 | Bacteria | −10.39 | 4.25 |
| 765420 | Oscillochloris trichoides DG-6 | Bacteria | −8.59 | 3.78 |
| 926562 | Owenweeksia hongkongensis DSM 17368 | Bacteria | −4.12 | 2.93 |
| 765952 | Parachlamydia acanthamoebae UV-7 | Bacteria | −3.74 | 2.70 |
| 153151 | Parageobacillus toebii | Bacteria | −4.25 | 2.97 |
| 1618821 | Parcubacteria group bacterium | Bacteria | −4.38 | 3.44 |
| | GW2011_GWA2_42_18 | | | |
| 1618840 | Parcubacteria group bacterium | Bacteria | −5.21 | 3.50 |
| | GW2011_GWA2_47_10b | | | |
| 1618841 | Parcubacteria group bacterium | Bacteria | −5.02 | 3.45 |
| | GW2011_GWA2_47_12 | | | |
| 1618924 | Parcubacteria group bacterium | Bacteria | −3.99 | 3.21 |
| | GW2011_GWC2_40_31 | | | |
| 402881 | Parvibaculum lavamentivorans DS-1 | Bacteria | −9.88 | 4.00 |
| 314260 | Parvularcula bermudensis HTCC2503 | Bacteria | −9.23 | 4.02 |
| 747 | Pasteurella multocida str. ATCC 43137 | Bacteria | −4.05 | 2.64 |
| 123214 | Persephonella marina EX-H1 | Bacteria | −3.52 | 2.55 |
| 403833 | Petrotoga mobilis SJ95 | Bacteria | −3.01 | 2.37 |
| 298386 | Photobacterium profundum SS9 | Bacteria | −4.79 | 2.96 |
| 243265 | Photorhabdus luminescens subsp. laumondii TTO1 | Bacteria | −4.70 | 3.00 |
| 1142394 | Phycisphaera mikurensis NBRC 102666 | Bacteria | −13.64 | 4.91 |
| 1227812 | Piscirickettsia salmonis LF-89 = ATCC VR-1361 | Bacteria | −4.39 | 2.99 |
| 521674 | Planctopirus limnophila DSM 3776 | Bacteria | −6.98 | 3.44 |
| 431947 | Porphyromonas gingivalis ATCC 33277 | Bacteria | −5.21 | 3.29 |
| 167546 | Prochlorococcus marinus str. MIT 9301 | Bacteria | −2.99 | 2.40 |
| 208964 | Pseudomonas aeruginosa PAO1 | Bacteria | −10.98 | 4.39 |
| 96563 | Pseudomonas stutzeri | Bacteria | −10.16 | 4.07 |
| 1123384 | Pseudothermotoga hypogea DSM 11164 = NBRC | Bacteria | −6.20 | 3.10 |
| | 106472 | | | |
| 259536 | Psychrobacter arcticus 273-4 | Bacteria | −5.05 | 2.92 |
| 335284 | Psychrobacter cryohalolentis K5 | Bacteria | −5.07 | 2.92 |
| 1189619 | Psychroflexus gondwanensis ACAM 44 | Bacteria | −3.07 | 2.42 |
| 267608 | Ralstonia solanacearum GMI1000 | Bacteria | −11.19 | 4.59 |
| 365046 | Ramlibacter tataouinensis TTB310 | Bacteria | −12.59 | 4.76 |
| 145458 | Rathayibacter toxicus | Bacteria | −9.34 | 4.01 |
| 288705 | Renibacterium salmoninarum ATCC 33209 | Bacteria | −8.11 | 3.57 |
| 1033991 | Rhizobium leguminosarum bv. trifolii CB782 | Bacteria | −9.32 | 3.94 |
| 243090 | Rhodopirellula baltica SH 1 | Bacteria | −7.43 | 3.56 |
| 258594 | Rhodopseudomonas palustris CGA009 | Bacteria | −11.12 | 4.18 |
| 518766 | Rhodothermus marinus DSM 4252 | Bacteria | −9.31 | 4.08 |
| 1165094 | Richelia intracellularis HH01 | Bacteria | −3.85 | 2.64 |
| 313596 | Robiginitalea biformata HTCC2501 | Bacteria | −6.79 | 3.91 |
| 585394 | Roseburia hominis A2-183 | Bacteria | −5.49 | 3.35 |
| 383372 | Roseiflexus castenholzii DSM 13941 | Bacteria | −9.01 | 3.87 |
| 762948 | Rothia dentocariosa ATCC 17931 | Bacteria | −7.10 | 3.61 |
| 582515 | Rubidibacter lacunae KORDI 51-2 | Bacteria | −7.66 | 3.55 |
| 405948 | Saccharopolyspora erythraea NRRL 2338 | Bacteria | −12.24 | 4.31 |
| 435906 | Salegentibacter salarius | Bacteria | −3.35 | 2.57 |
| 407035 | Salinicoccus halodurans | Bacteria | −4.30 | 2.93 |
| 45670 | Salinicoccus roseus | Bacteria | −5.07 | 3.19 |
| 1432562 | Salinicoccus sediminis | Bacteria | −5.00 | 3.23 |
| 1033802 | Salinisphaera shabanensis E1L3A | Bacteria | −9.62 | 3.96 |
| 1307761 | Salinispira pacifica | Bacteria | −6.92 | 3.65 |
| 99287 | Salmonella enterica subsp. enterica serovar | Bacteria | −6.80 | 3.59 |
| | Typhimurium str. LT2 | | | |
| 526218 | Sebaldella termitidis ATCC 33386 | Bacteria | −2.75 | 2.43 |
| 211586 | Shewanella oneidensis MR-1 | Bacteria | −5.53 | 3.03 |
| 1454006 | Siansivirga zeaxanthinifaciens CC-SAMT-1 | Bacteria | −2.82 | 2.35 |
| 331113 | Simkania negevensis Z | Bacteria | −4.24 | 3.00 |
| 886293 | Singulisphaera acidiphila DSM 18658 | Bacteria | −8.97 | 3.92 |
| 266834 | Sinorhizobium meliloti 1021 | Bacteria | −9.62 | 3.85 |
| 742818 | Slackia piriformis YIT 12062 | Bacteria | −8.20 | 3.62 |
| 929556 | Solitalea canadensis DSM 3403 | Bacteria | −3.58 | 2.60 |
| 479434 | Sphaerobacter thermophilus DSM 20745 | Bacteria | −11.47 | 4.10 |
| 158189 | Sphaerochaeta globosa str. Buddy | Bacteria | −5.97 | 3.12 |
| 446470 | Stackebrandtia nassauensis DSM 44728 | Bacteria | −10.58 | 4.21 |
| 93061 | Staphylococcus aureus subsp. aureus NCTC 8325 | Bacteria | −2.78 | 2.33 |
| 176280 | Staphylococcus epidermidis ATCC 12228 | Bacteria | −2.75 | 2.34 |
| 519441 | Streptobacillus moniliformis DSM 12112 | Bacteria | −2.03 | 2.09 |
| 160490 | Streptococcus pyogenes M1 GAS | Bacteria | −3.84 | 2.61 |
| 227882 | Streptomyces avermitilis MA-4680 = NBRC 14893 | Bacteria | −11.81 | 4.23 |
| 100226 | Streptomyces coelicolor A3(2) | Bacteria | −12.42 | 4.41 |
| 1469144 | Streptomyces thermoautotrophicus | Bacteria | −12.24 | 4.23 |
| 762983 | Succinatimonas hippei YIT 12066 | Bacteria | −4.37 | 2.94 |
| 204536 | Sulfurihydrogenibium azorense Az-Fu1 | Bacteria | −2.84 | 2.27 |
| 432331 | Sulfurihydrogenibium yellowstonense SS-5 | Bacteria | −2.92 | 2.44 |
| 326298 | Sulfurimonas denitrificans DSM 1251 | Bacteria | −3.37 | 2.53 |
| 269084 | Synechococcus elongatus PCC 6301 | Bacteria | −7.57 | 3.41 |
| 316279 | Synechococcus sp. CC9902 | Bacteria | −7.55 | 3.67 |
| 1148 | Synechocystis sp. PCC 6803 | Bacteria | −5.51 | 3.22 |
| 1209989 | Tepidanaerobacter acetatoxydans Re1 | Bacteria | −3.49 | 2.52 |
| 1208320 | Thalassolituus oleivorans R6-15 | Bacteria | −5.81 | 3.21 |
| 1177928 | Thalassospira profundimaris WP0211 | Bacteria | −7.93 | 3.52 |
| 525903 | Thermanaerovibrio acidaminovorans DSM 6589 | Bacteria | −10.26 | 4.23 |
| 525904 | Thermobaculum terrenum ATCC BAA-798 | Bacteria | −7.29 | 4.09 |
| 269800 | Thermobifida fusca YX | Bacteria | −10.75 | 4.12 |
| 469371 | Thermobispora bispora DSM 43833 | Bacteria | −12.92 | 4.56 |
| 638303 | Thermocrinis albus DSM 14484 | Bacteria | −5.52 | 3.11 |
| 667014 | Thermodesulfatator indicus DSM 15286 | Bacteria | −4.52 | 3.16 |
| 289377 | Thermodesulfobacterium commune DSM 2178 | Bacteria | −3.55 | 2.61 |
| 795359 | Thermodesulfobacterium geofontis OPF15 | Bacteria | −2.76 | 2.37 |
| 289376 | Thermodesulfovibrio yellowstonii DSM 11347 | Bacteria | −3.23 | 2.54 |
| 309801 | Thermomicrobium roseum DSM 5159 | Bacteria | −10.32 | 3.73 |
| 484019 | Thermosipho africanus TCF52B | Bacteria | −2.83 | 2.38 |
| 391009 | Thermosipho melanesiensis BI429 | Bacteria | −2.70 | 2.29 |
| 1298851 | Thermosulfidibacter takaii ABI70S6 | Bacteria | −4.55 | 2.80 |
| 243274 | Thermotoga maritima MSB8 | Bacteria | −5.41 | 3.10 |
| 648996 | Thermovibrio ammonificans HB-1 | Bacteria | −6.18 | 3.46 |
| 580340 | Thermovirga lienii DSM 17291 | Bacteria | −5.35 | 3.18 |
| 498848 | Thermus aquaticus Y51MC23 | Bacteria | −11.50 | 4.16 |
| 751945 | Thermus oshimai JL-2 | Bacteria | −11.68 | 4.25 |
| 300852 | Thermus thermophilus HB8 | Bacteria | −11.93 | 4.26 |
| 768671 | Thiocapsa marina 5811 | Bacteria | −10.02 | 3.95 |
| 381306 | Thiohalorhabdus denitrificans | Bacteria | −11.69 | 4.44 |
| 1177931 | Thiovulum sp. ES | Bacteria | −3.26 | 2.46 |
| 1245935 | Tolypothrix campylonemoides VB511288 | Bacteria | −5.54 | 3.78 |
| 243275 | Treponema denticola ATCC 35405 | Bacteria | −3.85 | 2.96 |
| 203124 | Trichodesmium erythraeum IMS101 | Bacteria | −3.60 | 2.60 |
| 203267 | Tropheryma whipplei str. Twist | Bacteria | −5.95 | 3.22 |
| 649638 | Truepera radiovictrix DSM 17093 | Bacteria | −11.76 | 4.46 |
| 1157490 | Tumebacillus flagellatus | Bacteria | −7.08 | 3.58 |
| 883169 | Turicella otitidis ATCC 51513 | Bacteria | −12.43 | 4.67 |
| 505682 | Ureaplasma parvum serovar 3 str. ATCC 27815 | Bacteria | −2.15 | 2.12 |
| 263358 | Verrucosispora maris AB-18-032 | Bacteria | −11.52 | 4.54 |
| 388396 | Vibrio fischeri MJ11 | Bacteria | −4.07 | 2.68 |
| 223926 | Vibrio parahaemolyticus RIMD 2210633 | Bacteria | −5.27 | 3.00 |
| 196600 | Vibrio vulnificus YJ016 | Bacteria | −5.54 | 3.15 |
| 641526 | Winogradskyella psychrotolerans RS-3 | Bacteria | −3.06 | 2.40 |
| 1116230 | Wolbachia pipientis wAlbB | Bacteria | −3.40 | 2.50 |
| 273121 | Wolinella succinogenes DSM 1740 | Bacteria | −5.74 | 3.51 |
| 1304892 | Xanthomonas axonopodis Xac29-1 | Bacteria | −10.57 | 4.13 |
| 190485 | Xanthomonas campestris pv. campestris str. ATCC | Bacteria | −10.86 | 4.24 |
| | 33913 | | | |
| 160492 | Xylella fastidiosa 9a5c | Bacteria | −6.73 | 3.74 |
| 155920 | Xylella fastidiosa subsp. sandyi Ann-1 | Bacteria | −6.99 | 3.76 |
| 655815 | Zunongwangia profunda SM-A87 | Bacteria | −3.26 | 2.62 |
| 1257118 | Acanthamoeba castellanii str. Neff | Eukaryotes | −7.39 | 3.96 |
| 104782 | Adineta vaga | Eukaryotes | −3.01 | 2.40 |
| 65357 | Albugo candida | Eukaryotes | −4.80 | 2.78 |
| 578462 | Allomyces macrogynus ATCC 38327 | Eukaryotes | −9.88 | 4.21 |
| 400682 | Amphimedon queenslandica | Eukaryotes | −4.15 | 3.05 |
| 5061 | Aspergillus niger | Eukaryotes | −6.42 | 3.40 |
| 44056 | Aureococcus anophagefferens | Eukaryotes | −11.25 | 4.93 |
| 484906 | Babesia bovis T2Bo | Eukaryotes | −4.96 | 3.11 |
| 753081 | Bigelowiella natans | Eukaryotes | −5.16 | 3.09 |
| 930990 | Botryobasidium botryosum FD-172 SS1 | Eukaryotes | −6.74 | 3.52 |
| 237561 | Candida albicans SC5314 | Eukaryotes | −3.29 | 2.47 |
| 595528 | Capsaspora owczarzaki ATCC 30864 | Eukaryotes | −7.02 | 3.37 |
| 3055 | Chlamydomonas reinhardtii | Eukaryotes | −11.16 | 4.64 |
| 2769 | Chondrus crispus (carragheen) | Eukaryotes | −6.42 | 3.60 |
| 574566 | Coccomyxa subellipsoidea C-169 | Eukaryotes | −8.32 | 3.91 |
| 214684 | Cryptococcus neoformans var. neoformans JEC21 | Eukaryotes | −5.74 | 3.17 |
| 2898 | Cryptomonas paramecium | Eukaryotes | −2.31 | 2.12 |
| 353152 | Cryptosporidium parvum Iowa II | Eukaryotes | −2.94 | 2.40 |
| 280699 | Cyanidioschyzon merolae | Eukaryotes | −7.85 | 3.60 |
| 6669 | Daphnia pulex | Eukaryotes | −4.90 | 3.20 |
| 352472 | Dictyostelium discoideum AX4 | Eukaryotes | −2.16 | 2.20 |
| 420778 | Diplodia seriata | Eukaryotes | −7.70 | 3.71 |
| 3046 | Dunaliella salina | Eukaryotes | −7.35 | 3.64 |
| 280463 | Emiliania huxleyi CCMP1516 | Eukaryotes | −10.83 | 4.40 |
| 885318 | Entamoeba histolytica HM-1: IMSS-A | Eukaryotes | −2.60 | 2.24 |
| 931890 | Eremothecium cymbalariae DBVPG#7215 | Eukaryotes | −4.31 | 2.84 |
| 284811 | Eremothecium gossypii ATCC 10895 (assembly | Eukaryotes | −6.55 | 3.76 |
| | ASM9102v4) | | | |
| 1519565 | Fistulifera solans | Eukaryotes | −5.51 | 3.02 |
| 691883 | Fonticula alba | Eukaryotes | −9.87 | 4.39 |
| 635003 | Fragilariopsis cylindrus CCMP1102 | Eukaryotes | −4.24 | 2.88 |
| 130081 | Galdieria sulphuraria | Eukaryotes | −4.09 | 2.61 |
| 184922 | Giardia lamblia ATCC 50803 | Eukaryotes | −6.14 | 3.53 |
| 905079 | Guillardia theta CCMP2712 | Eukaryotes | −6.60 | 3.44 |
| 944289 | Gymnopus luxurians FD-317 M1 | Eukaryotes | −5.42 | 3.04 |
| 945553 | Hypholoma sublateritium FD-334 SS-4 | Eukaryotes | −6.70 | 3.80 |
| 486041 | Laccaria bicolor S238N-H82 | Eukaryotes | −5.70 | 3.28 |
| 347515 | Leishmania major strain Friedlin | Eukaryotes | −8.47 | 3.77 |
| 242507 | Magnaporthe oryzae | Eukaryotes | −7.63 | 3.59 |
| 564608 | Micromonas pusilla CCMP1545 | Eukaryotes | −10.27 | 4.48 |
| 27923 | Mnemiopsis leidyi | Eukaryotes | −4.77 | 2.98 |
| 554373 | Moniliophthora pemiciosa FA553 | Eukaryotes | −5.80 | 3.05 |
| 431895 | Monosiga brevicollis MX1 | Eukaryotes | −7.22 | 3.67 |
| 744533 | Naegleria gruberi strain NEG-M | Eukaryotes | −3.07 | 2.35 |
| 45351 | Nematostella vectensis | Eukaryotes | −5.20 | 3.22 |
| 1287680 | Neofusicoccum parvum UCRNP2 | Eukaryotes | −7.69 | 3.74 |
| 436017 | Ostreococcus lucimarinus | Eukaryotes | −8.48 | 4.17 |
| 412030 | Paramecium tetraurelia strain d4-2 | Eukaryotes | −2.57 | 2.14 |
| 423536 | Perkinsus marinus ATCC 50983 | Eukaryotes | −6.38 | 3.25 |
| 556484 | Phaeodactylum tricornutum CCAP 1055/1 | Eukaryotes | −5.89 | 3.20 |
| 3218 | Physcomitrella patens | Eukaryotes | −5.80 | 3.21 |
| 164328 | Phytophthora ramorum | Eukaryotes | −7.76 | 3.56 |
| 36329 | Plasmodium falciparum 3D7 | Eukaryotes | −2.32 | 2.28 |
| 4781 | Plasmopara halstedii | Eukaryotes | −5.31 | 3.00 |
| 1069680 | Pneumocystis murina b123 | Eukaryotes | −2.66 | 2.26 |
| 561896 | Postia placenta Mad-698-R | Eukaryotes | −7.34 | 3.56 |
| 418459 | Puccinia graminis f. sp. tritici | Eukaryotes | −5.16 | 3.46 |
| 1223560 | Pythium vexans DAOM BR484 | Eukaryotes | −8.78 | 3.83 |
| 559292 | Saccharomyces cerevisiae S288c | Eukaryotes | −3.99 | 2.69 |
| 946362 | Salpingoeca rosetta | Eukaryotes | −7.17 | 3.71 |
| 695850 | Saprolegnia parasitica CBS 223.65 | Eukaryotes | −8.19 | 3.74 |
| 578458 | Schizophyllum commune H4-8 | Eukaryotes | −7.94 | 3.80 |
| 284812 | Schizosaccharomyces pombe (strain 972/ATCC | Eukaryotes | −4.09 | 2.67 |
| | 24843) | | | |
| 29656 | Spirodela polyrhiza | Eukaryotes | −7.46 | 3.98 |
| 645134 | Spizellomyces punctatus DAOM BR117 | Eukaryotes | −5.67 | 3.09 |
| 1397361 | Sporothrix schenckii 1099-18 | Eukaryotes | −8.06 | 3.75 |
| 312017 | Tetrahymena thermophila SB210 | Eukaryotes | −2.39 | 2.18 |
| 296543 | Thalassiosira pseudonana | Eukaryotes | −5.44 | 2.87 |
| 353154 | Theileria annulata strain Ankara | Eukaryotes | −2.79 | 2.51 |
| 508771 | Toxoplasma gondii ME49 | Eukaryotes | −6.86 | 3.49 |
| 412133 | Trichomonas vaginalis G3 | Eukaryotes | −3.13 | 2.60 |
| 10228 | Trichoplax adhaerens | Eukaryotes | −3.69 | 2.54 |
| 5693 | Trypanosoma cruzi | Eukaryotes | −7.29 | 4.32 |
| 436907 | Vanderwaltozyma polyspora DSM 70294 | Eukaryotes | −3.36 | 2.53 |
| 3067 | Volvox carteri | Eukaryotes | −8.85 | 4.22 |
| 4927 | Wickerhamomyces anomalus NRRL Y-366-8 | Eukaryotes | −3.55 | 2.44 |
| 1041607 | Wickerhamomyces ciferrii | Eukaryotes | −3.09 | 2.31 |
| 1047168 | Zymoseptoria brevis | Eukaryotes | −6.68 | 3.35 |
| 336722 | Zymoseptoria tritici | Eukaryotes | −6.69 | 3.31 |

In some embodiments, the threshold is species-specific. In some embodiments, the threshold is domain-specific. In some embodiments, the threshold is kingdom specific. In some embodiments, the threshold is a prokaryotic threshold. In some embodiments, the threshold is a eukaryotic threshold. In some embodiments, the threshold is a archaea threshold. In some embodiments, the threshold is a bacteria threshold.

In some embodiments, the first region comprises at least one codon substituted to another codon. In some embodiments, the first region comprises at plurality of codons substituted to another codon. In some embodiments, each substitution increases folding energy of the first region or RNA encoded by the first region. In some embodiments, the plurality of mutations in combination increases folding energy of the first region or RNA encoded by the first region.

In some embodiments, at least 1, at least 2, at least 3, at least 4, at least 5, at least 10, at least 15, at least 20, at least 25, or at least 30 codons of the first region have been substituted. Each possibility represents a separate embodiment of the present invention. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of all codons in the region have been substituted. Each possibility represents a separate embodiment of the present invention. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of codons in the region that have synonymous codons that increase the folding energy of the region have been substituted. Each possibility represents a separate embodiment of the present invention.

In some embodiments, all possible codons with the first region are substituted to synonymous codons that increase folding energy of the region or RNA encoded by the region. In some embodiments, codons are substituted to synonymous codons to produce a region with the highest possible folding energy while maintaining the amino acid sequence of a peptide encoded by the region. In some embodiments, all possible combinations of synonymous mutations are examined and the combination with the highest folding energy is selected. In some embodiments, the region comprise synonymous codons substituted to increase folding energy to a maximum possible for the region.

In some embodiments, the coding sequence comprises a second region. In some embodiments, the second region is from the translational start site (TSS) to 20 nucleotides downstream of the TSS. In some embodiments, the TSS is a start codon. It will be understood by a skilled artisan that the first base of the start codon is considered base 1, and so bases 1 to 3 of the region are the start codon. In some embodiments, the second region comprises the start codon. In some embodiments, the second region is from the TSS to 10 nucleotides downstream. In some embodiments, the second region is from the TSS to 150 nucleotides downstream. In some embodiments, the second region does not include the start codon. In some embodiments, the second region comprises at least one codon substituted to another codon. In some embodiments, the another codon is a synonymous codon. In some embodiments, the substitution increases folding energy in the second region or of RNA encoded by the second region. In some embodiments, the second region comprises synonymous mutations that increase the folding energy of the region or of RNA encoded by the region to a maximum possible while retaining the amino acid sequence encoded by the region.

In some embodiments, the coding sequence comprises a third region. In some embodiments, the third region is from the first region to the second region. In some embodiments, the third region is between the first region and the second region. In some embodiments, the third region is from the end of the second region to the beginning of the first region. In some embodiments, the third region is between the end of the second region to the beginning of the first region. In some embodiments, the third region does not overlap with the first region, the second region or both. In some embodiments, the third region does not overlap with the first region. In some embodiments, the third region does not overlap with the second region. In some embodiments, the third region overlaps with the second region. In some embodiments, the third region is from 20 to 50 nucleotides downstream of the TSS. In some embodiments, the third region is from 21 to 50 nucleotides downstream of the TSS. In some embodiments, the third region is from 20 to 70 nucleotides downstream of the TSS. In some embodiments, the third region is from 21 to 70 nucleotides downstream of the TSS. In some embodiments, the third region is from 20 to 150 nucleotides downstream of the TSS. In some embodiments, the third region is from 21 to 150 nucleotides downstream of the TSS. In some embodiments, the third region is from 20 to 300 nucleotides downstream of the TSS. In some embodiments, the third region is from 21 to 300 nucleotides downstream of the TSS. In some embodiments, the third region is from 300 to 90 nucleotides upstream of the stop codon. In some embodiments, the third region is from 300 to 70 nucleotides upstream of the stop codon. In some embodiments, the third region is from 300 to 50 nucleotides upstream of the stop codon. In some embodiments, the third region is from 300 to 40 nucleotides upstream of the stop codon. In some embodiments, the third region comprises at least one codon substituted to another codon. In some embodiments, the another codon is a synonymous codon. In some embodiments, the substitution decreases folding energy in the third region or of RNA encoded by the third region. In some embodiments, the third region comprises synonymous mutations that decrease the folding energy of the region or of RNA encoded by the region to a minimum possible while retaining the amino acid sequence encoded by the region.

In some embodiments, the first region is the second region. In some embodiments, the first region is the third region. In some embodiments, the coding sequence comprises only the second region. In some embodiments, the coding region comprises only the third region. In some embodiments, the coding region comprises the second and third regions and not the first region.

Whether a mutation increase or decreases local folding energy can be determined by modeling or empirically. Methods of determining local folding energy are well known in the art and any such method may be employed. Methods are also provided herein and any of these methods may be employed. In some embodiments, the method comprises determining the local folding energy for a region, generating at least one mutation in the region, determining the local folding energy in the mutated region and selecting the mutation if it increases the local folding energy. In some embodiments, the method comprises determining the local folding energy for a region, generating at least one mutation in the region, determining the local folding energy in the mutated region and selecting the mutation if it decreases the local folding energy. In some embodiments, determining local folding energy comprises inputting the sequence into a folding program. In some embodiments, a folding program is a program that predicts RNA folding. In some embodiments, a folding program is a program that models RNA folding. In some embodiments, a folding program provides a folding energy for a sequence. In some embodiments, the folding energy is local folding energy. In some embodiments, local is over a given window. In some embodiments, the window is 40 nt. In some embodiments, the sequence is the sequence of the region. Examples of folding programs are well known in the art and include for example, Mfold, RNAfold, RNA123, RNAshapes, RNAstructure, RNAstructureWeb, RNAslider and UNAFold to name but a few. In some embodiments, local folding energy is determined with RNAfold. Once the local folding energy is found for a given sequence over a given window various mutations can be tested for their effect on local folding energy. A mutation that increases folding energy or a mutation that decreases folding energy can be selected. Multiple mutations can be tested at once, or one at a time. When the folding architecture of a window is known, the mutations can be designed rationally, as generating mismatches in areas of secondary structure will reduce the secondary structure and thus increase local folding energy. Similarly, generating secondary structure where there was none will decrease local folding energy. Since the G-C bonds is stronger than the T-A bond, substituting one for the other can decrease local folding energy (T-A to G-C) or increase local folding energy (G-C to T-A). The predicted local folding energy can be compared to a null model to detect/predict meaningful levels of folding energy changes. A mutant region can also be tested empirically by methods such as are described herein. The region can be inserted into a reporter plasmid comprising a detectable protein (e.g., a fluorescent protein). The detectable protein may be for example GFP or RFP. Changes in expression of the reporter (e.g., GFP) can be monitored. Increases in expression of the reporter indicate that the folding energy just before the stop codon has been increased (i.e., weaker folding) leading to increased translation. Decreases in expression of the reporter indicate that the folding energy just before the stop codon has been decreased leading to decreased translation. Changes made in any of the regions can be measured in this way as well. Weaking folding just after the start codon will improve translation and increasing/decreasing folding in the middle of the CDS will affect translation in different ways depending on the domain/species of the coding/region target cell.

By another aspect, there is provided a vector comprising a nucleic acid molecule of the invention.

In some embodiments, the vector is an expression vector. In some embodiments, the vector is configured for expression in a target cell. In some embodiments, the vector comprises at least one regulatory element for expression in the target cell. In some embodiments, the regulatory element is configured for producing expression in the target cell. In some embodiments, the regulatory element produces expression in the target cell. In some embodiments, the regulatory element regulates expressing on the target cell.

By another aspect, there is provided a cell comprising the expression vector or nucleic acid molecule of the invention.

In some embodiments, the cell is a target cell. In some embodiments, the cell is a archeal cell. In some embodiments, the cell is a bacterial cell. In some embodiments, the cell is a eukaryotic cell. In some embodiments, the eukaryotic cell is anot a fungal cell. In some embodiments, the cell is in culture. In some embodiments, the cell is in vivo. In some embodiments, the cell is ex vivo. In some embodiments, the nucleic acid molecule is optimized for expression in the cell.

According to another aspect, there is provided a method for optimizing a coding sequence, the method comprising introducing a mutation into a first region of the coding sequence, wherein the mutation increases or decreases folding energy of the first region or RNA encoded by the first region.

In some embodiments, the first region is upstream and proximal to the stop codon and the mutation increases folding energy of the first region or RNA encoded by the first region. In some embodiments, the first region is downstream and proximal to the start codon and the mutation increases folding energy of the first region or RNA encoded by the first region. In some embodiments, the first region is in the gene body not proximal to the start codon or stop codon and the mutation decreases folding energy of the first region or RNA encoded by the first region.

In some embodiments, optimizing comprises optimizing expression of a protein encoded by the coding sequence. In some embodiments, optimizing is optimizing in a target cell. In some embodiments, optimizing is optimizing protein expression in a target cell. In some embodiments, optimizing is optimizing expression of a protein from a heterologous transgene in a target cell. In some embodiments, the heterologous transgene is not native to the target cell. In some embodiments, the target cell is a prokaryotic cell. In some embodiments, the target cell is a bacterial cell. In some embodiments, the target cell is an archaeal cell. In some embodiments, the target cell is a eukaryotic cell. In some embodiments, the target cell is a mammalian cell. In some embodiments, the target cell is a human cell. In some embodiments, the coding sequence is a viral, bacterial, archaeal, or eukaryotic sequence. In some embodiments, the coding sequence is exogenous to the target cell.

In some embodiments, the target cell is an archaeal cell and the first region is from 90 nucleotides upstream of the stop codon of the coding sequence to the stop codon. In some embodiments, the target cell is a bacterial cell and the first region is from 50 nucleotides upstream of the stop codon of the coding sequence to the stop codon. In some embodiments, the target cell is a eukaryotic cell and the first region is from 40 nucleotides upstream of the stop codon of the coding sequence to the stop codon.

In some embodiments, the mutation is a synonymous mutation. In some embodiments, the mutation is a silent mutation. In some embodiments, introducing comprises providing a mutated sequence. In some embodiments, introducing comprises providing a mutation or a list of mutations to be made in the coding sequence. In some embodiments, introducing is introducing a plurality of mutations. In some embodiments, each mutation of the plurality of mutations increases folding energy in the first region or RNA encoded by the first region. In some embodiments, a plurality of mutations in combination increases folding energy of the first region or of RNA encoded by the first region.

In some embodiments, the method comprises introducing at least 1, 2, 3, 4, 5, 6, 7, 8, 9, 10, 15, 20, 25 or 30 mutation into the first region. Each possibility represents a separate embodiment of the invention. In some embodiments, the method comprises introducing all possible synonymous mutation that increase folding energy of the first region or RNA encoded by the first region. In some embodiments, the method comprises mutating all possible codons with synonymous codons that increase folding energy of the first region or RNA encoded by the first region. In some embodiments, the method comprises introducing synonymous mutation to produce a first region or RNA encoded by the first region with the maximum possible folding energy. Thus, the method may include calculating all possible synonymous mutations that increase folding energy, and all possible combinations of mutations that increase folding energy and selecting the combination of synonymous mutations that increase the folding energy of the region or RNA encoded by the region the most.

In some embodiments, folding energy is increased. In some embodiments, folding energy is decreased. In some embodiments, the folding energy is folding energy of the coding sequence. In some embodiments, the folding energy is folding energy of the region. In some embodiments, the folding energy is folding energy of the RNA encoded.

In some embodiments, the method further comprises introducing a mutation into a second region. In some embodiments, the second region is from the TSS to 20 nucleotides downstream of the TSS. In some embodiments, the cell is an archaeal cell the second region is from the TSS to 10 nucleotides downstream of the TSS. In some embodiments, the cell is selected from a bacterial cell and a eukaryotic cell and the second region is from the TSS to 20 nucleotides downstream of the TSS. In some embodiments, the mutation increases folding energy of the second region or of RNA encoded by the second region. In some embodiments, the second region is mutated with synonymous mutation such that the folding energy is increased to the maximum while retaining the amino acid sequence encoded by the region.

In some embodiments, the method further comprises introducing a mutation into a third region. In some embodiments, the third region is from the second region to the first region. In some embodiments, the third region is from 20 to 50 nucleotides downstream of the TSS. In some embodiments, the size of the region is organism specific. In some embodiments, the size of the region is domain-specific. In some embodiments, the size of the region is specific to bacteria. In some embodiments, the size of the region is specific to archaea. In some embodiments, the size of the region is specific to prokaryotes. In some embodiments, the size of the region is specific to eukaryotes. In some embodiments, the mutation decreases folding energy of the third region or of RNA encoded by the third region. In some embodiments, the third region is mutated with synonymous mutation such that the folding energy is decreased to the minimum while retaining the amino acid sequence encoded by the region.

In some embodiments, the method is an ex vivo method. In some embodiments, the method is an in vitro method. In some embodiments, the method is performed in a cell.

According to another aspect, there is provided a computer program product comprising a non-transitory computer-readable storage medium having program instructions embodied therewith, the program instructions executable by at least one hardware processor to execute a genetic-type machine learning algorithm configured to perform a method of the invention.

According to another aspect, there is provided a computer program product comprising a non-transitory computer-readable storage medium having program instructions embodied therewith, the program instructions executable by at least one hardware processor to execute a genetic-type machine learning algorithm configured to:
a. receive a coding sequence;
b. determine within a first region of the coding sequence at least one mutation that increases folding energy of the first region or RNA encoded by the first region; and
c. output a mutated coding sequence comprising the at least one mutation.

According to another aspect, there is provided a computer program product comprising a non-transitory computer-readable storage medium having program instructions embodied therewith, the program instructions executable by at least one hardware processor to execute a genetic-type machine learning algorithm configured to:
a. receive a coding sequence;
b. determine within a first region of the coding sequence at least one mutation that increases folding energy of the first region or RNA encoded by the first region; and
c. output a list of possible mutations in the first region that increase folding energy of the first region or RNA encoded by the first region.

In some embodiments, the computer program product optimizes the region for expression in a target cell. In some embodiments, the computer program product determines the combination of mutations that increases folding energy to a maximum while retaining the amino acid sequence of the encoded by the region.

In some embodiments, the computer program product also determines within a second region of the coding sequence at least one mutation that increases folding energy of the second region or RNA encoded by the second region and outputs a mutated coding sequence that further comprises at least one mutation in the second region. In some embodiments, the computer program product also determines within a second region of the coding sequence at least one mutation that increases folding energy of the second region or RNA encoded by the second region and outputs a list of possible mutations that further comprises mutations in the second region that increase folding energy of the second region or of RNA encoded by the second region. In some embodiments, the computer program product determines the combination of mutations in the second region that produces the maximum folding energy while retaining the amino acid sequence encoded by the second region.

In some embodiments, the computer program product also determines within a third region of the coding sequence at least one mutation that decreases folding energy of the third region or RNA encoded by the third region and outputs a mutated coding sequence that further comprises at least one mutation in the third region. In some embodiments, the computer program product also determines within a third region of the coding sequence at least one mutation that decreases folding energy of the third region or RNA encoded by the third region and outputs a list of possible mutations that further comprises mutations in the third region that decreases folding energy of the third region or of RNA encoded by the third region. In some embodiments, the computer program product determines the combination of mutations in the third region that produces the minimum folding energy while retaining the amino acid sequence encoded by the third region.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention may be described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

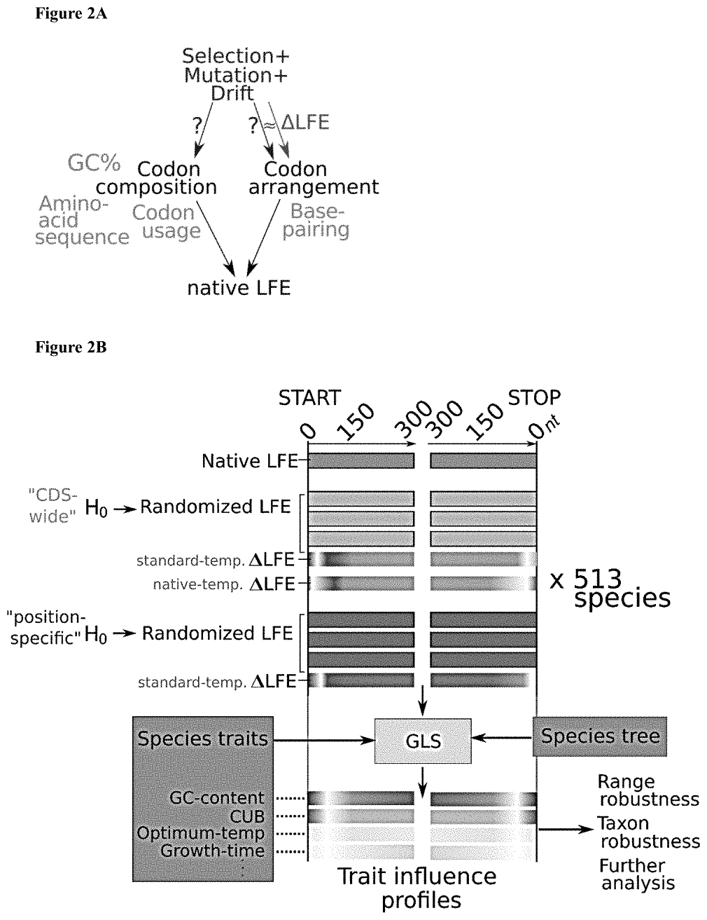

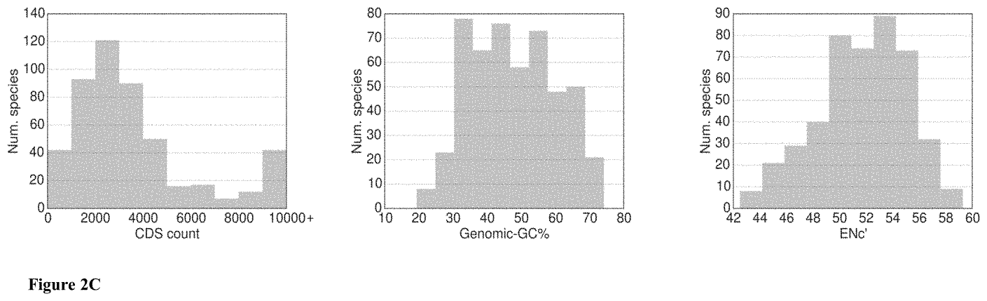

Species selection and sequence filtering: The set of species included in the dataset (Table 2) was chosen to maximize taxonomic coverage, include closely related species which differ in GC-contents and other traits (FIG. 2C), and take advantage of the limited overlap between available annotated genomes, NCBI environmental traits data, and the phylogenetic tree (see below). The set of species and their characteristics including growth conditions and genomic data are also provided in Peeri and Tuller, 2020, “High-resolution modeling of the selection on local mRNA folding strength in coding sequences across the tree of life”, Genome Biology, herein incorporated by reference in its entirety. To prevent under-representation of taxa in the dataset, included species were tabulated by phylum and species from missing phyla and classes were added if possible (Table 3). Over-representation of closely related species is controlled by GLS (see below).

CDS sequences and gene annotations for all species were obtained from Ensembl genomes, NCBI, JGI and SGD (Table 4). CDS sequences were matched with their GFF3 annotations to filter suspect sequences, as follows. The dataset excludes CDSs marked as pseudo-genes or suspected pseudo-genes, incomplete CDSs and those with sequencing ambiguities, as well as CDSs of length <150 nt. If multiple isoforms were available, only the primary (or first) transcript was included. Genes annotated as belonging to organelle genomes were also excluded. Genomic GC-content, optimum growth temperatures and translation tables were extracted from NCBI Entrez automatically, using a combination of Entrez and E-utilities requests (Table 4). A few general characteristics of the included CDSs are shown in FIG. 2C.

The taxonomic hierarchy and classifications used to analyze and present the data were obtained from NCBI Taxonomy. Endosymbionts were annotated using a literature survey (Table 4). Growth rates were extracted from Vieira-Silva S, Rocha EPC. The Systemic Imprint of Growth and Its Uses in Ecological (Meta)Genomics. PLOS Genet. 2010 Jan. 15; 6(1):e1000808 herein incorporated by reference.

TABLE 2
Species in the data set and basic data

| | | Ann. | CDS | Num | | |
|---|---|---|---|---|---|---|
| TaxId | Species | GC % | GC % | CDSs | Phylum | Domain |
| 747 | Pasteurella multocida str. ATCC 43137 | 40.3 | 41.03 | 2036 | Proteobacteria | Bacteria |
| 882 | Desulfovibrio vulgaris str. Hildenborough | 67.1 | 63.53 | 3510 | Proteobacteria | Bacteria |
| 979 | Cellulophaga lytica | 32.1 | 32.67 | 3168 | Bacteroidetes | Bacteria |
| 1148 | Synechocystis sp. PCC 6803 | 47.35 | 48.22 | 3564 | Cyanobacteria | Bacteria |
| 2769 | Chondrus crispus (carragheen) | 52.86 | 53.68 | 8815 | | Eukaryota |
| 2898 | Cryptomonas paramecium | 27.81 | 25.98 | 465 | | Eukaryota |
| 3046 | Dunaliella salina | 40.1 | 58.19 | 16005 | Chlorophyta | Eukaryota |
| 3055 | Chlamydomonas reinhardtii | 61.95 | 70.24 | 17741 | Chlorophyta | Eukaryota |
| 3067 | Volvox carteri | 55.3 | 63.34 | 14241 | Chlorophyta | Eukaryota |
| 3218 | Physcomitrella patens | 34.3 | 49.31 | 32108 | Streptophyta | Eukaryota |
| 4781 | Plasmopara halstedii | 45.7 | 45.97 | 14306 | | Eukaryota |
| 4927 | Wickerhamomyces anomalus | 35 | 34.54 | 6262 | Ascomycota | Eukaryota |
| | NRRL Y-366-8 | | | | | |
| 5061 | Aspergillus niger | 50.3 | 53.72 | 13713 | Ascomycota | Eukaryota |
| 5693 | Trypanosoma cruzi | 51.7 | 53.16 | 18456 | | Eukaryota |
| 6669 | Daphnia pulex | 42.4 | 47.3 | 30162 | Arthropoda | Eukaryota |
| 10228 | Trichoplax adhaerens | 34.5 | 37.71 | 11435 | Placozoa | Eukaryota |
| 27923 | Mnemiopsis leidyi | 39.1 | 45.66 | 15557 | Ctenophora | Eukaryota |
| 28892 | Methanofollis liminatans DSM 4140 | 61 | 61.95 | 2422 | Euryarchaeota | Archaea |
| 29290 | Candidatus Magnetobacterium bavaricum | 47.3 | 48.21 | 5870 | Nitrospirae | Bacteria |
| 29656 | Spirodela polyrhiza | 42.72 | 55.64 | 19462 | Streptophyta | Eukaryota |
| 36329 | Plasmodium falciparum 3D7 | 19.36 | 23.74 | 5356 | Apicomplexa | Eukaryota |
| 44056 | Aureococcus anophagefferens | 67.4 | 70.8 | 11189 | | Eukaryota |
| 45351 | Nematostella vectensis | 41.9 | 47.35 | 24239 | Cnidaria | Eukaryota |
| 45670 | Salinicoccus roseus | 50 | 51.23 | 2399 | Firmicutes | Bacteria |
| 46234 | Anabaena sp. 90 | 38.09 | 38.76 | 4501 | Cyanobacteria | Bacteria |
| 49280 | Gelidibacter algens | 37.3 | 38.19 | 3654 | Bacteroidetes | Bacteria |
| 59374 | Fibrobacter succinogenes subsp. | 48 | 48.89 | 3079 | Fibrobacteres | Bacteria |
| | succinogenes S85 | | | | | |
| 63737 | Nostoc punctiforme PCC 73102 | 41.34 | 42.59 | 6620 | Cyanobacteria | Bacteria |
| 64091 | Halobacterium salinarum NRC-1 | 65.7 | 66.88 | 2586 | Euryarchaeota | Archaea |
| 65357 | Albugo candida | 43.2 | 44.63 | 13222 | | Eukaryota |
| 70601 | Pyrococcus horikoshii 0T3 | 41.9 | 42.32 | 2061 | Euryarchaeota | Archaea |
| 83332 | Mycobacterium tuberculosis H37Rv | 65.6 | 65.9 | 4016 | Actinobacteria | Bacteria |
| 85962 | Helicobacter pylori 26695 | 38.9 | 39.61 | 1554 | Proteobacteria | Bacteria |
| 93061 | Staphylococcus aureus subsp. aureus | 32.9 | 33.51 | 2625 | Firmicutes | Bacteria |
| | NCTC 8325 | | | | | |
| 96563 | Pseudomonas stutzeri | 60.6 | 64.52 | 4052 | Proteobacteria | Bacteria |
| 99287 | Salmonella enterica subsp. enterica | 51.88 | 53.35 | 4545 | Proteobacteria | Bacteria |
| | serovar Typhimurium str. LT2 | | | | | |
| 100226 | Streptomyces coelicolor A3(2) | 71.98 | 72.34 | 8109 | Actinobacteria | Bacteria |
| 104782 | Adineta vaga | 31.2 | 33.33 | 47746 | Rotifera | Eukaryota |
| 107806 | Buchnera aphidicola str. APS | 25.3 | 27.43 | 574 | Proteobacteria | Bacteria |
| | (Acyrthosiphon pisum) | | | | | |
| 115713 | Chlamydophila pneumoniae CWL029 | 40.6 | 41.34 | 1052 | Chlamydiae | Bacteria |
| 122586 | Neisseria meningitidis MC58 | 51.5 | 53.08 | 2048 | Proteobacteria | Bacteria |
| 123214 | Persephonella marina EX-H1 | 37.12 | 37.31 | 2048 | Aquificae | Bacteria |
| 130081 | Galdieria sulphuraria | 37.9 | 39.68 | 7089 | | Eukaryota |
| 138677 | Chlamydophila pneumoniae J138 | 40.6 | 41.36 | 1068 | Chlamydiae | Bacteria |
| 145458 | Rathayibacter toxicus | 61.5 | 61.94 | 1740 | Actinobacteria | Bacteria |
| 153151 | Parageobacillus toebii | 42.1 | 42.95 | 3780 | Firmicutes | Bacteria |
| 155920 | Xylella fastidiosa subsp. sandyi Ann-1 | 52.64 | 53.57 | 2626 | Proteobacteria | Bacteria |
| 156889 | Magnetococcus marinus MC-1 | 54.2 | 54.79 | 3716 | Proteobacteria | Bacteria |
| 158189 | Sphaerochaeta globosa str. Buddy | 48.9 | 49.41 | 3017 | Spirochaetes | Bacteria |
| 160490 | Streptococcus pyogenes M1 GAS | 38.5 | 39.15 | 1686 | Firmicutes | Bacteria |
| 160492 | Xylella fastidiosa 9a5c | 52.64 | 53.72 | 2823 | Proteobacteria | Bacteria |
| 163003 | Thermococcus cleftensis | 55.8 | 56.66 | 1989 | Euryarchaeota | Archaea |
| 164328 | Phytophthora ramorum | 53 | 58.02 | 15109 | | Eukaryota |
| 167546 | Prochlorococcus marinus str. MIT 9301 | 36.4 | 32.06 | 1891 | Cyanobacteria | Bacteria |
| 169963 | Listeria monocytogenes EGD-e | 38 | 38.44 | 2843 | Firmicutes | Bacteria |
| 176280 | Staphylococcus epidermidis ATCC 12228 | 32.05 | 32.9 | 2429 | Firmicutes | Bacteria |
| 176299 | Agrobacterium fabrum str. C58 | 59.06 | 59.82 | 5352 | Proteobacteria | Bacteria |
| 178306 | Pyrobaculum aerophilum str. IM2 | 51.4 | 51.9 | 2594 | Crenarchaeota | Archaea |
| 184922 | Giardia lamblia ATCC 50803 | 49.2 | 49.02 | 7313 | | Eukaryota |
| 186497 | Pyrococcus furiosus DSM 3638 | 40.8 | 41.09 | 2060 | Euryarchaeota | Archaea |
| 187272 | Alkalilimnicola ehrlichii MLHE-1 | 67.5 | 67.82 | 2863 | Proteobacteria | Bacteria |
| 187420 | Methanothermobacter | 49.5 | 50.56 | 1867 | Euryarchaeota | Archaea |
| | thermautotrophicus str. Delta H | | | | | |
| 188937 | Methanosarcina acetivorans C2A | 42.7 | 45.17 | 4539 | Euryarchaeota | Archaea |
| 190192 | Methanopyrus kandleri AV19 | 61.2 | 61.2 | 1687 | Euryarchaeota | Archaea |
| 190304 | Fusobacterium nucleatum subsp. | 27.2 | 27.39 | 2036 | Fusobacteria | Bacteria |
| | nucleatum ATCC 25586 | | | | | |
| 190485 | Xanthomonas campestris pv. campestris | 65.1 | 65.58 | 4177 | Proteobacteria | Bacteria |
| | str. ATCC 33913 | | | | | |
| 190650 | Caulobacter crescentus CB15 | 67.2 | 67.68 | 3728 | Proteobacteria | Bacteria |
| 192222 | Campylobacter jejuni subsp. jejuni NCTC | 30.5 | 30.83 | 1610 | Proteobacteria | Bacteria |
| | 11168 = ATCC 700819 | | | | | |
| 194439 | Chlorobium tepidum TLS | 56.5 | 57.63 | 2220 | Chlorobi | Bacteria |
| 195522 | Thermococcus nautili | 54.8 | 55.51 | 2161 | Euryarchaeota | Archaea |
| 196162 | Nocardioides sp. JS614 | 71.48 | 71.67 | 4888 | Actinobacteria | Bacteria |
| 196164 | Corynebacterium efficiens YS-314 | 62.93 | 63.68 | 2996 | Actinobacteria | Bacteria |
| 196600 | Vibrio vulnificus YJ016 | 46.67 | 47.48 | 5024 | Proteobacteria | Bacteria |
| 196627 | Corynebacterium glutamicum ATCC 13032 | 53.8 | 54.78 | 3053 | Actinobacteria | Bacteria |
| 203123 | Oenococcus oeni PSU-1 | 37.9 | 38.88 | 1677 | Firmicutes | Bacteria |
| 203124 | Trichodesmium erythraeum IMS101 | 34.1 | 36.77 | 4440 | Cyanobacteria | Bacteria |
| 203267 | Tropheryma whipplei str. Twist | 46.3 | 46.46 | 808 | Actinobacteria | Bacteria |
| 203907 | Candidatus Blochmannia floridanus | 27.4 | 28.9 | 582 | Proteobacteria | Bacteria |
| 204536 | Sulfurihydrogenibium azorense Az-Fu1 | 32.8 | 32.8 | 1720 | Aquificae | Bacteria |
| 208964 | Pseudomonas aeruginosa PAO1 | 66.6 | 67.16 | 5523 | Proteobacteria | Bacteria |
| 211586 | Shewanella oneidensis MR-1 | 45.93 | 46.94 | 4191 | Proteobacteria | Bacteria |
| 212717 | Clostridium tetani E88 | 28.59 | 29 | 2432 | Firmicutes | Bacteria |
| 213585 | Methanosarcina mazei S-6 | 41.4 | 44.14 | 3335 | Euryarchaeota | Archaea |
| 214684 | Cryptococcus neoformans var. neoformans | 48.54 | 51.16 | 6570 | Basidiomycota | Eukaryota |
| | JEC21 | | | | | |
| 216432 | Croceibacter atlanticus HTCC2559 | 33.9 | 34.33 | 2696 | Bacteroidetes | Bacteria |
| 218497 | Chlamydia abortus S26-3 | 39.9 | 40.49 | 932 | Chlamydiae | Bacteria |
| 220668 | Lactobacillus plantarum WCFS1 | 44.45 | 45.47 | 3101 | Firmicutes | Bacteria |
| 221109 | Oceanobacillus iheyensis HTE831 | 35.7 | 36.1 | 3490 | Firmicutes | Bacteria |
| 223926 | Vibrio parahaemolyticus RIMD 2210633 | 45.4 | 46.28 | 4522 | Proteobacteria | Bacteria |
| 224308 | Bacillus subtilis subsp. subtilis str. 168 | 43.5 | 44.22 | 4120 | Firmicutes | Bacteria |
| 224324 | Aquifex aeolicus VF5 | 43.32 | 43.58 | 1553 | Aquificae | Bacteria |
| 224325 | Archaeoglobus fulgidus DSM 4304 | 48.6 | 49.36 | 2405 | Euryarchaeota | Archaea |
| 224914 | Brucella melitensis bv. 1 str. 16M | 57.24 | 58.28 | 3194 | Proteobacteria | Bacteria |
| 226185 | Enterococcus faecalis V583 | 37.35 | 37.95 | 3241 | Firmicutes | Bacteria |
| 226186 | Bacteroides thetaiotaomicron VPI-5482 | 42.82 | 43.91 | 4825 | Bacteroidetes | Bacteria |
| 227377 | Coxiella burnetii RSA 493 | 42.34 | 43.22 | 1828 | Proteobacteria | Bacteria |
| 227882 | Streptomyces avermitilis MA-4680 = NBRC | 70.6 | 71.12 | 7661 | Actinobacteria | Bacteria |
| | 14893 | | | | | |
| 228410 | Nitrosomonas europaea ATCC 19718 | 50.7 | 51.57 | 2462 | Proteobacteria | Bacteria |
| 228908 | Nanoarchaeum equitans | 31.6 | 31.2 | 536 | Nanoarchaeota | Archaea |
| 233412 | Haemophilus ducreyi 35000HP | 38.2 | 38.74 | 1694 | Proteobacteria | Bacteria |
| 234267 | Candidatus Solibacter usitatus Ellin6076 | 61.9 | 62.43 | 7825 | Acidobacteria | Bacteria |
| 235909 | Geobacillus kaustophilus HTA426 | 51.99 | 52.84 | 3531 | Firmicutes | Bacteria |
| 237561 | Candida albicans SC5314 | 33.48 | 35.23 | 14102 | Ascomycota | Eukaryota |
| 240015 | Acidobacterium capsulatum ATCC 51196 | 60.5 | 61.1 | 3376 | Acidobacteria | Bacteria |
| 242507 | Magnaporthe oryzae | 51.59 | 57.72 | 12746 | Ascomycota | Eukaryota |
| 243090 | Rhodopirellula baltica SH 1 | 55.4 | 55.46 | 7325 | Planctomycetes | Bacteria |
| 243159 | Acidithiobacillus ferrooxidans ATCC 23270 | 58.8 | 59.32 | 3129 | Proteobacteria | Bacteria |
| 243230 | Deinococcus radiodurans RI | 66.61 | 67.23 | 3050 | Deinococcus-Thermus | Bacteria |
| 243232 | Methanocaldococcus jannaschii DSM 2661 | 31.27 | 31.85 | 1755 | Euryarchaeota | Archaea |
| 243233 | Methylococcus capsulatus str. Bath | 63.6 | 63.96 | 2959 | Proteobacteria | Bacteria |
| 243265 | Photorhabdus luminescens subsp. | 42.8 | 44.16 | 4680 | Proteobacteria | Bacteria |
| | laumondii TTO1 | | | | | |
| 243273 | Mycoplasma genitalium G37 | 31.7 | 31.55 | 476 | Tenericutes | Bacteria |
| 243274 | Thermotoga maritima MSB8 | 46.2 | 46.4 | 1800 | Thermotogae | Bacteria |
| 243275 | Treponema denticola ATCC 35405 | 37.9 | 38.27 | 2726 | Spirochaetes | Bacteria |
| 243365 | Chromobacterium violaceum ATCC 12472 | 64.8 | 65.71 | 4399 | Proteobacteria | Bacteria |
| 251221 | Gloeobacter violaceus PCC 7421 | 62 | 62.86 | 4357 | Cyanobacteria | Bacteria |
| 255470 | Dehalococcoides mccartyi CBDB1 | 48.9 | 47.85 | 1456 | Chloroflexi | Bacteria |
| 257314 | Lactobacillus johnsonii NCC 533 | 34.6 | 34.96 | 1819 | Firmicutes | Bacteria |
| 258594 | Rhodopseudomonas palustris CGA009 | 66 | 65.53 | 4814 | Proteobacteria | Bacteria |
| 259536 | Psychrobacter arcticus 273-4 | 42.8 | 44.59 | 2119 | Proteobacteria | Bacteria |
| 262768 | Onion yellows phytoplasma OY-M | 27.8 | 29.07 | 744 | Tenericutes | Bacteria |
| 263358 | Verrucosispora maris AB-18-032 | 70.89 | 71.28 | 5978 | Actinobacteria | Bacteria |
| 263820 | Picrophilus torridus DSM 9790 | 36 | 37.08 | 1534 | Euryarchaeota | Archaea |
| 264462 | Bdellovibrio bacteriovorus HD100 | 43.3 | 51.01 | 3581 | Proteobacteria | Bacteria |
| 266834 | Sinorhizobium meliloti 1021 | 62.16 | 62.86 | 6228 | Proteobacteria | Bacteria |
| 266940 | Kineococcus radiotolerans SRS30216 = | 74.21 | 74.34 | 4653 | Actinobacteria | Bacteria |
| | ATCC BAA-149 | | | | | |
| 267377 | Methanococcus maripaludis S2 | 33.3 | 34.01 | 1712 | Euryarchaeota | Archaea |
| 267608 | Ralstonia solanacearum GMI1000 | 66.96 | 67.56 | 5097 | Proteobacteria | Bacteria |
| 267671 | Leptospira interrogans serovar | 35.01 | 36.68 | 3658 | Spirochaetes | Bacteria |
| | Copenhageni str. Fiocruz L1-130 | 55.5 | 56.13 | 2485 | Cyanobacteria | Bacteria |
| 269084 | Synechococcus elongatus PCC 6301 | | | | | |
| 269800 | Thermobifida fusca YX | 67.5 | 68.13 | 3107 | Actinobacteria | Bacteria |
| 272557 | Aeropyrum pernix K1 | 56.3 | 56.97 | 1695 | Crenarchaeota | Archaea |
| 272558 | Bacillus halodurans C-125 | 43.7 | 44.32 | 4039 | Firmicutes | Bacteria |
| 272567 | Geobacillus stearothermophilus 10 | 52.61 | 53.68 | 3303 | Firmicutes | Bacteria |
| 272623 | Lactococcus lactis subsp. lactis ll1403 | 35.3 | 36.18 | 2258 | Firmicutes | Bacteria |
| 272626 | Listeria innocua Clip11262 | 37.35 | 37.79 | 3040 | Firmicutes | Bacteria |
| 272631 | Mycobacterium leprae TN | 57.8 | 60.12 | 1605 | Actinobacteria | Bacteria |
| 272632 | Mycoplasma mycoides subsp. mycoides SC | 24 | 24.09 | 1012 | Tenericutes | Bacteria |
| | str. PG1 | | | | | |
| 272633 | Mycoplasma penetrans HF-2 | 25.7 | 26.48 | 1033 | Tenericutes | Bacteria |
| 272634 | Mycoplasma pneumoniae M129 | 40 | 40.75 | 688 | Tenericutes | Bacteria |
| 272635 | Mycoplasma pulmonis UAB CTIP | 26.6 | 27.29 | 775 | Tenericutes | Bacteria |
| 272844 | Pyrococcus abyssi GE5 | 44.7 | 45.14 | 1782 | Euryarchaeota | Archaea |
| 273063 | Sulfolobus tokodaii str. 7 | 32.8 | 33.52 | 2811 | Crenarchaeota | Archaea |
| 273075 | Thermoplasma acidophilum DSM 1728 | 46 | 47.28 | 1478 | Euryarchaeota | Archaea |
| 273116 | Thermoplasma volcanium GSS1 | 39.9 | 40.99 | 1525 | Euryarchaeota | Archaea |
| 273121 | Wolinella succinogenes DSM 1740 | 48.5 | 48.91 | 2044 | Proteobacteria | Bacteria |
| 280463 | Emiliania huxleyi CCMP1516 | 64.5 | 69.09 | 36050 | | Eukaryota |
| 280699 | Cyanidioschyzon merolae | 55.02 | 56.72 | 4951 | | Eukaryota |
| 281090 | Leifsonia xyli subsp. xyli str. CTCB07 | 68.3 | 68.39 | 2019 | Actinobacteria | Bacteria |
| 283166 | Bartonella henselae str. Houston-1 | 38.2 | 40.03 | 1488 | Proteobacteria | Bacteria |
| 284811 | Eremothecium gossypii ATCC 10895 | 51.69 | 52.8 | 4748 | Ascomycota | Eukaryota |
| | (assembly ASM9102v4) | | | | | |
| 284812 | Schizosaccharomyces pombe (strain 972/ | 36.04 | 39.61 | 5141 | Ascomycota | Eukaryota |
| | ATCC 24843) | | | | | |
| 288705 | Renibacterium salmoninarum ATCC 33209 | 56.3 | 56.61 | 3505 | Actinobacteria | Bacteria |
| 289376 | Thermodesulfovibrio yellowstonii | 34.1 | 34.17 | 2030 | Nitrospirae | Bacteria |
| | DSM 11347 | | | | | |
| 289377 | Thermodesulfobacterium commune | 37 | 37.33 | 1453 | Thermodesulfobacteria | Bacteria |
| | DSM 2178 | | | | | |
| 290633 | Gluconobacter oxydans 621H | 60.84 | 61.47 | 2662 | Proteobacteria | Bacteria |
| 295405 | Bacteroides fragilis YCH46 | 43.24 | 44.16 | 4414 | Bacteroidetes | Bacteria |
| 296543 | Thalassiosira pseudonana | 46.91 | 47.95 | 11061 | Bacillariophyta | Eukaryota |
| 298386 | Photobacterium profundum SS9 | 41.75 | 42.67 | 5469 | Proteobacteria | Bacteria |
| 300852 | Thermus thermophilus HB8 | 69.49 | 69.66 | 2221 | Deinococcus-Thermus | Bacteria |
| 309799 | Dictyoglomus thermophilum H-6-12 | 33.7 | 33.81 | 1908 | Dictyoglomi | Bacteria |
| 309801 | Thermomicrobium roseum DSM 5159 | 64.26 | 64.18 | 2856 | Chloroflexi | Bacteria |
| 312017 | Tetrahymena thermophila SB210 | 22.3 | 27.72 | 24128 | | Eukaryota |
| 313596 | Robiginitalea biformata HTCC2501 | 55.3 | 56.07 | 3192 | Bacteroidetes | Bacteria |
| 313628 | Lentisphaera araneosa HTCC2155 | 41 | 41.63 | 5042 | Lentisphaerae | Bacteria |
| 314225 | Erythrobacter litoralis HTCC2594 | 63.1 | 63.43 | 3000 | Proteobacteria | Bacteria |
| 314260 | Parvularcula bermudensis HTCC2503 | 60.7 | 60.96 | 2677 | Proteobacteria | Bacteria |
| 314278 | Nitrococcus mobilis Nb-231 | 59.9 | 60.75 | 3482 | Proteobacteria | Bacteria |
| 316274 | Herpetosiphon aurantiacus DSM 785 | 50.89 | 51.41 | 5278 | Chloroflexi | Bacteria |
| 316279 | Synechococcus sp. CC9902 | 54.2 | 54.87 | 2302 | Cyanobacteria | Bacteria |
| 316407 | Escherichia coli str. K-12 substr. W3110 | 50.45 | 51.9 | 4222 | Proteobacteria | Bacteria |
| 319795 | Deinococcus geothermalis DSM 11300 str. | 66.57 | 66.86 | 3051 | Deinococcus-Thermus | Bacteria |
| | DSM11300 | | | | | |
| 322098 | Aster yellows witches'-broom phytoplasma | 26.83 | 28.41 | 683 | Tenericutes | Bacteria |
| | AYWB | | | | | |
| 324602 | Chloroflexus aurantiacus J-10-fl | 56.7 | 57.13 | 3852 | Chloroflexi | Bacteria |
| 326298 | Sulfurimonas denitrificans DSM 1251 | 34.5 | 34.78 | 2096 | Proteobacteria | Bacteria |
| 326427 | Chloroflexus aggregans DSM 9485 | 56.4 | 56.77 | 3730 | Chloroflexi | Bacteria |
| 330214 | Nitrospira defluvii | 59 | 59.27 | 4262 | Nitrospirae | Bacteria |
| 331104 | Blattabacterium sp. (Blattella germanica) | 23.84 | 27.25 | 589 | Bacteroidetes | Bacteria |
| | str. Bge | | | | | |
| 331113 | Simkania negevensis Z | 41.62 | 42.26 | 2466 | Chlamydiae | Bacteria |
| 333146 | Ferroplasma acidarmanus fer1 | 36.5 | 37.56 | 1942 | Euryarchaeota | Archaea |
| 335284 | Psychrobacter cryohalolentis K5 | 42.25 | 43.98 | 2511 | Proteobacteria | Bacteria |
| 336722 | Zymoseptoria tritici | 52.12 | 55.56 | 10780 | Ascomycota | Eukaryota |
| 339860 | Methanosphaera stadtmanae DSM 3091 | 27.6 | 29.1 | 1507 | Euryarchaeota | Archaea |
| 345663 | Chryseobacterium greenlandense | 34.1 | 35.1 | 3587 | Bacteroidetes | Bacteria |
| 347257 | Mycoplasma agalactiae PG2 | 29.7 | 30.11 | 751 | Tenericutes | Bacteria |
| 347515 | Leishmania major strain Friedlin | 59.71 | 62.45 | 8299 | | Eukaryota |
| 349741 | Akkermansia muciniphila ATCC BAA-835 | 55.8 | 56.76 | 2137 | Verrucomicrobia | Bacteria |
| 351607 | Acidothermus cellulolyticus 11B | 66.9 | 66.76 | 2156 | Actinobacteria | Bacteria |
| 352472 | Dictyostelium discoideum AX4 | 22.46 | 27.4 | 12859 | | Eukaryota |
| 353152 | Cryptosporidium parvum Iowa II | 30.25 | 31.88 | 3761 | Apicomplexa | Eukaryota |
| 353154 | Theileria annulata strain Ankara | 32.55 | 35.72 | 3792 | Apicomplexa | Eukaryota |
| 358681 | Brevibacillus brevis NBRC 100599 | 47.3 | 47.88 | 5934 | Firmicutes | Bacteria |
| 360911 | Exiguobacterium sp. AT1b | 48.5 | 49.1 | 3015 | Firmicutes | Bacteria |
| 362976 | Haloquadratum walsbyi DSM 16790 | 47.69 | 48.75 | 2548 | Euryarchaeota | Archaea |
| 365046 | Ramlibacter tataouinensis TTB310 | 70 | 70.36 | 3854 | Proteobacteria | Bacteria |
| 373903 | Halothermothrix orenii H 168 | 37.9 | 38.89 | 2341 | Firmicutes | Bacteria |
| 374847 | Candidatus Korarchaeum cryptofilum OPF8 | 49 | 49.54 | 1602 | Candidatus | Archaea |
| | | | | | Korarchaeota | |
| 379066 | Gemmatimonas aurantiaca T-27 | 64.3 | 64.49 | 3934 | Gemmatimonadetes | Bacteria |
| 381306 | Thiohalorhabdus denitrificans | 68.9 | 69.71 | 2403 | Proteobacteria | Bacteria |
| 381764 | Fervidobacterium nodosum Rtl7-Bl | 35 | 35.23 | 1746 | Thermotogae | Bacteria |
| 383372 | Roseiflexus castenholzii DSM 13941 | 60.7 | 60.94 | 4330 | Chloroflexi | Bacteria |
| 388396 | Vibrio fischeri MJ11 | 38.37 | 38.85 | 4039 | Proteobacteria | Bacteria |
| 391009 | Thermosipho melanesiensis BI429 | 31.4 | 31.23 | 1875 | Thermotogae | Bacteria |
| 391165 | Granulibacter bethesdensis CGDNIH1 | 59.1 | 59.62 | 2435 | Proteobacteria | Bacteria |
| 391603 | Flavobacteriales bacterium ALC-1 | 32.4 | 32.87 | 3428 | Bacteroidetes | Bacteria |
| 391623 | Thermococcus barophilus MP | 41.71 | 42.08 | 2173 | Euryarchaeota | Archaea |
| 393595 | Alcanivorax borkumensis SK2 | 54.7 | 55.24 | 2755 | Proteobacteria | Bacteria |
| 398720 | Leeuwenhoekiella blandensis MED217 | 39.8 | 40.39 | 3715 | Bacteroidetes | Bacteria |
| 398767 | Geobacter lovleyi SZ | 54.77 | 55.33 | 3200 | Proteobacteria | Bacteria |
| 400667 | Acinetobacter baumannii ATCC 17978 | 39 | 40.13 | 3826 | Proteobacteria | Bacteria |
| 400682 | Amphimedon queenslandica | 37.5 | 41.36 | 27593 | Porifera | Eukaryota |
| 402612 | Flavobacterium psychrophilum JIP02/86 | 32.5 | 33.24 | 2397 | Bacteroidetes | Bacteria |
| 402881 | Parvibaculum lavamentivorans DS-1 | 62.3 | 62.74 | 3635 | Proteobacteria | Bacteria |
| 403833 | Petrotoga mobilis SJ95 | 34.1 | 34.2 | 1896 | Thermotogae | Bacteria |
| 405948 | Saccharopolyspora erythraea NRRL 2338 | 71.1 | 71.6 | 7164 | Actinobacteria | Bacteria |
| 407035 | Salinicoccus halodurans | 44.5 | 45.55 | 2643 | Firmicutes | Bacteria |
| 410358 | Methanocorpusculum labreanumZ | 50 | 51.1 | 1738 | Euryarchaeota | Archaea |
| 411154 | Gramella forsetii KT0803 | 36.6 | 37.26 | 3573 | Bacteroidetes | Bacteria |
| 412030 | Paramecium tetraurelia strain d4-2 | 28.2 | 30.13 | 39433 | | Eukaryota |
| 412133 | Trichomonas vaginalis G3 | 32.9 | 35.55 | 56271 | | Eukaryota |
| 414004 | Cenarchaeum symbiosum A | 57.4 | 57.79 | 2010 | Thaumarchaeota | Archaea |
| 418459 | Puccinia graminis f. sp. tritici | 43.8 | 49.67 | 15958 | Basidiomycota | Eukaryota |
| 419610 | Methylobacterium extorquens PA1 | 68.2 | 69.02 | 4819 | Proteobacteria | Bacteria |
| 420247 | Methanobrevibacter smithii ATCC 35061 | 31 | 32.05 | 1731 | Euryarchaeota | Archaea |
| 420778 | Diplodia seriata | 56.5 | 60.75 | 9343 | Ascomycota | Eukaryota |
| 420890 | Lactococcus garvieae Lg2 | 38.8 | 39.63 | 1963 | Firmicutes | Bacteria |
| 423536 | Perkinsus marinus ATCC 50983 | 47.4 | 51.21 | 20630 | | Eukaryota |
| 429572 | Sulfolobus islandicus L.S.2.15 | 35.1 | 35.57 | 2735 | Crenarchaeota | Archaea |
| 431895 | Monosiga brevicollis MX1 | 54.33 | 57.25 | 9049 | | Eukaryota |
| 431947 | Porphyromonas gingivalis ATCC 33277 | 48.4 | 49.41 | 2082 | Bacteroidetes | Bacteria |
| 432331 | Sulfurihydrogenibium yellowstonense SS-5 | 32.8 | 32.69 | 1570 | Aquificae | Bacteria |
| 435906 | Salegentibarter salarius | 37 | 37.75 | 2932 | Bacteroidetes | Bacteria |
| 436017 | Ostreococcus lucimarinus | 60.44 | 59.01 | 7571 | Chlorophyta | Eukaryota |
| 436308 | Nitrosopumilus maritimus SCM1 | 34.2 | 34.59 | 1792 | Thaumarchaeota | Archaea |
| 436907 | Vanderwaltozyma polyspora DSM 70294 | 33 | 34.95 | 5332 | Ascomycota | Eukaryota |
| 439292 | Bacillus selenitireducens MLS10 | 48.7 | 49.43 | 2819 | Firmicutes | Bacteria |
| 441768 | Acholeplasma laidlawii PG-8A | 31.9 | 32.23 | 1377 | Tenericutes | Bacteria |
| 443254 | Marinitoga piezophila KA3 | 29.18 | 29.1 | 2034 | Thermotogae | Bacteria |
| 443906 | Clavibacter michiganensis subsp. | 72.42 | 72.71 | 3059 | Actinobacteria | Bacteria |
| | michiganensis NCPPB 382 | | | | | |
| 445932 | Elusimicrobium minutum Pei191 | 40 | 40.69 | 1526 | Elusimicrobia | Bacteria |
| 446470 | Stackebrandtia nassauensis DSM 44728 | 68.1 | 68.66 | 6366 | Actinobacteria | Bacteria |
| 449447 | Microcystis aeruginosa NIES-843 | 42.3 | 42.9 | 6306 | Cyanobacteria | Bacteria |
| 452637 | Opitutus terrae PB90-1 | 65.3 | 65.47 | 4610 | Verrucomicrobia | Bacteria |
| 452652 | Kitasatospora setae KM-6054 | 74.2 | 74.44 | 7477 | Actinobacteria | Bacteria |
| 456481 | Leptospira biflexa serovar Patoc strain | 38.9 | 39.07 | 2678 | Spirochaetes | Bacteria |
| | ‘Patoc 1 (Paris)’ | | | | | |
| 457570 | Natranaerobius thermophilus JW/NM-WN- | 36.29 | 36.77 | 2903 | Firmicutes | Bacteria |
| | LF | | | | | |
| 469371 | Thermobispora bispora DSM 43833 | 72.4 | 72.48 | 3535 | Actinobacteria | Bacteria |
| 469382 | Halogeometricum borinquense DSM 11551 | 59.97 | 61.05 | 3890 | Euryarchaeota | Archaea |
| 469383 | Conexibacter woesei DSM 14684 | 72.4 | 72.93 | 5902 | Actinobacteria | Bacteria |
| 469599 | Fusobacterium periodonticum 2_1_31 | 28.6 | 28.28 | 2327 | Fusobacteria | Bacteria |
| 469615 | Fusobacterium gonidiaformans | 32.9 | 32.79 | 1600 | Fusobacteria | Bacteria |
| | ATCC 25563 | | | | | |
| 476282 | Bradyrhizobium japonicum SEMIA 5079 | 63.7 | 64.41 | 8646 | Proteobacteria | Bacteria |
| | Candidatus Desulforudis audaxviator | | | | | |
| 477974 | MP104C | 60.8 | 62.05 | 2157 | Firmicutes | Bacteria |
| 478009 | Halobacterium salinarum R1 | 65.92 | 66.81 | 2701 | Euryarchaeota | Archaea |
| 479433 | Catenulispora acidiphila DSM 44928 | 69.8 | 70.24 | 8884 | Actinobacteria | Bacteria |
| 479434 | Sphaerobacter thermophilus DSM 20745 | 68.1 | 68.34 | 3484 | Chloroflexi | Bacteria |
| 481448 | Methylacidiphilum infernorum V4 | 45.5 | 45.85 | 2451 | Verrucomicrobia | Bacteria |
| 484019 | Thermosipho africanus TCF52B | 30.8 | 30.73 | 1954 | Thermotogae | Bacteria |
| 484906 | Babesia bovis T2Bo | 41.61 | 43.87 | 3699 | Apicomplexa | Eukaryota |
| 485913 | Ktedonobacter racemifer DSM 44963 | 53.8 | 55.11 | 11437 | Chloroflexi | Bacteria |
| 486041 | Laccaria bicolor S238N-H82 | 47.1 | 50.56 | 18172 | Basidiomycota | Eukaryota |
| 491915 | Anoxybacillus flavithermus WK1 | 41.8 | 42.02 | 2824 | Firmicutes | Bacteria |
| 498848 | Thermus aquaticus Y51MC23 | 68.04 | 68.36 | 2521 | Deinococcus-Thermus | Bacteria |
| 500635 | Mitsuokella multacida DSM 20544 | 58 | 59.41 | 2541 | Firmicutes | Bacteria |
| 504728 | Meiothermus ruber DSM 1279 | 63.4 | 64.12 | 3014 | Deinococcus-Thermus | Bacteria |
| 505682 | Ureaplasma parvum serovar 3 str. | 25.5 | 25.69 | 609 | Tenericutes | Bacteria |
| | ATCC 27815 | | | | | |
| 507754 | Acidiplasma aeolicum str. VT | 34.2 | 35.21 | 1663 | Euryarchaeota | Archaea |
| 508771 | Toxoplasma gondii ME49 | 52.29 | 58.1 | 7917 | Apicomplexa | Eukaryota |
| 511051 | Caldisericum exile AZM16c01 | 35.4 | 35.51 | 1578 | Caldiserica | Bacteria |
| 511145 | Escherichia coli str. K-12 substr. MG1655 | 50.45 | 51.97 | 4031 | Proteobacteria | Bacteria |
| 515635 | Dictyoglomus turgidum DSM 6724 | 34 | 33.99 | 1744 | Dictyoglomi | Bacteria |
| 517417 | Chlorobaculum parvum NCIB 8327 | 55.8 | 57.18 | 2042 | Chlorobi | Bacteria |
| 517418 | Chloroherpeton thalassium ATCC 35110 | 45 | 46.14 | 2709 | Chlorobi | Bacteria |
| 518766 | Rhodothermus marinus DSM 4252 | 64.27 | 65.07 | 2860 | Bacteroidetes | Bacteria |
| 519441 | Streptobacillus moniliformis DSM 12112 | 26.27 | 26.16 | 1420 | Fusobacteria | Bacteria |
| 521011 | Methanosphaerula palustris E1-9c | 55.4 | 56.79 | 2650 | Euryarchaeota | Archaea |
| 521045 | Kosmotoga olearia TBF 19.5.1 | 41.5 | 41.55 | 2115 | Thermotogae | Bacteria |
| 521097 | Capnocytophaga ochracea DSM 7271 | 39.6 | 40.57 | 2164 | Bacteroidetes | Bacteria |
| 521674 | Planctopirus limnophila DSM 3776 | 53.72 | 54.43 | 4258 | Planctomycetes | Bacteria |
| 522772 | Denitrovibrio acetiphilus DSM 12809 | 42.5 | 43.2 | 2964 | Deferribacteres | Bacteria |
| 523841 | Haloferax mediterranei ATCC 33500 | 60.26 | 61.67 | 3825 | Euryarchaeota | Archaea |
| 525903 | Thermanaerovibrio acidaminovorans DSM | 63.8 | 64.38 | 1733 | Synergistetes | Bacteria |
| | 6589 | | | | | |
| 525904 | Thermobaculum terrenum ATCC BAA-798 | 53.54 | 53.82 | 2832 | | Bacteria |
| 525909 | Acidimicrobium ferrooxidans DSM 10331 | 68.3 | 68.37 | 1963 | Actinobacteria | Bacteria |
| 525919 | Anaerococcus prevotii DSM 20548 | 35.67 | 36.09 | 1801 | Firmicutes | Bacteria |
| 526218 | Sebaldella termitidis ATCC 33386 | 33.42 | 34.62 | 4128 | Fusobacteria | Bacteria |
| 526224 | Brachyspira murdochii DSM 12563 | 27.8 | 29 | 2800 | Spirochaetes | Bacteria |
| 543302 | Alicyclobacillus acidocaldarius LAA1 | 61.86 | 62.32 | 3006 | Firmicutes | Bacteria |
| 547144 | Hydrogenobaculum sp. HO | 34.8 | 34.88 | 1577 | Aquificae | Bacteria |
| 548479 | Mobiluncus curtisii ATCC 43063 | 55.4 | 55.89 | 1841 | Actinobacteria | Bacteria |
| 552811 | Dehalogenimonas lykanthroporepellens | 55 | 55.99 | 1655 | Chloroflexi | Bacteria |
| | BL-DC-9 | | | | | |
| 553190 | Gardnerella vaginalis 409-05 | 42 | 42.77 | 1258 | Actinobacteria | Bacteria |
| 554373 | Moniliophthora perniciosa FA553 | 47.7 | 49.78 | 9748 | Basidiomycota | Eukaryota |
| 555500 | Galbibacter marinus | 37 | 37.9 | 3079 | Bacteroidetes | Bacteria |
| 555778 | Halothiobacillus neapolitanus c2 | 54.7 | 55.49 | 2354 | Proteobacteria | Bacteria |
| 555779 | Desulfonatronospira thiodismutans | 51.3 | 52.52 | 3660 | Proteobacteria | Bacteria |
| | ASO3-1 | | | | | |
| 556484 | Phaeodactylum tricornutum CCAP 1055/1 | 48.84 | 50.96 | 12172 | Bacillariophyta | Eukaryota |
| 559292 | Saccharomyces cerevisiae S288c | 38.16 | 39.67 | 5787 | Ascomycota | Eukaryota |
| 561896 | Postia placenta Mad-698-R | 52.7 | 56.71 | 8904 | Basidiomycota | Eukaryota |
| 564608 | Micromonas pusilia CCMP1545 | 65.7 | 67.4 | 10615 | Chlorophyta | Eukaryota |
| 572478 | Vulcanisaeta distributa DSM 14429 | 45.4 | 46.26 | 2491 | Crenarchaeota | Archaea |
| 572544 | Ilyobacter polytropus DSM 2926 | 34.36 | 35.28 | 2870 | Fusobacteria | Bacteria |
| 573065 | Asticcacaulis excentricus CB 48 | 59.53 | 60.39 | 3761 | Proteobacteria | Bacteria |
| 574087 | Acetohalobium arabaticum DSM 5501 | 36.6 | 37.34 | 2278 | Firmicutes | Bacteria |
| 574566 | Coccomyxa subellipsoidea C-169 | 52.9 | 61.34 | 9603 | Chlorophyta | Eukaryota |
| 575540 | Isosphaera pallida ATCC 43644 | 62.45 | 63.04 | 3722 | Planctomycetes | Bacteria |
| 578458 | Schizophyllum commune H4-8 | 57.4 | 60.03 | 13171 | Basidiomycota | Eukaryota |
| 578462 | Allomyces macrogynus ATCC 38327 | 60.5 | 64.94 | 16745 | Blastocladiomycota | Eukaryota |
| 580340 | Thermovirga lienii DSM 17291 | 47.1 | 47.43 | 1874 | Synergistetes | Bacteria |
| 582515 | Rubidibacter lacunae KORDI 51-2 | 56.2 | 57.45 | 3411 | Cyanobacteria | Bacteria |
| 583355 | Coraliomargarita akajimensis DSM 45221 | 53.6 | 53.93 | 3118 | Verrucomicrobia | Bacteria |
| 583356 | Ignisphaera aggregans DSM 17230 | 35.7 | 36.01 | 1927 | Crenarchaeota | Archaea |
| 585394 | Roseburia hominis A2-183 | 48.5 | 49.34 | 3351 | Firmicutes | Bacteria |
| 589924 | Ferroglobus placidus DSM 10642 | 44.1 | 44.71 | 2478 | Euryarchaeota | Archaea |
| 592010 | Abiotrophia defectiva ATCC 49176 | 47 | 47.6 | 1943 | Firmicutes | Bacteria |
| 592029 | Nonlabens dokdonensis DSW-6 | 35.3 | 35.94 | 3613 | Bacteroidetes | Bacteria |
| 593117 | Thermococcus gammatolerans EJ3 | 53.6 | 54.14 | 2156 | Euryarchaeota | Archaea |
| 595528 | Capsaspora owczarzaki ATCC 30864 | 53.7 | 58.01 | 8627 | | Eukaryota |
| 596323 | Leptotrichia goodfellowii F0264 | 31.6 | 32.2 | 2266 | Fusobacteria | Bacteria |
| 608538 | Hydrogenobacter thermophilus TK-6 | 44 | 44.13 | 1894 | Aquificae | Bacteria |
| 633147 | Olsenella uli DSM 7084 | 64.7 | 65.18 | 1735 | Actinobacteria | Bacteria |
| 633149 | Brevundimonas subvibrioides ATCC 15264 | 68.4 | 68.81 | 3243 | Proteobacteria | Bacteria |
| 635003 | Fragilariopsis cylindrus CCMP1102 | 39 | 41.66 | 2790 | Bacillariophyta | Eukaryota |
| 638303 | Thermocrinis albus DSM 14484 | 46.9 | 47.01 | 1593 | Aquificae | Bacteria |
| 639282 | Deferribarter desulfuricans SSM1 | 30.3 | 30.48 | 2374 | Deferri bacteres | Bacteria |
| 641526 | Winogradskyella psychrotolerans RS-3 | 33.5 | 34.03 | 4001 | Bacteroidetes | Bacteria |
| 642492 | Clostridium lentocellum DSM 5427 | 34.3 | 34.83 | 4166 | Firmicutes | Bacteria |
| 644295 | Methanohalobium evestigatum Z-7303 | 36.4 | 37.58 | 2251 | Euryarchaeota | Archaea |
| 645134 | Spizellomyces punctatus DAOM BR117 | 47.6 | 49.84 | 9421 | Chytridiomycota | Eukaryota |
| 648996 | Thermovibrio ammonificans HB-1 | 52.12 | 52.26 | 1812 | Aquificae | Bacteria |
| 649638 | Truepera radiovictrix DSM 17093 | 68.1 | 68.71 | 2940 | Deinococcus-Thermus | Bacteria |
| 651182 | Desulfobacula toluolica Tol2 | 41.4 | 42.28 | 4374 | Proteobacteria | Bacteria |
| 653733 | Desulfurispirillum indicum S5 | 56.1 | 56.8 | 2570 | Chrysiogenetes | Bacteria |
| 655815 | Zunongwangia profunda SM-A87 | 36.2 | 37.1 | 4617 | Bacteroidetes | Bacteria |
| 660470 | Mesotoga prima MesGl.Ag.4.2 | 45.5 | 45.7 | 2565 | Thermotogae | Bacteria |
| 661478 | Fimbriimonas ginsengisoli Gsoil 348 | 60.8 | 61.32 | 4819 | Armatimonadetes | Bacteria |
| 667014 | Thermodesulfatator indicus DSM 15286 | 42.4 | 42.61 | 2195 | Thermodesulfobacteria | Bacteria |
| 670487 | Oceanithermus profundus DSM 14977 | 69.79 | 70.31 | 2370 | Deinococcus-Thermus | Bacteria |
| 691883 | Fonticula alba | 64.3 | 68.38 | 6306 | | Eukaryota |
| 694429 | PyroIobus fumarii 1A | 54.9 | 54.95 | 1967 | Crenarchaeota | Archaea |
| 695850 | Saprolegnia parasitica CBS 223.65 | 57.5 | 62.29 | 19578 | | Eukaryota |
| 696747 | Arthrospira platensis NIES-39 | 44.3 | 44.57 | 6625 | Cyanobacteria | Bacteria |
| 703613 | Bifidobacterium animalis subsp. animalis | 60.5 | 61.4 | 1537 | Actinobacteria | Bacteria |
| | ATCC 25527 | | | | | |
| 742818 | Slackia piriformis YIT 12062 | 57.6 | 58.19 | 1792 | Actinobacteria | Bacteria |
| 743299 | Acidithiobacillus ferrivorans SS3 | 56.6 | 57.27 | 3090 | Proteobacteria | Bacteria |
| 743718 | Isoptericola variabilis 225 | 73.9 | 74.05 | 2868 | Actinobacteria | Bacteria |
| 744533 | Naegleria gruberi strain NEG-M | 35 | 34.47 | 15571 | | Eukaryota |
| 746697 | Aequorivita sublithincola DSM 14238 | 36.2 | 36.9 | 3137 | Bacteroidetes | Bacteria |
| 751945 | Thermus oshimai JL-2 | 68.6 | 68.84 | 2119 | Deinococcus-Thermus | Bacteria |
| 753081 | Bigelowiella natans | 44.9 | 49.1 | 21512 | | Eukaryota |
| 754035 | Mesorhizobium australicum WSM2073 | 65 | 63.48 | 5786 | Proteobacteria | Bacteria |
| 755732 | Fluviicola taffensis DSM 16823 | 36.5 | 36.96 | 4030 | Bacteroidetes | Bacteria |
| 760142 | Hippea maritima DSM 10411 | 37.5 | 37.48 | 1675 | Proteobacteria | Bacteria |
| 762948 | Rothia dentocariosa ATCC 17931 | 53.7 | 54.79 | 2213 | Actinobacteria | Bacteria |
| 762983 | Succinatimonas hippei YIT 12066 | 40.3 | 41.31 | 2148 | Proteobacteria | Bacteria |
| 765420 | Oscillochloris trichoides DG-6 | 59.1 | 60.04 | 3231 | Chloroflexi | Bacteria |
| 765952 | Parachlamydia acanthamoebae UV-7 | 39 | 39.73 | 2544 | Chlamydiae | Bacteria |
| 767434 | Frateuria aurantia DSM 6220 | 63.4 | 63.85 | 3097 | Proteobacteria | Bacteria |
| 768670 | Calditerrivibrio nitroreducens DSM 19672 | 35.68 | 35.92 | 2099 | Deferribacteres | Bacteria |
| 768671 | Thiocapsa marina 5811 | 64.1 | 64.57 | 4893 | Proteobacteria | Bacteria |
| 768679 | Thermoproteus tenax Kra 1 | 55.1 | 55.57 | 2048 | Crenarchaeota | Archaea |
| 768706 | Desulfosporosinus orientis DSM 765 | 42.9 | 43.71 | 5232 | Firmicutes | Bacteria |
| 795359 | Thermodesulfobacterium geofontis OPF15 | 30.6 | 30.67 | 1593 | Thermodesulfobacteria | Bacteria |
| 797114 | Halosimplex carlsbadense 2-9-1 | 67.7 | 68.81 | 4390 | Euryarchaeota | Archaea |
| 797210 | Halopiger xanaduensis SH-6 | 65.2 | 66.33 | 4205 | Euryarchaeota | Archaea |
| 797304 | Natronobacterium gregoryi SP2 | 62.2 | 63.19 | 3650 | Euryarchaeota | Archaea |
| 859192 | Candidatus Nitrosoarchaeum limnia BG20 | 32.5 | 33.08 | 2434 | Thaumarchaeota | Archaea |
| 861299 | Gemmatirosa kalamazoonesis | 72.64 | 72.88 | 6105 | Gemmatimonadetes | Bacteria |
| 862908 | Halobacteriovorax marinus SJ | 36.7 | 37.01 | 2787 | Proteobacteria | Bacteria |
| 866499 | Cloacibacillus evryensis DSM 19522 | 56 | 58.05 | 1082 | Synergistetes | Bacteria |
| 866895 | Halobacillus halophilus DSM 2266 | 41.8 | 42.42 | 4108 | Firmicutes | Bacteria |
| 867904 | Methanomethylovorans hollandica | 41.84 | 43.15 | 2554 | Euryarchaeota | Archaea |
| | DSM 15978 | | | | | |
| 868864 | Desulfurobacterium thermolithotrophum | 34.9 | 34.75 | 1507 | Aquificae | Bacteria |
| | DSM 11699 | | | | | |
| 869210 | Marinithermus hydrothermalis DSM 14884 | 68.1 | 68.53 | 2202 | Deinococcus-Thermus | Bacteria |
| 880073 | Caldithrix abyssi DSM 13497 | 45.1 | 46.13 | 3746 | Calditrichaeota | Bacteria |
| 883169 | Turicella otitidis ATCC 51513 | 71 | 71.26 | 1445 | Actinobacteria | Bacteria |
| 885318 | Entamoeba histolytica HM-1:IMSS-A | 24.3 | 27.67 | 5998 | | Eukaryota |
| 886293 | Singulisphaera acidiphila DSM 18658 | 62.27 | 63.26 | 7248 | Planctomycetes | Bacteria |
| 886377 | Muricauda ruestringensis DSM 13258 | 41.4 | 42.09 | 3428 | Bacteroidetes | Bacteria |
| 891968 | Anaerobaculum mobile DSM 13181 | 48 | 48.55 | 2013 | Synergistetes | Bacteria |
| 903503 | Candidatus Moranella endobia PCIT | 43.5 | 45.25 | 406 | Proteobacteria | Bacteria |
| 905079 | Guillardia theta CCMP2712 | 52.9 | 54.77 | 24237 | | Eukaryota |
| 910314 | Dialister microaerophilus UPII 345-E | 35.6 | 36.43 | 1298 | Firmicutes | Bacteria |
| 911008 | Leclercia adecarboxylata ATCC 23216 = | 55.8 | 56.85 | 4592 | Proteobacteria | Bacteria |
| | NBRC102595 | | | | | |
| 926550 | Caldilinea aerophila DSM 14535 = | 58.8 | 59.99 | 4119 | Chloroflexi | Bacteria |
| | NBRC 104270 | | | | | |
| 926559 | Joostella marina DSM 19592 | 33.6 | 34.26 | 3848 | Bacteroidetes | Bacteria |
| 926562 | Owenweeksia hongkongensis DSM 17368 | 40.2 | 40.69 | 3485 | Bacteroidetes | Bacteria |
| 926569 | Anaerolinea thermophila UNI-1 | 53.8 | 54.37 | 3167 | Chloroflexi | Bacteria |
| 926571 | Nitrososphaera viennensis EN76 | 52.7 | 54.07 | 3099 | Thaumarchaeota | Archaea |
| 929556 | Solitalea canadensis DSM 3403 | 37.3 | 38.07 | 4302 | Bacteroidetes | Bacteria |
| 930946 | Fructobacillus fructosus KCTC 3544 | 44.6 | 45.56 | 1439 | Firmicutes | Bacteria |
| 930990 | Botryobasidium botryosum FD-172 SSI | 52.3 | 55.43 | 16391 | Basidiomycota | Eukaryota |
| 931890 | Eremothecium cymbalariae DBVPG#7215 | 40.32 | 41.38 | 4432 | Ascomycota | Eukaryota |
| 937777 | Deinococcus peraridilitoris DSM 19664 | 63.71 | 64.41 | 4176 | Deinococcus-Thermus | Bacteria |
| 944289 | Gymnopus luxurians FD-317 M1 | 45.1 | 48.37 | 14499 | Basidiomycota | Eukaryota |
| 945553 | Hypholoma sublateritium FD-334 SS-4 | 51 | 54.6 | 17010 | Basidiomycota | Eukaryota |
| 945713 | Ignavibacterium album JCM 16511 | 33.9 | 34.31 | 3188 | Ignavibacteriae | Bacteria |
| 946077 | Imtechella halotolerans K1 | 35.5 | 36.13 | 2687 | Bacteroidetes | Bacteria |
| 946362 | Salpingoeca rosetta | 55.5 | 60.4 | 11648 | | Eukaryota |
| 983544 | Lacinutrix sp. 5H-3-7-4 | 30.8 | 31.35 | 2963 | Bacteroidetes | Bacteria |
| 997884 | Bacteroides nordii | 40.8 | 41.8 | 4275 | Bacteroidetes | Bacteria |
| 999415 | Eggerthia catenaformis OT 569 = DSM | 32.8 | 32.7 | 1861 | Firmicutes | Bacteria |
| | 20559 | | | | | |
| 1002672 | Candidatus Pelagibacter sp. IMCC9063 | 31.7 | 31.86 | 1443 | Proteobacteria | Bacteria |
| 1006000 | Kluyvera ascorbata ATCC 33433 | 54.3 | 55.69 | 4561 | Proteobacteria | Bacteria |
| 1009370 | Acetonema longum DSM 6540 | 50.4 | 51.42 | 4197 | Firmicutes | Bacteria |
| 1028800 | Neorhizobium galegae bv. orientalis str. | 61.25 | 62 | 6163 | Proteobacteria | Bacteria |
| | HAMBI 540 | | | | | |
| 1033802 | Salinisphaera shabanensis E1L3A | 61.6 | 62.04 | 3515 | Proteobacteria | Bacteria |
| 1033810 | Haloplasma contractile SSD-17B | 32.3 | 33.41 | 3017 | | Bacteria |
| 1033991 | Rhizobium leguminosarum bv. trifolii | 61.17 | 61.84 | 6480 | Proteobacteria | Bacteria |
| 1041607 | CB782 | 30.4 | 30.81 | 6702 | Ascomycota | Eukaryota |
| | Wickerhamomyces ciferrii | | | | | |
| 1046627 | Bizionia argentinensis JUB59 | 33.8 | 34.56 | 3088 | Bacteroidetes | Bacteria |
| 1047168 | Zymoseptoria brevis | 51.2 | 55.67 | 10475 | Ascomycota | Eukaryota |
| 1055104 | Cobetia amphilecti str. KMM 296 | 62.5 | 63.51 | 2704 | Proteobacteria | Bacteria |
| 1056495 | Caldisphaera lagunensis DSM 15908 | 30 | 30.78 | 1475 | Crenarchaeota | Archaea |
| 1069680 | Pneumocystis murina b123 | 27 | 30.91 | 3602 | Ascomycota | Eukaryota |
| 1072681 | Candidatus Haloredivivus sp. G17 | 42 | 42.7 | 1863 | Candidatus | Archaea |
| | | | | | Nanohaloarchaeota | |
| 1116230 | Wolbachia pipientis wAIbB | 33.8 | 34.36 | 961 | Proteobacteria | Bacteria |
| 1121088 | Bacillus coagulans DSM 1 = ATCC 7050 | 46.9 | 47.65 | 3236 | Firmicutes | Bacteria |
| 1121915 | Geoalkalibacter ferrihydriticus DSM 17813 | 57.9 | 58.86 | 2897 | Proteobacteria | Bacteria |
| 1123384 | Pseudothermotoga hypogea DSM 11164 = | 49.5 | 49.63 | 2094 | Thermotogae | Bacteria |
| | NBRC 106472 | | | | | |
| 1125630 | Klebsiella pneumoniae subsp. pneumoniae | 57.14 | 58.25 | 5378 | Proteobacteria | Bacteria |
| | HS11286 | | | | | |
| 1129897 | Nitrolancea hollandica Lb | 62.6 | 62.93 | 3954 | Chloroflexi | Bacteria |
| 1142394 | Phycisphaera mikurensis NBRC 102666 | 73.23 | 73.13 | 3283 | Planctomycetes | Bacteria |
| 1157490 | Tumebacillus flagellatus | 56.5 | 57.75 | 4434 | Firmicutes | Bacteria |
| 1165094 | Richelia intracellularis HH01 | 33.7 | 38.26 | 2258 | Cyanobacteria | Bacteria |
| 1172194 | Hydrocarboniphaga effusa AP103 | 65.2 | 65.72 | 4680 | Proteobacteria | Bacteria |
| 1177928 | Thalassospira profundimaris WP0211 | 55.2 | 55.94 | 4034 | Proteobacteria | Bacteria |
| 1177931 | Thiovulum sp. ES | 33 | 33.25 | 2022 | Proteobacteria | Bacteria |
| 1182568 | Deinococcus puniceus | 62.6 | 63.72 | 2336 | Deinococcus-Thermus | Bacteria |
| 1183438 | Gloeobacter kilaueensis JS1 | 60.5 | 61.37 | 4395 | Cyanobacteria | Bacteria |
| 1185651 | Enterovibrio norvegicus FF-454 | 47.6 | 48.17 | 4276 | Proteobacteria | Bacteria |
| 1189619 | Psychroflexus gondwanensis ACAM 44 | 35.8 | 36.41 | 2895 | Bacteroidetes | Bacteria |
| 1189621 | Nitritalea halalkaliphila LW7 | 48.6 | 49.35 | 3035 | Bacteroidetes | Bacteria |
| 1198115 | Thaumarchaeota archaeon SCGC | 43.3 | 44.52 | 605 | Thaumarchaeota | Archaea |
| | AB-539-E09 | | | | | |
| 1198449 | Aeropyrum camini SY1 = JCM 12091 | 56.7 | 57.31 | 1645 | Crenarchaeota | Archaea |
| 1201294 | Methanoculleus bourgensis MS2 | 60.6 | 61.54 | 2579 | Euryarchaeota | Archaea |
| 1208320 | Thalassolituus oleivorans R6-15 | 46.6 | 46.98 | 3368 | Proteobacteria | Bacteria |
| 1208660 | Bordetella parapertussis Bpp5 | 67.78 | 68.14 | 4174 | Proteobacteria | Bacteria |
| 1208920 | Candidatus Kinetoplastibacterium | 31.2 | 31.87 | 694 | Proteobacteria | Bacteria |
| | oncopeltii TCC290E | | | | | |
| 1209989 | Tepidanaerobacter acetatoxydans Re1 | 37.5 | 38.31 | 2524 | Firmicutes | Bacteria |
| 1223560 | Pythium vexans DAOM BR484 | 58.7 | 61.38 | 11851 | | Eukaryota |
| 1227812 | Piscirickettsia salmonis LF-89 = | 39.62 | 40.82 | 3127 | Proteobacteria | Bacteria |
| | ATCC VR-1361 | | | | | |
| 1229908 | Candidatus Nitrosopumilus koreensis AR1 | 34.2 | 34.69 | 1883 | Thaumarchaeota | Archaea |
| 1236689 | Candidatus Methanomethylophilus alvus | 55.6 | 56.62 | 1641 | Euryarchaeota | Archaea |
| | MX1201 | | | | | |
| 1236703 | Candidatus Photodesmus katoptron Akat1 | 31.06 | 31.78 | 854 | Proteobacteria | Bacteria |
| 1237085 | Candidatus Nitrososphaera gargensis | 48.3 | 49.8 | 3559 | Thaumarchaeota | Archaea |
| | Ga9.2 | | | | | |
| 1245935 | Tolypothrix campylonemoides VB511288 | 45.1 | 46.39 | 6844 | Cyanobacteria | Bacteria |
| 1257118 | Acanthamoeba castellanii str. Neff | 57.8 | 62.95 | 14229 | | Eukaryota |
| 1266370 | Nitrospina gracilis 3-211 | 56.1 | 56.92 | 2947 | Nitrospinae | Bacteria |
| 1266844 | Acetobacter pasteurianus 386B | 53.2 | 53.58 | 2865 | Proteobacteria | Bacteria |
| 1273541 | Pyrodictium delaneyi | 53.9 | 54.37 | 2035 | Crenarchaeota | Archaea |
| 1287680 | Neofusicoccum parvum UCRNP2 | 56.7 | 60.86 | 10366 | Ascomycota | Eukaryota |
| 1292022 | Curtobacterium flaccumfaciens UCD-AKU | 70.8 | 71.02 | 3365 | Actinobacteria | Bacteria |
| 1295009 | Candidatus Methanomassiliicoccus | 41.3 | 42.14 | 1826 | Euryarchaeota | Archaea |
| | intestinalis Issoire-Mx1 str. Mx1-Issoire | | | | | |
| 1298851 | Thermosulfidibacter takaii ABI70S6 | 43 | 42.99 | 1757 | Aquificae | Bacteria |
| 1303518 | Chthonomonas calidirosea T49 | 54.6 | 55.16 | 2805 | Armatimonadetes | Bacteria |
| 1304892 | Xanthomonas axonopodis Xac29-1 | 64.72 | 65.21 | 3289 | Proteobacteria | Bacteria |
| 1307761 | Salinispira pacifica | 51.9 | 52.3 | 3397 | Spirochaetes | Bacteria |
| 1313172 | llumatobacter coccineus YM16-304 | 67.3 | 67.47 | 4289 | Actinobacteria | Bacteria |
| 1319815 | Cetobacterium somerae ATCC BAA-474 | 28.6 | 28.95 | 2889 | Fusobacteria | Bacteria |
| 1321371 | Holospora undulata HU1 | 36.1 | 37.52 | 1218 | Proteobacteria | Bacteria |
| 1330330 | Kosmotoga pacifica | 42.5 | 42.81 | 1897 | Thermotogae | Bacteria |
| 1341181 | Flavobacterium limnosediminis JC2902 | 38.5 | 39.45 | 2901 | Bacteroidetes | Bacteria |
| 1343739 | Palaeococcus pacificus DY20341 | 43 | 43.55 | 1988 | Euryarchaeota | Archaea |
| 1347342 | Formosa agariphila KMM 3901 | 33.6 | 34.27 | 3567 | Bacteroidetes | Bacteria |
| 1379270 | Gemmatimonas phototrophica | 64.4 | 64.58 | 3388 | Gemmatimonadetes | Bacteria |
| 1379858 | Mucispirillum schaedleri ASF457 | 31.2 | 31.94 | 2124 | Deferribacteres | Bacteria |
| 1397361 | Sporothrix schenckii 1099-18 | 55 | 61.56 | 10288 | Ascomycota | Eukaryota |
| 1408204 | Candidatus Endomicrobium | 35.8 | 36.79 | 2768 | Elusimicrobia | Bacteria |
| | trichonymphae | | | | | |
| 1427984 | Candidatus Hepatoplasma crinochetorum | 22.5 | 22.73 | 567 | Tenericutes | Bacteria |
| | Av | | | | | |
| 1429438 | Candidatus Entotheonella sp. TSY1 | 55.3 | 56.83 | 8139 | Candidatus | Bacteria |
| | | | | | Tectomicrobia | |
| 1429439 | Candidatus Entotheonella sp. TSY2 | 55.3 | 56.69 | 8264 | Candidatus | Bacteria |
| | | | | | Tectomicrobia | |
| 1432061 | Dehalococcoides mccartyi CG5 | 48.9 | 48.04 | 1428 | Chloroflexi | Bacteria |
| 1432562 | Salinicoccus sediminis | 48.7 | 49.84 | 2485 | Firmicutes | Bacteria |
| 1432656 | Thermococcus guaymasensis DSM 11113 | 52.9 | 53.61 | 2085 | Euryarchaeota | Archaea |
| 1435057 | Agrobacterium tumefaciens LBA4213 | 59.87 | 59.37 | 5420 | Proteobacteria | Bacteria |
| | (Ach5) | | | | | |
| 1439331 | Lelliottia amnigena CHS 78 | 54.3 | 56.12 | 4511 | Proteobacteria | Bacteria |
| 1441628 | Leptospirillum ferriphilum YSK | 54.6 | 54.92 | 2260 | Nitrospirae | Bacteria |
| 1454006 | Siansivirga zeaxanthinifaciens CC-SAMT-1 | 33.5 | 34.33 | 2761 | Bacteroidetes | Bacteria |
| 1469144 | Streptomyces thermoautotrophicus | 69.2 | 70.88 | 3626 | Actinobacteria | Bacteria |
| 1502293 | Marine Group 1 thaumarchaeote SCGC | 34.2 | 34.72 | 1670 | Thaumarchaeota | Archaea |
| | AAA799-N04 | | | | | |
| 1514904 | Ahrensia marina str. LZD062 | 50.1 | 50.77 | 3143 | Proteobacteria | Bacteria |
| 1519565 | Fistulifera Solaris | 45.6 | 48.45 | 20365 | Bacillariophyta | Eukaryota |
| 1529318 | Cryobacterium sp. MLB-32 | 67.53 | 65.31 | 3045 | Actinobacteria | Bacteria |
| 1574623 | Lyngbya confervoides BDU141951 | 55 | 56.67 | 5685 | Cyanobacteria | Bacteria |
| 1577684 | Candidatus Nanopusillus acidilobi | 24.2 | 24.14 | 580 | Nanoarchaeota | Archaea |
| 1618331 | Berkelbacteria bacterium | 35.9 | 36.1 | 907 | Candidatus | Bacteria |
| | GW2011_GWA1_36_9 | | | | Berkelbacteria | |
| 1618369 | Candidatus Beckwithbacteria bacterium | 43 | 43.3 | 663 | Candidatus | Bacteria |
| | GW2011_GWA2_43_10 | | | | Beckwithbacteria | |
| 1618380 | Candidatus Collierbacteria bacterium | 43.8 | 44.05 | 733 | Candidatus | Bacteria |
| | GW2011_GWA2_44_99 | | | | Collierbacteria | |
| 1618405 | Candidatus Curtissbacteria bacterium | 40.8 | 41.15 | 1014 | Candidatus | Bacteria |
| | GW2011_GWAl_40_16 | | | | Curtissbacteria | |
| 1618443 | Candidatus Gottesmanbacteria bacterium | 43.2 | 43.69 | 1684 | Candidatus | Bacteria |
| | GW2011_GWA2_43_14 | | | | Gottesmanbacteria | |
| 1618595 | Candidatus Woesebacteria bacterium | 40.1 | 40.32 | 777 | Candidatus | Bacteria |
| | GW2011_GWD2_40_19 | | | | Woesebacteria | |
| 1618609 | Candidatus Azambacteria bacterium | 41.5 | 41.91 | 585 | Candidatus | Bacteria |
| | GW2011_GWAl_42_19 | | | | Azambacteria | |
| 1618623 | Candidatus Azambacteria bacterium | 46.1 | 46.72 | 582 | Candidatus | Bacteria |
| | GW2011_GWD2_46_48 | | | | Azambacteria | |
| 1618643 | Candidatus Falkowbacteria bacterium | 43.3 | 44.37 | 789 | Candidatus | Bacteria |
| | GW2011_GWF2_43_32 | | | | Falkowbacteria | |
| 1618662 | Candidatus Jorgensenbacteria bacterium | 45.2 | 46.02 | 631 | Candidatus | Bacteria |
| | GW2011_GWA2_45_13 | | | | Jorgensenbacteria | |
| 1618671 | Candidatus Kaiserbacteria bacterium | 52 | 52.62 | 966 | Candidatus | Bacteria |
| | GW2011_GWA2_52_12 | | | | Kaiserbacteria | |
| 1618673 | Candidatus Kaiserbacteria bacterium | 50 | 50.55 | 458 | Candidatus | Bacteria |
| | GW2011_GWBl_50_17 | | | | Kaiserbacteria | |
| 1618729 | Candidatus Nomurabacteria bacterium | 36.9 | 37.1 | 590 | Candidatus | Bacteria |
| | GW2011_GWAl_37_20 | | | | Nomurabacteria | |
| 1618742 | Candidatus Nomurabacteria bacterium | 36.7 | 37.24 | 783 | Candidatus | Bacteria |
| | GW2011_GWBl_37_5 | | | | Nomurabacteria | |
| 1618775 | Candidatus Nomurabacteria bacterium | 36.2 | 36.81 | 795 | Candidatus | Bacteria |
| | GW2011_GWF2_36_19 | | | | Nomurabacteria | |
| 1618777 | Candidatus Nomurabacteria bacterium | 39.6 | 39.96 | 578 | Candidatus | Bacteria |
| | GW2011_GWF2_40_31 | | | | Nomurabacteria | |
| 1618821 | Parcubacteria group bacterium | 41.6 | 42.09 | 584 | | Bacteria |
| | GW2011_GWA2_42_18 | | | | | |
| 1618840 | Parcubacteria group bacterium | 47.1 | 47.34 | 845 | | Bacteria |
| | GW2011_GWA2_47_10b | | | | | |
| 1618841 | Parcubacteria group bacterium | 46.8 | 47.44 | 753 | | Bacteria |
| | GW2011_GWA2_47_12 | | | | | |
| 1618924 | Parcubacteria group bacterium | 40.4 | 40.91 | 813 | | Bacteria |
| | GW2011_GWC2_40_31 | | | | | |
| 1619005 | Candidatus Wolfebacteria bacterium | 46.7 | 47.48 | 1053 | Candidatus | Bacteria |
| | GW2011_GWA2_47_9b | | | | Wolfebacteria | |
| 1619029 | Candidatus Yanofskybacteria bacterium | 41.3 | 41.76 | 640 | Candidatus | Bacteria |
| | GW2011_GWC2_41_9 | | | | Yanofskybacteria | |
| 1619051 | Candidatus Magasanikbacteria bacterium | 43 | 43.27 | 1142 | Candidatus | Bacteria |
| | GW2011_GWD2_43_18 | | | | Magasanikbacteria | |
| 1619068 | Candidatus Peregrinibacteria bacterium | 43.1 | 43.4 | 1124 | Candidatus | Bacteria |
| | GW2011_GWF2_43_17 | | | | Peregrinibacteria | |
| 1619079 | candidate division TM6 bacterium | 32.7 | 33.16 | 880 | | Bacteria |
| | GW2011_GWF2_32_72 | | | | | |
| 1630693 | Gemmata sp. SH-PL17 | 64.2 | 64.99 | 7691 | Planctomycetes | Bacteria |
| 1737403 | Nanohaloarchaea archaeon SG9 | 46.4 | 46.95 | 1183 | Candidatus | Archaea |

TABLE 3
Organisms by phylum

| | | | Num | Num | Num | Num |
|---|---|---|---|---|---|---|
| TaxId | Domain | Phylum | Families | Genera | Orders | Species |
| 51967 | Archaea | Candidatus Korarchaeota | 0 | 1 | 0 | 1 |
| 1462430 | Archaea | Candidatus Nanohaloarchaeota | 0 | 0 | 0 | 2 |
| 28889 | Archaea | Crenarchaeota | 5 | 9 | 4 | 11 |
| 28890 | Archaea | Euryarchaeota | 18 | 31 | 12 | 40 |
| 192989 | Archaea | Nanoarchaeota | 2 | 2 | 1 | 2 |
| 651137 | Archaea | Thaumarchaeota | 3 | 4 | 3 | 8 |
| | Archaea | [Total] | 0 | 0 | 0 | 64 |
| 57723 | Bacteria | Acidobacteria | 2 | 2 | 2 | 2 |
| 201174 | Bacteria | Actinobacteria | 20 | 31 | 17 | 35 |
| 200783 | Bacteria | Aquificae | 3 | 9 | 2 | 10 |
| 67819 | Bacteria | Armatimonadetes | 2 | 2 | 2 | 2 |
| 976 | Bacteria | Bacteroidetes | 9 | 31 | 5 | 35 |
| 67814 | Bacteria | Caldiserica | 1 | 1 | 1 | 1 |
| 1930617 | Bacteria | Calditrichaeota | 1 | 1 | 1 | 1 |
| 1752741 | Bacteria | Candidatus Azambacteria | 0 | 0 | 0 | 2 |
| 1752726 | Bacteria | Candidatus Beckwithbacteria | 0 | 0 | 0 | 1 |
| 1618330 | Bacteria | Candidatus Berkelbacteria | 0 | 0 | 0 | 1 |
| 1752725 | Bacteria | Candidatus Collierbacteria | 0 | 0 | 0 | 1 |
| 1752717 | Bacteria | Candidatus Curtissbacteria | 0 | 0 | 0 | 1 |
| 1752728 | Bacteria | Candidatus Falkowbacteria | 0 | 0 | 0 | 1 |
| 1752720 | Bacteria | Candidatus Gottesmanbacteria | 0 | 0 | 0 | 1 |
| 1752739 | Bacteria | Candidatus Jorgensenbacteria | 0 | 0 | 0 | 1 |
| 1752734 | Bacteria | Candidatus Kaiserbacteria | 0 | 0 | 0 | 2 |
| 1752731 | Bacteria | Candidatus Magasanikbacteria | 0 | 0 | 0 | 1 |
| 1752729 | Bacteria | Candidatus Nomurabacteria | 0 | 0 | 0 | 4 |
| 1619053 | Bacteria | Candidatus Peregrinibacteria | 0 | 0 | 0 | 1 |
| 1802339 | Bacteria | Candidatus Tectomicrobia | 0 | 1 | 0 | 2 |
| 1752722 | Bacteria | Candidatus Woesebacteria | 0 | 0 | 0 | 1 |
| 1752735 | Bacteria | Candidatus Wolfebacteria | 0 | 0 | 0 | 1 |
| 1752733 | Bacteria | Candidatus Yanofskybacteria | 0 | 0 | 0 | 1 |
| 204428 | Bacteria | Chlamydiae | 3 | 3 | 2 | 5 |
| 1090 | Bacteria | Chlorobi | 1 | 2 | 1 | 3 |
| 200795 | Bacteria | Chloroflexi | 10 | 12 | 8 | 14 |
| 200938 | Bacteria | Chrysiogenetes | 1 | 1 | 1 | 1 |
| 1117 | Bacteria | Cyanobacteria | 10 | 13 | 5 | 15 |
| 200930 | Bacteria | Deferribacteres | 1 | 4 | 1 | 4 |
| 1297 | Bacteria | Deinococcus-Thermus | 3 | 6 | 2 | 11 |
| 68297 | Bacteria | Dictyoglomi | 1 | 1 | 1 | 2 |
| 74152 | Bacteria | Elusimicrobia | 2 | 2 | 2 | 2 |
| 65842 | Bacteria | Fibrobacteres | 1 | 1 | 1 | 1 |
| 1239 | Bacteria | Firmicutes | 23 | 34 | 10 | 44 |
| 32066 | Bacteria | Fusobacteria | 2 | 6 | 1 | 8 |
| 142182 | Bacteria | Gemmatimonadetes | 1 | 2 | 1 | 3 |
| 1134404 | Bacteria | Ignavibacteriae | 1 | 1 | 1 | 1 |
| 256845 | Bacteria | Lentisphaerae | 1 | 1 | 1 | 1 |
| 1293497 | Bacteria | Nitrospinae | 1 | 1 | 1 | 1 |
| 40117 | Bacteria | Nitrospirae | 1 | 4 | 1 | 4 |
| 203682 | Bacteria | Planctomycetes | 4 | 6 | 2 | 6 |
| 1224 | Bacteria | Proteobacteria | 55 | 84 | 35 | 92 |
| 203691 | Bacteria | Spirochaetes | 3 | 5 | 2 | 6 |
| 508458 | Bacteria | Synergistetes | 1 | 4 | 1 | 4 |
| 544448 | Bacteria | Tenericutes | 2 | 5 | 2 | 11 |
| 200940 | Bacteria | Thermodesulfobacteria | 1 | 2 | 1 | 3 |
| 200918 | Bacteria | Thermotogae | 3 | 8 | 3 | 10 |
| 74201 | Bacteria | Verrucomicrobia | 4 | 4 | 4 | 4 |
| | Bacteria | [Unknown] | 0 | 0 | 0 | 7 |
| | Bacteria | [Total] | 0 | 0 | 0 | 371 |
| 5794 | Eukaryota | Apicomplexa | 5 | 5 | 2 | 5 |
| 6656 | Eukaryota | Arthropoda | 1 | 1 | 1 | 1 |
| 4890 | Eukaryota | Ascomycota | 10 | 13 | 8 | 16 |
| 2836 | Eukaryota | Bacillariophyta | 4 | 4 | 3 | 4 |
| 5204 | Eukaryota | Basidiomycota | 9 | 9 | 5 | 9 |
| 451459 | Eukaryota | Blastocladiomycota | 1 | 1 | 1 | 1 |
| 3041 | Eukaryota | Chlorophyta | 6 | 6 | 2 | 6 |
| 4761 | Eukaryota | Chytridiomycota | 1 | 1 | 1 | 1 |
| 6073 | Eukaryota | Cnidaria | 1 | 1 | 1 | 1 |
| 10197 | Eukaryota | Ctenophora | 1 | 1 | 1 | 1 |
| 10226 | Eukaryota | Placozoa | 0 | 1 | 0 | 1 |
| 6040 | Eukaryota | Porifera | 1 | 1 | 1 | 1 |
| 10190 | Eukaryota | Rotifera | 1 | 1 | 1 | 1 |
| 35493 | Eukaryota | Streptophyta | 2 | 2 | 2 | 2 |
| | Eukaryota | [Unknown] | 0 | 0 | 0 | 28 |
| | Eukaryota | [Total] | 0 | 0 | 0 | 78 |
| | [All] | [Total] | 245 | 384 | 169 | 513 |

TABLE 4
Genomic properties

| | | Gen- | Gen- | In | | | Gen- | Gen- | In |
|---|---|---|---|---|---|---|---|---|---|
| Tax | | omic | omic | Phylo | Tax | | omic | omic | Phylo |
| Id | Species | ENc' | GC % | Tree | Id | Species | ENc' | GC % | Tree |
| 592010 | Abiotrophia defectiva | 53.33 | 47 | + | 257314 | Lactobacillus | 52.22 | 34.6 | |
| | ATCC 49176 | | | | | johnsonii NCC 533 | | | |
| 1257118 | Acanthamoeba castellanii | 49.81 | 57.8 | + | 220668 | Lactobacillus | 53.3 | 44.45 | |
| | str. Neff | | | | | plantarum WCFS1 | | | |
| 1266844 | Acetobacter pasteurianus | 50.76 | 53.2 | + | 420890 | Lactococcus garvieae | 52.24 | 38.8 | + |
| | 386B | | | | | Lg2 | | | |
| 574087 | Acetohalobium arabaticum | 53.49 | 36.6 | + | 272623 | Lactococcus lactis | 51.51 | 35.3 | |
| | DSM 5501 | | | | | subsp. lactis ll1403 | | | |
| 1009370 | Acetonema longum | 50.94 | 50.4 | + | 911008 | Leclercia | 46.92 | 55.8 | + |
| | DSM 6540 | | | | | adecarboxylata ATCC | | | |
| | | | | | | 23216 = | | | |
| | | | | | | NBRC10 2595 | | | |
| 441768 | Acholeplasma laidlawii | 51.76 | 31.9 | + | 398720 | Leeuwenhoekiella | 54.68 | 39.8 | + |
| | PG-8A | | | | | blandensis MED217 | | | |
| 525909 | Acidimicrobium | 50.33 | 68.3 | + | 281090 | Leifsonia xyli subsp. | 49.36 | 68.3 | + |
| | ferrooxidans DSM 10331 | | | | | xyli str. CTCB07 | | | |
| 507754 | Acidiplasma aeolicum str. | 49.45 | 34.2 | | 347515 | Leishmania major | 53.46 | 59.71 | |
| | VT | | | | | strain Friedlin | | | |
| 743299 | Acidithiobacillus | 53.39 | 56.6 | + | 1439331 | Lelliottia amnigena | 47.6 | 54.3 | + |
| | ferrivorans SS3 | | | | | CHS 78 | | | |
| 243159 | Acidithiobacillus | 52.52 | 58.8 | | 313628 | Lentisphaera | 54.23 | 41 | + |
| | ferrooxidans ATCC 23270 | | | | | araneosa HTCC2155 | | | |
| 240015 | Acidobacterium | 49.92 | 60.5 | | 456481 | Leptospira biflexa | 55.31 | 38.9 | + |
| | capsulatum ATCC 51196 | | | | | serovar Patoc strain | | | |
| | | | | | | ‘Patoc 1 (Paris)’ | | | |
| 351607 | Acidothermus cellulolyticus | 53.02 | 66.9 | + | 267671 | Leptospira | 54.65 | 35.01 | |
| | 11B | | | | | interrogans serovar | | | |
| | | | | | | Copenhageni str. | | | |
| | | | | | | Fiocruz Li-130 | | | |
| 400667 | Acinetobacter baumannii | 50.71 | 39 | | 1441628 | Leptospirillum | 51.77 | 54.6 | + |
| | ATCC 17978 | | | | | ferriphilum YSK | | | |
| 104782 | Adineta vaga | 47.36 | 31.2 | | 596323 | Leptotrichia | 51.46 | 31.6 | + |
| | | | | | | goodfellowii F0264 | | | |
| 746697 | Aequorivita sublithincola | 55.48 | 36.2 | + | 272626 | Listeria innocua | 53.51 | 37.35 | + |
| | DSM 14238 | | | | | Clip11262 | | | |
| 1198449 | Aeropyrum camini SY1 = | 47.68 | 56.7 | | 169963 | Listeria | 53.37 | 38 | |
| | JCM 12091 | | | | | monocytogenes | | | |
| | | | | | | EGD-e | | | |
| 272557 | Aeropyrum pernix K1 | 48.11 | 56.3 | | 1574623 | Lyngbya | 52.75 | 55 | |
| | | | | | | confervoides | | | |
| | | | | | | BDU141951 | | | |
| 176299 | Agrobacterium fabrum str. | 49.35 | 59.06 | | 242507 | Magnaporthe oryzae | 56.33 | 51.59 | |
| | C58 | | | | | | | | |
| 1435057 | Agrobacterium | 49.96 | 59.87 | | 156889 | Magnetococcus | 49.97 | 54.2 | + |
| | tumefaciens LBA4213 | | | | | marinus MC-1 | | | |
| | (Ach5) | | | | | | | | |
| 1514904 | Ahrensia marina str. | 50.9 | 50.1 | | 1502293 | Marine Group 1 | 51.73 | 34.2 | + |
| | LZD062 | | | | | thaumarchaeote | | | |
| | | | | | | SCGC AAA799-N04 | | | |
| 349741 | Akkermansia muciniphila | 48.02 | 55.8 | + | 869210 | Marinithermus | 48.3 | 68.1 | + |
| | ATCC BAA-835 | | | | | hydrothermalis | | | |
| | | | | | | DSM 14884 | | | |
| 65357 | Albugo candida | 57.43 | 43.2 | | 443254 | Marinitoga | 53.34 | 29.18 | + |
| | | | | | | piezophila KA3 | | | |
| 393595 | Alcanivorax borkumensis | 51.3 | 54.7 | + | 504728 | Meiothermus ruber | 46.92 | 63.4 | + |
| | SK2 | | | | | DSM 1279 | | | |
| 543302 | Alicyclobacillus | 51.58 | 61.86 | + | 754035 | Mesorhizobium | 47.82 | 65 | + |
| | acidocaldarius LAA1 | | | | | australicum | | | |
| | | | | | | WSM2073 | | | |
| 187272 | Alkalilimnicola ehrlichii | 47.12 | 67.5 | + | 660470 | Mesotoga prima | 54.94 | 45.5 | + |
| | MLHE-1 | | | | | MesG1.Ag.4.2 | | | |
| 578462 | Allomyces macrogynus | 50.11 | 60.5 | + | 420247 | Methanobrevibacter | 52.58 | 31 | + |
| | ATCC 38327 | | | | | smithii ATCC 35061 | | | |
| 400682 | Amphimedon | 56.04 | 37.5 | + | 243232 | Methanocaldococcus | 52.24 | 31.27 | + |
| | queenslandica | | | | | jannaschii DSM 2661 | | | |
| 46234 | Anabaena sp. 90 | 54 | 38.09 | | 267377 | Methanococcus | 52.5 | 33.3 | + |
| | | | | | | maripaludis S2 | | | |
| 891968 | Anaerobaculum mobile | 55.05 | 48 | + | 410358 | Methanocorpusculum | 52.38 | 50 | + |
| | DSM 13181 | | | | | labreanum Z | | | |
| 525919 | Anaerococcus prevotii | 53.01 | 35.67 | + | 1201294 | Methanoculleus | 50.63 | 60.6 | + |
| | DSM 20548 | | | | | bourgensis MS2 | | | |
| 926569 | Anaerolinea thermophila | 51.81 | 53.8 | + | 28892 | Methanofollis | 50 | 61 | + |
| | UNI-1 | | | | | liminatans DSM 4140 | | | |
| 491915 | Anoxybacillus flavithermus | 50.61 | 41.8 | + | 644295 | Methanohalobium | 54.62 | 36.4 | + |
| | WK1 | | | | | evestigatum Z-7303 | | | |
| 224324 | Aquifex aeolicus VF5 | 48.34 | 43.32 | + | 867904 | Methanomethylovorans | 55.09 | 41.84 | + |
| | | | | | | hollandica | | | |
| | | | | | | DSM 15978 | | | |
| 224325 | Archaeoglobus fulgidus | 49.67 | 48.6 | + | 190192 | Methanopyrus | 52.31 | 61.2 | + |
| | DSM 4304 | | | | | kandleri AV19 | | | |
| 696747 | Arthrospira platensis | 55.65 | 44.3 | + | 188937 | Methanosarcina | 54.78 | 42.7 | |
| | NIES-39 | | | | | acetivorans C2A | | | |
| 5061 | Aspergillus niger | 58.4 | 50.3 | | 213585 | Methanosarcina | 53.11 | 41.4 | |
| | | | | | | mazei S-6 | | | |
| 322098 | Aster yellows witches’ | 51.65 | 26.83 | + | 339860 | Methanosphaera | 50.55 | 27.6 | + |
| | broom phytoplasma AYWB | | | | | stadtmanae | | | |
| | | | | | | DSM 3091 | | | |
| 573065 | Asticcacaulis excentricus | 49.49 | 59.53 | + | 521011 | Methanosphaerula | 51.93 | 55.4 | + |
| | CB 48 | | | | | palustris E1-9c | | | |
| 44056 | Aureococcus | 46.19 | 67.4 | + | 187420 | Methanothermobacter | 47.42 | 49.5 | + |
| | anophagefferens | | | | | thermautotrophicus | | | |
| | | | | | | str. Delta H | | | |
| 484906 | Babesia bovis T2Bo | 57.75 | 41.61 | + | 481448 | Methylacidiphilum | 54.5 | 45.5 | + |
| | | | | | | infernorum V4 | | | |
| 1121088 | Bacillus coagulans DSM 1 = | 50.66 | 46.9 | | 419610 | Methylobacterium | 48.13 | 68.2 | + |
| | ATCC 7050 | | | | | extorquens PA1 | | | |
| 272558 | Bacillus halodurans C-125 | 56.37 | 43.7 | | 243233 | Methylococcus | 49.27 | 63.6 | + |
| | | | | | | capsulatus str. Bath | | | |
| 439292 | Bacillus selenitireducens | 53.93 | 48.7 | + | 449447 | Microcystis | 54.59 | 42.3 | |
| | MLS10 | | | | | aeruginosa NIES-843 | | | |
| 224308 | Bacillus subtilis subsp. | 54.95 | 43.5 | | 564608 | Micromonas pusilia | 48.66 | 65.7 | |
| | subtilis str. 168 | | | | | CCMP1545 | | | |
| 295405 | Bacteroides fragilis YCH46 | 54.64 | 43.24 | | 500635 | Mitsuokella | 43.29 | 58 | + |
| | | | | | | multacida | | | |
| | | | | | | DSM 20544 | | | |
| 997884 | Bacteroides nordii | 54.4 | 40.8 | | 27923 | Mnemiopsis leidyi | 57.3 | 39.1 | |
| 226186 | Bacteroides | 53.9 | 42.82 | | 548479 | Mobiluncus curtisii | 53.83 | 55.4 | + |
| | thetaiotaomicron VPI-5482 | | | | | ATCC 43063 | | | |
| 283166 | Bartonella henselae str. | 51.31 | 38.2 | | 554373 | Moniliophthora | 58.52 | 47.7 | |
| | Houston-1 | | | | | perniciosa FA553 | | | |
| 264462 | Bdellovibrio bacteriovorus | 49.57 | 43.3 | + | 431895 | Monosiga brevicollis | 53.88 | 54.33 | + |
| | HD100 | | | | | MX1 | | | |
| 1618331 | Berkelbacteria bacterium | 56.75 | 35.9 | + | 1379858 | Mucispirillum | 50.08 | 31.2 | + |
| | GW2011_GWA1_36_9 | | | | | schaedleri ASF457 | | | |
| 703613 | subsp. animalis | 47.53 | 60.5 | + | 886377 | ruestringensis | 53.98 | 41.4 | + |
| | ATCC 25527 | | | | | DSM 13258 | | | |
| 753081 | Bigelowiella natans | 58.83 | 44.9 | + | 272631 | Mycobacterium | 55.25 | 57.8 | |
| | | | | | | leprae TN | | | |
| 1046627 | Bizionia argentinensis | 54.42 | 33.8 | + | 83332 | Mycobacterium | 52.13 | 65.6 | |
| | JUB59 | | | | | tuberculosis H37Rv | | | |
| 331104 | Blattabacterium sp. | 50.77 | 23.84 | | 347257 | Mycoplasma | 52.2 | 29.7 | + |
| | (Blattella germanica) str. | | | | | agalactiae PG2 | | | |
| | Bge | | | | | | | | |
| 1208660 | Bordetella parapertussis | 43.93 | 67.78 | | 243273 | Mycoplasma | 54.12 | 31.7 | |
| | Bpp5 | | | | | genitalium G37 | | | |
| 930990 | Botryobasidium botryosum | 58.59 | 52.3 | + | 272632 | Mycoplasma | 49.28 | 24 | |
| | FD-172SS1 | | | | | mycoides subsp. | | | |
| | | | | | | mycoides SC str. PG1 | | | |
| 526224 | Brachyspira murdochii | 49.86 | 27.8 | + | 272633 | Mycoplasma | 50.21 | 25.7 | |
| | DSM 12563 | | | | | penetrans HF-2 | | | |
| 476282 | Bradyrhizobium japonicum | 47.94 | 63.7 | + | 272634 | Mycoplasma | 52.37 | 40 | |
| | SEMIA5079 | | | | | pneumoniae M129 | | | |
| 358681 | Brevibacillus brevis | 56.24 | 47.3 | + | 272635 | Mycoplasma | 50.52 | 26.6 | |
| | NBRC 100599 | | | | | pulmonis UAB CTIP | | | |
| 633149 | Brevundimonas | 45.68 | 68.4 | + | 744533 | Naegleria gruberi | 50.45 | 35 | + |
| | subvibrioides ATCC 15264 | | | | | strain NEG-M | | | |
| 224914 | Brucella melitensis bv. 1 | 48.02 | 57.24 | | 228908 | Nanoarchaeum | 53.05 | 31.6 | + |
| | str. 16M | | | | | equitans | | | |
| 107806 | Buchnera aphidicola str. | 52.03 | 25.3 | | 1737403 | Nanohaloarchaea | 51.11 | 46.4 | |
| | APS (Acyrthosiphon pisum) | | | | | archaeon SG9 | | | |
| 926550 | Caldilinea aerophila DSM | 51.5 | 58.8 | + | 457570 | Natranaerobius | 56.4 | 36.29 | + |
| | 14535 = NBRC 104270 | | | | | thermophilus | | | |
| | | | | | | JW/NM-WN-LF | | | |
| 511051 | Caldisericum exile | 52.74 | 35.4 | + | 797304 | Natronobacterium | 48.8 | 62.2 | + |
| | AZM16C01 | | | | | gregoryi SP2 | | | |
| 1056495 | Caldisphaera lagunensis | 52.55 | 30 | + | 122586 | Neisseria | 48.07 | 51.5 | |
| | DSM 15908 | | | | | meningitidis MC58 | | | |
| 768670 | Calditerrivibrio | 54.86 | 35.68 | + | 45351 | Nematostella | 59.19 | 41.9 | + |
| | nitroreducens DSM 19672 | | | | | vectensis | | | |
| 880073 | Caldithrix abyssi | 49.13 | 45.1 | + | 1287680 | Neofusicoccum | 50.99 | 56.7 | |
| | DSM 13497 | | | | | parvum UCRNP2 | | | |
| | Campylobacter jejuni | | | | | Neorhizobium | | | |
| 192222 | subsp. jejuni NCTC 11168 = | 51.61 | 30.5 | + | 1028800 | galegae bv. orientalis | 47.94 | 61.25 | + |
| | ATCC 700819 | | | | | str. HAMBI 540 | | | |
| 237561 | Candida albicans SC5314 | 53.57 | 33.48 | | 1189621 | Nitritalea | 55.4 | 48.6 | + |
| | | | | | | halalkaliphila LW7 | | | |
| 1618609 | Candidatus Azambacteria | 52.24 | 41.5 | + | 314278 | Nitrococcus mobilis | 53.69 | 59.9 | + |
| | bacterium | | | | | Nb-231 | | | |
| | G W2011_G WAl_42_19 | | | | | | | | |
| 1618623 | Candidatus Azambacteria | 51.16 | 46.1 | + | 1129897 | Nitrolancea | 52.82 | 62.6 | + |
| | bacterium | | | | | hollandica Lb | | | |
| | GW2011_GWD2_46_48 | | | | | | | | |
| 1618369 | Candidatus | 51.74 | 43 | + | 228410 | Nitrosomonas | 53.08 | 50.7 | + |
| | Beckwithbacteria | | | | | europaea | | | |
| | bacterium | | | | | ATCC 19718 | | | |
| | GW2011_GWA2_43_10 | | | | | | | | |
| 203907 | Candidatus Blochmannia | 51.66 | 27.4 | + | 436308 | Nitrosopumilus | 51.08 | 34.2 | |
| | floridanus | | | | | maritimus SCM1 | | | |
| 1618380 | Candidatus Collierbacteria | 56.02 | 43.8 | + | 926571 | Nitrososphaera | 50.75 | 52.7 | + |
| | bacterium | | | | | viennensis EN76 | | | |
| | GW2011_GWA2_44_99 | | | | | | | | |
| 1618405 | Candidatus Curtissbacteria | 57.57 | 40.8 | + | 1266370 | Nitrospina gracilis | 48.61 | 56.1 | |
| | bacterium | | | | | 3-211 | | | |
| | GW2011_GWA1_40_16 | | | | | | | | |
| 477974 | Candidatus Desulforudis | 50.46 | 60.8 | + | 330214 | Nitrospira defluvii | 53.65 | 59 | + |
| | audaxviator MP104C | | | | | | | | |
| 1408204 | Candidatus Endomicrobium | 54.02 | 35.8 | + | 196162 | Nocardioides sp. | 46.58 | 71.48 | + |
| | trichonymphae | | | | | JS614 | | | |
| 1429438 | Candidatus Entotheonella | 52.78 | 55.3 | + | 592029 | Nonlabens | 55.55 | 35.3 | + |
| | sp. TSY1 | | | | | dokdonensis DSW-6 | | | |
| 1429439 | Candidatus Entotheonella | 53.13 | 55.3 | + | 63737 | Nostoc punctiforme | 55.96 | 41.34 | |
| | sp. TSY2 | | | | | PCC73102 | | | |
| | Candidatus Falkowbacteria | | | | | Oceanithermus | | | |
| 1618643 | bacterium | 47.89 | 43.3 | + | 670487 | profundus | 45.17 | 69.79 | + |
| | GW2011_GWF2_43_32 | | | | | DSM 14977 | | | |
| 1618443 | Candidatus | 53.84 | 43.2 | + | 221109 | Oceanobacillus | 54.93 | 35.7 | + |
| | Gottesmanbacteria | | | | | iheyensis HTE831 | | | |
| | bacterium | | | | | | | | |
| | GW2011_GWA2_43_14 | | | | | | | | |
| 1072681 | Candidatus Haloredivivus | 54.59 | 42 | + | 203123 | Oenococcus oeni | 54.56 | 37.9 | + |
| | sp. G17 | | | | | PSU-1 | | | |
| 1427984 | Candidatus Hepatoplasma | 52.06 | 22.5 | + | 633147 | Olsenella uli | 48.31 | 64.7 | + |
| | crinochetorum Av | | | | | DSM 7084 | | | |
| | Candidatus | | | | | | | | |
| 1618662 | Jorgensenbacteria | 54.68 | 45.2 | + | 262768 | Onion yellows | 51.44 | 27.8 | |
| | bacterium | | | | | phytoplasma OY-M | | | |
| | GW2011_GWA2_45_13 | | | | | | | | |
| 1618671 | Candidatus Kaiserbacteria | 53.52 | 52 | + | 452637 | Opitutus terrae | 49.55 | 65.3 | + |
| | bacterium | | | | | PB90-1 | | | |
| | GW2011_GWA2_52_12 | | | | | | | | |
| 1618673 | Candidatus Kaiserbacteria | 55.64 | 50 | + | 765420 | Oscillochloris | 50.42 | 59.1 | + |
| | bacterium | | | | | trichoides DG-6 | | | |
| | GW2011_GWB1_50_17 | | | | | | | | |
| 1208920 | Candidatus | 53.13 | 31.2 | + | 436017 | Ostreococcus | 50.73 | 60.44 | |
| | Kinetoplastibacterium | | | | | lucimarinus | | | |
| | oncopeltii TCC290E | | | | | | | | |
| 374847 | Candidatus Korarchaeum | 47.16 | 49 | + | 926562 | Owenweeksia | 55.54 | 40.2 | + |
| | cryptofilum OPF8 | | | | | hongkongensis | | | |
| | | | | | | DSM 17368 | | | |
| 1619051 | Candidatus | 53.69 | 43 | + | 1343739 | Palaeococcus | 54 | 43 | + |
| | Magasanikbacteria | | | | | pacificus DY20341 | | | |
| | bacterium | | | | | | | | |
| | GW2011_GWD2_43_18 | | | | | | | | |
| 29290 | Candidatus | 56.19 | 47.3 | | 765952 | Parachlamydia | 55.72 | 39 | + |
| | Magnetobacterium | | | | | acanthamoebae | | | |
| | bavaricum | | | | | UV-7 | | | |
| 1295009 | Candidatus | 54.62 | 41.3 | + | 153151 | Parageobacillus | 51.77 | 42.1 | |
| | Methanomassiliicoccus | | | | | toebii | | | |
| | intestinalis Issoire-Mx1 str. | | | | | | | | |
| | Mx1-Issoire | | | | | | | | |
| | Candidatus | | | | | Paramecium | | | |
| 1236689 | Methanomethylophilus | 45.32 | 55.6 | + | 412030 | tetraurelia strain | 57.73 | 28.2 | + |
| | alvus Mx1201 | | | | | d4-2 | | | |
| 903503 | Candidatus Moranella | 53.19 | 43.5 | + | 1618821 | Parcubacteria group | 52.8 | 41.6 | + |
| | endobia PCIT | | | | | bacterium | | | |
| | | | | | | GW2011_GWA2_ | | | |
| | | | | | | 42_18 | | | |
| 1577684 | Candidatus Nanopusillus | 50.92 | 24.2 | | 1618840 | Parcubacteria group | 53.23 | 47.1 | + |
| | acidilobi | | | | | bacterium | | | |
| | | | | | | GW2011_GWA2_ | | | |
| | | | | | | 47_10b | | | |
| 859192 | Candidatus | 52.76 | 32.5 | | 1618841 | Parcubacteria group | 53.01 | 46.8 | + |
| | Nitrosoarchaeum limnia | | | | | bacterium | | | |
| | BG20 | | | | | GW2011_GWA2_ | | | |
| | | | | | | 47_12 | | | |
| 1229908 | Candidatus Nitrosopumilus | 52.2 | 34.2 | + | 1618924 | Parcubacteria group | 53.67 | 40.4 | + |
| | koreensis AR1 | | | | | bacterium | | | |
| | | | | | | GW2011_GWC2_ | | | |
| | | | | | | 40_31 | | | |
| 1237085 | Candidatus Nitrososphaera | 53.82 | 48.3 | | 402881 | Parvibaculum | 48.61 | 62.3 | + |
| | gargensis Ga9.2 | | | | | lavamentivorans | | | |
| | | | | | | DS-1 | | | |
| 1618729 | Candidatus | 55.7 | 36.9 | + | 314260 | Parvularcula | 52.99 | 60.7 | + |
| | Nomurabacteria bacterium | | | | | bermudensis | | | |
| | GW2011_GWA1_37_20 | | | | | HTCC2503 | | | |
| | Candidatus | | | | | Pasteurella | | | |
| 1618742 | Nomurabacteria bacterium | 57.03 | 36.7 | + | 747 | multocida str. | 49.34 | 40.3 | + |
| | GW2011_GWB1_37_5 | | | | | ATCC 43137 | | | |
| 1618775 | Candidatus | 55.88 | 36.2 | | 423536 | Perkinsus marinus | 57.36 | 47.4 | + |
| | Nomurabacteria bacterium | | | | | ATCC 50983 | | | |
| | GW2011_GWF2_36_19 | | | | | | | | |
| 1618777 | Candidatus | 56.95 | 39.6 | + | 123214 | Persephonella | 46.05 | 37.12 | + |
| | Nomurabacteria bacterium | | | | | marina EX-H1 | | | |
| | GW2011_GWF2_40_31 | | | | | | | | |
| 1002672 | Candidatus Pelagibacter sp. | 54.7 | 31.7 | + | 403833 | Petrotoga mobilis | 56.26 | 34.1 | + |
| | IMCC9063 | | | | | SJ95 | | | |
| 1619068 | Candidatus | 54.69 | 43.1 | + | 556484 | Phaeodactylum | 57.66 | 48.84 | |
| | Peregrinibacteria | | | | | tricornutum CCAP | | | |
| | bacterium | | | | | 1055/1 | | | |
| | GW2011_GWF2_43_17 | | | | | | | | |
| 1236703 | Candidatus Photodesmus | 50.44 | 31.06 | + | 298386 | Photobacterium | 53.42 | 41.75 | + |
| | katoptron Akat1 | | | | | profundum SS9 | | | |
| 234267 | Candidatus Solibacter | 50.63 | 61.9 | | 243265 | Photorhabdus | 54.82 | 42.8 | + |
| | usitatus Ellin6076 | | | | | luminescens subsp. | | | |
| | | | | | | laumondii TTO1 | | | |
| 1618595 | Candidatus Woesebacteria | 55.5 | 40.1 | + | 1142394 | Phycisphaera | 46.81 | 73.23 | + |
| | bacterium | | | | | mikurensis | | | |
| | GW2011_GWD2_40_19 | | | | | NBRC 102666 | | | |
| 1619005 | Candidatus Wolfebacteria | 56.02 | 46.7 | + | 3218 | Physcomitrella | 58.62 | 34.3 | |
| | bacterium | | | | | patens | | | |
| | GW2011_GWA2_47_9b | | | | | | | | |
| 1619029 | Candidatus | 53.07 | 41.3 | + | 164328 | Phytophthora | 52.82 | 53 | + |
| | Yanofskybacteria | | | | | ramorum | | | |
| | bacterium | | | | | | | | |
| | GW2011_GWC2_41_9 | | | | | | | | |
| 521097 | Capnocytophaga ochracea | 51.52 | 39.6 | + | 263820 | Picrophilus torridus | 46.65 | 36 | + |
| | DSM 7271 | | | | | DSM 9790 | | | |
| 595528 | Capsaspora owczarzaki | 53.71 | 53.7 | + | 1227812 | Piscirickettsia | 53.32 | 39.62 | + |
| | ATCC 30864 | | | | | salmonis LF-89 = | | | |
| | | | | | | ATCC VR-1361 | | | |
| 479433 | Catenulispora acidiphila | 47.12 | 69.8 | + | 521674 | Planctopirus | 54.76 | 53.72 | + |
| | DSM 44928 | | | | | limnophila | | | |
| | | | | | | DSM 3776 | | | |
| 190650 | Caulobacter crescentus | 45.55 | 67.2 | | 36329 | Plasmodium | 57.62 | 19.36 | + |
| | CB15 | | | | | falciparum 3D7 | | | |
| 979 | Cellulophaga lytica | 51.33 | 32.1 | + | 4781 | Plasmopara halstedii | 56.75 | 45.7 | |
| 414004 | Cenarchaeum symbiosum A | 51.98 | 57.4 | + | 1069680 | Pneumocystis | 53.09 | 27 | |
| | | | | | | murina b123 | | | |
| 1319815 | Cetobacterium somerae | 50.26 | 28.6 | + | 431947 | Porphyromonas | 55.17 | 48.4 | |
| | ATCC BAA-474 | | | | | gingivalis | | | |
| | | | | | | ATCC 33277 | | | |
| 218497 | Chlamydia abortus S26-3 | 55.75 | 39.9 | + | 561896 | Postia placenta Mad- | 58.13 | 52.7 | + |
| | | | | | | 698-R | | | |
| 3055 | Chlamydomonas reinhardtii | 51.49 | 61.95 | | 167546 | Prochlorococcus | 53.78 | 36.4 | + |
| | | | | | | marinus str. | | | |
| | | | | | | MIT 9301 | | | |
| 115713 | Chlamydophila | 55.8 | 40.6 | | 208964 | Pseudomonas | 43.26 | 66.6 | |
| | pneumoniae CWL029 | | | | | aeruginosa PAO1 | | | |
| 138677 | Chlamydophila | 55.82 | 40.6 | | 96563 | Pseudomonas | 45.32 | 60.6 | |
| | pneumoniae J138 | | | | | stutzeri | | | |
| 517417 | Chlorobaculum parvum | 49.88 | 55.8 | + | 1123384 | Pseudothermotoga | 52.39 | 49.5 | |
| | NCIB8327 | | | | | hypogea DSM 11164 = | | | |
| | | | | | | NBRC 106472 | | | |
| 194439 | Chlorobium tepidum TLS | 49.98 | 56.5 | | 259536 | Psychrobacter | 50.6 | 42.8 | |
| | | | | | | arcticus 273-4 | | | |
| 326427 | Chloroflexus aggregans | 53.71 | 56.4 | | 335284 | Psychrobacter | 50.87 | 42.25 | + |
| | DSM 9485 | | | | | cryohalolentis K5 | | | |
| 324602 | Chloroflexus aurantiacus | 53.19 | 56.7 | + | 1189619 | Psych roflexus | 56.9 | 35.8 | + |
| | J-10-fl | | | | | gondwanensis | | | |
| | | | | | | ACAM 44 | | | |
| 517418 | Chloroherpeton thalassium | 50.46 | 45 | + | 418459 | Puccinia graminis f. | 58.01 | 43.8 | |
| | ATCC 35110 | | | | | sp. tritici | | | |
| 2769 | Chondrus crispus | 59 | 52.86 | | 178306 | Pyrobaculum | 53.55 | 51.4 | + |
| | (carragheen) | | | | | aerophilum str. IM2 | | | |
| 243365 | Chromobacterium | 43.58 | 64.8 | + | 272844 | Pyrococcus abyssi | 50.78 | 44.7 | |
| | violaceum ATCC 12472 | | | | | GE5 | | | |
| 345663 | Chryseobacterium | 54.24 | 34.1 | | 186497 | Pyrococcus furiosus | 53.7 | 40.8 | + |
| | greenlandense | | | | | DSM 3638 | | | |
| 1303518 | Chthonomonas calidirosea | 56.15 | 54.6 | + | 70601 | Pyrococcus | 52.96 | 41.9 | |
| | T49 | | | | | horikoshii OT3 | | | |
| 443906 | Clavibacter michiganensis | 45 | 72.42 | | 1273541 | Pyrodictium delaneyi | 54 | 53.9 | |
| | subsp. michiganensis | | | | | | | | |
| | NCPPB382 | | | | | | | | |
| 866499 | Cloacibacillus evryensis | 49.66 | 56 | + | 694429 | PyroIobus fumarii 1A | 54.07 | 54.9 | + |
| | DSM 19522 | | | | | | | | |
| 642492 | Clostridium lentocellum | 54.09 | 34.3 | + | 1223560 | Pythium vexans | 50.15 | 58.7 | |
| | DSM 5427 | | | | | DAOM BR484 | | | |
| 212717 | Clostridium tetani E88 | 52.83 | 28.59 | | 267608 | Ralstonia | 44.93 | 66.96 | + |
| | | | | | | solanacearum | | | |
| | | | | | | GMI1000 | | | |
| 1055104 | Cobetia amphilecti str. | 45.14 | 62.5 | + | 365046 | Ramlibacter | 42.5 | 70 | + |
| | KMM 296 | | | | | tataouinensis | | | |
| | | | | | | TTB310 | | | |
| 574566 | Coccomyxa subellipsoidea | 52.76 | 52.9 | | 145458 | Rathayibacter | 55.18 | 61.5 | |
| | C-169 | | | | | toxicus | | | |
| 469383 | Conexibacter woesei | 44.37 | 72.4 | + | 288705 | Renibacterium | 55.88 | 56.3 | + |
| | DSM 14684 | | | | | salmoninarum | | | |
| | | | | | | ATCC 33209 | | | |
| 583355 | Coraliomargarita | 53.84 | 53.6 | + | 1033991 | Rhizobium | 48.1 | 61.17 | + |
| | akajimensis DSM 45221 | | | | | leguminosarum bv. | | | |
| | | | | | | trifolii CB782 | | | |
| 196164 | Corynebacterium efficiens | 47.89 | 62.93 | | 243090 | Rhodopirellula | 52.94 | 55.4 | + |
| | YS-314 | | | | | baltica SH 1 | | | |
| 196627 | Corynebacterium | 52.51 | 53.8 | | 258594 | Rhodopseudomonas | 45.97 | 66 | |
| | glutamicum ATCC 13032 | | | | | palustris CGA009 | | | |
| 227377 | Coxiella burnetii RSA493 | 54.47 | 42.34 | | 518766 | Rhodothermus | 48.08 | 64.27 | + |
| | | | | | | marinus DSM 4252 | | | |
| 216432 | Croceibacter atlanticus | 53.28 | 33.9 | + | 1165094 | Richelia | 55.08 | 33.7 | + |
| | HTCC2559 | | | | | intracellularis HH01 | | | |
| 1529318 | Cryobacterium sp. MLB-32 | 51.31 | 67.53 | + | 313596 | Robiginitalea | 49.01 | 55.3 | + |
| | | | | | | biformata HTCC2501 | | | |
| 214684 | Cryptococcus neoformans | 56.73 | 48.54 | | 585394 | Roseburia hominis | 49.7 | 48.5 | + |
| | var. neoformans JEC21 | | | | | A2-183 | | | |
| 2898 | Cryptomonas paramecium | 58.46 | 27.81 | | 383372 | Roseiflexus | 51.69 | 60.7 | + |
| | | | | | | castenholzii | | | |
| | | | | | | DSM 13941 | | | |
| 353152 | Cryptosporidium parvum | 54.92 | 30.25 | + | 762948 | Rothia dentocariosa | 53.87 | 53.7 | + |
| | Iowa II | | | | | ATCC 17931 | | | |
| 1292022 | Curtobacterium | 45.69 | 70.8 | + | 582515 | Rubidibacter lacunae | 54.56 | 56.2 | + |
| | flaccumfaciens UCD-AKU | | | | | KORDI 51-2 | | | |
| 280699 | Cyanidioschyzon merolae | 58.02 | 55.02 | + | 559292 | Saccharomyces | 56.61 | 38.16 | |
| | | | | | | cerevisiae S288c | | | |
| 6669 | Daphnia pulex | 57.94 | 42.4 | + | 405948 | Saccharopolyspora | 46.03 | 71.1 | + |
| | | | | | | erythraea NRRL2338 | | | |
| 639282 | Deferribacter desulfuricans | 54.66 | 30.3 | + | 435906 | Salegentibacter | 55.41 | 37 | |
| | SSMI | | | | | salarius | | | |
| 255470 | Dehalococcoides mccartyi | 51.38 | 48.9 | + | 407035 | Salinicoccus | 52.87 | 44.5 | |
| | CBDB1 | | | | | halodurans | | | |
| 1432061 | Dehalococcoides mccartyi | 51.27 | 48.9 | | 45670 | Salinicoccus roseus | 51.05 | 50 | |
| | CG5 | | | | | | | | |
| 552811 | Dehalogenimonas | 50.82 | 55 | + | 1432562 | Salinicoccus | 50.88 | 48.7 | |
| | lykanthroporepellens BL- | | | | | sediminis | | | |
| | DC-9 | | | | | | | | |
| 319795 | Deinococcus geothermalis | 49.99 | 66.57 | + | 1033802 | Salinisphaera | 48.43 | 61.6 | + |
| | DSM 11300 str. DSM11300 | | | | | shabanensis E1L3A | | | |
| 937777 | Deinococcus peraridilitoris | 50.08 | 63.71 | | 1307761 | Salinispira pacifica | 50.38 | 51.9 | + |
| | DSM 19664 | | | | | | | | |
| 1182568 | Deinococcus puniceus | 48.03 | 62.6 | | 99287 | Salmonella enterica | 48.94 | 51.88 | |
| | | | | | | subsp. enterica | | | |
| | | | | | | serovar | | | |
| | | | | | | Typhimurium str. LT2 | | | |
| 243230 | Deinococcus radiodurans | 48.45 | 66.61 | | 946362 | Salpingoeca rosetta | 52.04 | 55.5 | + |
| | RI | | | | | | | | |
| 522772 | Denitrovibrio acetiphilus | 52.97 | 42.5 | + | 695850 | Saprolegnia | 46.48 | 57.5 | + |
| | DSM 12809 | | | | | parasitica | | | |
| | | | | | | CBS 223.65 | | | |
| 651182 | Desulfobacula toluolica | 53.14 | 41.4 | + | 578458 | Schizophyllum | 55.02 | 57.4 | + |
| | Tol2 | | | | | commune H4-8 | | | |
| 555779 | Desulfonatronospira | 50.21 | 51.3 | + | 284812 | Schizosaccharomyces | 55.7 | 36.04 | + |
| | thiodismutans ASO3-1 | | | | | pombe (strain 972/ | | | |
| | | | | | | ATCC 24843) | | | |
| 768706 | Desulfosporosinus orientis | 56.91 | 42.9 | + | 526218 | Sebaldella termitidis | 51.66 | 33.42 | + |
| | DSM 765 | | | | | ATCC 33386 | | | |
| 882 | Desulfovibrio vulgaris str. | 51.11 | 67.1 | | 211586 | Shewanella | 52.66 | 45.93 | + |
| | Hildenborough | | | | | oneidensis MR-1 | | | |
| 653733 | Desulfurispirillum indicum | 48.29 | 56.1 | + | 1454006 | Siansivirga | 53.62 | 33.5 | |
| | S5 | | | | | zeaxanthinifaciens | | | |
| | | | | | | CC-SAMT-1 | | | |
| 868864 | Desulfurobacterium | 50.12 | 34.9 | + | 331113 | Simkania negevensis | 55.21 | 41.62 | + |
| | thermolithotrophum DSM | | | | | Z | | | |
| | 11699 | | | | | | | | |
| 910314 | Dialister microaerophilus | 51.76 | 35.6 | + | 886293 | Singulisphaera | 53.18 | 62.27 | + |
| | UPH 345-E | | | | | acidiphila | | | |
| | | | | | | DSM 18658 | | | |
| 309799 | Dictyoglomus | 52.02 | 33.7 | + | 266834 | Sinorhizobium | 49.74 | 62.16 | |
| | thermophilum H-6-12 | | | | | meliloti 1021 | | | |
| 515635 | Dictyoglomus turgidum | 51.47 | 34 | + | 742818 | Slackia piriformis | 50.11 | 57.6 | + |
| | DSM 6724 | | | | | YIT 12062 | | | |
| 352472 | Dictyostelium discoideum | 47.44 | 22.46 | + | 929556 | Solitalea canadensis | 55.87 | 37.3 | + |
| | AX4 | | | | | DSM 3403 | | | |
| 420778 | Diplodia seriata | 51.2 | 56.5 | | 479434 | Sphaerobacter | 49.14 | 68.1 | + |
| | | | | | | thermophilus | | | |
| | | | | | | DSM 20745 | | | |
| 3046 | Dunaliella salina | 54.15 | 40.1 | | 158189 | Sphaerochaeta | 55.24 | 48.9 | + |
| | | | | | | globosa str. Buddy | | | |
| 999415 | Eggerthia catenaformis OT | 52.64 | 32.8 | + | 29656 | Spirodela polyrhiza | 56.18 | 42.72 | |
| | 569 = DSM 20559 | | | | | | | | |
| 445932 | Elusimicrobium minutum | 50.23 | 40 | + | 645134 | Spizellomyces | 58.96 | 47.6 | + |
| | Pei191 | | | | | punctatus DAOM | | | |
| | | | | | | BR117 | | | |
| 280463 | Emiliania huxleyi | 51.18 | 64.5 | + | 1397361 | Sporothrix schenckii | 52.84 | 55 | |
| | CCMP1516 | | | | | 1099-18 | | | |
| 885318 | Entamoeba histolytica | 49.55 | 24.3 | | 446470 | Stackebrandtia | 46.75 | 68.1 | + |
| | HM-1:IMSS-A | | | | | nassauensis | | | |
| | | | | | | DSM 44728 | | | |
| 226185 | Enterococcus faecalis V583 | 52.84 | 37.35 | | 93061 | Staphylococcus | 51.57 | 32.9 | |
| | | | | | | aureus subsp. aureus | | | |
| | | | | | | NCTC8325 | | | |
| 1185651 | Enterovibrio norvegicus | 53.22 | 47.6 | | 176280 | Staphylococcus | 52.65 | 32.05 | |
| | FF-454 | | | | | epidermidis | | | |
| | | | | | | ATCC 12228 | | | |
| 931890 | Eremothecium cymbalariae | 57.74 | 40.32 | + | 519441 | Streptobacillus | 50.81 | 26.27 | + |
| | DBVPG#7215 | | | | | moniliformis | | | |
| | | | | | | DSM 12112 | | | |
| 284811 | Eremothecium gossypii | 56.86 | 51.69 | | 160490 | Streptococcus | 53.41 | 38.5 | |
| | ATCC 10895 (assembly | | | | | pyogenes M1 GAS | | | |
| | ASM9102v4) | | | | | | | | |
| 314225 | Erythrobacter litoralis | 48.36 | 63.1 | + | 227882 | Streptomyces | 48.18 | 70.6 | |
| | HTCC2594 | | | | | avermitilis MA-4680 = | | | |
| | | | | | | NBRC 14893 | | | |
| 511145 | Escherichia coli str. K-12 | 48.83 | 50.45 | | 100226 | Streptomyces | 46.9 | 71.98 | + |
| | substr. MG1655 | | | | | coelicolor A3(2) | | | |
| 316407 | Escherichia coli str. K-12 | 48.97 | 50.45 | + | 1469144 | Streptomyces | 46.55 | 69.2 | |
| | substr. W3110 | | | | | thermoautotrophicus | | | |
| 360911 | Exiguobacterium sp. AT1b | 50.44 | 48.5 | + | 762983 | Succinatimonas | 51.99 | 40.3 | + |
| | | | | | | hippei YIT 12066 | | | |
| 589924 | Ferroglobus placidus | 50.05 | 44.1 | + | 429572 | Sulfolobus islandicus | 55.84 | 35.1 | |
| | DSM 10642 | | | | | L.S.2.15 | | | |
| 333146 | Ferroplasma acidarmanus | 52.66 | 36.5 | + | 273063 | Sulfolobus tokodaii | 54.82 | 32.8 | |
| | fer1 | | | | | str. 7 | | | |
| 381764 | Fervidobacterium nodosum | 55 | 35 | + | 204536 | Sulfurihydrogenibiu | 50.81 | 32.8 | + |
| | Rt17-Bl | | | | | m azorense Az-Fu1 | | | |
| 59374 | Fibrobacter succinogenes | 48.94 | 48 | | 432331 | Sulfurihydrogenibium | 53.08 | 32.8 | |
| | subsp. succinogenes 585 | | | | | yellowstonense | | | |
| | | | | | | SS-5 | | | |
| 661478 | Fimbriimonas ginsengisoli | 52.65 | 60.8 | + | 326298 | Sulfurimonas | 52.73 | 34.5 | + |
| | Gsoil 348 | | | | | denitrificans | | | |
| | | | | | | DSM 1251 | | | |
| 1519565 | Fistulifera Solaris | 56.79 | 45.6 | | 269084 | Synechococcus | 53.98 | 55.5 | |
| | | | | | | elongatus PCC 6301 | | | |
| 391603 | Flavobacteriales bacterium | 54.07 | 32.4 | | 316279 | Synechococcus sp. | 55.61 | 54.2 | + |
| | ALC-1 | | | | | CC9902 | | | |
| 1341181 | Flavobacterium | 54.91 | 38.5 | | 1148 | Synechocystis sp. | 51.92 | 47.35 | |
| | limnosediminis JC2902 | | | | | PCC 6803 | | | |
| 402612 | Flavobacterium | 55.34 | 32.5 | + | 1209989 | Tepidanaerobacter | 57.16 | 37.5 | + |
| | psychrophilum JIP02/86 | | | | | acetatoxydans Re1 | | | |
| 755732 | Fluviicola taffensis | 54.77 | 36.5 | + | 312017 | Tetrahymena | 56.34 | 22.3 | + |
| | DSM 16823 | | | | | thermophila SB210 | | | |
| 691883 | Fonticula alba | 51.31 | 64.3 | + | 296543 | Thalassiosira | 56.81 | 46.91 | + |
| | | | | | | pseudonana | | | |
| 1347342 | Formosa agariphila | 53.7 | 33.6 | + | 1208320 | Thalassolituus | 52.37 | 46.6 | + |
| | KMM 3901 | | | | | oleivorans R6-15 | | | |
| 635003 | Fragilariopsis cylindrus | 55.19 | 39 | | 1177928 | Thalassospira | 47.49 | 55.2 | + |
| | CCMP1102 | | | | | profundimaris | | | |
| | | | | | | WP0211 | | | |
| 767434 | Frateuria aurantia | 46.11 | 63.4 | + | 1198115 | Thaumarchaeota | 58.56 | 43.3 | + |
| | DSM 6220 | | | | | archaeon SCGC AB- | | | |
| | | | | | | 539-E09 | | | |
| 930946 | Fructobacillus fructosus | 52.35 | 44.6 | + | 353154 | Theileria annulata | 57.63 | 32.55 | |
| | KCTC 3544 | | | | | strain Ankara | | | |
| | Fusobacterium | | | | | Thermanaerovibrio | | | |
| 469615 | gonidiaformans | 52.17 | 32.9 | | 525903 | acidaminovorans | 43.3 | 63.8 | + |
| | ATCC 25563 | | | | | DSM 6589 | | | |
| | Fusobacterium nucleatum | | | | | Thermobaculum | | | |
| 190304 | subsp. nucleatum | 49.86 | 27.2 | + | 525904 | terrenum ATCC | 55.88 | 53.54 | + |
| | ATCC 25586 | | | | | BAA-798 | | | |
| 469599 | Fusobacterium | 49.53 | 28.6 | | 269800 | Thermobifida fusca | 49.85 | 67.5 | + |
| | periodonticum 2_1_31 | | | | | YX | | | |
| 555500 | Galbibacter marinus | 57.03 | 37 | + | 469371 | Thermobispora | 45.66 | 72.4 | + |
| | | | | | | bispora DSM 43833 | | | |
| 130081 | Galdieria sulphuraria | 56.06 | 37.9 | | 391623 | Thermococcus | 53.84 | 41.71 | |
| | | | | | | barophilus MP | | | |
| 553190 | Gardnerella vaginalis | 49.61 | 42 | + | 163003 | Thermococcus | 45.96 | 55.8 | |
| | 409-05 | | | | | cleftensis | | | |
| 49280 | Gelidibacter algens | 56.43 | 37.3 | | 593117 | Thermococcus | 48.55 | 53.6 | |
| | | | | | | gammatolerans EJ3 | | | |
| | | | | | | Thermococcus | | | |
| 1630693 | Gemmata sp. SH-PL17 | 49.95 | 64.2 | | 1432656 | guaymasensis | 48.89 | 52.9 | |
| | | | | | | DSM 11113 | | | |
| 379066 | Gemmatimonas aurantiaca | 50.34 | 64.3 | + | 195522 | Thermococcus | 46.43 | 54.8 | |
| | T-27 | | | | | nautili | | | |
| 1379270 | Gemmatimonas | 51.07 | 64.4 | | 638303 | Thermocrinis albus | 49.57 | 46.9 | + |
| | phototrophica | | | | | DSM 14484 | | | |
| 861299 | Gemmatirosa | 43.9 | 72.64 | + | 667014 | Thermodesulfatator | 53.76 | 42.4 | + |
| | kalamazoonesis | | | | | indicus DSM 15286 | | | |
| 1121915 | Geoalkalibacter | 49.77 | 57.9 | + | 289377 | Thermodesulfobacterium | 50.53 | 37 | + |
| | ferrihydriticus DSM 17813 | | | | | commune | | | |
| | | | | | | DSM 2178 | | | |
| 235909 | Geobacillus kaustophilus | 48.08 | 51.99 | + | 795359 | Thermodesulfobacterium | 49.84 | 30.6 | + |
| | HTA426 | | | | | geofontis | | | |
| | | | | | | OPF15 | | | |
| 272567 | Geobacillus | 47.54 | 52.61 | | 289376 | Thermodesulfovibrio | 50.81 | 34.1 | + |
| | stearothermophilus 10 | | | | | yellowstonii | | | |
| | | | | | | DSM 11347 | | | |
| 398767 | Geobacter lovleyi SZ | 50.06 | 54.77 | + | 309801 | Thermomicrobium | 53.14 | 64.26 | + |
| | | | | | | roseum DSM 5159 | | | |
| | | | | | | Thermoplasma | | | |
| 184922 | Giardia lamblia ATCC 50803 | 58.54 | 49.2 | + | 273075 | acidophilum | 51.06 | 46 | + |
| | | | | | | DSM 1728 | | | |
| 1183438 | Gloeobacter kilaueensis JS1 | 51.52 | 60.5 | | 273116 | Thermoplasma | 55 | 39.9 | |
| | | | | | | volcanium GSS1 | | | |
| 251221 | Gloeobacter violaceus | 50.38 | 62 | + | 768679 | Thermoproteus | 51.18 | 55.1 | |
| | PCC7 421 | | | | | tenax Kra 1 | | | |
| 290633 | Gluconobacter oxydans | 49.9 | 60.84 | + | 484019 | Thermosipho | 53.57 | 30.8 | + |
| | 621H | | | | | africanus TCF52B | | | |
| 411154 | Gramella forsetii KT0803 | 56.12 | 36.6 | + | 391009 | Thermosipho | 55.29 | 31.4 | |
| | | | | | | melanesiensis BI429 | | | |
| 391165 | Granulibacter bethesdensis | 50.36 | 59.1 | + | 1298851 | Thermosulfidibacter | 53.49 | 43 | |
| | CGDNIH1 | | | | | takaii ABI70S6 | | | |
| 905079 | Guillardia theta CCMP2712 | 54.9 | 52.9 | + | 243274 | Thermotoga | 50.62 | 46.2 | + |
| | | | | | | maritima MSB8 | | | |
| 944289 | Gymnopus luxurians FD- | 58.85 | 45.1 | + | 648996 | Thermovibrio | 45.66 | 52.12 | + |
| | 317 M1 | | | | | ammonificans HB-1 | | | |
| 233412 | Haemophilus ducreyi | 50.03 | 38.2 | | 580340 | Thermovirga lienii | 54.93 | 47.1 | + |
| | 35000HP | | | | | DSM 17291 | | | |
| 866895 | Halobacillus halophilus | 56.94 | 41.8 | + | 498848 | Thermus aquaticus | 44.81 | 68.04 | |
| | DSM 2266 | | | | | Y51MC23 | | | |
| 862908 | Halobacteriovorax marinus | 52.67 | 36.7 | + | 751945 | Thermus oshimai | 44.39 | 68.6 | + |
| | SJ | | | | | JL-2 | | | |
| 64091 | Halobacterium salinarum | 49.99 | 65.7 | | 300852 | Thermus | 44.13 | 69.49 | |
| | NRC-1 | | | | | thermophilus HB8 | | | |
| 478009 | Halobacterium salinarum | 49.94 | 65.92 | + | 768671 | Thiocapsa marina 5811 | 50.99 | 64.1 | + |
| | R1 | | | | | | | | |
| 523841 | Haloferax mediterranei | 49.56 | 60.26 | + | 381306 | Thiohalorhabdus | 44.19 | 68.9 | + |
| | ATCC 33500 | | | | | denitrificans | | | |
| 469382 | Halogeometricum | 50.95 | 59.97 | + | 1177931 | Thiovulum sp. ES | 51.48 | 33 | + |
| | borinquense DSM 11551 | | | | | | | | |
| 797210 | Halopiger xanaduensis SH-6 | 46.79 | 65.2 | + | 1245935 | Tolypothrix | 56.42 | 45.1 | |
| | | | | | | campylonemoides | | | |
| | | | | | | VB511288 | | | |
| 1033810 | Haloplasma contractile | 55.86 | 32.3 | + | 508771 | Toxoplasma gondii | 56.4 | 52.29 | + |
| | SSD-17B | | | | | ME49 | | | |
| 362976 | Haloquadratum walsbyi | 52.24 | 47.69 | + | 243275 | Treponema | 55.05 | 37.9 | + |
| | DSM 16790 | | | | | denticola | | | |
| | | | | | | ATCC 35405 | | | |
| 797114 | Halosimplex carlsbadense | 47.11 | 67.7 | + | 203124 | Trichodesmium | 54.62 | 34.1 | + |
| | 2-9-1 | | | | | erythraeum IMS101 | | | |
| 373903 | Halothermothrix orenii | 51.33 | 37.9 | + | 412133 | Trichomonas | 53.67 | 32.9 | |
| | H 168 | | | | | vaginalis G3 | | | |
| 555778 | Halothiobacillus | 52.68 | 54.7 | + | 10228 | Trichoplax | 57.34 | 34.5 | + |
| | neapolitanus c2 | | | | | adhaerens | | | |
| 85962 | Helicobacter pylori 26695 | 48.19 | 38.9 | | 203267 | Tropheryma | 57.37 | 46.3 | |
| | | | | | | whipplei str. Twist | | | |
| 316274 | Herpetosiphon aurantiacus | 47.4 | 50.89 | + | 649638 | +pera radiovictrix | 47.28 | 68.1 | + |
| | DSM 785 | | | | | DSM 17093 | | | |
| 760142 | Hippea maritima | 54.39 | 37.5 | + | 5693 | Trypanosoma cruzi | 57.03 | 51.7 | |
| | DSM 10411 | | | | | | | | |
| 1321371 | Holospora undulata HU1 | 55.06 | 36.1 | + | 1157490 | Tumebacillus | 46.11 | 56.5 | + |
| | | | | | | flagellatus | | | |
| 1172194 | Hydrocarboniphaga effusa | 45.27 | 65.2 | + | 883169 | Turicella otitidis | 44.36 | 71 | + |
| | AP103 | | | | | ATCC 51513 | | | |
| 608538 | Hydrogenobacter | 50.6 | 44 | + | 505682 | Ureaplasma parvum | 47.77 | 25.5 | + |
| | thermophilus TK-6 | | | | | serovar 3 str. | | | |
| | | | | | | ATCC 27815 | | | |
| 547144 | Hydrogenobaculum sp. HO | 51.57 | 34.8 | + | 436907 | Vanderwaltozyma | 51.58 | 33 | + |
| | | | | | | polyspora | | | |
| | | | | | | DSM 70294 | | | |
| 945553 | Hypholoma sublateritium | 58.69 | 51 | + | 263358 | Verrucosispora maris | 46.99 | 70.89 | + |
| | FD-334 SS-4 | | | | | AB-18-032 | | | |
| 945713 | Ignavibacterium album | 53.23 | 33.9 | + | 388396 | Vibrio fischeri MJ11 | 50.71 | 38.37 | + |
| | JCM 16511 | | | | | | | | |
| 583356 | Ignisphaera aggregans | 51.32 | 35.7 | + | 223926 | Vibrio | 51.82 | 45.4 | |
| | DSM 17230 | | | | | parahaemolyticus | | | |
| | | | | | | RIMD 2210633 | | | |
| 1313172 | Ilumatobacter coccineus | 46.63 | 67.3 | + | 196600 | Vibrio vulnificus | 52.79 | 46.67 | |
| | YM16-304 | | | | | YJ016 | | | |
| 572544 | Ilyobacter polytropus | 52.99 | 34.36 | + | 3067 | Volvox carteri | 57.51 | 55.3 | |
| | DSM 2926 | | | | | | | | |
| 946077 | Imtechella halotolerans K1 | 55.9 | 35.5 | + | 572478 | Vulcanisaeta | 49.56 | 45.4 | + |
| | | | | | | distributa | | | |
| | | | | | | DSM 14429 | | | |
| 743718 | Isoptericola variabilis 225 | 44.32 | 73.9 | + | 4927 | Wickerhamomyces | 48.08 | 35 | |
| | | | | | | anomalus NRRL | | | |
| | | | | | | Y-366-8 | | | |
| 575540 | Isosphaera pallida | 53.13 | 62.45 | + | 1041607 | Wickerhamomyces | 46.02 | 30.4 | |
| | ATCC 43644 | | | | | ciferrii | | | |
| 926559 | Joostella marina | 55.36 | 33.6 | + | 641526 | Winogradskyella | 54.66 | 33.5 | + |
| | DSM 19592 | | | | | psychrotolerans RS-3 | | | |
| 266940 | Kineococcus radiotolerans | 46.24 | 74.21 | + | 1116230 | Wolbachia pipientis | 56.57 | 33.8 | |
| | SRS30216 = ATCC BAA-149 | | | | | wAIbB | | | |
| 452652 | Kitasatospora setae | 44.67 | 74.2 | + | 273121 | Wolinella | 50.32 | 48.5 | + |
| | KM-6054 | | | | | succinogenes | | | |
| | | | | | | DSM 1740 | | | |
| 1125630 | Klebsiella pneumoniae | 46.34 | 57.14 | | 1304892 | Xanthomonas | 45.38 | 64.72 | + |
| | subsp. pneumoniae | | | | | axonopodis Xac29-1 | | | |
| | HS11286 | | | | | | | | |
| 1006000 | Kluyvera ascorbata | 47.11 | 54.3 | + | 190485 | Xanthomonas | 45.06 | 65.1 | |
| | ATCC 33433 | | | | | campestris pv. | | | |
| | | | | | | campestris str. | | | |
| | | | | | | ATCC 33913 | | | |
| 521045 | Kosmotoga olearia TBF | 56.34 | 41.5 | + | 160492 | Xylella fastidiosa | 54.7 | 52.64 | |
| | 19.5.1 | | | | | 9a5c | | | |
| 1330330 | Kosmotoga pacifica | 56.58 | 42.5 | | 155920 | Xylella fastidiosa | 54.52 | 52.64 | + |
| | | | | | | subsp. sandyi Ann-1 | | | |
| 485913 | Ktedonobacter racemifer | 55.04 | 53.8 | + | 655815 | Zunongwangia | 56.34 | 36.2 | + |
| | DSM 44963 | | | | | profunda SM-A87 | | | |
| 486041 | Laccaria bicolor S238N-H82 | 59.01 | 47.1 | + | 1047168 | Zymoseptoria brevis | 56.5 | 51.2 | |
| 983544 | Lacinutrix sp. 5H-3-7-4 | 51.53 | 30.8 | + | 336722 | Zymoseptoria tritici | 56.39 | 52.12 | |
| | | | | | 1619079 | candidate division | 54.19 | 32.7 | + |
| | | | | | | TM6 bacterium | | | |
| | | | | | | GW2011_ | | | |
| | | | | | | GWF2_32_72 | | | |

Randomization procedures: To test different hypotheses regarding local folding-energy (LFE), native sequences were compared against randomized sequences preserving attributes as defined by each null hypothesis, as follows (FIG. 2A-B):

To test the hypothesis that the native arrangement of synonymous codons causes a significant bias in LFE, synonymous codons were randomly permuted within each CDS (i.e., all codons encoding for the same amino acid within a given CDS are randomly rearranged). This “CDS-wide” randomization preserves the encoded proteins sequence, nucleotide frequencies (including GC-content) and codon frequencies of each CDS (but generally disrupts longer-range dependencies). Synonymous codons were determined according to the nuclear genetic code annotated for each species in NCBI genomes.

To test the contribution of position-specific biases in amino-acid composition, nucleotide frequencies and codon frequencies including CUB (factors that are equalized at the CDS level by the CDS-wide randomization) on the observed LFE, a second “position-specific” randomization was used. In this randomization, synonymous codons were randomly permuted between codons found at the same position (relative to the CDS start) across all CDSs in each genome. This randomization preserves the amino-acid sequence of each CDS, while nucleotide (including GC-content) and codon frequencies are preserved at each position across a genome.

LFE profile calculation: Local folding-energy (LFE) profiles were created by calculating the folding-energy of all 40 nt-long windows, at 10 nt intervals, relative to the CDS start and end, on each native and randomized sequence. This measure estimates local secondary-structure strength (ignoring the specific structures) and reflects (among other considerations) the structure of mRNA during translation, which prevents long-range structures but allows formation of local secondary-structure and generally agrees with existing large-scale experimental validation results. Previous studies showed that this measure is robust to changes in the window size. The coordinates shown always refer to the window start position relative to the CDS start (e.g., window 0 includes the first 40 nt in the CDS) or to the window end position relative to the CDS end. Estimated folding-energies were calculated for each window using RNAfold from the ViennaRNA package 2.3.0, with the default settings. All folding-energies were estimated at 37° C. so as to compare equivalent quantities between all genomes (but see below under native-temperature profiles). The ΔLFE profile for each protein, defined as the estimated excess local folding-energy caused by the arrangement of synonymous codons at any CDS position, was created by subtracting the average profile of 20 randomized sequences for that protein from the native LFE profile:

$$Δ L F E ( i ) = native LFE ( i ) - \frac{1}{N} {\sum }_{n \leq N} randomized LFE ( n , i )$$

(i—CDS position, N—number of randomized sequences)

The mean ΔLFE profile for each species was created by averaging each position i over all proteins of sufficient length (so a different number of sequences may be averaged at each position). Note that while the native LFE of different CDSs within each genome vary considerably, the LFE of each native CDS is compared to its own set of randomized sequences.

To determine if the mean ΔLFE for a species in position i (relative to CDS start or end) is significantly different than 0, the differences dᵢ(p, n) between LFE of the native and randomized sequences for each CDS at that position were collected:

dᵢ(p,n)=nativeLFEᵢ−randomizedLFEᵢ(p,n)

(p—CDS index, n≤N=20—number of randomized sequences) The Wilcoxon signed-rank test was used on all values d(p, n) (with the null hypothesis implying that the distribution is symmetrical).

Native-temperature profiles: The predicted folding-energy calculations for native and randomized sequences for a sample of N=71 bacterial and archaeal species were repeated using the same procedure but with folding predicted at the optimal growth temperature specified for that species (instead of 37° C.).

Phylogenetic tree preparation: To study the relation between ΔLFE profiles and other traits, the profiles were analyzed using a phylogenetic tree as follows. The phylogenetic tree is based on Hug L A, Baker B J, Anantharaman K, Brown C T, Probst A J, Castelle C J, et al. A new view of the tree of life. Nat Microbiol. 2016 Apr. 11; 1:16048, herein incorporated by reference in its entirety see Tables 2-4) and contains species from our dataset across the three domains of life. Since there are slight discrepancies in some node identifiers between the tree and accessions table, species names were matched by hand. Tree nodes and profiles were then matched by NCBI tax-id at the species or lower level between the available genomes and phylogenetic tree nodes (e.g., when the tree species a species, and there is only one genome available for a specific strain of this species). The tree distances were converted to approximate relative ultrametric distances using PATHd8 version 1.9.8 with the default settings. Finally, the tree was pruned to the set of leaf nodes found in the dataset (or a subset of them which has data for both variables being correlated), by removing unused inner and leaf nodes and merging single-child inner nodes by summing distances. The resulting ultrametric tree was used to create a covariance matrix using a Brownian process (to reflect the null hypothesis that a trait is not under selection), using the ape package in R.

Phylogenetically-controlled regression: To test for correlations between traits among species while controlling for the similarity expected to exist between related species even in the absence of selection on either trait, generalized least-squared (GLS) regression was performed with the nlme package in R and using REML optimization. Each regression included the subset of species for which data for both correlated traits was available, and which were also included in the tree. Regression p-values are based on the null-hypothesis that the slope of the explanatory variable is 0 (i.e., that the variables are independent), and estimated using the t-test. Coefficient of determination (R²) values were calculated according to:

$${R}^{2} = 1 - \frac{{\hat{u}}^{′} {V}^{- 1} \hat{u}}{{( Y - \overline{Y} e )}^{′} {V}^{- 1} ( Y - \overline{Y} e )}$$

û—residuals, V—variance-covariance matrix, Y—observations, Y̅—intercept of equivalent intercept-only model, e—first column of design matrix.

For continuous traits, regression formulas included an intercept term. Discrete traits were represented by ordered or unordered factors and the intercept term was omitted from the regression formula. For discrete traits, values of the explained variable (such as ΔLFE) were centered to have mean 0 (so regression is based on a null hypothesis that all levels have the same mean).

Regression robustness verification: To test the robustness of a correlation between traits at different CDS regions, the regression was repeated at all profile positions starting between 0-300 nt (relative to CDS start and end) and all contiguous subranges (using the mean ΔLFE value in each range) and reported only if consistent over the relevant range of positions (FIG. 27).

To test for specific trait correlations in individual taxa, the regression procedure was repeated for each taxonomic group (at any rank) containing at least 9 species (FIG. 20). For each taxonomic group, the value shown is the median R² value for positions within the relevant range. The significance p-value threshold was determined by applying FDR correction according to the number of taxonomic groups (treating them as independent to get a “worst-case” result). In some embodiments, the p-value threshold is the threshold of the invention.

### Model Element Definition Rules:

Elements of the ΔLFE profile model were formalized as follows to allow estimation of their prevalence (FIG. 1A). Significance for all rules is defined using the Wilcoxon signed-rank test (see above) having p-value<0.05 at all positions within the range specified.

### Model 1 (Positive Ends)

A. Positive start: ΔLFE value at positions 0-10 nt relative to CDS start is positive and significant.
B. Transition peak: the position of the minimum ΔLFE value in the range 0-300 nt, i*, is located in the range 20-80 nt relative to CDS start, and is significantly lower compared to all points in the ranges 0-10 nt, 100-200 nt relative to CDS start.
   To determine if the mean ΔLFE for a species in a given position i is significantly higher than the minimum (i*), the differences wᵢ(p, n) between ΔLFE at the peak position and ΔLFE at the tested position were collected:

wᵢ(p,n)=d_{i*}(p,n)−dᵢ(p,n)
(p—CDS index, N≤20—number of randomized sequences, i—position in CDS relative to start)
The Wilcoxon signed-rank test was used on all values wᵢ(p,n).
C. Negative mid: ΔLFE values at each position in the range 200-300 nt relative to CDS start and in the range 300-200 nt relative to CDS end are all negative and significant.
D. Positive end: ΔLFE value at positions 10-0 nt relative to CDS end is positive and significant.
E. Model structure: A+C+D

### Model 2 (Weak Ends)

A. Weak start: ΔLFE value at position 0 nt relative to CDS start is significantly higher than at positions 200-300 nt.
B. Same as in Model 1.
C. Same as in Model 1.
D. Weak end: ΔLFE value at position 0 nt relative to CDS end is significantly higher than at positions 200-300 nt.
E. Model Structure: A+C+D

### Binary Classifier for ΔLFE Strength

To measure the performances of several criteria in predicting ΔLFE strength, the following simple model was used. ΔLFE values for all species were divided into weak and strong groups based on the standard-deviation of the mean ΔLFE at positions 0-300 nt. Species with standard-deviation <0.14 were included in the “weak ΔLFE” group. The binary classification of each species is based on 4 species traits as inputs, using the following rule (optimized using grid search):

PredictedWeakLFE=(Endosymbiont=True) or (Genomic-GC<38%) or (Genomic-ENc′>56.5) or (Optimum-temp>58° C.)

### Maximal Information Coefficient (MIC)

Maximal Information Coefficient (MIC) is a statistical measure of general (not necessarily linear) dependence between two variables. Informally, it is a generalization of R², and also has values in the range 0.0-1.0, with high values indicating knowing the value of one variable allows inferring the value of the other. MIC was calculated using the minerva package in R. p-values were estimated using 10,000 random samples.

### Correlogram Plot

Correlogram plot (FIG. 12) was prepared using the phylosignal package in R.

### Codon-Bias Metrics

Codon-bias metrics (CAI, CBI, Nc, Fop) were calculated for each genome using codonW version 1.4.4. ENc′ was calculated using ENCprime (github user jnovembre, commit 0ead568, October 2016) using the default settings. I_TE was calculated using DAMBE7, based on the included codon frequency tables for each species. DCBS was calculated according to Sabi R, Tuller T. Modelling the Efficiency of Codon-tRNA Interactions Based on Codon Usage Bias. DNA Res. 2014 Oct. 1; 21(5):511-26, herein incorporated by reference.

### Shine-Dalgarno Binding Strength

Shine-Dalgarno (SD) strength for each gene was calculated according to Bahiri Elitzur S, et al. “Prokaryotic rRNA-mRNA interactions are involved in all translation steps and shape bacterial transcripts.” Rev. 2020, herein incorporated by reference in its entirety, based on the minimal anti-SD hybridization energy found in the 20 nt region upstream of the start codon.

### Visualization

Taxon characteristic profiles chart: The mean ΔLFE profiles for CDS positions 0-300 nt relative to the CDS start and end within each taxon were summarized (FIG. 3A) by grouping species with similar profiles and plotting one profile representing each group. The grouping was achieved by clustering the ΔLFE profiles (as vectors of length 31) using K-nearest neighbors agglomerative clustering with correlation distances, using SciKit Learn. The profile plotted to represent each group is the centroid (mean) of each cluster. To allow easy viewing of the region of interest, only positions 0-150 nt are shown for each cluster. K, the number of clusters for each taxon, was chosen (separately for the start end end profiles) to be the smallest value for which the maximum distance of any profile to the centroid cluster mean (i.e., the profile shown) was smaller than 0.8 for the start-referenced profiles and 1.3 for the end-referenced profiles. The full ΔLFE profiles for all species appear in FIG. 17.

PCA display for ΔLFE profiles: To summarize ΔLFE profiles and show how different values related to different profile types, we used PCA analysis to obtain a two-dimensional arrangement in which similar ΔLFE profiles are mapped to nearby positions. (see for example FIG. 3B). Also shown are the amounts of variance explained by each of the first two principal components.

PCA analysis for the ΔLFE profiles (treated as vectors of length 31) was performed using SciKit Learn. Analysis was limited to the first 3 components and only the first two components are displayed (FIG. 16A-B). To verify the robustness of the PCA results, they were repeated using 500 samples with replacement from the same PCA input vectors and of the same size, and the angles between the component were verified to be approximately equal (FIG. 16C). To reduce clutter, overlapping profiles are hidden and the relative density at each position is shown in the background as blue shading (estimated as bivariate KDE with bandwidth determined by Scott's rule using seaborn) and also plotted on the axes.

Evolutionary and taxonomic trees were plotted using ETE toolkit.

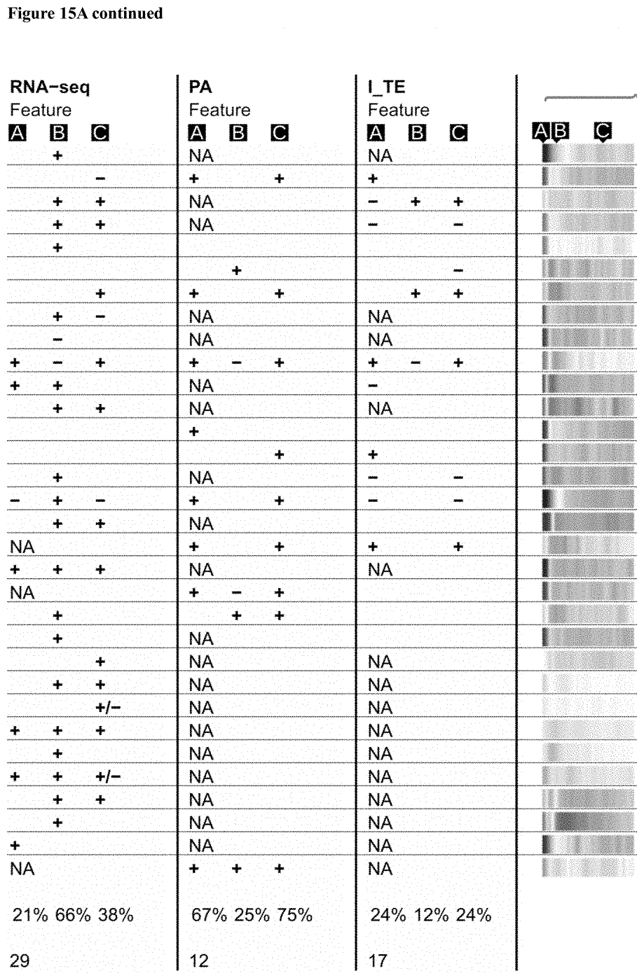

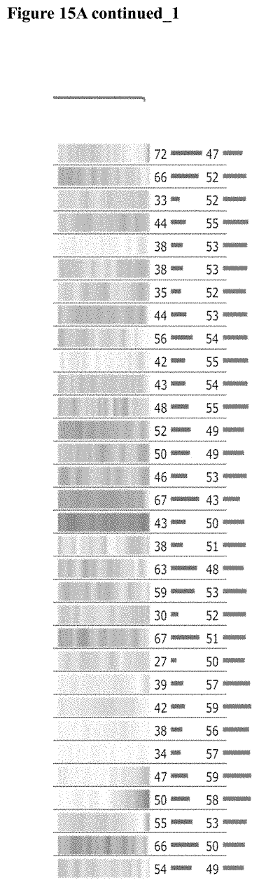

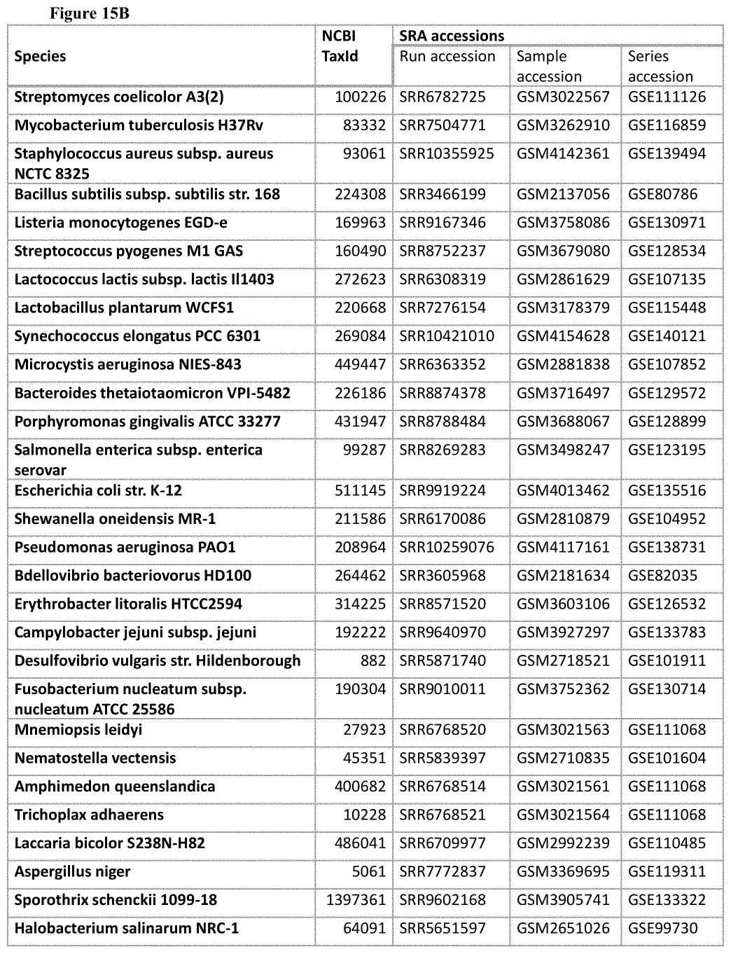

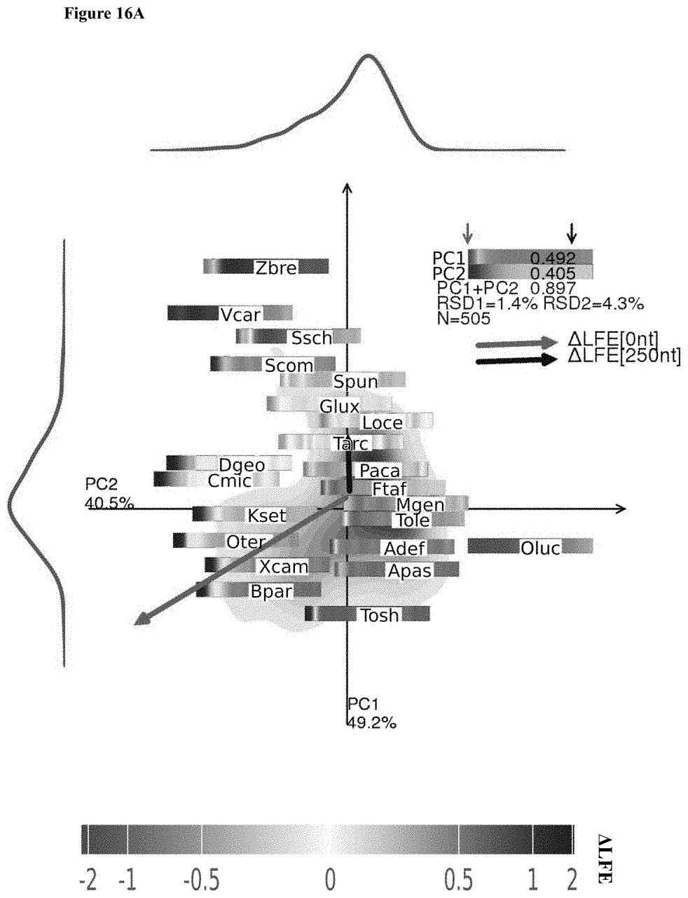

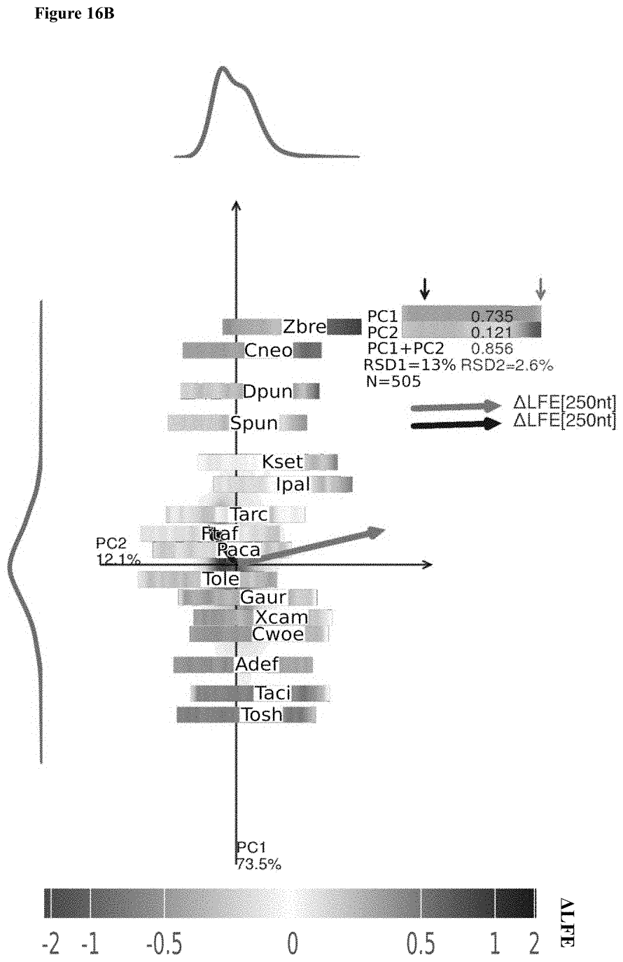

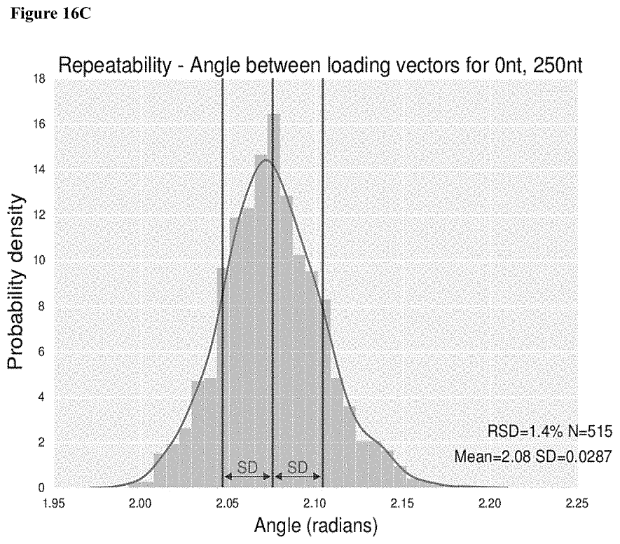

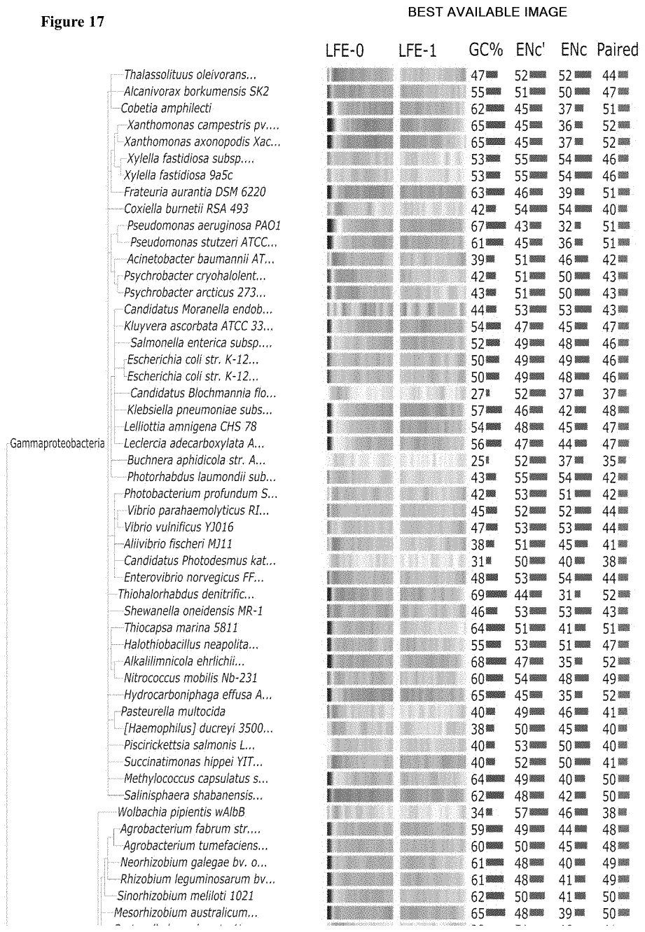

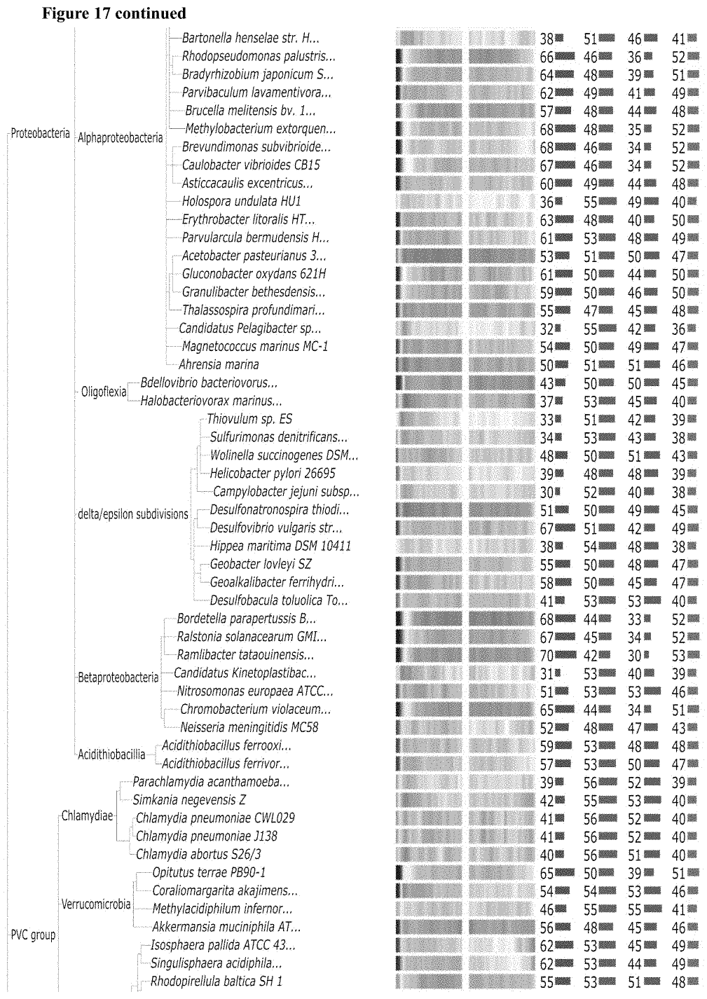

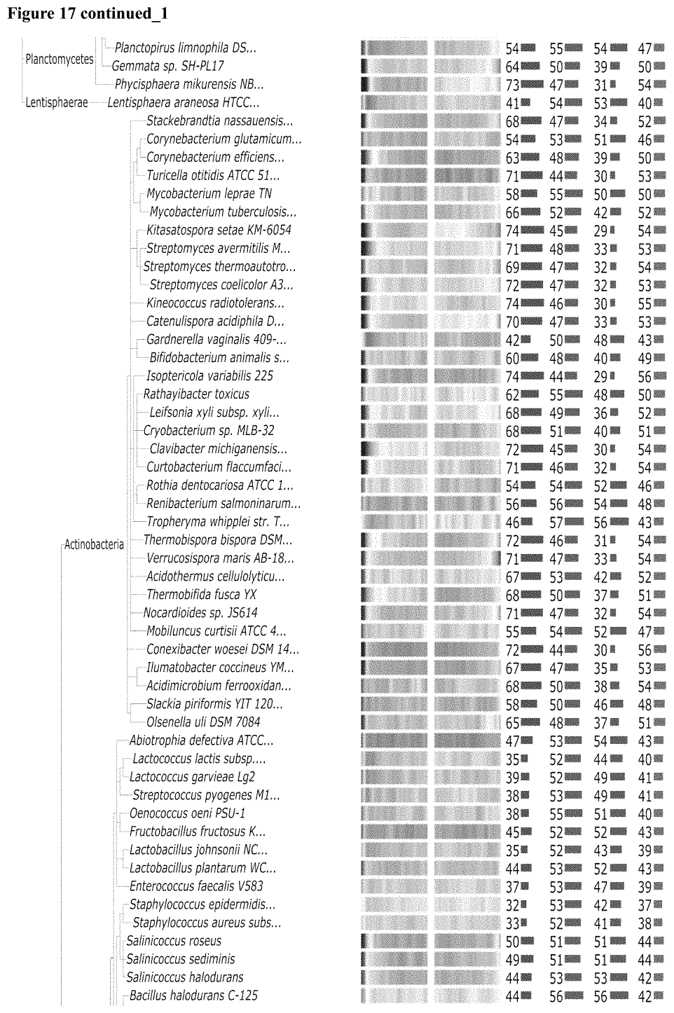

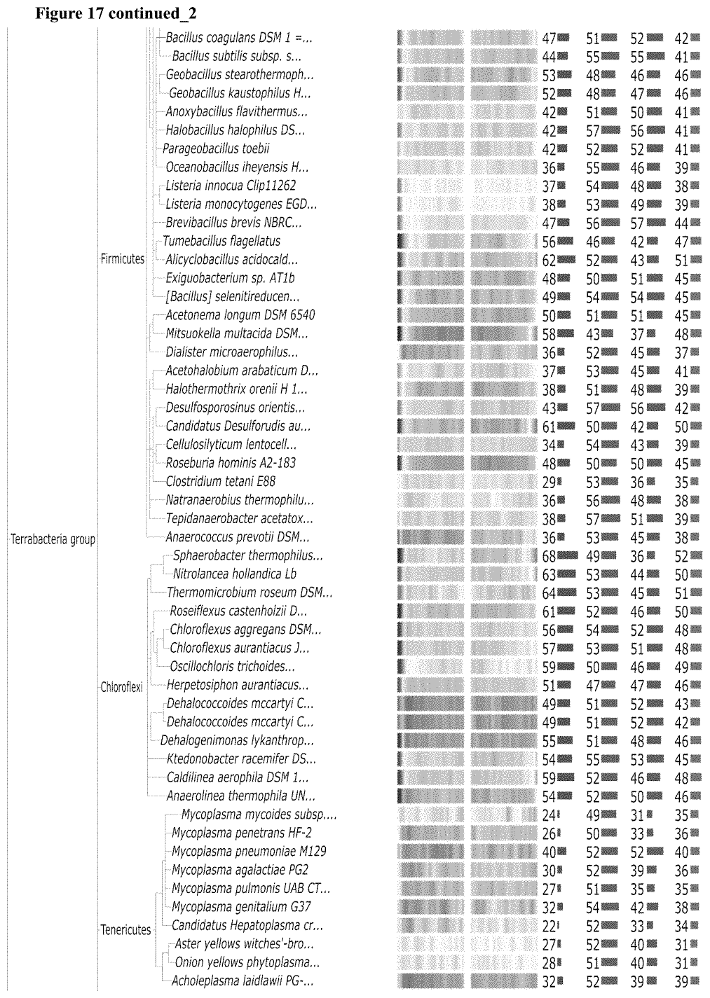

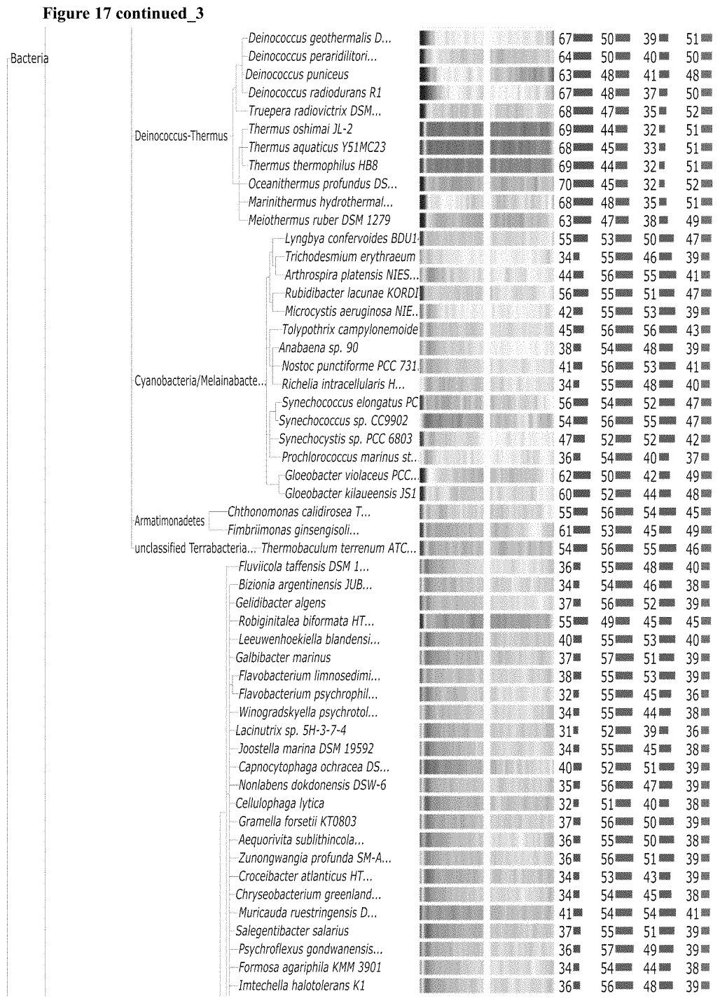

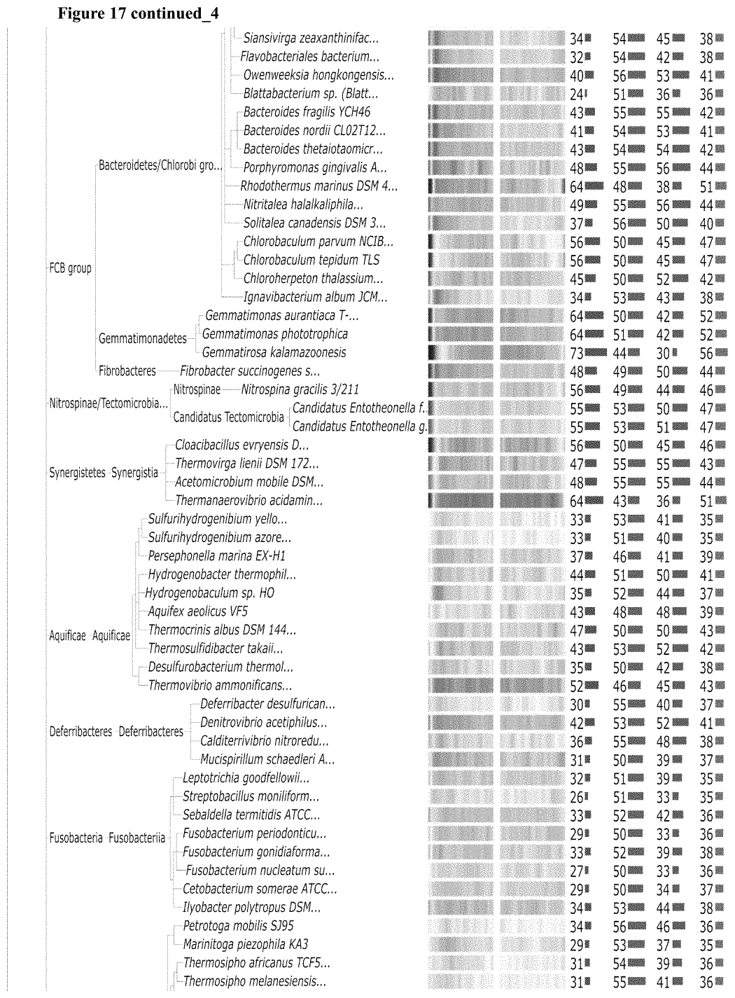

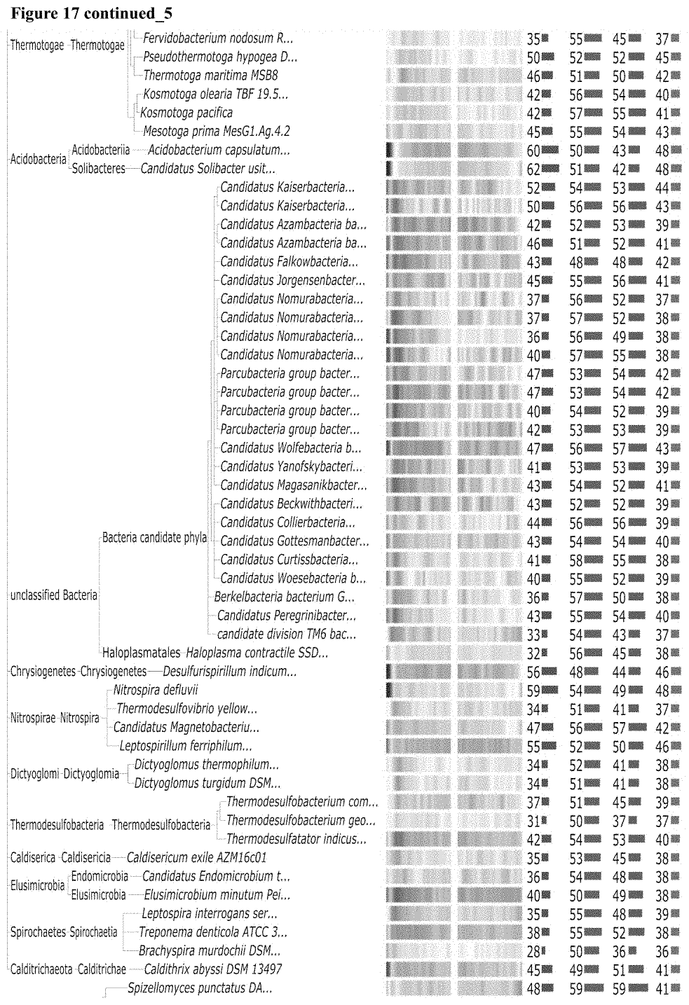

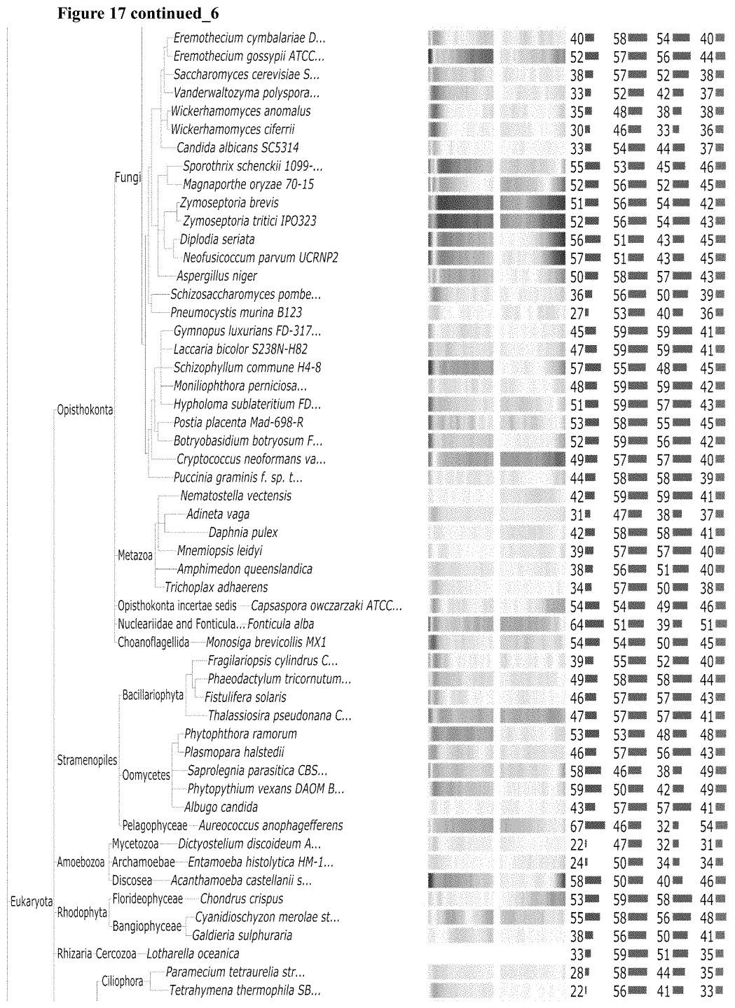

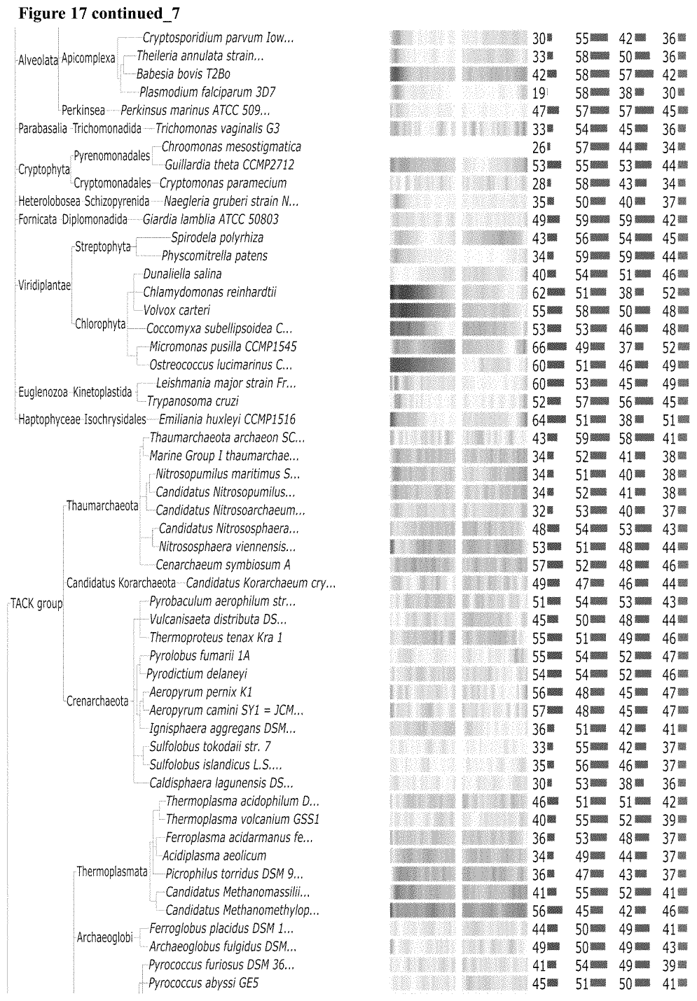

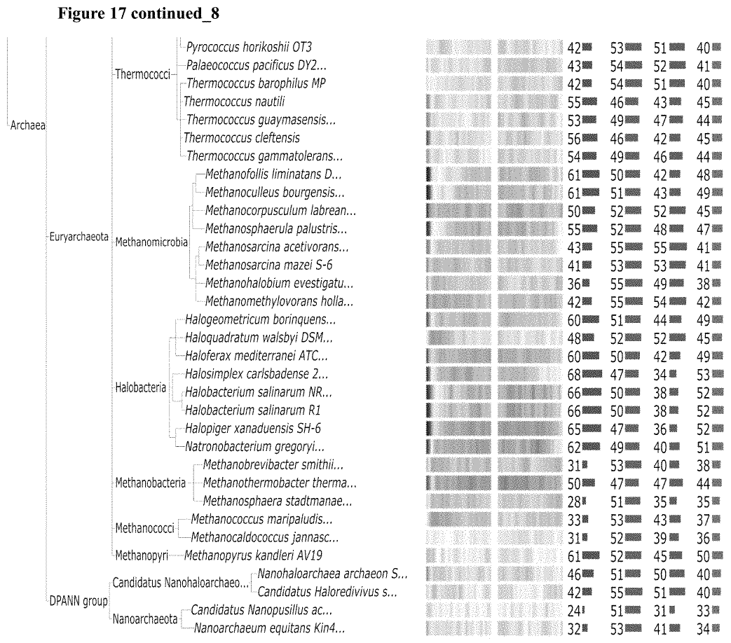

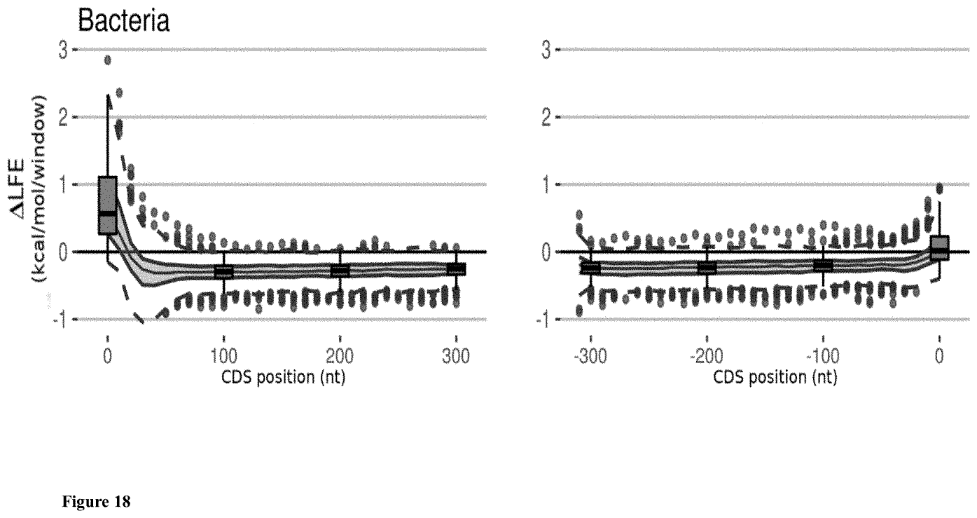

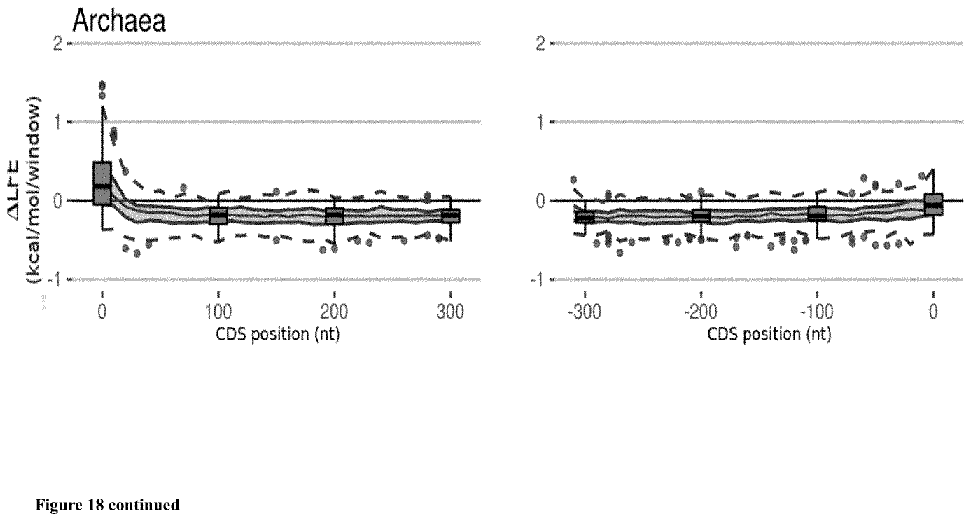

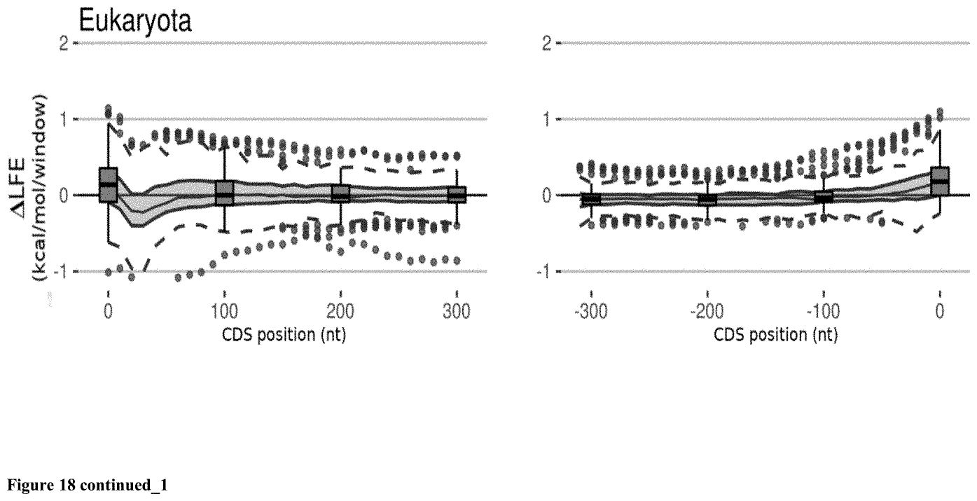

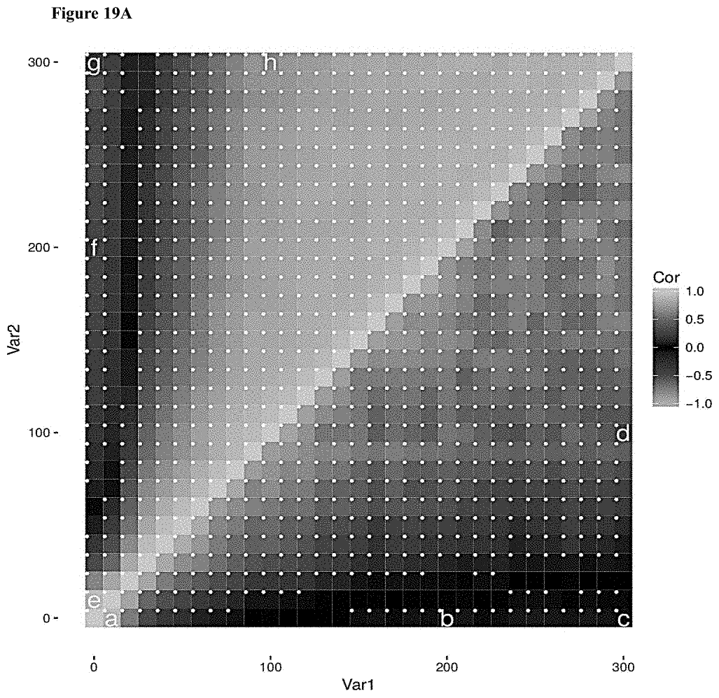

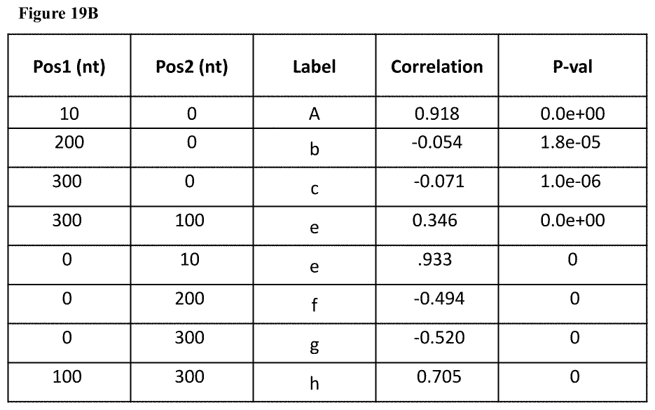

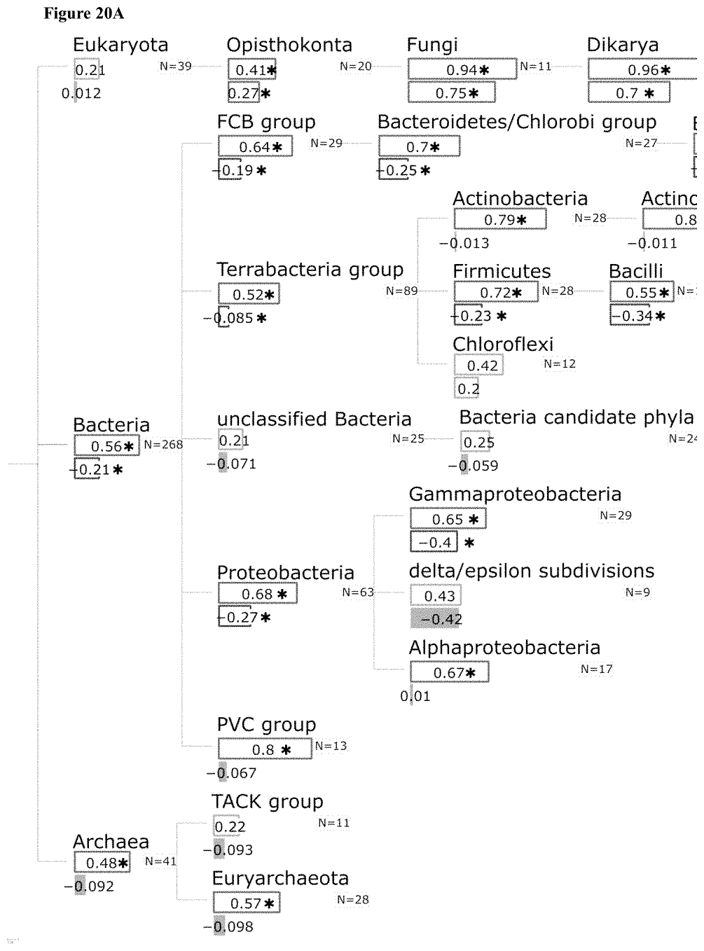

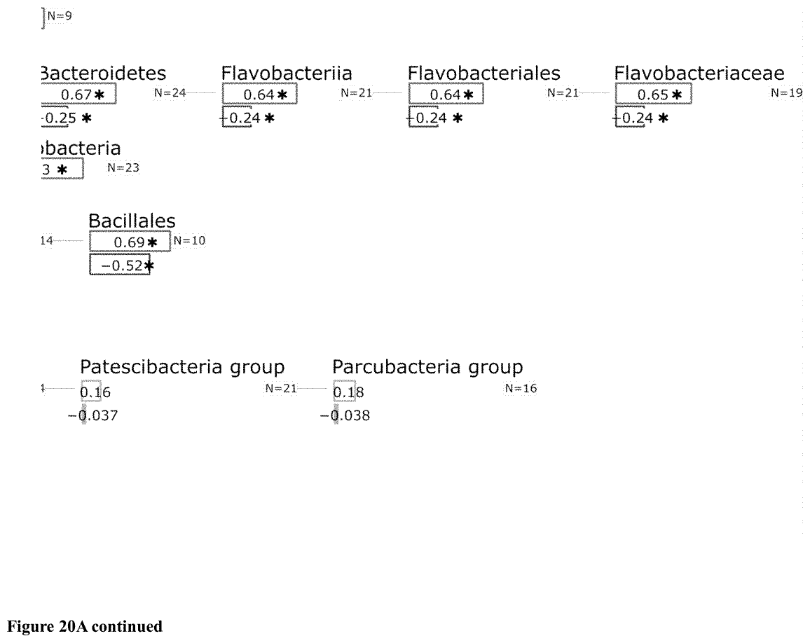

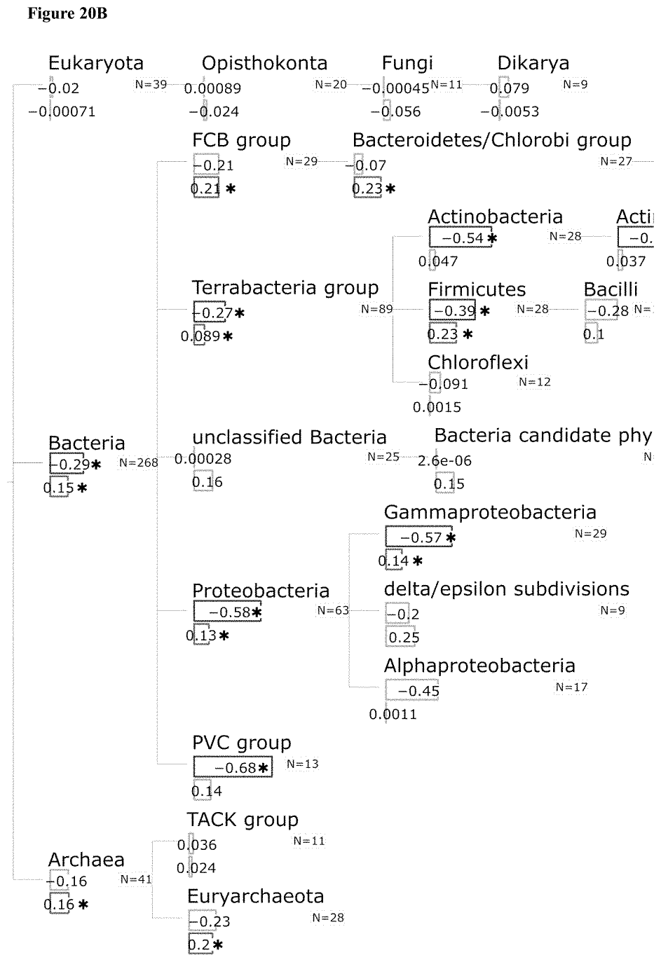

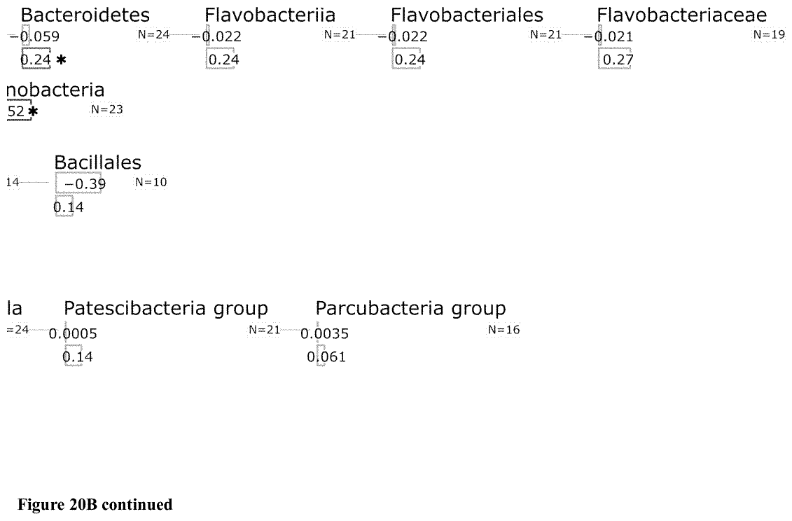

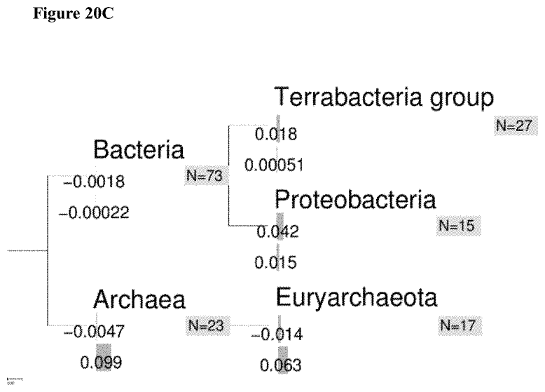

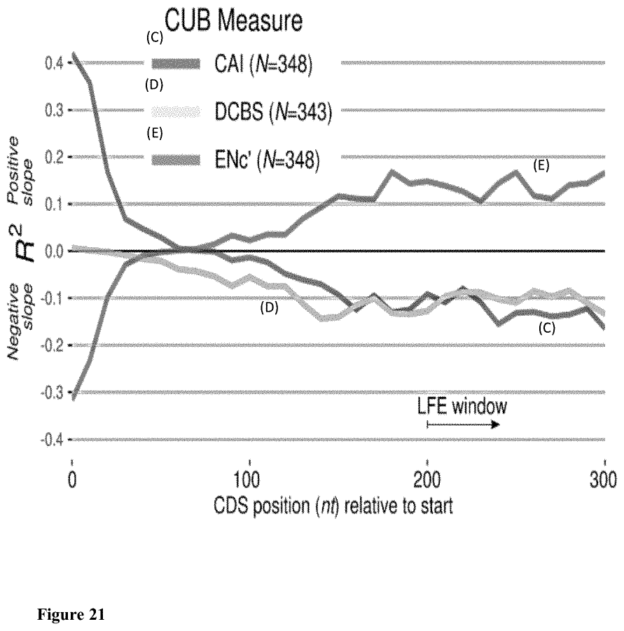

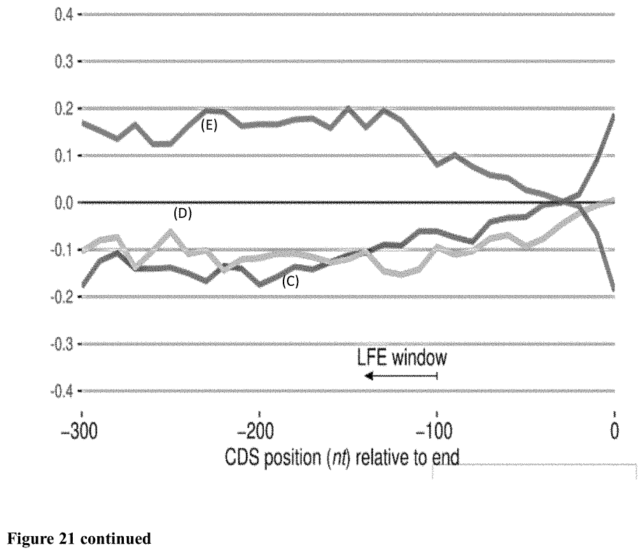

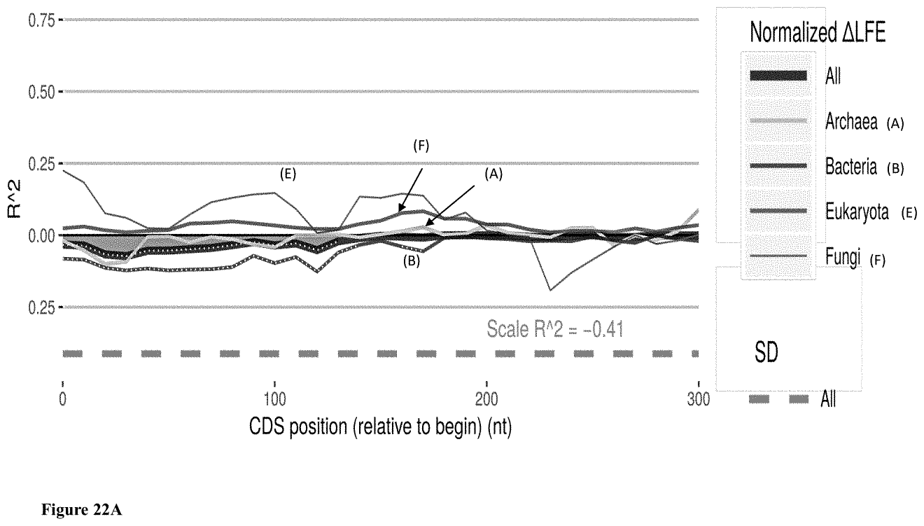

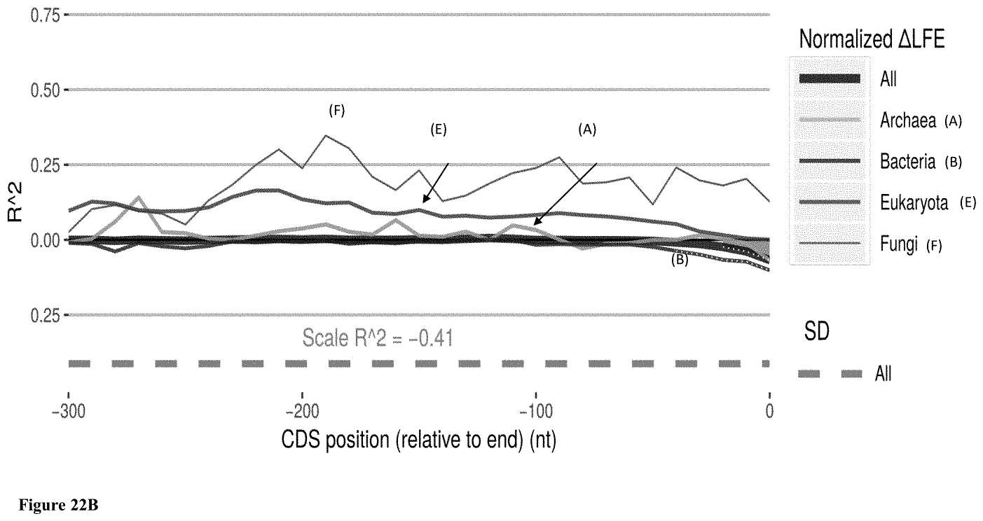

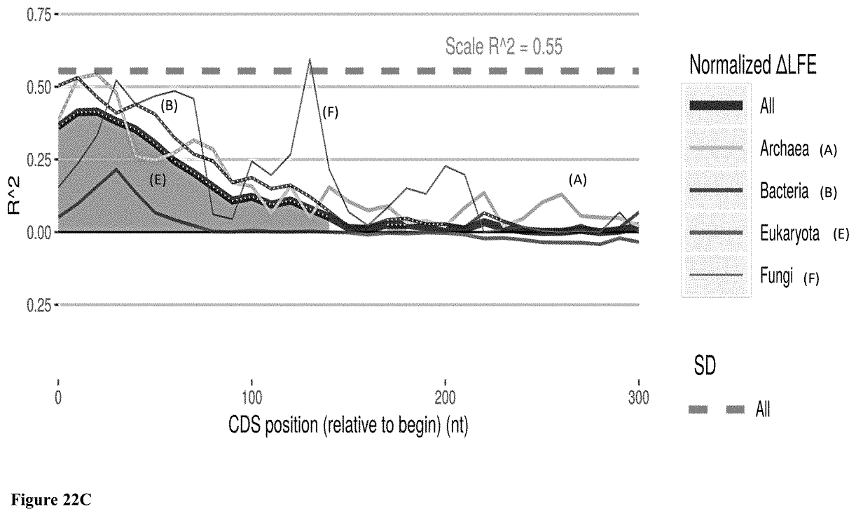

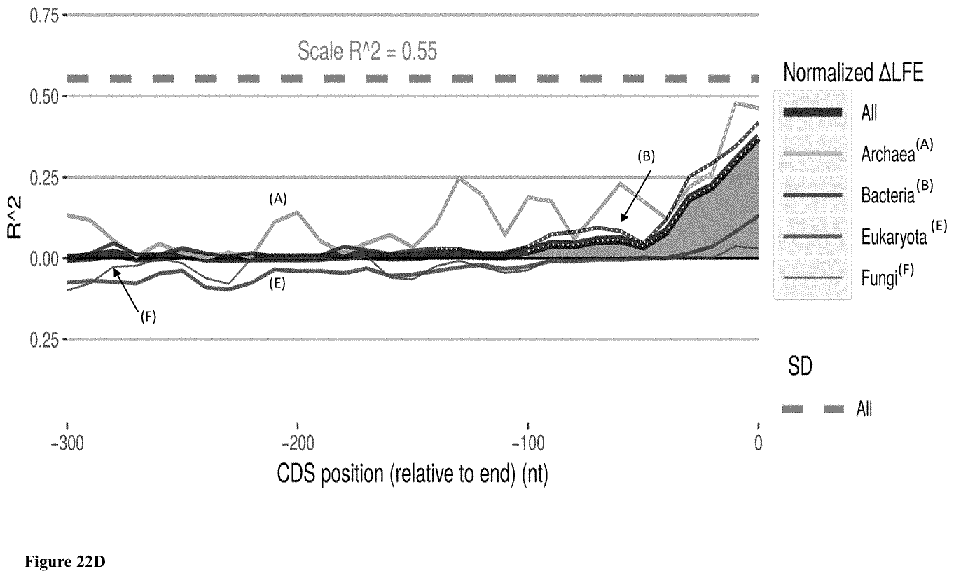

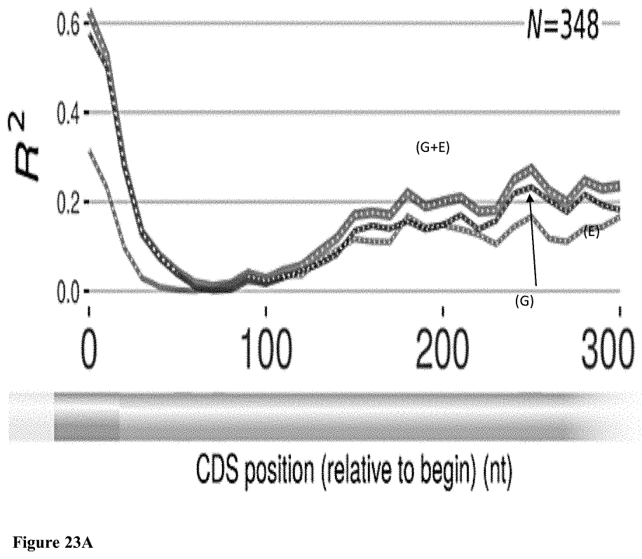

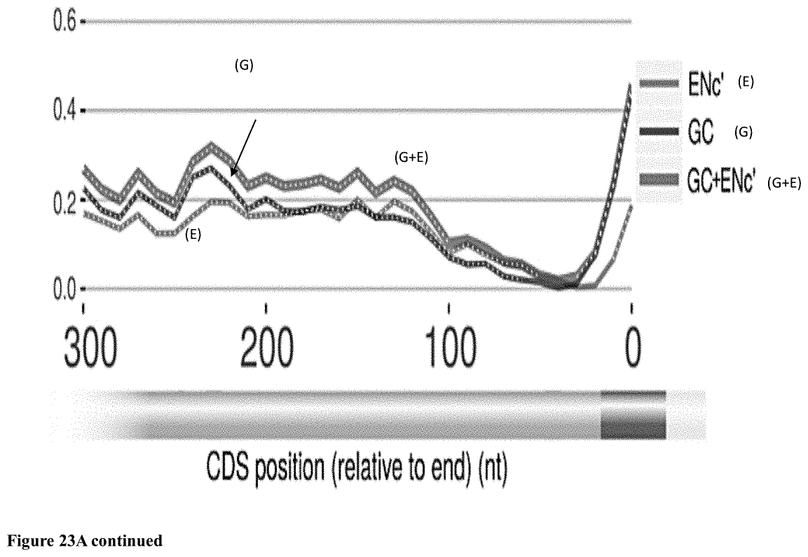

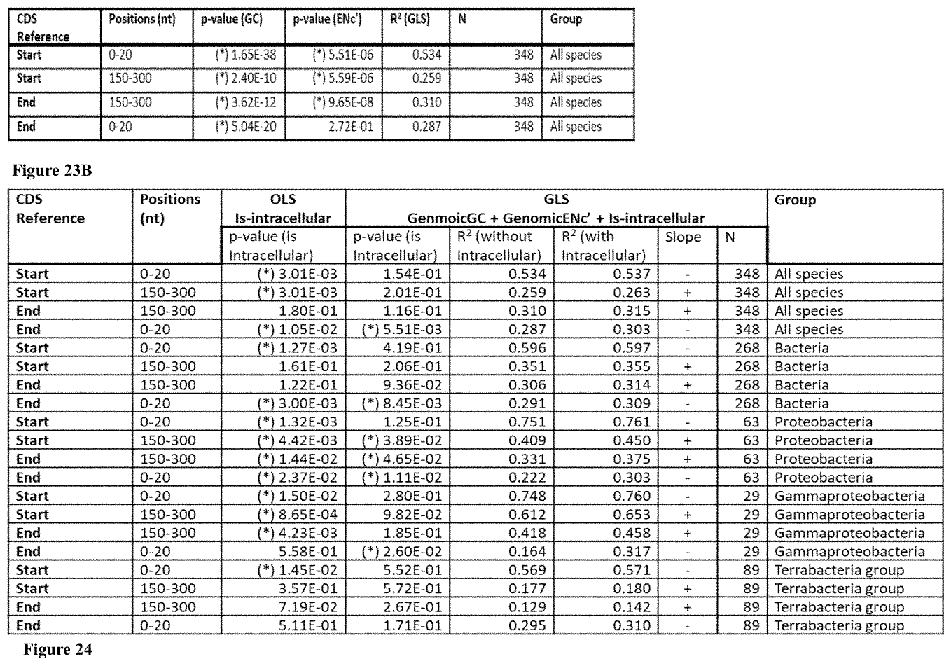

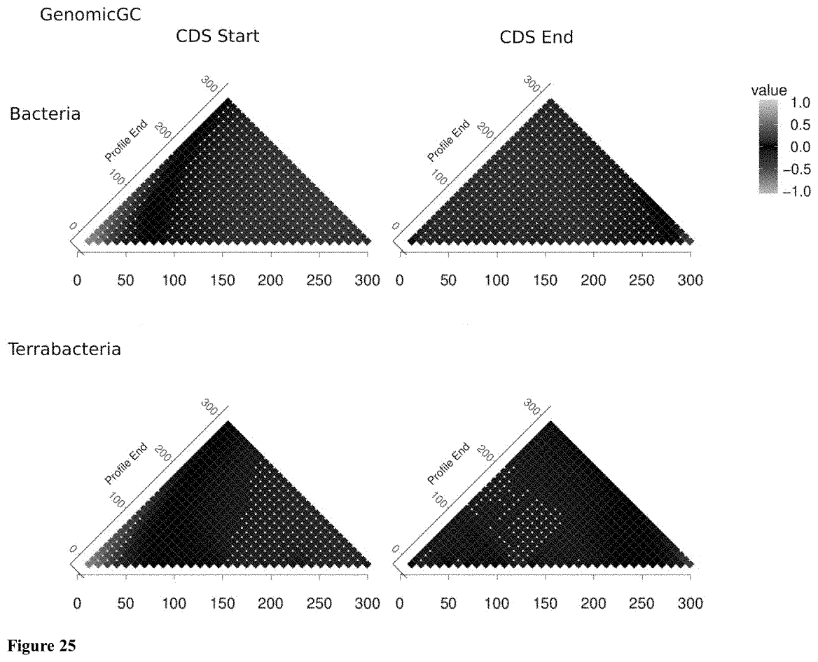

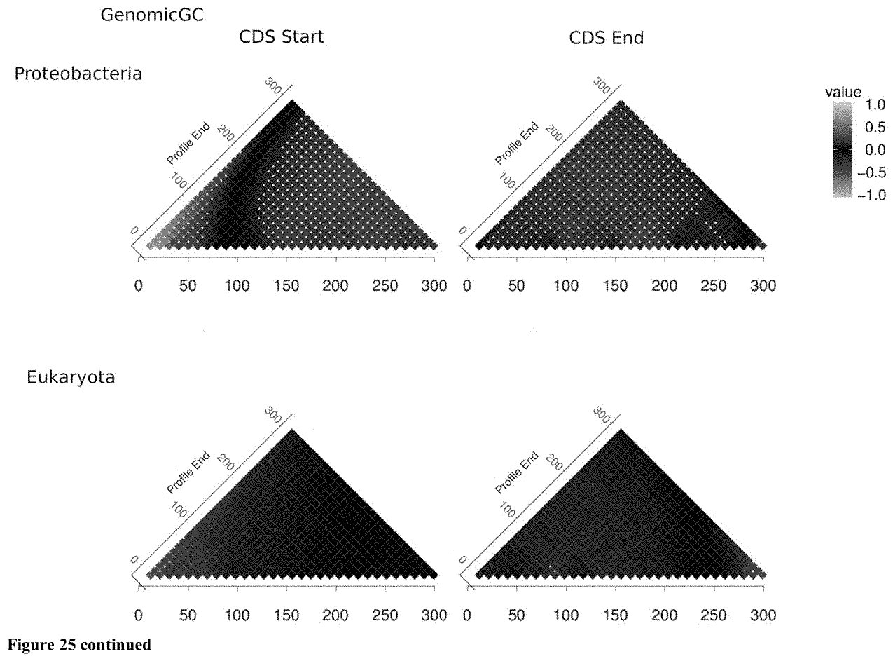

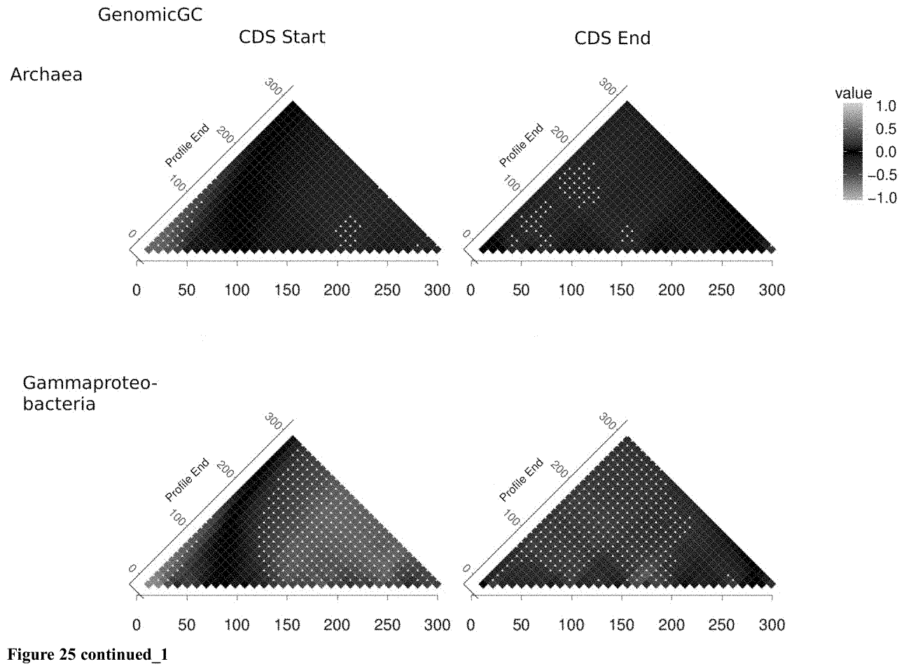

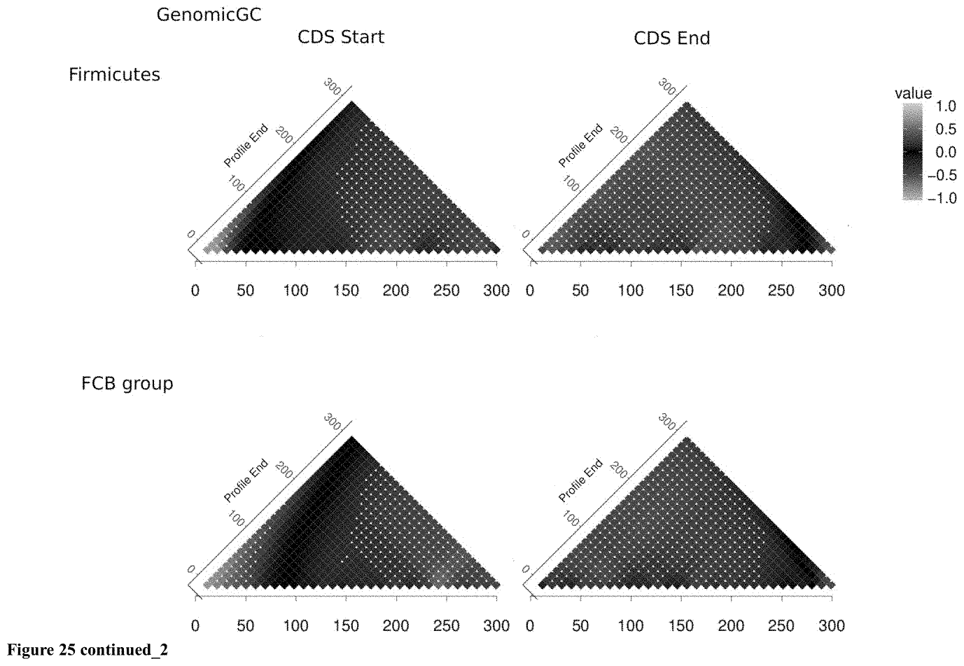

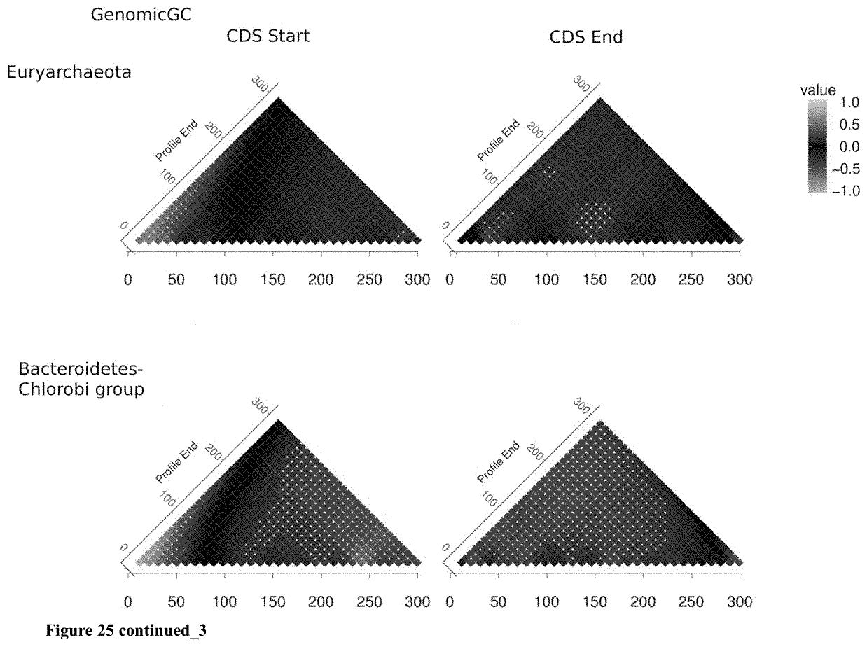

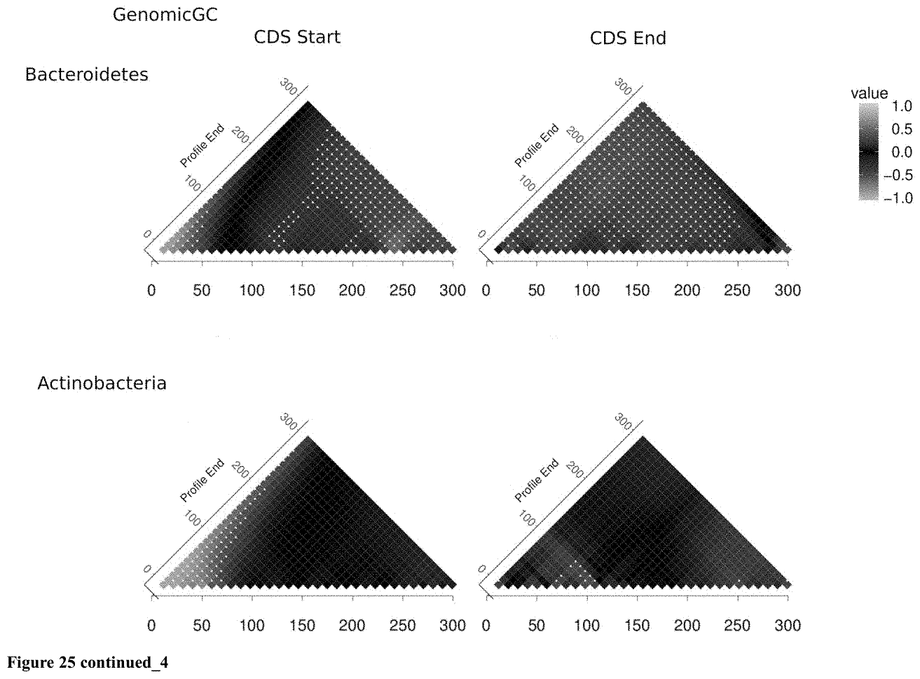

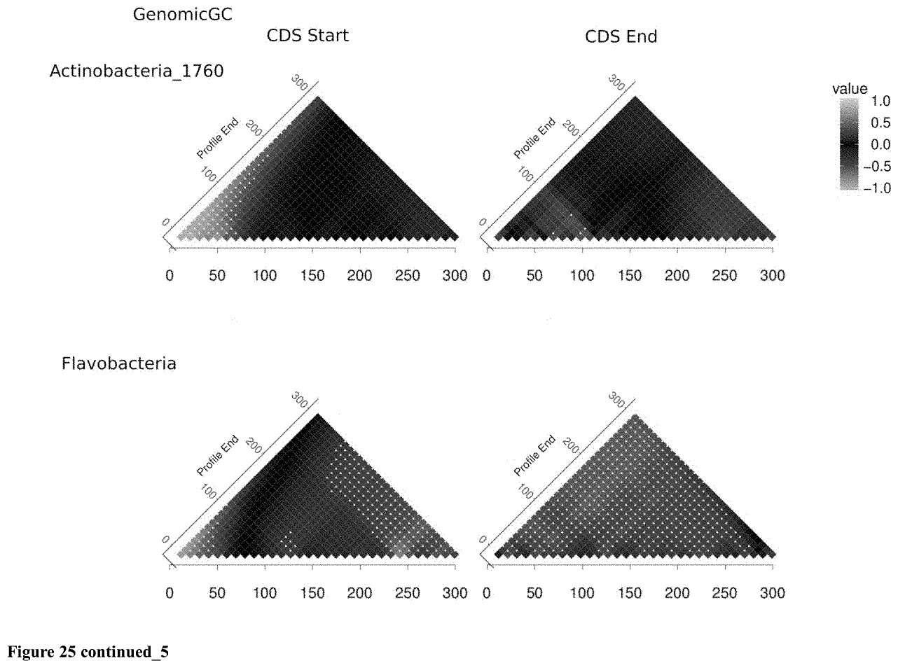

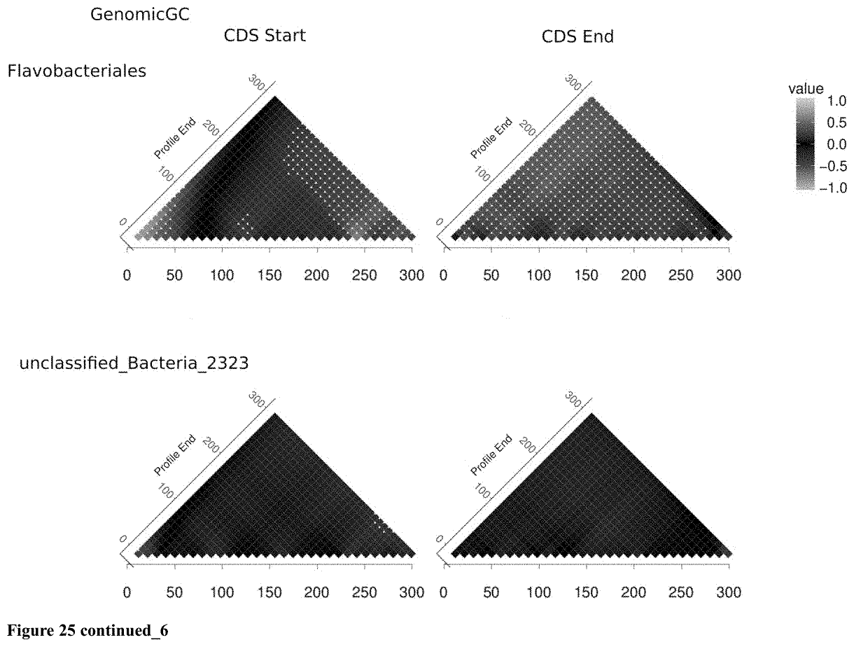

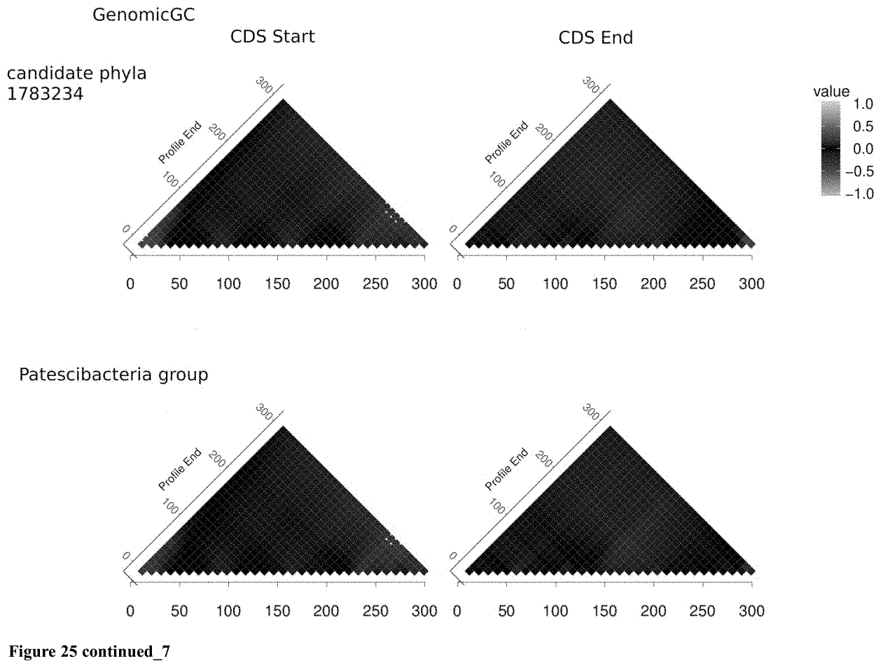

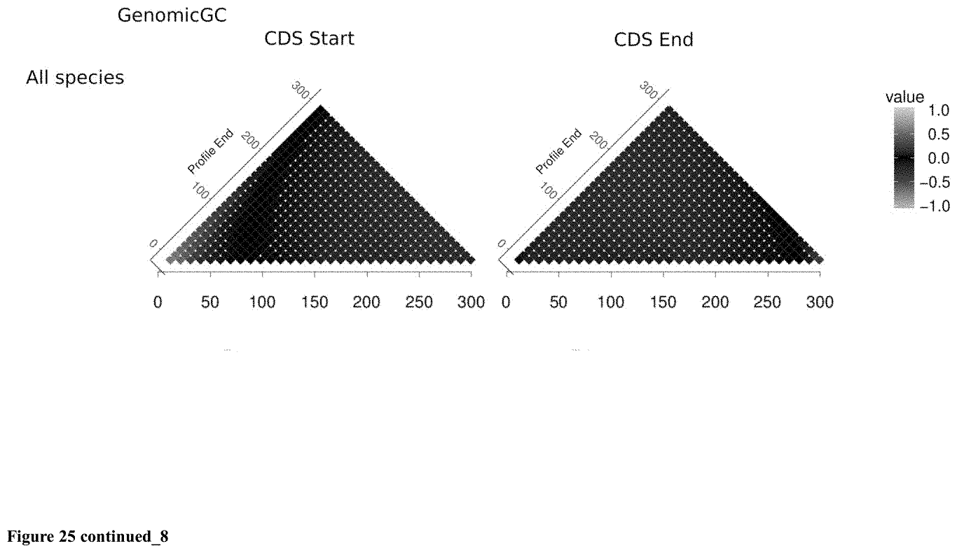

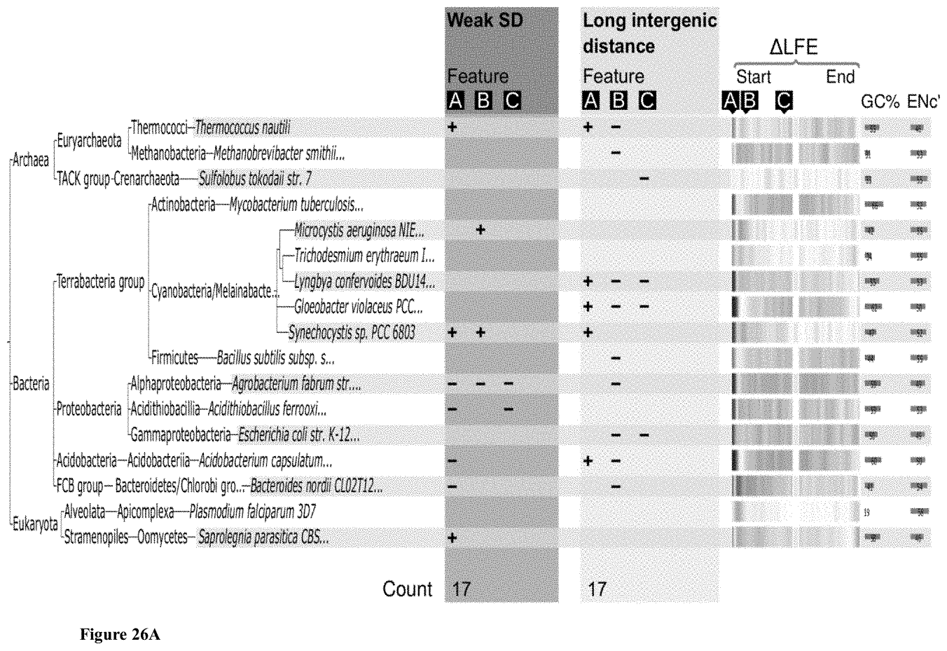

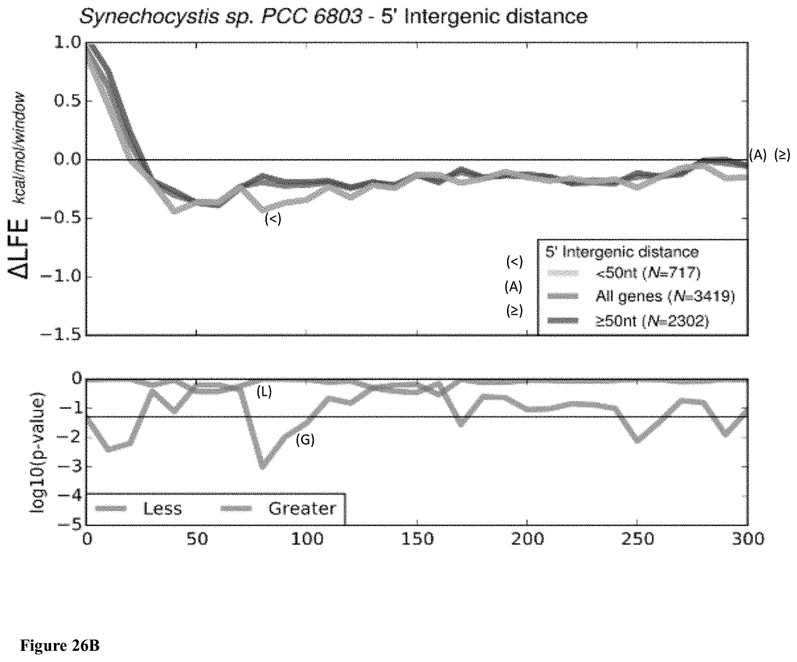

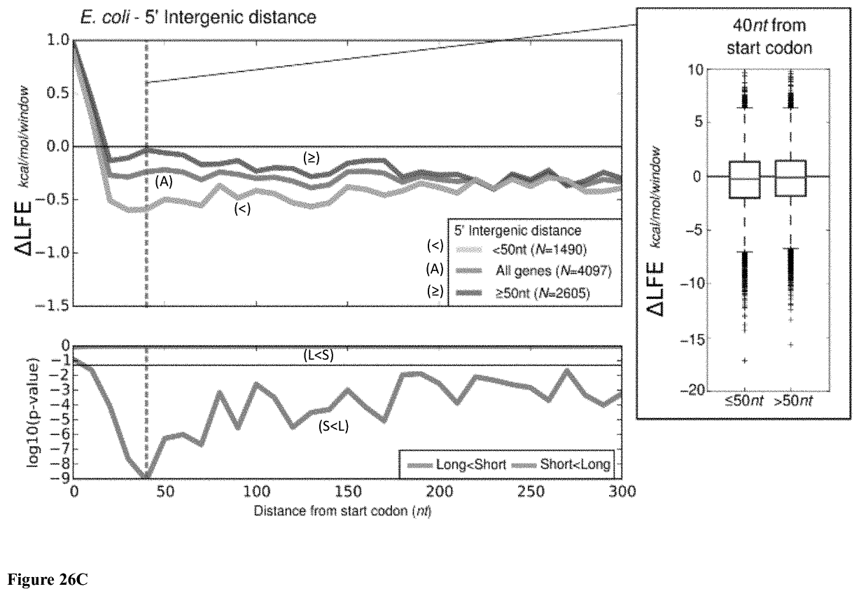

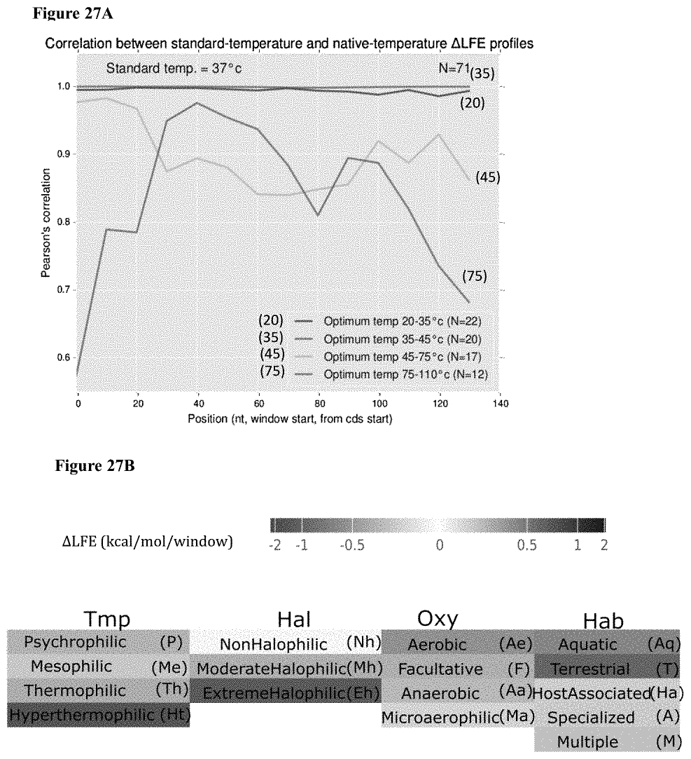

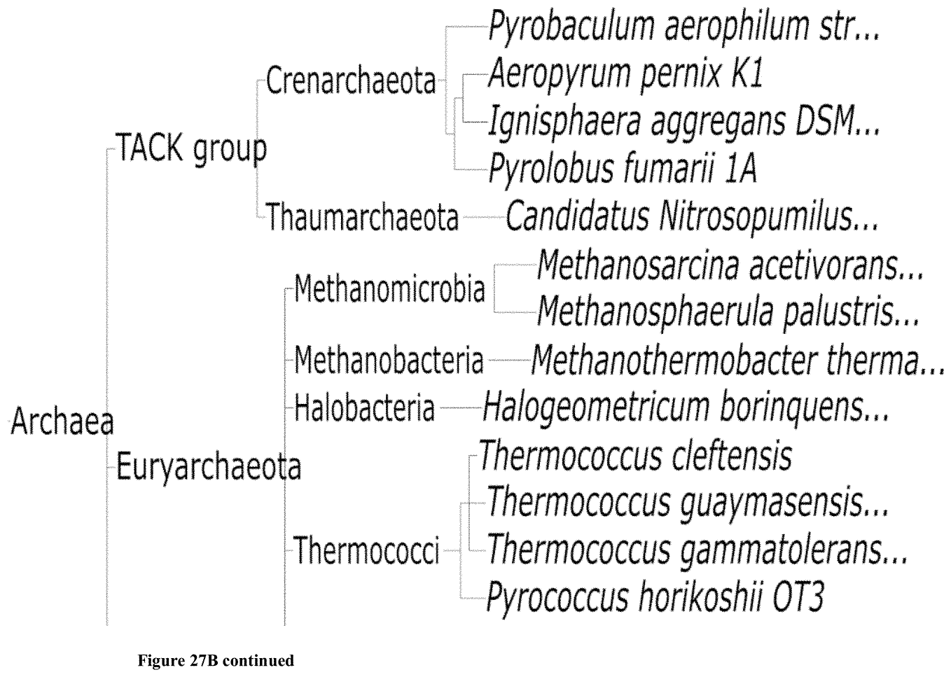

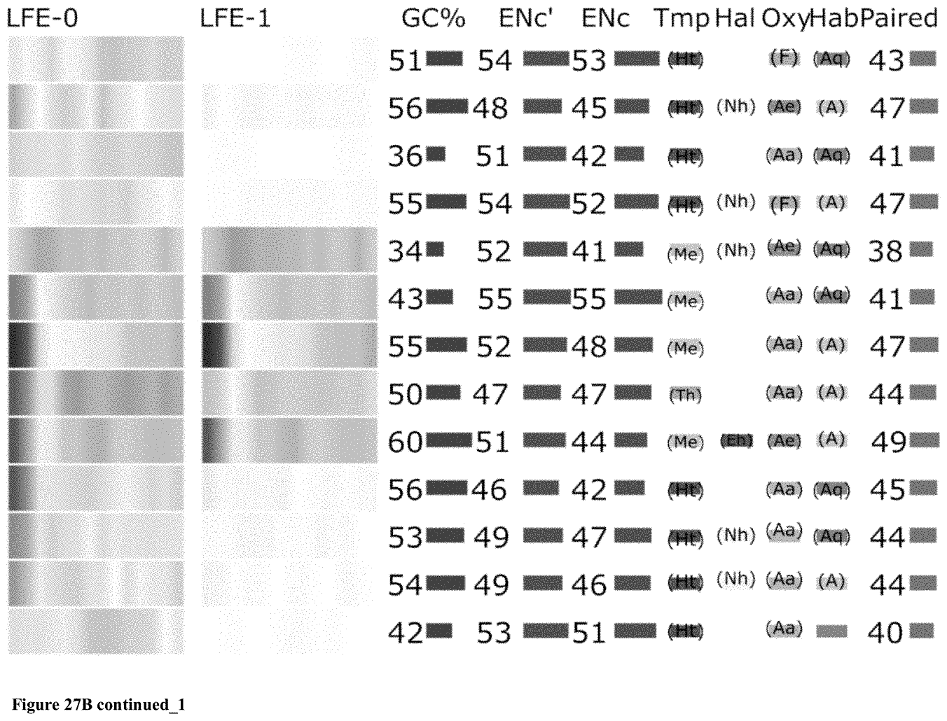

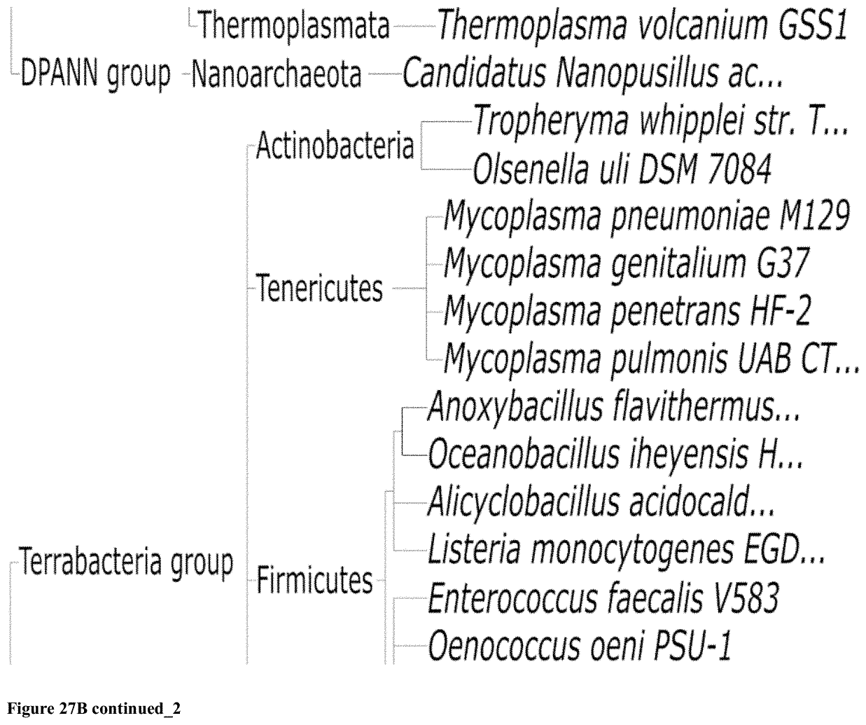

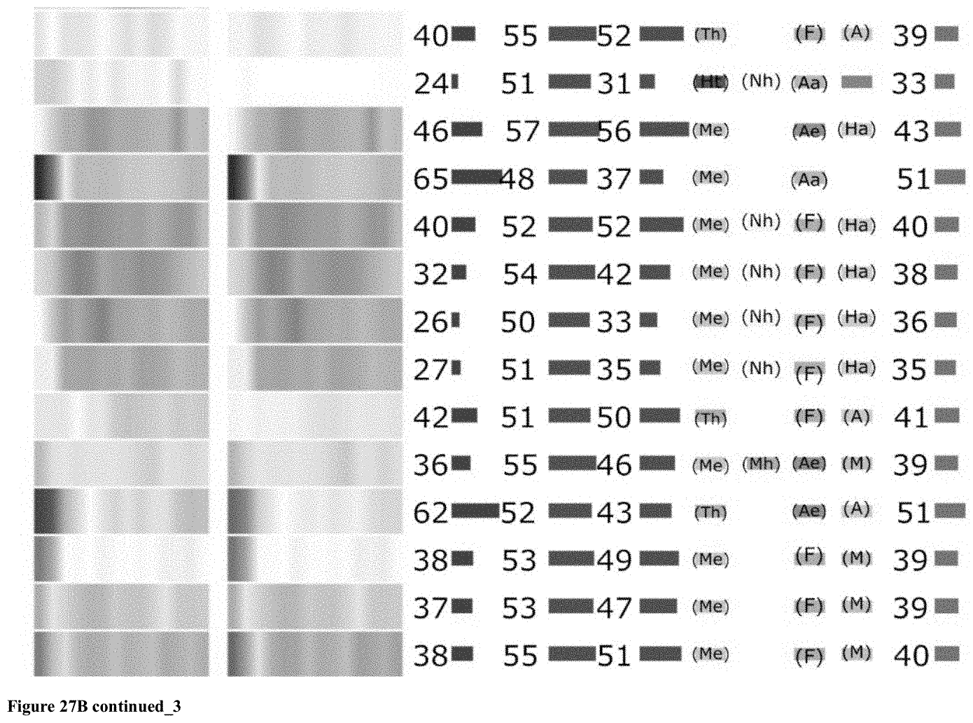

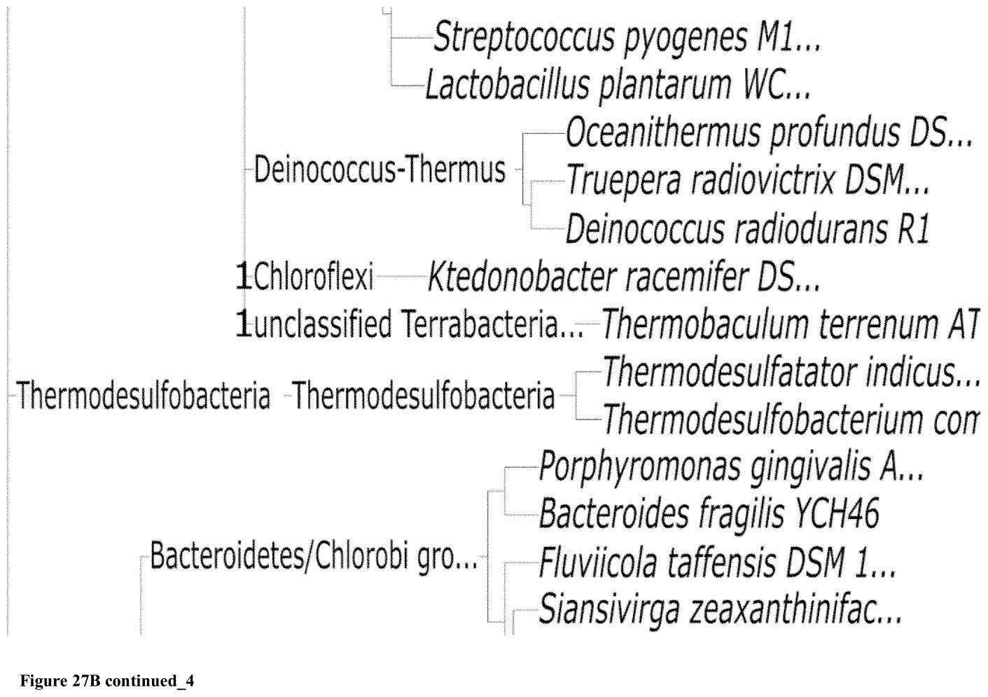

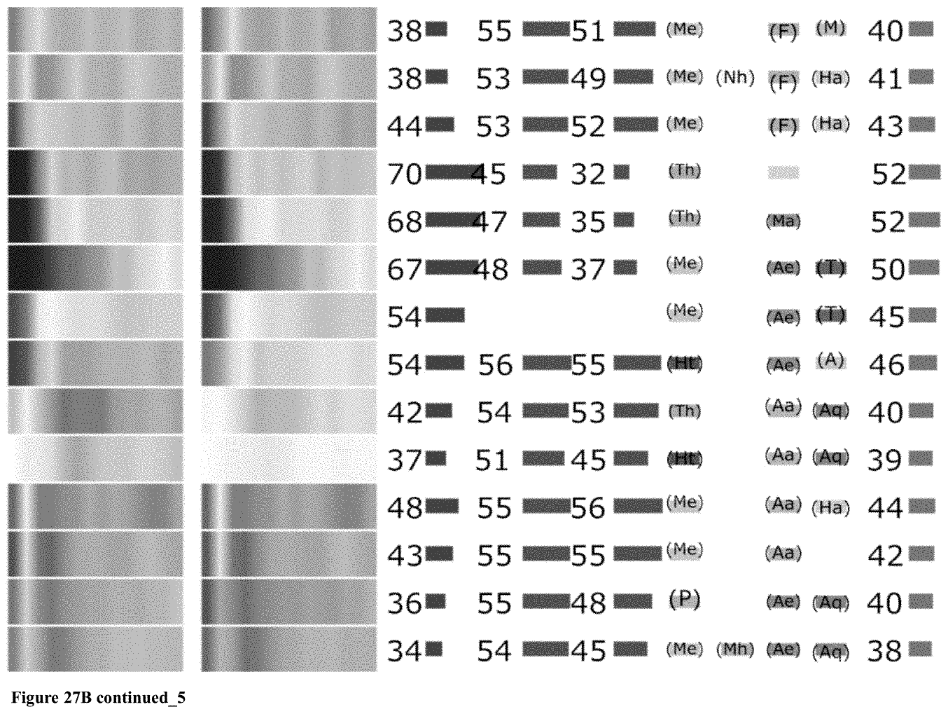

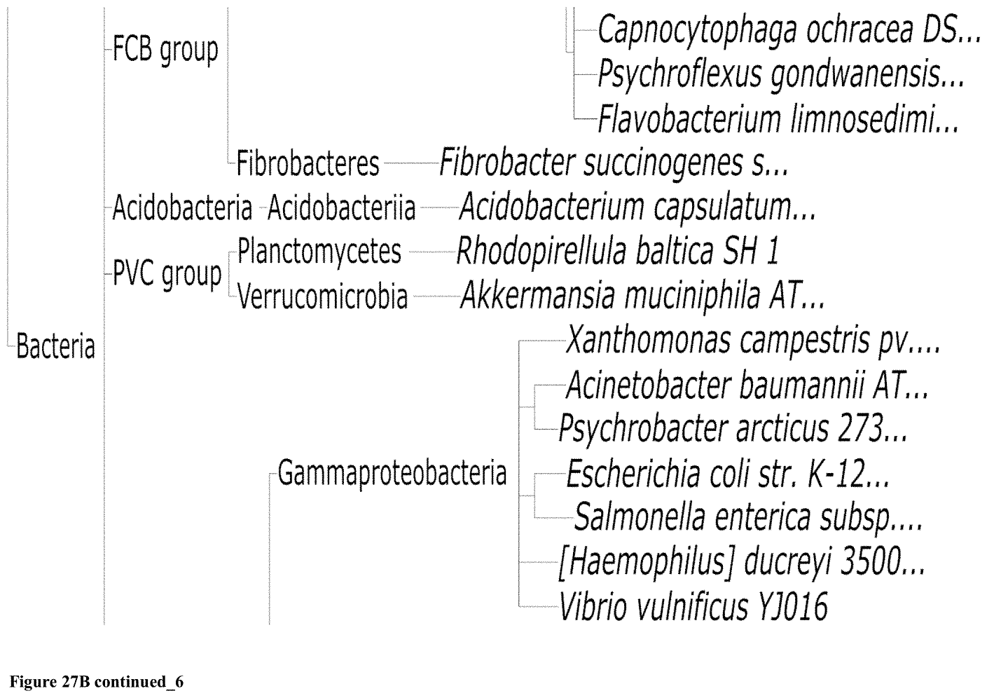

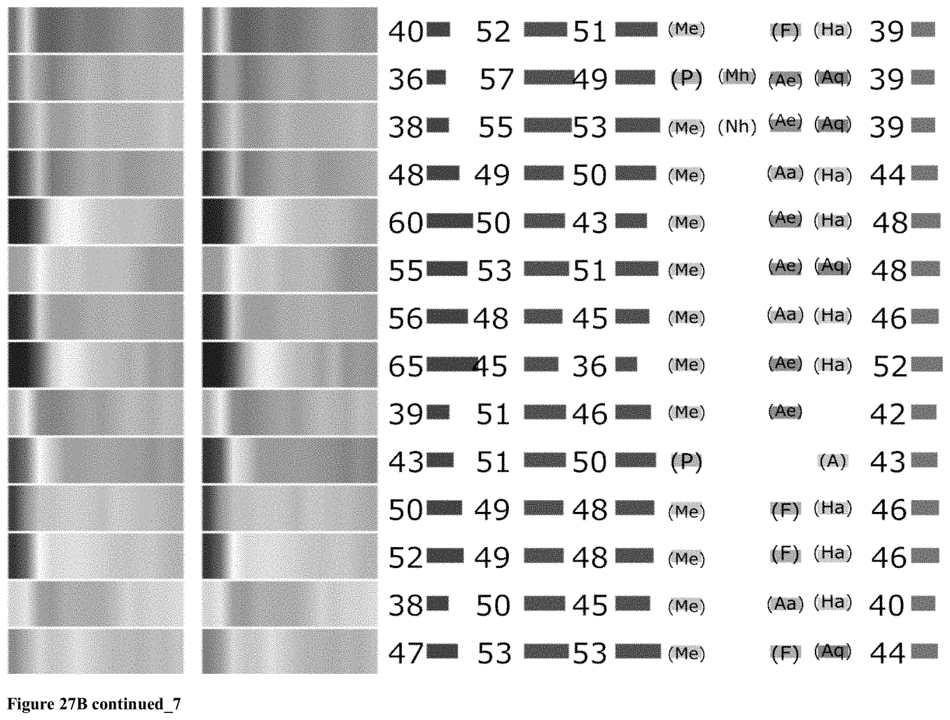

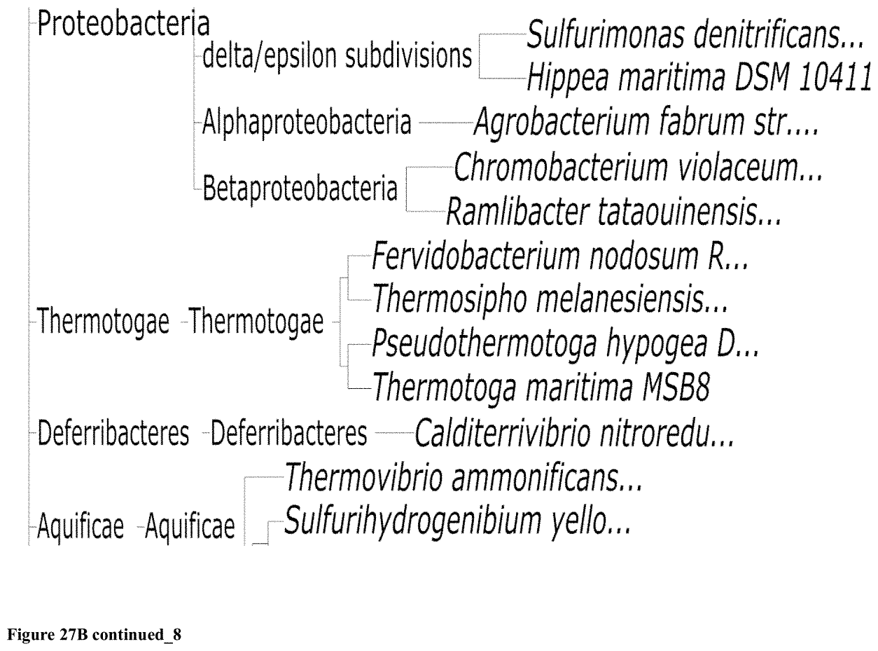

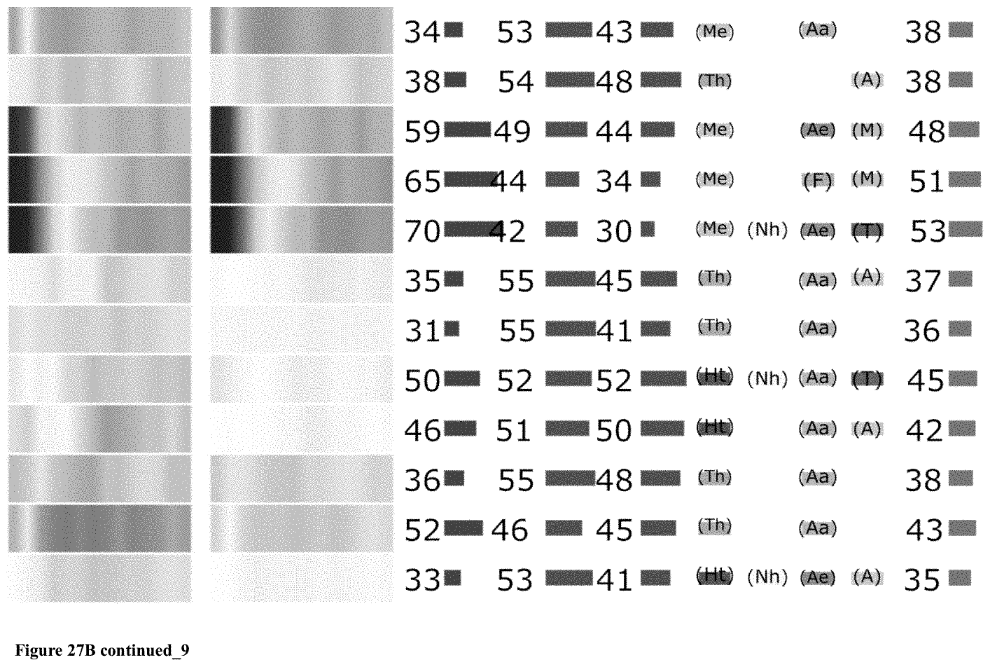

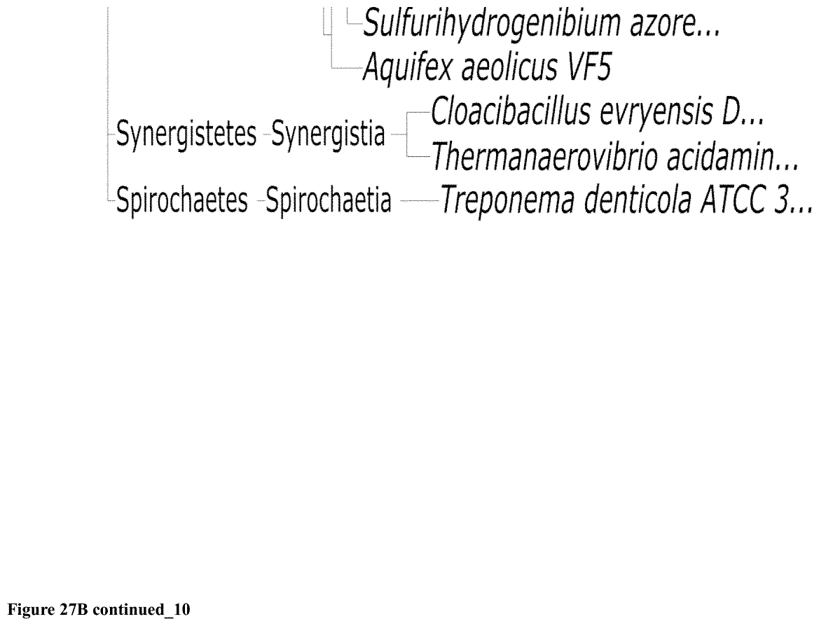

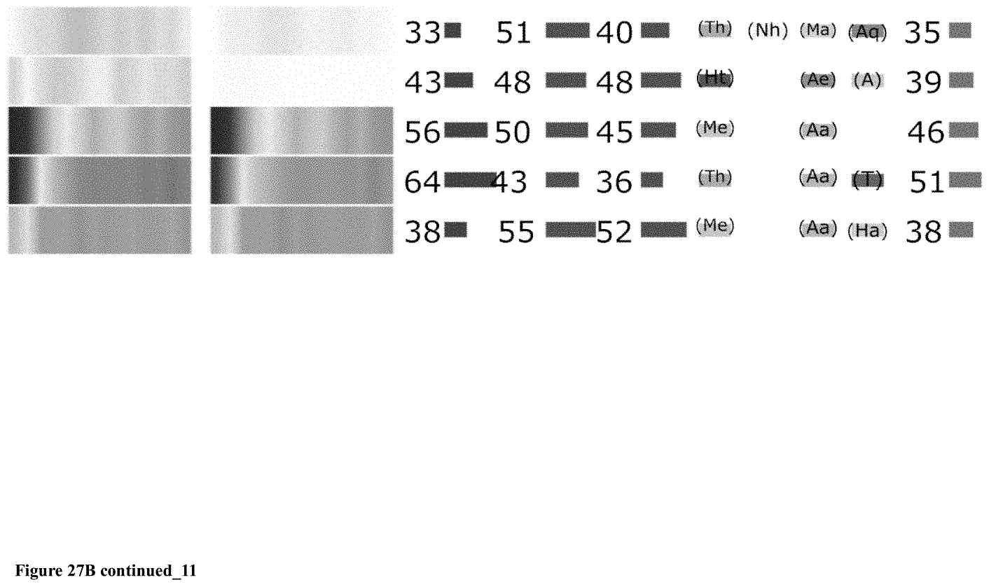

Methodology for FIGS. 15 and 26: Determination of each symbol (+/−) was based on results of a Mann-Whitney U test between the two groups of genes across the appropriate region, once for each direction (with the null hypothesis being that a value sampled from one group is not likely to be greater than an item from the other group). Fraction of positive species and total number of species are shown below for each evidence type.

Methodology for FIG. 15: On the right side, the table shows a summary of relevant characteristics for each species. From right to left—the average ΔLFE “heat-map” for this species, for the 300 nt region at the beginning (left) and end (right) of the CDS, the average GC % for the genome, and the average ENc′ (CUB) for the genome.

RNA sequencing data was obtained through ENA from the experiments detailed in the table below. Species were chosen based on availability of data using for the same strain or a closely related strain and using short-read sequencing technology compatible with the pipeline described here. Experiments are transcriptomic in their design and the control sample from each experiment was used (from the logarithmic growth phase if possible).

Normalized read counts were calculated as follows. Trimmomatic version 0.38, using the single-end or paired-end mode and the Illumina adapters, sliding window with window size 4 nt and quality threshold 15, leading and trailing below 3 and minimum length of 36 nt. Reads were mapped to reference genomes obtained from Ensemble genomes, except for E. coli that was obtained from NCBI. Reads were mapped to genomic positions with Bowtie2 version 2.3.4.3 using local alignment with the default settings. Read were then assigned to coding sequences using htseq-count version 0.11.2 in union mode with non-unique matches included and ignoring expected strand. Normalized counts for each CDS were finally obtained by dividing by the CDS length. Genes were divided to the “low” and “high” groups based on the median normalized read count for each species, with genes having no reads counted as 0.

PA results were obtained from PaxDB using the “Integrated” dataset. Genes were divided to the “low” and “high” groups based on the median count for each species, with genes having no reads counted as 0. I_TE, a CUB measure designed to measure codon optimization for translation elongation, was computed using DAMBE7 based on the included codon frequency tables for each species.

### Example 1

To test different hypotheses related to direct selection acting on the local folding-energy (LFE) in different regions of the coding sequence, the mean deviation in LFE between the native and randomized sequences was measured (maintaining the amino-acid sequence of all CDSs as well as codon and nucleotide composition including the GC-content, see Materials and Methods for more details). The resulting deviation values, denoted ΔLFE, measure the increase or decrease in local mRNA folding-energy relative to what would be expected based on the encoded protein and codon frequencies. Any significant deviation from random can be attributed to a specific arrangement of codons that supports increased or decreased base-pairing and folding strength along the mRNA strand (FIG. 2A).

Specifically, if the null hypothesis used to generate the randomized sequences holds for the native sequences at some position, the expected ΔLFE is 0. Otherwise, a significant deviation from ΔLFE=0 indicates that the local folding-energy values cannot be explained by selection on amino-acid content, codon bias or GC-content alone and serves as evidence for direct selection on local folding-energy (FIG. 2A). Positive ΔLFE indicates putative selection for weaker secondary-structure, while negative ΔLFE corresponds with selection for stronger secondary-structure. A specific aim was to find nearly universal patterns in ΔLFE, as well as groups of organisms and specific organisms with profiles deviating from such patterns. The resulting ΔLFE profiles were subsequently used with the evolutionary tree of the analyzed organisms to detect association between ΔLFE and genomic and environmental traits that cannot be explained by taxonomic relatedness alone and therefore may hint at underlying causal relations. The influence of genomic features such as codon usage bias (CUB, Example 4), GC-content (Example 5) and genome size (Example 7), and of environmental features like intracellular life (Example 6) and growth temperature (Example 7) was investigated.

### Example 2: Conserved Regions of Folding Bias (ΔLFE)

It was observed that significant ΔLFE is present in most species and in most regions of the CDS (FIG. 3A-B, FIG. 1A, 1C). The mean ΔLFE profiles of most species share the same structure (FIG. 3A, FIG. 1B-C), as follows. The region immediately following the CDS start (typically extending through the windows starting at positions 0-20 nt (FIG. 1A, region A), with a median of 20 nt/10 nt/20 nt in bacteria/archaea/eukaryotes respectively) has positive mean ΔLFE (evidence of selection for weak folding), usually followed by a transition to negative mean ΔLFE (indicating selection for strong folding) within the first 50 nt and maintained throughout most of the CDS (FIG. 1A region C, FIG. 1C-D). The negative ΔLFE tends to weaken in the area immediately preceding the last codon (typically nucleotides 50-0 nt before the stop codon with median of 50/90/40 nt in bacteria/archaea/eukaryotes respectively, FIG. 1D) in 83% of the species, and ΔLFE becomes positive there (indicating weaker-than-expected folding) in 37% of the species (including 68% of eukaryotes). This evidence of selection for weak mRNA folding near the stop codon in many organisms across the tree of life is reported here for the first time; two previous studies reported that the local folding-energy (LFE) is weak near the start codon in three organisms and without showing that it cannot be explained by direct selection on the amino-acid sequence (e.g., using computation of ΔLFE as was done here).

To measure how frequently these elements appear together within the same species, they were tested against a model, based on two variants. The stricter variant, Model 1, counts species in which the regions of weak folding at the beginning and end of the CDS have, on average, weaker than expected folding, i.e., significantly positive ΔLFE. The less restrictive Model 2 requires folding in these regions to be significantly weaker than in the middle of the CDS, but not necessarily significantly weaker than random (see Materials and Methods for details). Since the models are applied to the mean ΔLFE of a population of genes which may vary greatly in their individual values, both estimates of the adherence to the model are informative. The combined models (composed of the three regions described) are found in 23% (Model 1) and 69% (Model 2) of the species analyzed (FIG. 1A), appearing very frequently in bacteria but also commonly in archaea and eukaryotes. The conservation of the ΔLFE profile structure in species across the tree of life is evidence of its biological significance.

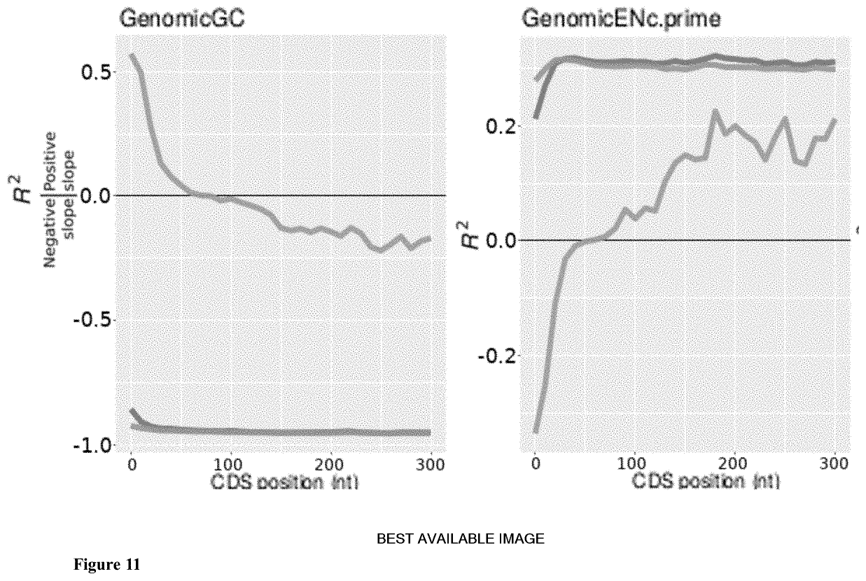

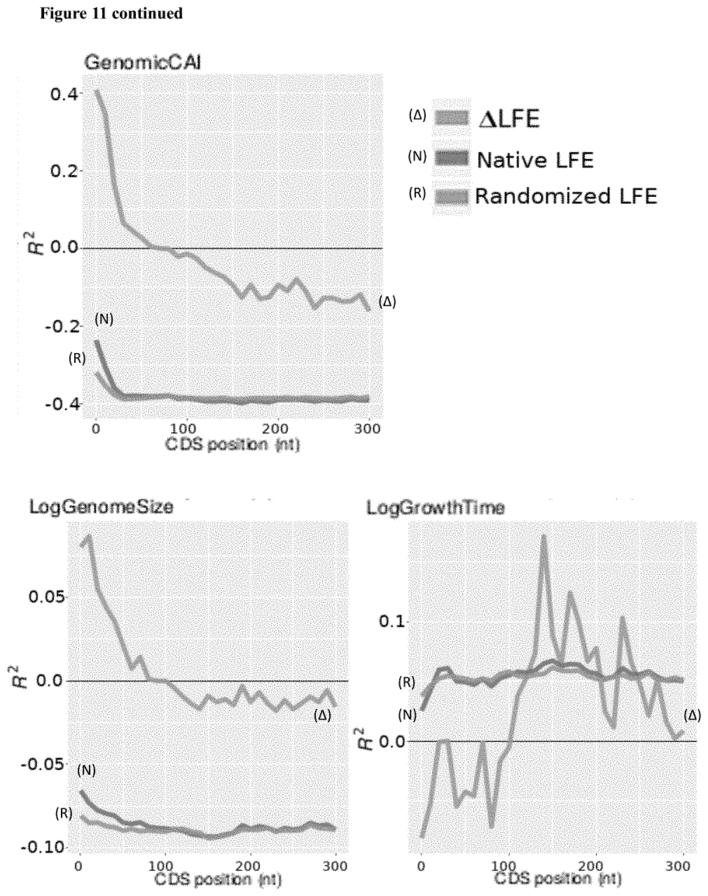

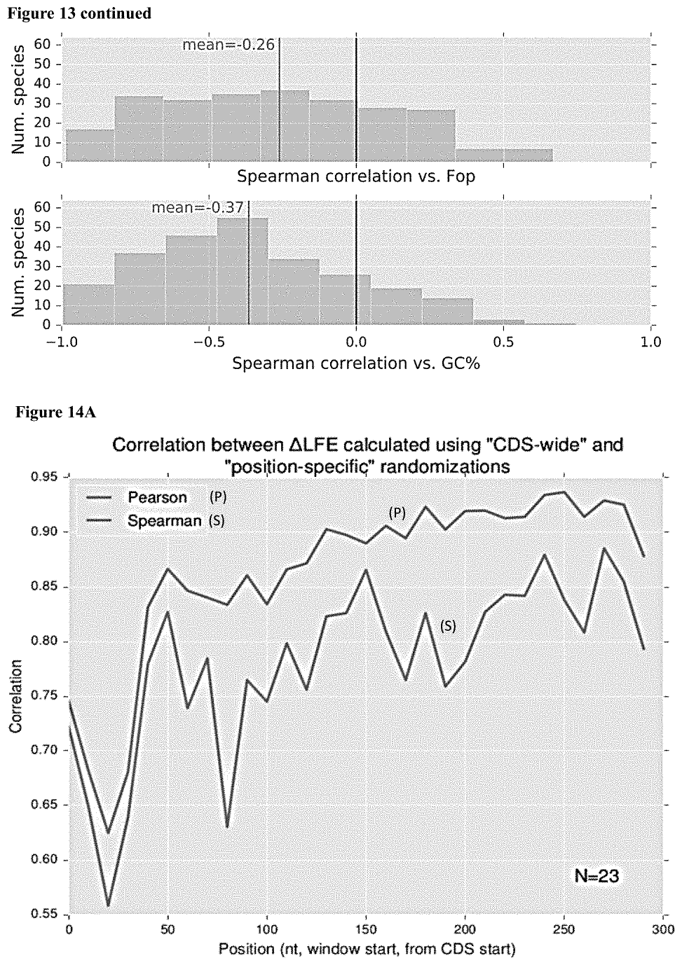

GC-content and LFE both change during evolution, and it is worthwhile to compare their level of conservation in related species. LFE is to a large degree determined by GC-content (as evident by the almost perfect correlations found between GC-content and native or randomized LFE, FIG. 11), so one might argue the observed ΔLFE is a side-effect of selection acting on GC-content. However, it was found that the ΔLFE profile is more conserved than genomic GC-content at any phylogenetic distance within the same domain (FIG. 12). It was also found that the profile does not consistently correlate with local variation in CUB (FIG. 13), demonstrating that the results reported here are not side effects of selection on codon bias (e.g., due to adaptation to the tRNA pool).

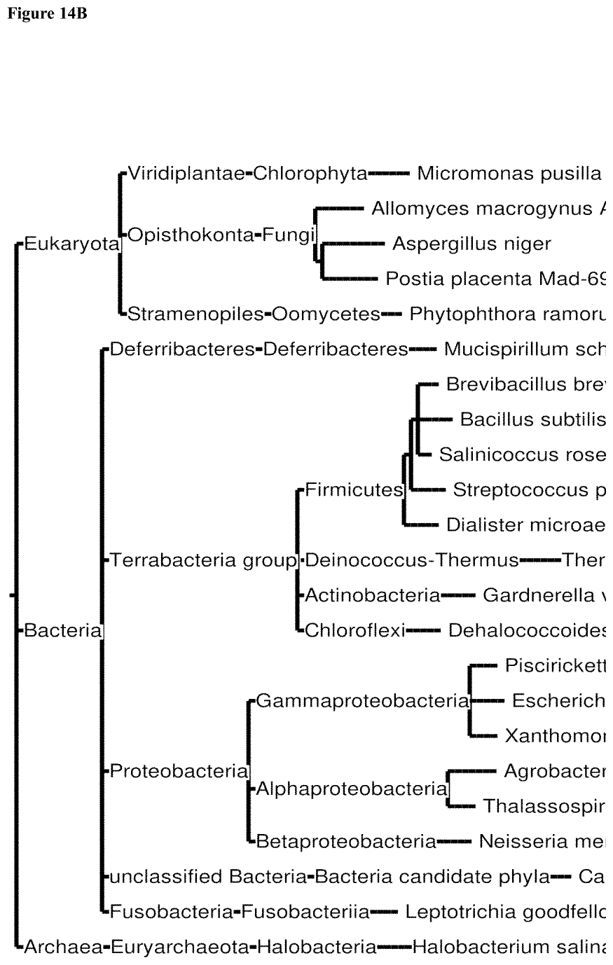

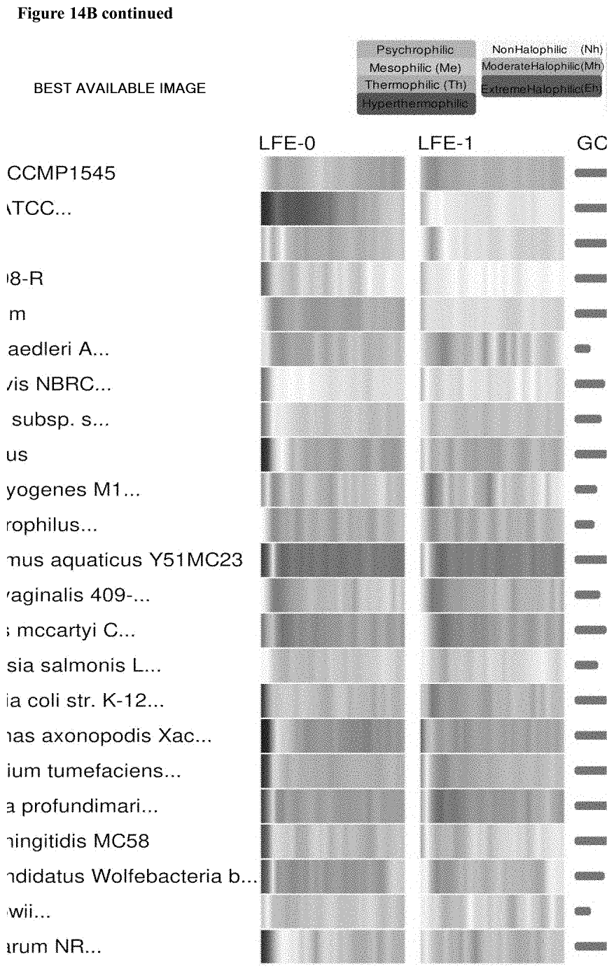

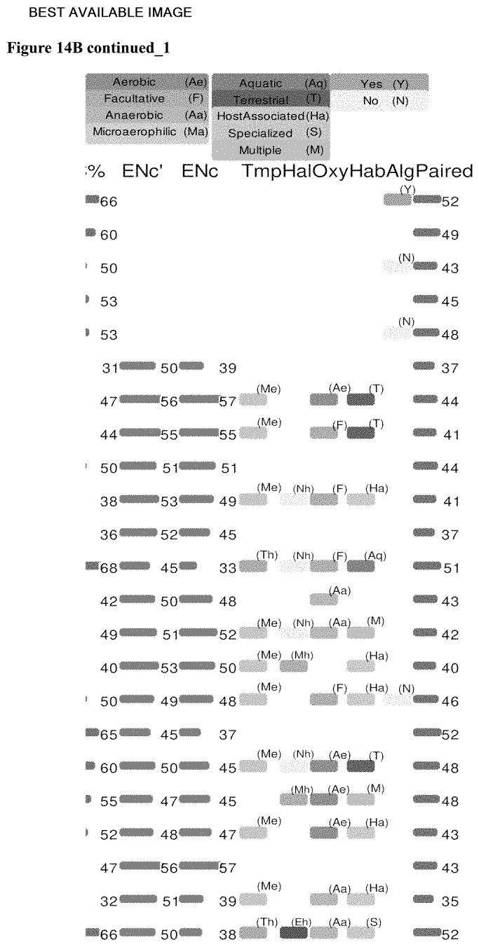

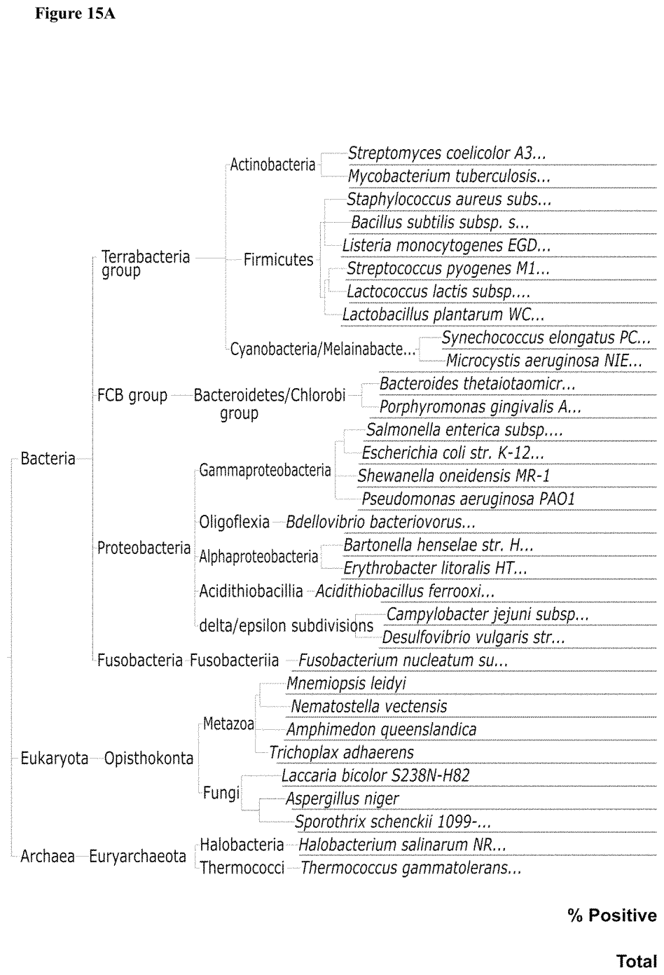

Additional tests also support direct selection acting to maintain folding strength. ΔLFE profile features are also preserved when calculated using a null distribution that maintains the codon distribution at any position in the CDS relative to the CDS start; thus, local (position-specific) genomic amino-acid or codon distributions are not enough to explain the ΔLFE profile (FIG. 14). These features appear in many cases to be stronger in highly expressed genes, genes coding for highly abundant proteins and genes with a strong codon adaptation to translation elongation, I_TE (see FIG. 15). Finally, these results remain after controlling for the strength of Shine-Dalgarno binding in the 5′-UTR and for genes with short or overlapping 5′-UTRs. Together, these results show that the ΔLFE profiles are unlikely to be explained as side-effects of selection for a genomic or CDS-position dependent compositional bias in nucleotide, codon or amino-acids acting alone, although many such biases have been reported and are believed to have important biological effects.

It should be noted, that the randomized LFE profiles also aren't always flat, revealing some residual influence on LFE, caused by the amino-acid frequencies at different regions, remains even after randomization. ΔLFE controls for this by separately measuring the folding-energy biases found in each position.

The different elements making up the model profile structure have functions associated with them. The weak folding region at the beginning of the coding region may improve access to the regulatory signals in this region (e.g., the start codon). The region of positive ΔLFE preceding the CDS end may help recognition of the stop codon and ribosomal dissociation from the mRNA and prevent ribosomal read-through. Strong folding in the middle of the coding sequence may assist co-translational folding by slowing down translation in specific positions to allow protein folding or other co-translational processes to take place, as well as regulate mRNA stability or prevent mRNA aggregation.

The division of the profile into the three regions described here is also apparent when the data is analyzed in an unsupervised manner via Principal Components Analysis (PCA) (FIG. 3B and FIG. 16). This arranges species on a 2-dimensional plane according to their ΔLFE profiles, so species with more similar ΔLFE profiles are placed closer together. The resulting plots (for the beginning and end of the coding sequence) show the majority of species have similar ΔLFE profiles (located very close to each other near the center of the plot), with positive ΔLFE near the ends of the coding sequence and negative ΔLFE in the middle of the coding sequence. Groups of species containing other types of profiles are arranged around them on the plots. At either end of the coding sequence, 2 variables (principal components) are sufficient to describe at least 85% of the variability between all ΔLFE profiles, supporting the division of the ΔLFE into three regions (since the mid-CDS region appears in both analyses, see FIG. 1E).

In 45% of the organisms there was found an additional feature: a peak of selection for strong mRNA folding around 30-70 nt downstream of the start codon (FIG. 1A region B). It has been suggested, based solely on evidence in Escherichia coli and Saccharomyces cerevisiae, that this peak is responsible for increasing translation throughput, by minimizing ribosomal traffic jams occurring because of uneven translation elongation rates throughout the CDS. There is also some evidence that strong secondary structure downstream of the start codon can enhance translation. Whatever the mechanism responsible for it, the results here show that this feature is common across the tree of life. This feature was also shown previously to be stronger in highly expressed genes in 3 species, and our results extend this claim (see FIG. 15).

The ΔLFE profiles of eukaryotes are much more diverse than those found in prokaryotes. One striking observation is that significant positive ΔLFE throughout the mid-CDS region, present in 13% of the eukaryotes tested, is not observed in any of the 371 bacterial species tested except in Deinococcus puniceus (FIG. 18, see also FIG. 1A). This seemingly universal rule hints at a constraint on bacterial CDSs not obeyed in eukaryotes and is one of two major differences observed between the domains (along with the correlation with genomic-GC, discussed in Example 4).

Despite these general trends, there is also significant variation in the ΔLFE profiles across and within taxonomic groups. Examples 4-7 discuss genomic and environmental factors that explain some of the variation between mean ΔLFE profiles in different species.

### Example 3: Correlations Between ΔLFE Regions

The strengths of the three major regions of the ΔLFE profile described above are strongly correlated (FIG. 1E): organisms with relatively stronger ΔLFE (in absolute value) in one model region appear to also have stronger ΔLFE in other regions. For example, the 0-20 nt region has strong negative correlation with the 150-300 nt region (Spearman's ρ=−0.46; p-value<1e-8). This correlation remains highly significant for different ranges and when testing using GLS, FIG. 19). The two mid-CDS regions (relative to CDS start and end) are positively correlated (ρ=0.84, p-value<1e-8), as are the CDS-start and end regions (ρ=0.52, p-value<1e-8). These correlations indicate ΔLFE profiles of different species can generally be ordered by magnitude from species having strong (positive or negative) ΔLFE features throughout the CDS to those showing weak or no ΔLFE. In Eukaryotes, the negative correlation between the CDS start and mid-CDS regions is not present (results not shown), but in this case neither do the ΔLFE profiles generally follow the structure of positive start ΔLFE and negative mid-CDS ΔLFE and the profile values may continue to change farther away from the CDS edges.

Together these results suggest that the different elements making up the typical profile structure are influenced at the genome level by a factor or combination of factors acting jointly on all regions and strengthening or weakening |ΔLFE|, as well distinct factors acting on each region differently. Some factors contributing to this scaling effect are discussed in Examples 4-7.

### Example 4: Correlation Between Codon Usage Bias (CUB) and ΔLFE

Codon usage bias is generally correlated with adaptation to translation efficiency. If ΔLFE is also related to selection for translation efficiency, it is reasonable to expect it would correlate with CUB. To test this hypothesis. ENc′ (ENc prime), a measure of codon usage bias (CUB) that compensates for the influence of extreme GC-content values that skews standard ENc (Effective Number of Codons) scores was used. Indeed, such a correlation is found (FIG. 4, FIG. 20B)—ΔLFE tends to be stronger (in absolute value) in species having strong CUB (low ENc′), and this holds both near the CDS edges and in the mid-CDS regions. Similar results were obtained when using other measures of CUB, (CAI and DCBS, FIG. 21), and these correlations persist within many individual taxa (FIG. 9, FIG. 20B). In addition, species with strong CUB tend to have ΔLFE profiles that closely match the model elements (FIG. 4B-C), and further analysis shows the correlation of CUB with the ΔLFE profiles is due to correlation with the magnitude of the profiles and not due to specific profile regions (FIG. 22). Since ΔLFE is computed while controlling for the CUB of each sequence, the reported results suggest that organisms with higher selection on CUB also have, “independently” from a statistical point of view, higher selection on ΔLFE.

Using genomic CUB as a measure of optimization for efficient translation elongation, it was found that it is also a good predictor of the strength of ΔLFE. One interpretation of this is that the genomic variation in ΔLFE can largely be explained not by different species having distinct ‘target’ ΔLFE levels, but by different species having varying ‘abilities’ to maintain ΔLFE in the presence of mutations and drift because the selection pressure is insufficient under their effective population size (either because the selection pressure is low or because the effective population size is low).

### Example 5: Correlation Between GC-Content and ΔLFE

GC-content is a fundamental genomic feature and is correlated with many other genomic traits and environmental aspects. It might be a trait maintained under direct selection, or merely a statistical measure of the genome that other traits evolve in response to because of its biological and thermodynamic consequences. GC-content is also the strongest factor determining the native LFE (FIG. 11A), since G-C base-pairs are more stable than A-T pairs (due to the increase in the number of hydrogen bonds and more stable base stacking). Selection on folding strength (measured by ΔLFE), also influences folding strength, and it is helpful to measure the correlation between these two factors that influence the folding strength (namely, GC-content and ΔLFE). This is made possible since ΔLFE is calculated relative to the baseline maintaining the GC-content of the original coding regions in the randomized ones (see Example 2 under “Randomization procedures” for a description of the null models). This controls for the direct effect of GC-content, allowing us to directly study the interaction between ΔLFE and GC-content (see also FIG. 11A).

The correlations (expressed as R²) between genomic GC-content and ΔLFE at different points near the CDS start and end are shown in FIG. 5A. This dependence shows a similar pattern to that seen in the ΔLFE profiles themselves (FIG. 1C, 5A) and for the correlation with CUB (see Example 4), with significant correlations appearing in roughly the same CDS regions described for the ΔLFE profiles. The correlation takes the opposite directions in the CDS edges than that maintained throughout the inner CDS region, which means GC-content is positively correlated with the strength of ΔLFE (in absolute value) throughout the CDS (like CUB is).

Near the CDS start, positive correlation (indicating a moderating effect) exists in the windows starting at 0-60 nt (FIG. 5A, 20A). This effect appears in almost all taxa analyzed, with R² values between 0.2-0.9 and significant p-values in most taxa and may be explained as counteracting the strengthening influence of GC-content on secondary structures to prevent them from hindering the translation initiation process.

The opposite effect exists in the mid-CDS: negative (reinforcing) dependence on genomic GC-content appears in the region at 70-300 nt after CDS start in most bacterial and archaeal taxa (FIGS. 5A-C, 9, and 20A) and is generally maintained throughout the length of the CDS (excluding the edge regions). As mentioned above, selection for strong mRNA folding and mRNA structures inside the coding may be related to transcription elongation, co-translational folding and mRNA stability. The observed ΔLFE in this region is indeed negative in nearly all bacterial and archaeal species; it is possible that the folding is further reinforced in species higher GC-content since they are under stronger selection for these processes. Note that the effects of genomic GC-content and CUB see Example 4) are somewhat overlapping, but each factor significantly contributes to the total observed effect (FIG. 23).

In eukaryotes, there was observed a wider variation in mid-CDS ΔLFEs (which is not found in other groups), from strongly positive to strongly negative, with a non-linear dependence on genomic-GC (FIGS. 6A-B, and 9). Low-GC eukaryotes tend to have weak ΔLFE in the mid-CDS region, while high-GC eukaryotes tend to have strong positive or negative ΔLFE in the same region. To evaluate this relation, which is not linear, Maximal Information Coefficient (MIC) was used as a measure that can capture any statistical dependence including non-linear dependencies. This relation was found to be quite significant (MIC=0.54, p-value ≤2e-5; see Example 2 and Materials and Methods). Fungi, however, show a strong positive (moderating) correlation between genomic-GC and ΔLFE (FIG. 5A, 6A; Eremothecium gossyppi, GC %=51.7, is the only observed fungus with GC %>45 and negative ΔLFE in the mid-CDS region). There are also clear internal disparities in ΔLFE among fungi families (FIG. 17). It should be noted, that in some species (e.g., Zymoseptoria tritici) the positive ΔLFE seems to extend throughout the CDS. In other species, there is a transition to negative ΔLFE further downstream (as much as 500 nt from CDS start, results not shown).

The group of fungi and other eukaryotes having strong selection for weak local mRNA folding in the mid-CDS region (all of which have high genomic GC-content) runs counter to the general trend in prokaryotes. It is possible that these species are under selection for higher translation elongation speeds, which tend to be hindered by stronger mRNA folding; however, it is not clear why such cases are not observed in other groups like bacteria. The correlation with GC-content reported here may also be partially explained by the fact that both GC-content and ΔLFE are affected by common factors such as the ability to maintain the selected sequences under the effective population size. The wide range of ΔLFE values for eukaryotic species and the absence of linear correlation with GC-content (in general) reveals additional factors are involved in this aspect of gene expression.

### Example 6: Weak ΔLFE in Endosymbionts and Intracellular Organisms

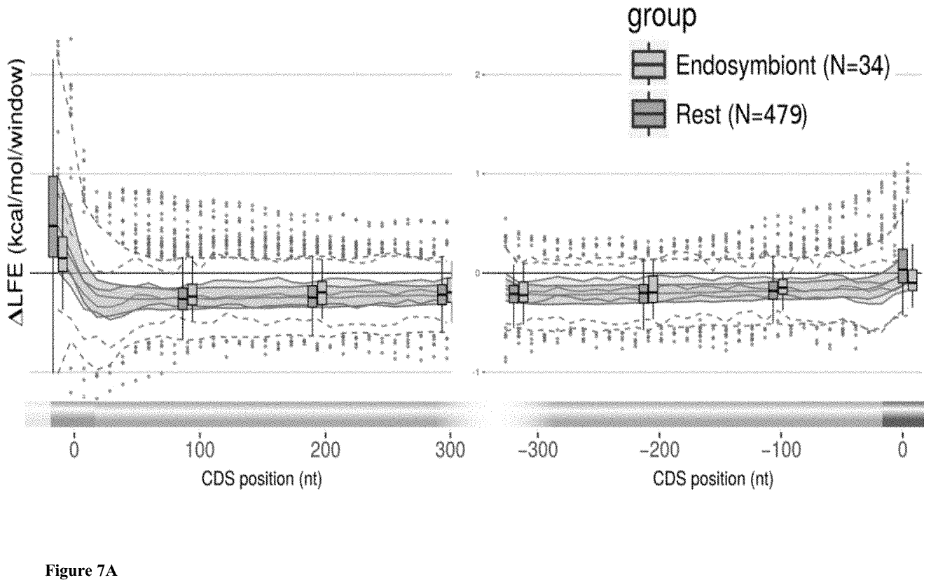

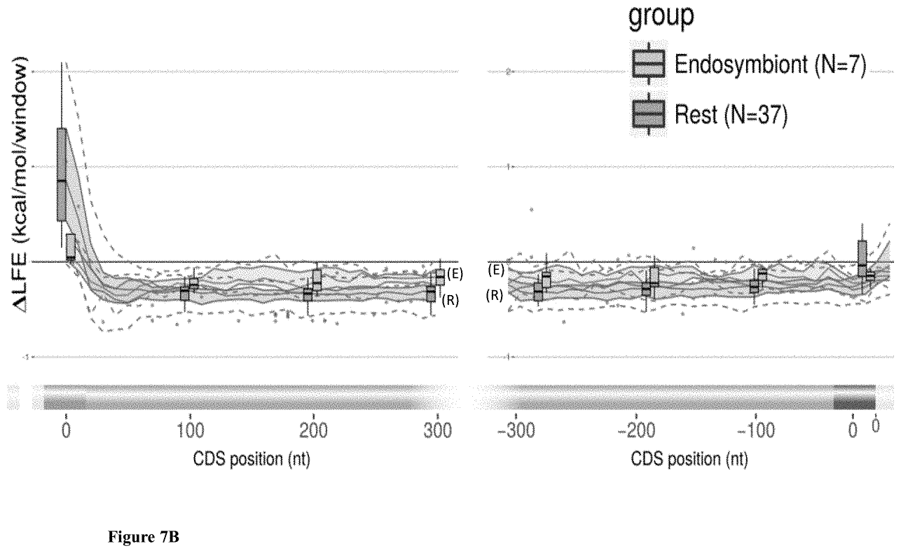

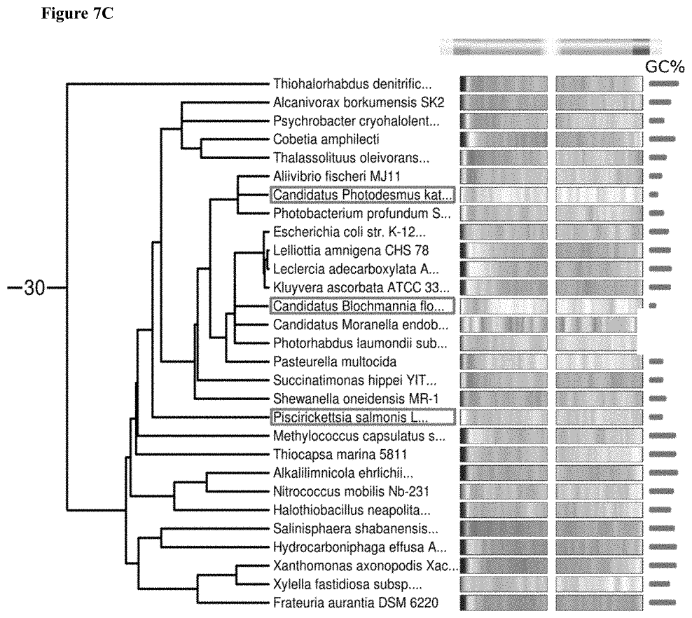

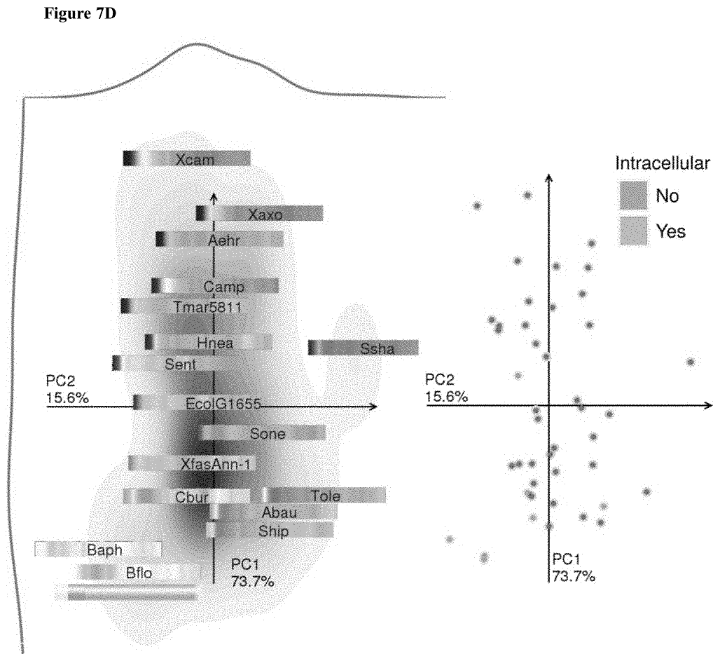

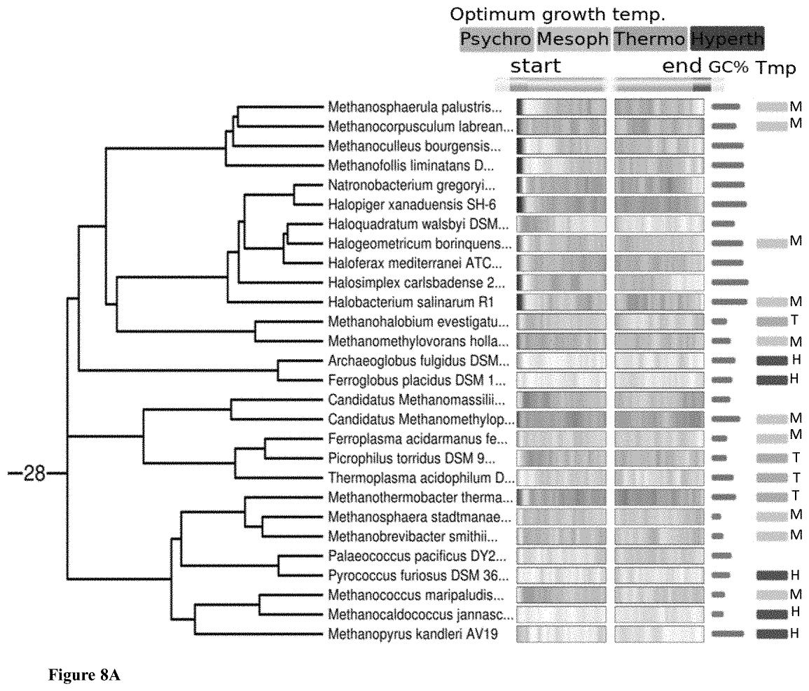

Many endosymbionts and other species with intracellular life stages have low effective population sizes, because their lifecycle includes recurring population bottlenecks or have lower selective pressure due to reliance on the host. These species generally have weaker ΔLFE compared to their relatives, as can be clearly seen from their ΔLFE profiles (FIG. 7A-D, also see FIG. 17, e.g., Richelia intracellularis, Blattabacterium sp.). The apparent disparity between endosymbionts and their relatives is strongest near the CDS start. Taken as a whole the difference in ΔLFE is small (FIG. 7A), but when comparing within smaller taxa the difference is much more noticeable (e.g., gammaproteobacteria in FIG. 7B-D). Endosymbionts also tend to have lower GC-content and CUB, but the results are still generally significant after considering this at least in proteobacteria, where we have a sufficient sample size (FIG. 24). The dichotomic grouping of species as endosymbionts is an oversimplification and ignores the variety of species with intracellular stages, including obligate and facultative intracellular parasites (and our annotation of species as endosymbionts, based on the literature, may not be complete). Indeed, some species we classify as endosymbionts (e.g., Halobacteriovorax marinus SJ) nevertheless have low genomic ENc′ and strong ΔLFE.

### Example 7: Weak ΔLFE in Hyperthermophiles

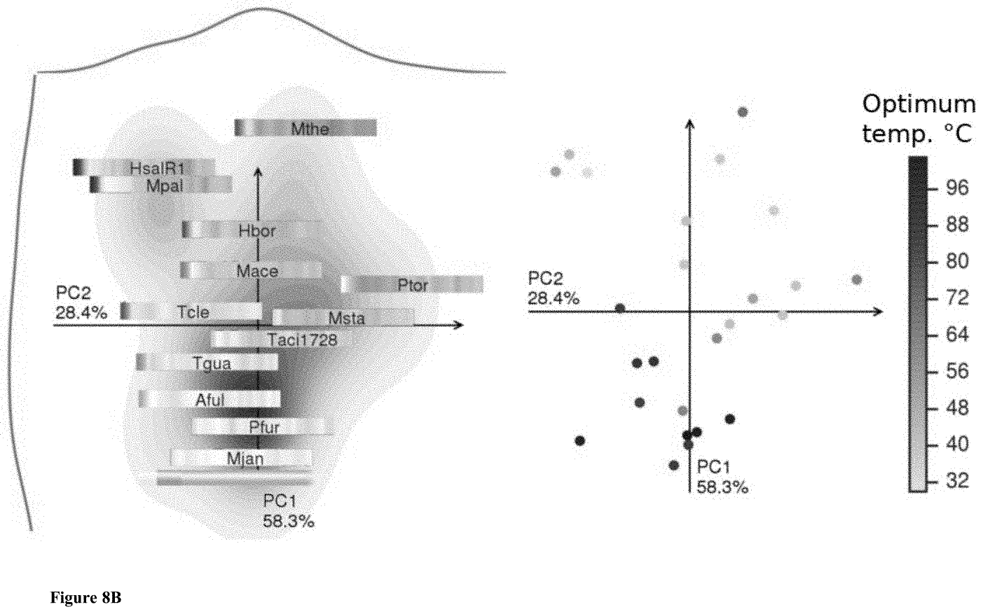

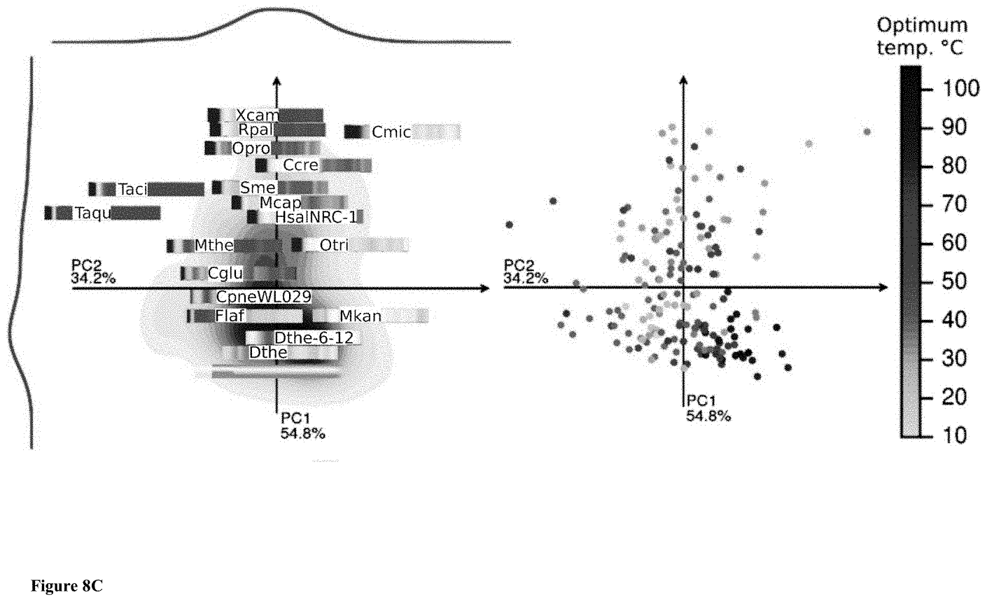

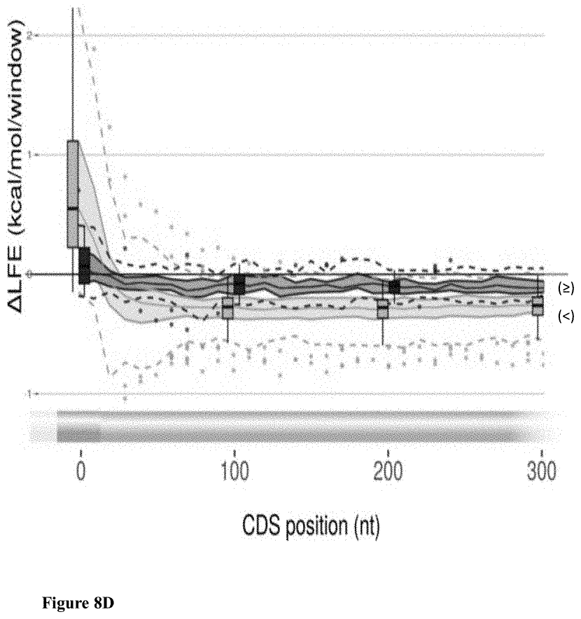

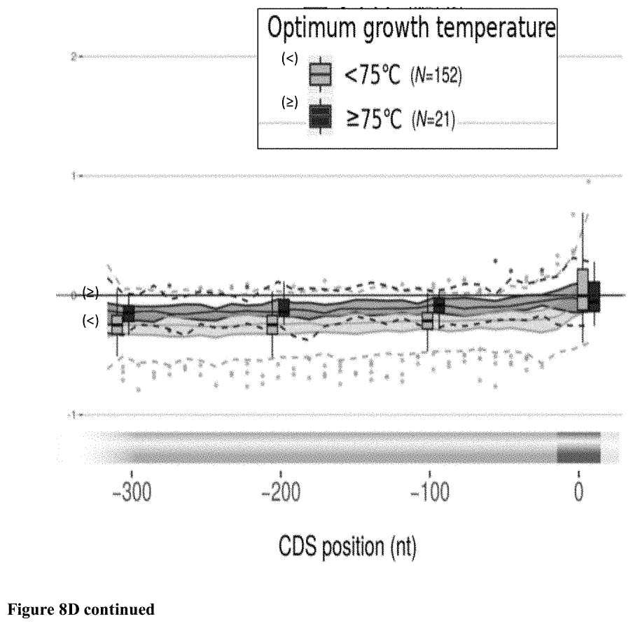

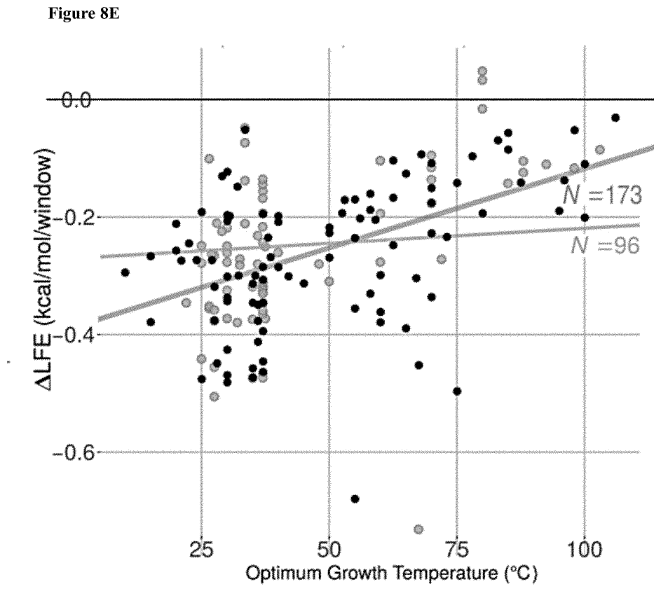

In temperatures approaching the RNA melting temperature base-pairing is destabilized and it is likely that codon arrangement and ΔLFE can no longer significantly affect the secondary-structure. It was found that hyperthermophilic archaea and bacteria have weaker (closer to 0) ΔLFE in the mid-CDS region (FIG. 8A-E). This effect is not apparent at lower temperatures (below 65° C.) or across all temperatures, with temperature having no significant correlation with ΔLFE (FIG. 8E, 9) when controlling for species relatedness. These results are consistent with what is known in that art and argue for negative correlation with growth temperature. However, previous work only analyzed the beginning of the coding region and did not control for the evolutionary relations among organisms. Based on this analysis the linear relation between temperature and ΔLFE is not generally supported by GLS (FIGS. 8E, 9, and 20C); however, since species tend to have similar temperature requirements as their close relatives, it is hard to conclusively decide if any similarity in ΔLFE is derived from association with temperature or the evolutionary relationship without having considerably more data. In hyperthermophiles (species with optimum growth temperature above 75° C.), however, there is a significant decrease in ΔLFE (even when the folding strengths are predicted at room temperature, FIG. 25). These results suggest LFE is not effective in higher temperatures and consequently ΔLFE is not preserved. In moderate thermophiles, ΔLFE may follow the precedence of genomic GC-content, which previous studied concluded is not an adaptation to high temperatures at the genomic level but may still be part of such an adaptation at specific rRNA and tRNA sites where secondary RNA structure is particularly important.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for optimizing a coding sequence, the method comprising introducing a mutation into a first region from 90 nucleotides upstream of a stop codon of said coding sequence to said stop codon; wherein said mutation increases folding energy of said first region or of RNA encoded by said first region, thereby optimizing a coding sequence.

2. The method of claim 1, wherein said optimizing comprises at least one of optimizing expression of protein encoded by said coding sequence and optimizing in a target cell.

3. (canceled)

4. The method of claim 2, wherein said optimizing is optimizing in a target cell and said target cells is selected from:
a. an archaea cell and said first region is from 90 nucleotides upstream of a stop codon of said coding sequence to said stop codon;
b. a bacteria cell and said first region is from 50 nucleotides upstream of a stop codon of said coding sequence to said stop codon; and
c. a eukaryote cell and said first region is from 40 nucleotides upstream of a stop codon of said coding sequence to said stop codon.

5. (canceled)

6. (canceled)

7. The method of claim 1, wherein said mutation increases folding energy of said first region to above a predetermined threshold, optionally wherein said predetermined threshold is a value above which the difference as compared to folding energy of said region without said substitution would be significant.

8. (canceled)

9. The method of claim 7, wherein said threshold is species-specific and is selected from a threshold provided in Tables 5 or said threshold is domain-specific and is selected from a threshold provided in Table 1.

10. The method of claim 1, comprising introducing a plurality of mutations wherein each mutation increases folding energy of said first region or of RNA encoded by said first region or wherein said plurality of mutations in combination increases folding energy of said first region or of RNA encoded by said first region.

11. The method of claim 1, wherein said mutation is a synonymous mutation and comprising at least one of:
a. mutating all possible codons within said region to a synonymous codon that increases folding energy of said first region or of RNA encoded by said first region; and
b. introducing synonymous mutations to produce a first region or RNA encoded by said first region with the maximum possible folding energy.

12. (canceled)

13. The method of claim 1, further comprising introducing a mutation into a second region from a translational start site (TSS) to 20 nucleotides downstream of said TSS, wherein said mutation increases folding energy of said second region or of RNA encoded by said second region.

14. The method of claim 13, wherein said method is a method for optimizing expression in a target cell, and wherein said target cells is selected from:
a. an archaea cell and said second region is from said TSS to 10 nucleotides downstream of said TSS; and
b. a bacteria cell or a eukaryote cell and said second region is from said TSS to 20 nucleotides downstream of said TSS.

15. The method of claim 13, wherein said method is a method for optimizing expression in a target cell, and wherein said target cell is:
a. a bacterial or archaeal cell and the method further comprises introducing a mutation into a third region between said first and said second regions, wherein said mutation decreases folding energy of said third region or of RNA encoded by said third region; or
b. a eukaryotic cell and the method further comprises introducing a mutation into a third region between said first and said second regions, wherein said mutation increases folding energy of said third region or of RNA encoded by said third region.

16. (canceled)

17. The method of claim 15, wherein said third region is selected from: from 20 to 50 nucleotides downstream of said TSS; from 20 to 300 nucleotides downstream of said TSS; and from 300 to 90 upstream of said stop codon.

18. (canceled)

19. A nucleic acid molecule comprising a coding sequence, said coding sequence comprises at least one codon substituted to a synonymous codon within a first region from 90 nucleotides upstream of a stop codon of said coding sequence to said stop codon, wherein said substitution increases folding energy of said first region or of RNA encoded by said first region.

20. (canceled)

21. (canceled)

22. (canceled)

23. The nucleic acid molecule of claim 19, wherein said substitution increases folding energy of said first region to above a predetermined threshold, optionally wherein said predetermined threshold is a value above which the difference as compared to folding energy of said region without said substitution would be significant.

24. (canceled)

25. The nucleic acid molecule of claim 23 or 211, wherein said threshold is species-specific and is selected from a threshold provided in Tables 5 or said threshold is domain-specific and is selected from a threshold provided in Table 1.

26. The nucleic acid molecule of claim 19, wherein at least one of:
a. said nucleic acid molecule comprises a plurality of synonymous substitutions, wherein each substitution increases folding energy of said first region or of RNA encoded by said first region or wherein said plurality of synonymous substitutions in combination increases folding energy of said first region or of RNA encoded by said first region;
b. all possible codons within said first region are substituted to a synonymous codon that increases folding energy of said first region or of RNA encoded by said first region; and
c. said region comprises synonymous codons substituted to increase folding energy to a maximum possible.

27. (canceled)

28. (canceled)

29. (canceled)

30. The nucleic acid molecule of claim 19, wherein said coding sequence
a. comprises a second region of said coding sequence from a translational start site (TSS) to 20 nucleotides downstream of said TSS comprises at least one codon substituted to a synonymous codon, and wherein said substitution increases folding energy of said second region or of RNA encoded by said second region;
b. encodes a bacterial or archaeal gene, comprises a second region of said coding sequence from a translational start site (TSS) to 20 nucleotides downstream of said TSS comprises at least one codon substituted to a synonymous codon, and wherein said substitution increases folding energy of said second region or of RNA encoded by said second region and further comprises a third region of said coding sequence between said first region and said second region comprises at least one codon substituted to a synonymous codon, and wherein said substitution decreases folding energy of said third region or of RNA encoded by said third region; or
c. encodes a eukaryotic gene, comprises a second region of said coding sequence from a translational start site (TSS) to 20 nucleotides downstream of said TSS comprises at least one codon substituted to a synonymous codon, and wherein said substitution increases folding energy of said second region or of RNA encoded by said second region and further comprises a third region of said coding sequence between said first region and said second region comprises at least one codon substituted to a synonymous codon, and wherein said substitution increases folding energy of said third region or of RNA encoded by said third region.

31. (canceled)

32. The nucleic acid molecule of claim 30, wherein said third region is selected from: from 20 to 50 nucleotides downstream of said TSS; from 20 to 300 nucleotides downstream said TSS; and from 300 to 90 upstream of said stop codon.

33. (canceled)

34. (canceled)

35. An expression vector comprising the nucleic acid molecule of claim 19.

36. A cell comprising the expression vector of claim 35, optionally wherein said expression vector is optimized for expression in said cell.

37. (canceled)

38. A computer program product comprising a non-transitory computer-readable storage medium having program instructions embodied therewith, the program instructions executable by at least one hardware processor to execute a genetic-type machine learning algorithm configured to:
a. receive a coding sequence;
b. determine within a first region from 90 nucleotides upstream of a stop codon of said coding sequence to said stop codon at least one mutation that increases folding energy of said first region or RNA encoded by said first region; and
c. output
i. a mutated coding sequence comprising said at least one mutation; or
ii. a list of possible mutations comprising said at least one mutation.
